(19) United States
(12) Patent Application Publication
(10) Pub. No.: US 2006/0275747 A1
(43) Pub. Date: Dec. 7, 2006

(54) Endogenous retrovirus up-regulated in prostate cancer

(76) Inventors: Stephen F. Hardy, Oakland, CA (US); Pablo Garcia, Oakland, CA (US); Lewis T. Williams, Mill Valley, CA (US); Jaime Escobedo, Alamo, CA (US)

Correspondence Address: NOVARTIS VACCINES AND DIAGNOSTICS INC., CORPORATE INTELLECTUAL PROPERTY R338, P.O. BOX 8097, Emeryville, CA, 94662-8097, US

(21) Appl. No.: 10/498,033

(22) PCT Filed: Dec. 9, 2002
(86) PCT No.: PCT/US02/39136

§ 371 Date: Dec. 22, 2005

Related U.S. Application Data
(63) Continuation-in-part of application No. 10/061,604, filed on Feb. 1, 2002, now Pat. No. 6,713,919.
(60) Provisional application No. 60/340,640, filed on Dec. 7, 2001.
(60) Provisional application No. 60/388,046, filed on Jun. 12, 2002.

(30) Foreign Application Priority Data
Dec. 7, 2001 (WO) PCT/US01/47824

(51) Int. Cl. C12Q 1/70

(52) U.S. Cl. 435/5

(57) ABSTRACT

A specific member of the HERV-K family located in chromosome 22 at 20.428 megabases (22q11.2) has been found to be preferentially and significantly up-regulated in prostate tumors. The invention provides methods for diagnosing prostate cancer, comprising the step of detecting in a patient sample the presence or absence of an expression product of the virus. The virus has five features not seen in other HERV-K members: (1) its own specific nucleotide sequence, and consequently amino acid sequences; (2) tandem 5′ LTRs; (3) a fragmented 3′ LTR; (4) an env gene interrupted by an alu insertion; and (5) unique gag sequences.

This application claims the benefit of: international patent application PCT/US01/47824 (published in English on Jun. 13, 2002, as WO02/46477), filed Dec. 7th 2001; U.S. patent application Ser. No. 10/016,604, filed Dec. 7th 2001; U.S. provisional patent application 60/340,064, filed Dec. 7, 2001; and U.S. provisional patent application 60/388,046, filed Jun. 12th 2002.

All publications and patent applications mentioned in this specification are incorporated herein by reference to the same extent as if each individual document were specifically and individually indicated to be incorporated by reference.

### TECHNICAL FIELD

The present invention relates to the diagnosis of cancer, particularly prostate cancer. In particular, it relates to a human endogenous retrovirus (HERV) located on chromosome 22 which shows up-regulated expression in tumors, particularly prostate tumors.

### BACKGROUND ART

Prostate cancer is the most common type of cancer in men in the USA. Benign prostatic hyperplasia (BPH) is the abnormal growth of benign prostate cells in which the prostate grows and pushes against the urethra and bladder, blocking the normal flow of urine. More than half of the men in the USA aged 60-70 and as many as 90% percent aged 70-90 have symptoms of BPH. Although BPH is seldom a threat to life, it may require treatment to relieve symptoms.

Cancer that begins in the prostate is called primary prostate cancer (or prostatic cancer). Prostate cancer may remain in the prostate gland, or it may spread to nearby lymph nodes and may also spread to the bones, bladder, rectum, and other organs. Prostate cancer is currently diagnosed by measuring levels of prostate-specific antigen (PSA) and prostatic acid phosphatase (PAP) in the blood. The level of PSA in blood may rise in men who have prostate cancer, BPH, or an infection in the prostate. The level of PAP rises above normal in many prostate cancer patients, especially if the cancer has spread beyond the prostate. However, prostate cancer cannot be diagnosed using these tests alone because elevated PSA or PAP levels may also indicate other, non-cancerous problems.

In order to help determine whether conditions of the prostate are benign or malignant further tests such as transrectal ultrasonography, intravenous pyelogram, and cystoscopy are usually performed. If these test results suggest that cancer may be present, the patient must undergo a biopsy as the only sure way of diagnosis. Consequently, it is desirable to provide a simple and direct test for the early detection and diagnosis of prostate cancer without having to undergo multiple rounds of cumbersome testing procedures. It is also desirable and necessary to provide compositions and methods for the prevention and/or treatment of prostate cancer.

References 1 and 2 disclose that human endogenous retroviruses (HERVs) of the HML-2 subgroup of the HERV-K family show up-regulated expression in prostate tumors. This finding is disclosed as being useful in prostate cancer screening, diagnosis and therapy. In particular, higher levels of an HML-2 expression product relative to normal tissue are said to indicate that the patient from whom the sample was taken has cancer.

It is an object of the invention to provide additional and improved materials and methods that can be used in the diagnosis, prevention and treatment of prostate cancer.

### DISCLOSURE OF THE INVENTION

A specific member of the HERV-K family located in chromosome 22 at 20.428 megabases (22q11.2) has been found to be preferentially and significantly up-regulated in prostate tumors. This endogenous retrovirus (named ‘PCAV’ herein) has several features not found in other members of the HERV-K family and these features can be exploited in prostate cancer screening, diagnosis and therapy (e.g. adjuvant therapy).

The invention provides a method for diagnosing cancer, especially prostate cancer, the method comprising the step of detecting in a patient sample the presence or absence of an expression product of a human endogenous retrovirus located at megabase 20.428 on chromosome 22. Higher levels of expression product relative to normal tissue indicate that the patient from whom the sample was taken has cancer.

The expression product which is detected is preferably a mRNA transcript, but may alternatively be a polypeptide translated from such a transcript. These expression products may be detected directly or indirectly. A direct test uses an assay which detects PCAV RNA or polypeptide in a patient sample. An indirect test uses an assay which detects biomolecules which are not directly expressed in vivo from PCAV e.g. an assay to detect cDNA which has been reverse-transcribed from PCAV mRNA, or an assay to detect an antibody which has been raised in response to a PCAV polypeptide.

A—The Human Chromosome 22 Endogenous Retrovirus

Many regions within the published human genome sequence are annotated as endogenous retroviruses and, even before its sequence was determined, it was known that the human genome contained multiple HERVs. One of the many HERVs is a HERV-K located at megabase 20.428 of chromosome 22, referred to herein as ‘PCAV’. Expression of this HERV has been found to be up-regulated in cancer tissue. Furthermore, PCAV has five specific features not found in other HERVs. These five features are manifested in PCAV mRNA transcripts and can be exploited in screening, diagnosis and therapy: (1) it has a specific nucleotide sequence which distinguishes it from other HERVs within the genome, although the sequence shares significant identity with the other HERVs; (2) it has tandem 5′ LTRs; (3) it has a fragmented 3′ LTR; (4) its env gene is interrupted by an alu insertion; and (5) its gag contains a unique insertion.

A.1—Nucleotide Sequence

PCAV is a member of the HERV-K sub-family HML2.0. There are roughly 30 to 50 copies of HML2.0 viruses per haploid human genome. HML2 viruses appear to have inserted at least twice in human ancestry: 30 million years ago, before the ape lineage (including humans) split off from monkeys; and 20 million years ago, after the split. The viruses from the 30 million year insertion are sometimes referred to as “old type” viruses and the 20 million insertion as “new type” {3}. Old and new virus proteins are very highly related at the amino acid sequence level, but there are some distinguishing epitopes. DNA sequence identity is high at some regions of the genome but in others, particularly the LTRs, conservation is only about 70%. Most of the differences between old and new LTRs are clustered near the start of transcription, where old viruses have oen or two insertions relative to the new viruses. Old and new LTRs cluster as two separate groups in phylogenetic analyses (FIG. 1). In keeping with their relative genetic ages, old viruses also contain more interruptions and deletions than new viruses.

PCAV appears to have arisen from a rearrangement between a new and an old virus. The 5′ region of the virus (FIG. 2) starts with a new LTR followed by 162 bp from a new virus. The rest of the new virus seems to be missing, as the 162 bp is followed by a 552 bp of non-viral sequence and then an almost-complete old virus. The 3′ LTR of the old virus (FIG. 3) is fragmented and includes a MER11a insertion.

SEQ ID 1 is the 12366 bp sequence of PCAV, based on available human chromosome 22 sequence {4}, from the beginning of its first 5′ LTR to the end of its fragmented 3′ LTR. It is the sense strand of the double-stranded genomic DNA. SEQ ID 10 is the 11101 bp sequence of PCAV from nucleotide 559 in SEQ ID 1 (a possible transcription start site) to its poly-adenylation site (up to nucleotide 11735 in SEQ ID 1), although a more downstream transcription start site (e.g. nucleotide 635±5) is more likely.

The specific sequence of PCAV is manifested at both the mRNA and amino acid levels, and can be used to distinguish it from other HERVs within the genome.

A.2—Tandem 5′ LTRs

Downstream of the 5′ LTR of a HERV-K, before the start of the gag open reading frame, there is a conserved splice donor site (5′SS). This splice donor can join to splice acceptor sites (3′SS) at the start of the env open reading frame (FIG. 4).

HERV-K genomes also include two splice acceptor sequences near the 3′ end of the LTR, but these are not ordinarily used because they have no upstream viral splice donor partner. However, PCAV has two LTRs at its 5′ end: the first is from a new HERV-K and the second is from an old HERV-K. The normally-unused splice acceptors in the old LTR can thus co-operate with the splice donor in the new LTR (FIG. 2), and transcripts resulting from these splice donor/acceptor pairings are specific to PCAV.

Transcripts formed by using a splice acceptor site near the 3′ end of the second 5′ LTR comprise (i) a sequence transcribed from the transcription start site in the first 5′ LTR, continuing to a splice donor site closely downstream of the first 5′ LTR, joined to (ii) a sequence transcribed from one of the splice acceptor sites near the 3′ end of the second 5′ LTR. Detection of such transcripts indicates that PCAV is being transcribed.

In SEQ ID 1: the transcription start site in the first 5′ LTR would be at nucleotide 559 by homology to other viruses, but seems to be further downstream (e.g. at around 635±2) empirically; the conserved splice donor site downstream of the first 5′ LTR is at nucleotides 1076-1081; the two splice acceptor sites near the 3′ end of the second 5′ LTR are at nucleotides 2593-2611 and 2680-2699. SEQ ID 2 is the sequence between the predicted transcription start site and the splice donor site. SEQ ID 3 is the first 10 nucleotides following the first splice acceptor site. SEQ ID 4 is the first 10 nucleotides following the second splice acceptor site. SEQ ID 5 is SEQ ID 2 fused to SEQ ID 3. SEQ ID 6 is SEQ ID 2 fused to SEQ ID 4.

A.3—Fragmented 3′ LTR

The 3′ LTR of PCAV is fragmented, including insertion of a MER11a repetitive element (FIG. 3). PCAV mRNAs terminate using a polyadenylation signal within the MER11a insertion, rather than using the signal within the viral LTR. Transcripts which terminate with a partial copy of a 3′ HERV-K LTR followed by a MER11a sequence are specific to PCAV.

The 3′ ends of transcripts from PCAV include copies of a partial LTR and a partial MER11a (FIG. 3). Detection of such transcripts indicates that PCAV is being transcribed.

In SEQ ID 1: the 3′ LTR begins at nucleotide 10520 and continues until nucleotide 10838, where it is interrupted by a MER11a insertion; the MER11a insertion starts at nucleotide 10839 and continues to nucleotide 11834; after nucleotides 11835-11928, the 3′ LTR continues from nucleotide 11929 to 12366. Within the MER11a insertion is its polyadenylation signal (located between nucleotides 11654 to 11659). SEQ ID 7 is the sequence of the first 319 nt fragment of the 3′ LTR. SEQ ID 8 is the sequence of the MER11a insertion up to its polyA site. SEQ ID 9 is SEQ ID 7 fused to SEQ ID 8.

A.4—Alu in env

As well as being disrupted by mutations due to genetic age, the env gene of PCAV is interrupted by an alu sequence. Detection of transcripts containing both env and alu sequence indicates that PCAV is being transcribed.

In SEQ ID 1, the alu is at nucleotides 9938 to 10244 (SEQ ID 32). The 100 nucleotides immediately preceding the alu sequence (9838-9937) are SEQ ID 37, the last 10 mer of which (9928-9937) is SEQ ID 33. The 100 nucleotides immediately following the alu sequence are SEQ ID 40, the first 10mer of which (10244-10253) is SEQ ID 34. The first 10 nucleotides of the alu sequence are SEQ ID 35 and the last 10 are SEQ ID 41. SEQ ID 36 is the 20mer bridging the alu/env boundary and SEQ ID 45 is the 20mer bridging the end of the alu sequence. SEQ ID 39 is the 8mer bridging the alu/env boundary, and SEQ ID 44 is the 8mer bridging the end of the alu sequence. SEQ ID 38 is SEQ ID 37+SEQ ID 32, SEQ ID 42 is SEQ ID 41+SEQ ID 40, and SEQ ID 43 is SEQ ID 32+SEQ ID 40.

A.5—Unique ag Sequences

The PCAV gag gene contains a 48 nucleotide sequence (SEQ ID 53) which is not found in other HERV-Ks. The 48mer encodes 16mer SEQ ID 110, which is not found in gag proteins from new or in other old HERV-Ks. Detection of transcripts containing SEQ ID 53, or of polypeptides containing SEQ ID 110, or antibodies which recognize epitope within or including SEQ ID 110 thus indicates that PCAV is being transcribed.

The PCAV gag gene also contains a 69 nucleotide sequence (SEQ ID 111) which is not found in new HERV-Ks. The 69mer encodes 23mer SEQ ID 55. Detection of transcripts containing SEQ ID 111, or of polypeptides containing SEQ ID 55, or antibodies which recognize epitope within or including SEQ ID 55 thus indicates that an old HERV-K, typically PCAV, is being transcribed.

B—Detecting mRNA Expression Products

The diagnostic method of the invention may be based on mRNA detection. PCAV mRNA may be detected directly or indirectly. It is preferred to detect a mRNA directly, thereby avoiding the need for separate preparation of mRNA-derived material (e.g. cDNA).

B.1—PCAV mRNA Transcripts of the Invention

mRNA transcripts for use according to the present invention are transcribed from PCAV. Three preferred types of transcript are: (1) transcripts spliced using a splice acceptor site near the 3′ end of the second 5′ LTR; (2) transcripts comprising both 3′ LTR and MER11a sequences; (3) transcripts comprising the alu-interrupted env gene; and (4) transcripts comprising a PCAV-specific gag sequence.

The invention provides a mRNA transcript transcribed from a human endogenous retrovirus located at megabase 20.428 on chromosome 22.

The invention also provides a mRNA transcript comprising a nucleotide sequence with n % or more sequence identity to SEQ ID 23, or to a nucleotide sequence lacking up to 100 nucleotides (e.g. 10, 20, 30, 40, 50, 60, 70, 71, 72, 73, 74, 75, 76, 77, 78, 79, 80, 81, 82, 83, 84, 85, 86, 87, 88, 89, 90 or 100) from the 5′ end of SEQ ID 23 e.g. n % or more sequence identity to SEQ ID 1197 or 1198. The nucleotide sequence is preferably at the 5′ end of the RNA, although upstream sequences may be present. The nucleotide sequence may be at the 3′ end of the RNA, but there will typically be further downstream elements such as a poly-A tail. These mRNA transcripts include, allelic variants, SNP variants, homologs, orthologs, paralogs, mutants, etc. of SEQ ID 23, SEQ ID 1197 and SEQ ID 1198.

The invention provides a mRNA transcript formed by splicing involving a splice acceptor site near the 3′ end of the second 5′ LTR. Thus the invention provides a mRNA transcript comprising the sequence —N₁—N₂— (e.g. SEQ ID 24, SEQ ID 25, SEQ ID 1199 or SEQ ID 1200), where: N₁ is a nucleotide sequence (e.g. SEQ ID 26, SEQ ID 1201) from (i) the 5′ end of a mRNA transcribed from the first 5′ LTR of a human endogenous retrovirus located at megabase 20.428 on chromosome 22, to (ii) a first splice donor site downstream of the U5 region of said mRNA transcribed from the first 5′ LTR; and N₂ is a nucleotide sequence (e.g. SEQ ID 27 or SEQ ID 28) immediately downstream of a splice acceptor site located (i) downstream of said first splice donor site and (ii) upstream of a second splice donor site, the second splice donor site being downstream of the second 5′ LTR of said endogenous retrovirus. The first splice donor site is preferably the site conserved in the HML2 sub-family, located about 100 nucleotides downstream of the first 5′ LTR (after nucleotide 1075 in SEQ ID 1). The second splice donor site is preferably the site conserved in the HML2 sub-family, located about 100 nucleotides downstream of the second 5′ LTR (after SEQ ID 1 nucleotide 2778). The splice acceptor is preferably downstream of the second 5′ LTR.

The invention also provides a mRNA transcript comprising the sequence —N₁—N₂—, where: N₁ is a nucleotide sequence with a % or more sequence identity to SEQ ID 26 and/or SEQ ID 1201 and N₂ is a nucleotide sequence with b % or more sequence identity to SEQ ID 27 or SEQ ID 28. These mRNA transcripts of the invention are illustrated in FIG. 5. Transcripts which use the second splice site (i.e. N₂ is SEQ ID 28) are preferred.

In both cases, N₁ is preferably at the 5′ end of the RNA, although upstream sequences may be present. N₂ may be at the 3′ end of the RNA, but downstream sequences will usually be present.

The invention also provides a mRNA transcript comprising a nucleotide sequence with c % or more sequence identity to SEQ ID 24, SEQ ID 25, SEQ ID 1199 or SEQ ID 1200.

The invention provides a mRNA transcript comprising the sequence —N₃—N₄— (e.g. SEQ ID 29), where: N₃ is a nucleotide sequence (e.g. SEQ ID 30) from the 3′ end of the 5′ fragment of the 3′ LTR of a human endogenous retrovirus located at megabase 20.428 on chromosome 22, and N₄ is a nucleotide sequence (e.g. SEQ ID 31) from 5′ end of the MER11a insertion in a human endogenous retrovirus located at megabase 20.428 on chromosome 22.

The invention also provides a mRNA transcript comprising the sequence —N₃—N₄—, where: N₃ is a nucleotide sequence with d % or more sequence identity to SEQ ID 30 and N₄ is a nucleotide sequence with e % or more sequence identity to SEQ ID 31. The RNA may comprise the sequence —N₃—N₄—N₅—N₆—, wherein: N₅ is a nucleotide sequence between the polyA signal and the polyA site of a MER11a sequence; and N₆ is a polyA tail.

In both cases, the transcript will generally include sequence upstream of N₃. The transcript will generally include sequence downstream of N₄, such as a polyA tail.

The invention also provides a mRNA transcript comprising a nucleotide sequence with f % or more sequence identity to SEQ ID 29.

The invention provides a mRNA transcript comprising the sequence —N₇—N₈— (e.g. SEQ ID 38), where: N₇ is a nucleotide sequence (e.g. SEQ ID 37) preceding the alu insertion within the env gene of a human endogenous retrovirus located at megabase 20.428 on chromosome 22, and N₈ is a nucleotide sequence (e.g. SEQ ID 32) beginning at the 5′ end of said alu insertion.

The invention also provides a mRNA transcript comprising the sequence —N₇—N₈—, where: N₇ is a nucleotide sequence with mm % or more sequence identity to SEQ ID 37 and N₈ is a nucleotide sequence with nn % or more sequence identity to SEQ ID 32.

The transcript will generally include sequence upstream of N₇ and downstream of N₈.

The invention also provides a mRNA transcript comprising a nucleotide sequence with pp % or more sequence identity to SEQ ID 38.

The invention provides a mRNA transcript comprising the sequence —N₉—N₁₀— (e.g. SEQ ID 43), where: N₉ is a nucleotide sequence (e.g. SEQ ID 32) at the end of the alu insertion within the env gene of a human endogenous retrovirus located at megabase 20.428 on chromosome 22, and N₁₀ is a nucleotide sequence (e.g. SEQ ID 40) immediately downstream of said alu insertion.

The invention also provides a mRNA transcript comprising the sequence —N₉—N₁₀—, where: N₉ is a nucleotide sequence with uu % or more sequence identity to SEQ ID 41 and N₁₀ is a nucleotide sequence with vv % or more sequence identity to SEQ ID 40.

The transcript will generally include sequence upstream of N₉ and downstream of N₁₀.

The invention also provides a mRNA transcript comprising a nucleotide sequence with ww % or more sequence identity to SEQ ID 42.

The invention provides a mRNA transcript comprising a nucleotide sequence with uu % or more sequence identity to SEQ ID 41.

The transcript will generally include sequence upstream of N₉ and downstream of N₁₀.

The invention also provides a mRNA transcript comprising a nucleotide sequence with ii % or more sequence identity to SEQ ID 53.

The invention also provides a mRNA transcript comprising a nucleotide sequence with ii % or more sequence identity to SEQ ID 111.

The invention also provides a mRNA transcript comprising a nucleotide sequence with ii % or more sequence identity to SEQ ID 1191. The invention also provides a mRNA transcript which encodes a polypeptide having at least ii % sequence identity to SEQ ID 98.

B.2—Direct and Indirect Detection of mRNA

PCAV mRNA transcripts of the invention may be detected directly, for example by sequencing of the mRNA or by hybridization to mRNA transcripts (e.g. by Northern blot). Various techniques are available for detecting the presence or absence of a particular RNA sequence in a sample {e.g. refs. 20 & 21}.

Indirect detection of mRNA transcripts is also possible and is performed on nucleic acid derived from a PCAV mRNA transcript e.g. detection of a cDNA copy of PCAV mRNA, detection of nucleic acids amplified from a PCAV mRNA template, etc.

A preferred method for detecting RNA is RT-PCR (reverse transcriptase polymerase chain reaction) {e.g. refs. 5 to 13}. RT-PCR of mRNA from prostate cells is reported in, for example, references 14 to 19. It is preferred to use PCAV-specific probes in RT-PCR.

Whether direct or indirect detection is used, the method of the invention involves detection of a single-stranded or double-stranded PCAV nucleic acid target, either (a) in the form of PCAV mRNA or (b) in the form of nucleic acid comprising a copy of at least a portion of a PCAV mRNA and/or a sequence complementary to at least a portion of a PCAV mRNA.

The method of the invention does not involve the detection of PCAV genomic DNA, as this is present in all human cells and its presence is therefore not characteristic of tumors. If a sample contains PCAV DNA, it is preferred to use a RNA-specific detection technique or to focus on sequences present in PCAV mRNA transcripts but not in PCAV genomic DNA (e.g. splice junctions, polyA tail etc.). The method of the invention may therefore comprise an initial step of: (a) extracting mRNA from a patient sample; (b) removing DNA from a patient sample without removing mRNA; and/or (c) removing or disrupting PCAV DNA, but not PCAV m-RNA, in a patient sample. As an alternative, a RNA-specific assay can be used which is not affected by the presence of homologous DNA. For RT-PCR, genomic DNA should be removed.

Methods for selectively extracting RNA from biological samples are well known {e.g. refs. 20 & 21} and include methods based on guanidinium buffers, lithium chloride, acid phenol:chloroform extraction, SDS/potassium acetate etc. After total cellular RNA has been extracted, mRNA may be enriched e.g. using oligo-dT techniques.

Methods for removing DNA from biological samples without removing mRNA are well known {e.g. appendix C of ref. 20} and include DNase digestion. If DNase is used then it must be removed or inactivated (e.g. by chelation with EDTA, by heating, or by proteinase K treatment followed by phenol/chloroform extraction and NH₄OAc/EtOH precipitation) prior to subsequent DNA synthesis or amplification, in order to avoid digestion of the newly-synthesized DNA.

Methods for removing PCAV DNA, but not PCAV RNA, will use a reagent which is specific to a sequence within a PCAV DNA e.g. a restriction enzyme which recognizes a DNA sequence within the PCAV genome, but which does not cleave the corresponding RNA sequence.

Methods for specifically purifying PCAV mRNAs from a sample may also be used. One such method uses an affinity support which binds to PCAV mRNAs. The affinity support may include a polypeptide sequence which binds to the PCAV mRNA e.g. the cORF polypeptide, which binds to the LTR of HERV-K mRNAs in a sequence-specific manner, or HIV Rev protein, which has been shown to recognize the HERV-K LTR in RNA transcripts {22}.

PCAV mRNA need not be maintained in a wild-type form for detection. It may, for example, be fragmented, provided that the fragmentation maintains PCAV-specific sequences within the mRNA.

B.3—PCAV Nucleic Acid Targets for Detection

The invention provides nucleic acid comprising (a) the nucleotide sequence of a mRNA transcript transcribed from a human endogenous retrovirus located at megabase 20.428 on chromosome 22, and/or (b) the complement of (a). The invention also provides nucleic acid comprising a nucleotide sequence with qq % or more sequence identity to SEQ ID 10, SEQ ID 1197 and/or SEQ ID 1198. PCAV is approximately 87.5% identical to the HERV-K found at megabase 47.1 on chromosome 6 and approximately 86% identical to the HERV-K found at megabase 103.75 on chromosome 3.

The invention provides nucleic acid comprising (a) nucleotide sequence —N₁—N₂— as defined above, and/or (b) the complement of (a). The invention also provides nucleic acid comprising (a) a nucleotide sequence with c % or more sequence identity to SEQ ID 5, SEQ ID 6, SEQ ID 1199 or SEQ ID 1200, and/or (b) the complement of (a).

The invention provides nucleic acid comprising (a) nucleotide sequence —N₃—N₄— as defined above, and/or (b) the complement of (a). The invention also provides nucleic acid comprising (a) a nucleotide sequence with f % or more sequence identity to SEQ ID 9, and/or (b) the complement of (a).

The invention also provides nucleic acid comprising (a) nucleotide sequence —N₃—N₄—N₅—N₆— as defined above, and/or (b) the complement of (a).

The invention provides nucleic acid comprising (a) nucleotide sequence —N₇N₈— as defined above, and/or (b) the complement of (a). The invention also provides nucleic acid comprising (a) a nucleotide sequence with aa % or more sequence identity to SEQ ID 38, and/or (b) the complement of (a).

The invention provides nucleic acid comprising (a) nucleotide sequence —N₉—N₁₀— as defined above, and/or (b) the complement of (a). The invention also provides nucleic acid comprising (a) a nucleotide sequence with hh % or more sequence identity to SEQ ID 42, and/or (b) the complement of (a).

The invention provides nucleic acid comprising a nucleotide sequence with bbb % or more sequence identity to SEQ ID 53, and/or (b) the complement of (a).

The invention provides nucleic acid comprising a nucleotide sequence with fff % or more sequence identity to SEQ ID 111, and/or (b) the complement of (a).

Specific nucleic acid targets include SEQ IDs 99 to 109, which are splice variant cDNA sequences assuming a transcription start site in SEQ ID 1 at 559 and including four A residues at the 3′ end. Assuming a more downstream transcription start site (e.g. nucleotide 635 of SEQ ID 1), these nucleic targets would not include a stretch of nucleotides at the 5′ end of SEQ IDs 99 to 109 e.g. they would not include 10, 20, 30, 40, 50, 60, 70, 71, 72, 73, 74, 75, 76, 77, 78, 79, 80, 81, 82, 83, 84, 85, 86, 87, 88, 89, 90, 100 or more of the 5′ nucleotides. 25mer sequences based on cDNA sequences are given as SEQ IDs 337 to 599.

B.4—Nucleic Acid Materials for Direct or Indirect mRNA Detection

The invention provides nucleic acid which can hybridize to a PCAV nucleic acid target.

Hybridization reactions can be performed under conditions of different “stringency”. Conditions that increase stringency of a hybridization reaction of widely known and published in the art {e.g. page 7.52 of reference 21}. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art {e.g. see references 20, 21, 23, 24, 28 etc.}.

In some embodiments, nucleic acid of the invention hybridizes to a target of the invention under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

Preferred nucleic acids of the invention hybridize to PCAV nucleic acid targets but not to nucleic acid targets from other HERV-Ks. PCAV-specific hybridization is favored by exploiting features found within PCAV transcripts but not in other HERV-K transcripts e.g. specific nucleotide sequences, features arising from the tandem 5′ LTRs, features arising from the MER11a insertion within the 3′ LTR, or features arising from the alu interruption of env. Sequence alignments can be used to locate regions of PCAV which are most divergent from other HERV-K genomes and in which PCAV-specific hybridization can occur. Specificity for PCAV is desirable in order to detect its up-regulation above the low-level of natural background expression of other new HERV-Ks seen in most cells.

One group of preferred nucleic acids of the invention can specifically detect PCAV products in which a splice acceptor site near the 3′ end of the second 5′ LTR has been used. As described above, such splicing brings together sequences N₁ and N₂, which are not juxtaposed in PCAV genomic DNA. Thus the invention provides a nucleic acid which hybridizes to sequence —N₁—N₂— (or the complement thereof) within a PCAV nucleic acid target, but which does not hybridize to sequences N₁ or N₂ alone (or to their complements alone). The nucleic acid comprises a first sequence which can hybridize to N₁ (or to its complement) and a second sequence which can hybridize to N₂ (or to its complement), such that it will hybridize to a target in which N₁ and N₂ are adjacent, but will not hybridize to targets in which splicing has not brought N₁ and N₂ together. Such nucleic acids can identify PCAV transcripts in the presence of PCAV genomic DNA because of the difference in relative locations of N₁ and N₂.

Another group of preferred nucleic acids of the invention can specifically detect mRNAs containing 3′ LTR and MER11a sequences. Thus the invention provides a nucleic acid which hybridizes to sequence —N₃—N₄— (or the complement thereof) within a PCAV nucleic acid target, but which does not hybridize to sequences N₃ or N₄ alone (or to their complements alone). The nucleic acid comprises a first sequence which can hybridize to N₃ (or to its complement) and a second sequence which can hybridize to N₄ (or to its complement), such that it will hybridize to targets which include both (i) a 3′ LTR sequence and (ii) a MER11a sequence, but not to targets which include only one of (i) and (ii). The nucleic acid may inherently be able to hybridize to genomic DNA, although this property is not useful for detecting transcripts.

Another group of preferred nucleic acids of the invention can specifically detect mRNAs containing the alu-interrupted env gene. Thus the invention provides a nucleic acid which hybridizes to sequence —N₇—N₈— (or the complement thereof) within a PCAV nucleic acid target, but which does not hybridize to sequences N₇ or N₈ alone (or to their complements alone). The nucleic acid comprises a first sequence which can hybridize to N₇ (or to its complement) and a second sequence which can hybridize to N₈ (or to its complement), such that it will hybridize to targets which include both (i) the env sequence immediately preceding the alu interruption and (ii) an alu interruption, but not to targets which include only one of (i) and (ii). The nucleic acid may inherently be able to hybridize to genomic DNA, although this property is not useful for detecting transcripts.

The invention also provides a nucleic acid which hybridizes to sequence —N₉—N₁₀— (or the complement thereof) within a PCAV nucleic acid target, but which does not hybridize to sequences N₉ or N₁₀ alone (or to their complements alone). The nucleic acid comprises a first sequence which can hybridize to N₉ (or to its complement) and a second sequence which can hybridize to N₁₀ (or to its complement), such that it will hybridize to targets which include both (i) the 3′ region of the alu interruption within env and (ii) the sequence immediately downstream of the alu interruption, but not to targets which include only one of (i) and (ii). The nucleic acid may inherently be able to hybridize to genomic DNA, although this property is not useful for detecting transcripts.

The ability of a nucleic acid to hybridize to a PCAV nucleic acid target is related to its intrinsic features (e.g. the degree of sequence identity to the target) as well as extrinsic features (e.g. temperature, salt concentration etc.). A group of preferred nucleic acids of the invention have a good intrinsic ability to hybridize to PCAV nucleic acid targets.

Thus the invention provides a nucleic acid comprising a nucleotide sequence with s % or more sequence identity to a fragment of a PCAV nucleic acid target or to the complement of a fragment of a PCAV nucleic acid target. The invention provides a nucleic acid comprising a nucleotide sequence with g % or more sequence identity to a fragment of SEQ ID 10 or to the complement of a fragment of SEQ ID 10. The invention also provides a nucleic acid comprising a nucleotide sequence with h % or more sequence identity to a fragment of SEQ ID 5 or to the complement of a fragment of SEQ ID 5. The invention also provides a nucleic acid comprising a nucleotide sequence with i % or more sequence identity to a fragment of SEQ ID 6 or to the complement of a fragment of SEQ ID 6. The invention also provides a nucleic acid comprising a nucleotide sequence with j % or more sequence identity to a fragment of SEQ ID 9 or to the complement of a fragment of SEQ ID 9. The invention also provides a nucleic acid comprising a nucleotide sequence with ccc % or more sequence identity to a fragment of SEQ ID 53 or to the complement of a fragment of SEQ ID 53. The invention also provides a nucleic acid comprising a nucleotide sequence with kkk % or more sequence identity to SEQ ID 1191. It also provides a nucleic acid comprising a nucleotide sequence which encodes a polypeptide having at least mmm % sequence identity to SEQ ID 98. The invention also provides a nucleic acid comprising a nucleotide sequence with nnn % or more sequence identity to SEQ ID 1198. It also provides a nucleic acid comprising a nucleotide sequence which encodes a polypeptide having at least qqq % sequence identity to SEQ ID 1199. It also provides a nucleic acid comprising a nucleotide sequence which encodes a polypeptide having at least rrr % sequence identity to SEQ ID 1200.

The invention provides a nucleic acid comprising a fragment of at least k contiguous nucleotides of SEQ ID 10 or of the complement of SEQ ID 10. The fragment is preferably located within SEQ ID 1197 and/or 1198.

The invention also provides a nucleic acid comprising a fragment of at least l contiguous nucleotides of SEQ ID 47 or of the complement of SEQ ID 47. The fragment preferably comprises nucleotide sequence B₁ₐ-B₂ₐ (or its complement), wherein B₁ₐ comprises m or more nucleotides from the 3′ end of SEQ ID 2 and B₂ₐ comprises p or more nucleotides from the 5′ end of SEQ ID 46. These nucleic acids thus span a splice junction which brings sequences N₁ and N₂ together and are thus able to identify PCAV transcripts in the presence of PCAV genomic DNA because of the difference in the relative locations of B₁ₐ and B₂ₐ. B₁ₐ-B₂ₐ preferably comprises SEQ ID 11 (or its complement), where m=p=4, and more preferably comprises SEQ ID 50 (or its complement), where m=p=10.

The invention also provides a nucleic acid comprising a fragment of at least q contiguous nucleotides of SEQ ID 49 or of the complement of SEQ ID 49. The fragment preferably comprises nucleotide sequence B_{1b}-B_{2b} (or its complement), wherein B_{1b} comprises r or more nucleotides from the 3′ end of SEQ ID 2 and B_{2b} comprises t or more nucleotides from the 5′ end of SEQ ID 48. These nucleic acids thus span the splice junction which brings sequences N₁ and N₂ together and are thus able to identify PCAV transcripts in the presence of PCAV genomic DNA because of the difference in the relative locations of B_{1b} and B_{2b}. B_{1b}-B_{2b} preferably comprises SEQ ID 12 (or its complement), where r=t=4, and more preferably comprises SEQ ID 51 (or its complement), where r=t=10.

The invention also provides a nucleic acid comprising a fragment of at least u contiguous nucleotides of SEQ ID 9 or of the complement of SEQ ID 9. The fragment preferably comprises nucleotide sequence B₃-B₄ (or its complement), wherein B₃ comprises v or more nucleotides from the 3′ end of SEQ ID 7 and B₄ comprises w or more nucleotides from the 5′ end of SEQ ID 8. These nucleic acids thus include part of both of N₃ and N₄. B₃-B₄ preferably comprises SEQ ID 13 (or its complement), where v=w=4, and more preferably comprises SEQ ID 52 (or its complement), where v=w=10.

The invention also provides a nucleic acid comprising a fragment of at least rr contiguous nucleotides of SEQ ID 38 or of the complement of SEQ ID 38. The fragment preferably comprises nucleotide sequence B₇-B₈ (or its complement), wherein B₇ comprises ss or more nucleotides from the 3′ end of SEQ ID 37 and B₄ comprises tt or more nucleotides from the 5′ end of SEQ ID 32. These nucleic acids thus include part of both of N₇ and N₈. B₇-B₈ preferably comprises SEQ ID 39 (or its complement), where ss=t=4, and more preferably comprises SEQ ID 36 (or its complement), where ss=tt=10.

The invention also provides a nucleic acid comprising a fragment of at least jj contiguous nucleotides of SEQ ID 43 or of the complement of SEQ ID 43. The fragment preferably comprises nucleotide sequence B₉-B₁₀, or its complement, and wherein B₉ comprises kk or more nucleotides from the 3′ end of SEQ ID 32 and B₁₀ comprises 11 or more nucleotides from the 5′ end of SEQ ID 40. These nucleic acids thus include part of both of N₉ and N₁₀. B₉-B₁₀ preferably comprises SEQ ID 44 (or its complement), where kk=ll=4, and more preferably comprises SEQ ID 45 (or its complement), where kk=ll=10.

The invention also provides a nucleic acid comprising a fragment of at least ddd contiguous nucleotides of SEQ ID 53 or of the complement of SEQ ID 53. The invention also provides a nucleic acid comprising a fragment of at least ggg contiguous nucleotides of SEQ ID 111 or of the complement of SEQ ID 111. The invention also provides a nucleic acid comprising a fragment of at least hhh contiguous nucleotides of SEQ ID 112 or of the complement of SEQ ID 112. The invention also provides a nucleic acid comprising a fragment of at least jjj contiguous nucleotides of SEQ ID 1191 or of the complement of SEQ ID 1191.

The invention provides a nucleic acid of formula 5′-X-Y-Z-3′, wherein: —X— is a nucleotide sequence consisting of x nucleotides; -Z- is a nucleotide sequence consisting of z nucleotides; —Y— is a nucleotide sequence consisting of either (a) a fragment of y nucleotides of any of SEQ IDs 1-13, 20-53, 57, 58, 63, 81, 86, 88-91, 99-109, 111, 112, 1191, 1197 or 1198, or (b) the complement of (a); and said nucleic acid 5′-X-Y-Z-3′ is neither (i) a fragment of SEQ IDs 1-13, 20-53, 57, 58, 63, 81, 86, 88-91, 99-109, 111, 112, 1191, 1197 or 1198 or (ii) the complement of (i).

Where —Y— is (a), the nucleotide sequence of —X— preferably shares less than bb % sequence identity to the x nucleotides which are 5′ of sequence —Y— in SEQ IDs 1-13, 20-53, 57, 58, 63, 81, 86, 88-91, 99-109, 111, 112, 1191, 1197 or 1198 and/or the nucleotide sequence of -Z- preferably shares less than cc % sequence identity to the z nucleotides which are 3′ of sequence -Z- in SEQ IDs 1-13, 20-53, 57, 58, 63, 81, 86, 88-91, 99-109, 111, 112, 1191, 1197 or 1198.

Where —Y— is (b), the nucleotide sequence of —X— preferably shares less than bb % sequence identity to the complement of the x nucleotides which are 5′ of the complement of sequence —Y— in SEQ IDs 1-13, 20-53, 57, 58, 63, 81, 86, 88-91, 99-109, 111, 112, 1191, 1197 or 1198 and/or the nucleotide sequence of -Z- preferably shares less than cc % sequence identity to the complement of the z nucleotides which are 3′ of the complement of sequence —Y— in SEQ IDs 1-13, 20-53, 57, 58, 63, 81, 86, 88-91, 99-109, 111, 112, 1191, 1197 or 1198.

The —X— and/or -Z- moieties may comprise a promoter sequence (or its complement).

The invention provides nucleic acid comprising nucleotide sequence SEQ ID 53. This sequence is specific within the human genome to PCAV. The invention also provides nucleic acid comprising nucleotide sequence SEQ ID 111.

The invention also provides nucleic acid comprising nucleotide sequence SEQ ID 1191.

Various PCAV nucleic acids are provided by the invention. 25mer fragments of PCAV sequences are given as SEQ IDs 120 to 1184. The invention provides these sequences as 25mers, as well as fragments thereof (e.g. the 2×24mers, the 3×23mers, the 4×22mers . . . the 19×7mers in each) and as longer PCAV fragments comprising these 25mers.

Preferred nucleic acids of the invention comprise one or more of SEQ IDs 53 and 842-1184.

Nucleic acids of the invention are particularly useful as probes and/or as primers for use in hybridization and/or amplification reactions.

More than one nucleic acid of the invention can hybridize to the same target (e.g. more than one can hybridize to a single mRNA or cDNA).

B.5—Nucleic Acid Amplification

Nucleic acid in a sample can conveniently and sensitively be detected by nucleic acid amplification techniques such as PCR, SDA, SSSR, LCR, TMA, NASBA, T7 amplification etc. The technique preferably gives exponential amplification. A preferred technique for use with RNA is RT-PCR (e.g. see chapter 15 of ref. 20). The technique may be quantitative and/or real-time.

Amplification techniques generally involve the use of two primers. Where a target sequence is single-stranded, the techniques generally involve a preliminary step in which a complementary strand is made in order to give a double-stranded target. The two primers hybridize to different strands of the double-stranded target and are then extended. The extended products can serve as targets for further rounds of hybridization/extension. The net effect is to amplify a template sequence within the target, the 5′ and 3′ termini of the template being defined by the locations of the two primers in the target.

The invention provides a kit comprising primers for amplifying a template sequence contained within a PCAV nucleic acid target, the kit comprising a first primer and a second primer, wherein the first primer comprises a sequence substantially complementary to a portion of said template sequence and the second primer comprises a sequence substantially complementary to a portion of the complement of said template sequence, wherein the sequences within said primers which have substantial complementarity define the termini of the template sequence to be amplified.

Kits of the invention may further comprise a probe which is substantially complementary to the template sequence and/or to its complement and which can hybridize thereto. This probe can be used in a hybridization technique to detect amplified template.

Kits of the invention may further comprise primers and/or probes for generating and detecting an internal standard, in order to aid quantitative measurements {e.g. 15, 25}.

Kits of the invention may comprise more than one pair of primers (e.g. for nested amplification), and one primer may be common to more than one primer pair. The kit may also comprise more than one probe.

The template sequence is preferably located within a transcript of a HERV-K located at megabase 20.428 of chromosome 22, and is more preferably a fragment of SEQ ID 10 (or SEQ ID 23). The template sequence is preferably at least 50 nucleotides long (e.g. 60, 70, 80, 90, 100, 125, 150, 175, 200, 250, 300, 400, 500, 600, 700, 800, 900, 1000, 1250, 1500, 2000, 3000 nucleotides or longer). The length of the template is inherently limited by the length of the target within which it is located, but the template sequence is preferably shorter than 500 nucleotides (e.g. 450, 400, 350, 300, 250, 200, 175, 150, 125, 100, 90, 80, 70 or shorter).

A preferred template comprises SEQ ID 53 and/or SEQ ID 111.

Primers and probes used in kits of the invention are preferably nucleic acids as described in section B.4 above. Particularly preferred primers are those based on SEQ IDs 600-1184, (or their complements) e.g. comprising primers comprising SEQ IDs 600-1184, or primers comprising fragments of ppp or more nucleotides from one of SEQ IDs 600-1184.

Further features of primers and probes are described in section B.6 below.

Preferred kits comprise (i) a first primer comprising a sequence which is substantially identical to a portion of SEQ ID 10 and (ii) a second primer comprising a sequence which is substantially complementary to a portion of SEQ ID 10, such that the primer pair (i) and (ii) defines a template sequence within SEQ ID 10. Other preferred kits comprise (i) a first primer comprising a sequence which is substantially identical to a portion of the complement of SEQ ID 10 and (ii) a second primer comprising a sequence which is substantially complementary to a portion of the complement of SEQ ID 10, such that the primer pair defines a template sequence within SEQ ID 10. The portion and template sequence preferably fall within SEQ ID 1197 or SEQ ID 1198.

It is preferred that one or both of the primers is not substantially complementary to a portion of a HERV-K other than PCAV (or its complement) such that the primer pair is specific for PCAV.

SEQ ID 10 may be divided into four exons: (1) nucleotides 1-517, containing sequences up to the conserved splice donor downstream of the first 5′ LTR; (2) nucleotides 2142-2209, containing sequences between the splice acceptor near the 3′ end of the second 5′ LTR and the conserved splice donor; (3) nucleotides 7608-7686; and (4) nucleotides 9866-11181 (assuming transcription start at nucleotide 559 of SEQ ID 1). Exon (2) arises because of the unique PCAV feature of tandem 5′ LTRs, but the other three exons exist in other HERV-Ks.

In preferred kits of the invention, the first and second primers are located in different exons. This arrangement means that the amplified template sequence is shorter than would be obtained from genomic DNA, because of the absence of introns. For example:

| | | | | | | |
|---|---|---|---|---|---|---|
| First primer in exon | 1 | 1 | 1 | 2 | 2 | 3 |
| Second primer in exon | 2 | 3 | 4 | 3 | 4 | 4 |

With reference to SEQ ID 10, therefore, the primers may comprise a fragment of SEQ ID 10 (or its complement) located between the following coordinates:

| | | | | | | |
|---|---|---|---|---|---|---|
| First primer | 1-517 | 1-517 | 1-517 | 2142-2219 | 2142-2219 | 7608-7686 |
| Second primer | 2142-2219 | 7608-7686 | 9866-11181 | 7608-7686 | 9866-11181 | 9866-11181 |

With reference to SEQ ID 1, these coordinates are:

| | | | | | | |
|---|---|---|---|---|---|---|
| First primer | 559-1075 | 559-1075 | 559-1075 | 2700-2777 | 2700-2777 | 8166-8244 |
| Second primer | 2700-2777 | 8166-8244 | 10424-11739 | 8166-8244 | 10424-11739 | 10424-11739 |

With a more-downstream transcription start site, however, the first exon may begin downstream of nucleotide 559 e.g. at around nucleotide 633, 635 or 637.

Example primers within exon 1 are SEQ IDs 120 to 219. Example primers within exons 2 to 4 are SEQ IDs 220 to 336.

In other preferred kits, one or both of the first and second primers comprise a first sequence from a first exon and a second sequence from a second exon, such that the primer bridges an exon-exon boundary after splicing. For example, a primer may comprise sequences from exons 1 & 2, exons 1 & 3, exons 1 & 4, exons 2 & 3, exons 2 & 4, or exons 3 & 4. These primers hybridize to transcripts where splicing has taken place.

With reference to SEQ ID 10, therefore, the primers may comprise a first sequence from the 3′ end of the following coordinates and second sequence from the 5′ end of the following coordinates (or complements thereof):

| | | | | | | |
|---|---|---|---|---|---|---|
| First sequence | 1-517 | 1-517 | 1-517 | 2142-2209 | 2142-2209 | 7608-7686 |
| Second sequence | 2142-2209 | 7608-7686 | 9866-11181 | 7608-7686 | 9866-11181 | 9866-11181 |

Taking a more-downstream transcription start site, however, the range ‘1-517’ for selecting the first sequence should be replaced with around ‘77-517’ e.g. 75-517 or 80-517.

In preferred kits for detecting PCAV nucleic acid targets in which a splice acceptor site near the 3′ end of the second 5′ LTR has been used, either (i) the first primer comprises a sequence which is substantially identical to a portion of N₁ and the second primer comprises a sequence which is substantially complementary to a portion of N₂, or (ii) the first primer comprises a sequence which is substantially identical to a portion of the complement of N₁ and the second primer comprises a sequence which is substantially complementary to a portion of the complement of N₂. This primer pair defines a template sequence which bridges the PCAV-specific splice junction. The amplified sequence will be shorter for targets where the splice junction has been used than for unspliced targets (FIG. 5) or for genomic DNA. For targets where transcription may start in the LTR immediately upstream of the splice acceptor sites (e.g. in the second 5′ LTR of PCAV, or in the single 5′ LTR of other HERVs), the amplified sequence will be shorter than for PCAV targets where transcription started in a more upstream 5′ LTR.

In other preferred kits for detecting PCAV products in which a splice acceptor site near the 3′ end of the second 5′ LTR has been used, either (i) the first primer comprises a sequence which is substantially identical to a portion of N₁ and the second primer comprises a sequence which is substantially complementary to a portion of PCAV sequence downstream of a splice donor which is itself downstream of the splice acceptors near the 3′ end of the second PCAV 5′ LTR, or (ii) the first primer comprises a sequence which is substantially identical to a portion of the complement of N₁ and the second primer comprises a sequence which is substantially complementary to a portion of the complement of a PCAV sequence downstream of a splice donor which is itself downstream of the splice acceptors near the 3′ end of the second PCAV 5′ LTR. The primers are located either side of exon 2 and thus define a template sequence which bridges exon 2. The amplified sequence will be longer in targets where the exon is present than in targets where the exon absent (FIG. 6A vs. 6B) and only PCAV targets can give the longer amplification product. All splice products, whether or not including the exon, will give shorter amplification products than unspliced mRNA or genomic DNA targets.

In other preferred kits for detecting PCAV products in which a splice acceptor site near the 3′ end of the second 5′ LTR has been used, either (i) the first primer comprises a sequence which is substantially identical to the splice junction site in N₁—N₂ and the second primer comprises a sequence which is substantially complementary to a portion of a PCAV sequence upstream or downstream of the splice junction site, or (ii) the first primer comprises a sequence which is substantially identical to the complement of the splice junction site in N₁—N₂ and the second primer comprises a sequence which is substantially complementary to a portion of a PCAV upstream or sequence downstream of the splice junction site. The first primer comprises a first sequence which is substantially complementary to a portion of N₁ and a second sequence which is substantially complementary to a portion of N₂ and can hybridize to targets where the splice junction has been used but not to targets where the splice junction has not been used. Amplification from such primer pairs will only occur where the target sequence has been formed by use of the splice junction, and will not occur with unspliced targets or genomic DNA.

In preferred kits for detecting the 3′ region of PCAV products, either (i) the first primer comprises a sequence which is substantially identical to a portion of N₃ and the second primer comprises a sequence which is substantially complementary to a portion of N₄, or (ii) the first primer comprises a sequence which is substantially identical to a portion of the complement of N₃ and the second primer comprises a sequence which is substantially complementary to a portion of the complement of N₄. The primer pair amplifies a template sequence which bridges the 3′ LTR/MER11a junction and amplification will occur only where the target sequence contains both a 3′ LTR sequence and a MER11a sequence (FIG. 7).

In other preferred kits for detecting the 3′ region of PCAV products, either (i) the first primer comprises a first sequence which is substantially identical to a portion of N₃ and a second sequence which is substantially identical to a portion of N₄, and the second primer comprises a sequence which is substantially complementary to a portion of an upstream or downstream PCAV sequence, or (ii) the first primer comprises a first sequence which is substantially identical to a portion of the complement of N₃ and a second sequence which is substantially identical to a portion of the complement of N₄, and the second primer comprises a sequence which is substantially complementary to a portion of the complement of an upstream or downstream PCAV sequence. The first primer hybridizes only to targets which contain both a 3′ LTR sequence and a MER11a sequence, such that amplification occurs only where the target sequence contains both a 3′ LTR sequence and a MER11a sequence (FIG. 7). The second primer is preferably located in exon 3, so the amplification product is shorter than in the genome.

In other preferred kits for detecting the 3′ region of PCAV products, either (i) the first primer comprises a sequence which is substantially identical to a portion of N₃ and the second primer comprises a sequence which is substantially complementary to a portion of a polyA tail, or (ii) the first primer comprises a sequence which is substantially identical to a portion of the complement of N₃ and the second primer comprises a sequence which is substantially complementary to a portion of the complement of polyA tail. The template sequence defined by this primer pair is longer in targets where the 31 LTR contains a MER11a insertion than in targets (e.g. other HERVs) where the 3′ LTR is intact (FIG. 8). PolyA-specificity means that genomic DNA is not amplified.

In preferred kits for detecting PCAV products containing alu-interrupted env, either (i) the first primer comprises a sequence which is substantially identical to a portion of N₇ and the second primer comprises a sequence which is substantially complementary to a portion of N₈, or (ii) the first primer comprises a sequence which is substantially identical to a portion of the complement of N₇ and the second primer comprises a sequence which is substantially complementary to a portion of the complement of N₈. The primer pair amplifies a template sequence which bridges the env/alu junction and amplification will occur only where the target sequence contains both an env sequence and an alu sequence.

In other preferred kits for detecting PCAV products containing alu-interrupted env, either (i) the first primer comprises a first sequence which is substantially identical to a portion of N₇ and a second sequence which is substantially identical to a portion of N₈, and the second primer comprises a sequence which is substantially complementary to a portion of an upstream or downstream PCAV sequence, or (ii) the first primer comprises a first sequence which is substantially identical to a portion of the complement of N₇ and a second sequence which is substantially identical to a portion of the complement of N₈, and the second primer comprises a sequence which is substantially complementary to a portion of the complement of an upstream or downstream PCAV sequence. The first primer hybridizes only to targets which contain both an alu sequence and an env sequence, such that amplification occurs only where the target sequence contains both an alu sequence and an env sequence.

In further preferred kits for detecting PCAV products containing alu-interrupted env, either (i) the first primer comprises a sequence which is substantially identical to a portion of N₉ and the second primer comprises a sequence which is substantially complementary to a portion of N₁₀, or (ii) the first primer comprises a sequence which is substantially identical to a portion of the complement of N₉ and the second primer comprises a sequence which is substantially complementary to a portion of the complement of N₁₀. The primer pair amplifies a template sequence which bridges the end of the alu interruption.

In other preferred kits for detecting PCAV products containing alu-interrupted env, either (i) the first primer comprises a first sequence which is substantially identical to a portion of N₉ and a second sequence which is substantially identical to a portion of N₁₀, and the second primer comprises a sequence which is substantially complementary to a portion of an upstream or downstream PCAV sequence, or (ii) the first primer comprises a first sequence which is substantially identical to a portion of the complement of N₉ and a second sequence which is substantially identical to a portion of the complement of N₁₀, and the second primer comprises a sequence which is substantially complementary to the complement of an upstream or downstream PCAV sequence. The first primer hybridizes only to targets which contain the alu-interrupted env.

Another prefer-red kit comprises either (i) a first primer comprising a sequence which is substantially identical to a first portion of SEQ ID 111, 112 or 53 and a second primer comprising a sequence which is substantially complementary to a second portion of SEQ II) 111, 112 or 53, or (ii) a first primer comprising a sequence which is substantially identical to a first portion of the complement of SEQ ID 111, 112 or 53 and a second primer comprising a sequence which is substantially complementary to a second portion of the complement of SEQ ID 111, 112 or 53, such that the primer pair defines a template sequence within, consisting of or comprising SEQ ID 111, 112 or 53.

B.6—General Features of Nucleic Acids of the Invention

Nucleic acids and transcripts of the invention are preferably provided in isolated or substantially isolated form i.e. substantially free from other nucleic acids (e.g. free from naturally-occurring nucleic acids), generally being at least about 50% pure (by weight), and usually at least about 90% pure.

Nucleic acids of the invention can take various forms.

Nucleic acids of the invention may be single-stranded or double-stranded. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention may be circular or branched, but will generally be linear.

Nucleic acid of the invention may be attached to a solid support (e.g. a bead, plate, filter, film, slide, microarray support, resin, etc.)

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

Nucleic acids of the invention may be carry a detectable label e.g. a radioactive or fluorescent label, or a biotin label. This is particularly useful where the nucleic acid is to be used in nucleic acid detection techniques e.g. where the nucleic acid is a probe or a primer.

Nucleic acids of the invention comprise PCAV sequences, but they may also comprise non-PCAV sequences (e.g. in nucleic acids of formula 5′-X-Y-Z-3′, as defined above). This is particularly useful for primers, which may thus comprise a first sequence complementary to a PCAV nucleic acid target and a second sequence which is not complementary to the nucleic acid target. Any such non-complementary sequences in the primer are preferably 5′ to the complementary sequences. Typical non-complementary sequences comprise restriction sites {26} or promoter sequences {27}.

Nucleic acids of the invention can be prepared in many ways e.g. by chemical synthesis (at least in part), by digesting longer nucleic acids using nucleases (e.g. restriction enzymes), by joining shorter nucleic acids (e.g. using ligases or polymerases), from genomic or cDNA libraries, etc.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. The term “nucleic acid” is not intended to be limiting as to the length or structure of a nucleic acid unless specifically indicated, and the following are non-limiting examples of nucleic acids: a gene or gene fragment, mRNA, tRNA, rRNA, ribozymes, cDNA, recombinant nucleic acids, branched nucleic acids, plasmids, vectors, DNA from any source, RNA from any source, probes, and primers. Where nucleic acid of the invention takes the form of RNA, it may have a 5′ cap.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “CU” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T). The terms also imply a direction—the complement of 5′-ACAGT-3′ is 5′-ACTGT-3′ rather than 5′-TGTCA-3′.

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides. The nucleic acids are preferably uses to detect PCAV nucleic acid targets such as PCAV mRNAs.

References to a percentage sequence identity between two nucleic acid sequences mean that, when aligned, that percentage of bases are the same in comparing the two sequences. This alignment and the percent homology or sequence identity can be determined using software programs known in the art, for example those described in section 7.7.18 of reference 28. A preferred alignment program is GCG Gap (Genetics Computer Group, Wisconsin, Suite Version 10.1), preferably using default parameters, which are as follows: open gap=3; extend gap=1.

The percentage values of a, aa, b, bbb, c, ccC, d, e, eee, f, fff, g, h, hh, i, ii, j, kkk, mm, mmm, n, nn, nnn, pp, qq, qqq, rrr, s, uu, vv and ww as used above may each independently be 50, 55, 60, 65, 70, 75, 80, 85, 90, 91, 92, 93, 94, 95, 96, 97, 98, 99, 99.5, 99.9 or 100. The values of each of a, aa, b, bbb, c, ccc, d, e, eee, f, fff, g, h, hh, i, ii, j, mm, n, nn, pp, qq, s, uu, vv and ww may be the same or different as each other. Nucleic acid sequences which include ‘silent’ changes (i.e. which do not affect the encoded amino acid for a codon) are examples of these nucleic acids.

The values of ddd, ggg, hhh, jj, jjj, k, kk, l, ll, m, p, ppp, q, r, rr, ss, t, tt, u, v, w and y as used above may each independently be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100 or more. The values of each of ddd, ggg, jj, k, kk, l, ll, m, p, q, r, rr, ss, t, tt, u, v, w and y may be the same or different as each other.

The value of x+z is at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100 etc.). It is preferred that the value of x+y+z is at least 8 (e.g. at least 9, 10, 11, 12, 13, 14, 15, 16, 17, 18, 19, 20, 21, 22, 23, 24, 25, 30, 35, 40, 45, 50, 60, 70, 80, 90, 100 etc.). It is preferred that the value of x+y+z is at most 500 (e.g. at most 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8).

The percentage values of bb and cc as used above are independently each preferably less than 60 (e.g. 50, 40, 30, 20, 10), or may even be 0. The values of bb and cc may be the same or different as each other.

Preferred nucleic acids of the invention comprise nucleotide sequences which remain unmasked following application of a masking program for masking low complexity (e.g. XBLAST).

Where a nucleic acid is said to “encode” a polypeptide, it is not necessarily implied that the polynucleotide is translated, but it will include a series of codons which encode the amino acids of the polypeptide.

It is preferred that the invention does not encompass: (i) nucleic acid comprising a nucleotide sequence disclosed in reference 1; (ii) nucleic acid comprising a nucleotide sequence within SEQ IDs 1 to 225 in reference 1; (iii) a known nucleic acid; (iv) nucleic acid comprising SEQ ID 505, 506, 507, 508 or 509 from reference 29; (v) nucleic acid comprising SEQ ID 407 from references 30, 31 or 32; (vi) nucleic acid comprising SEQ ID 591 from references 30, 31 or 32; (vii) nucleic acid comprising SEQ ID 2192 from reference 33; (viii) nucleic acid comprising diagnostic protein #19115 from reference 34; (ix) nucleic acid comprising SEQ ID 37169 from reference 35; (x) nucleic acid comprising probe nos. 11882, 12335, 12181, 11701 or 24114 from reference 36; (xi) nucleic acid comprising probe nos. 9239 or 9663 from reference 37; (xii) nucleic acid comprising SEQ ID 12094 or 12516 from reference 38; (xiii) nucleic acid comprising SEQ ID 12377 or 12795 from reference 39; (xiv) nucleic acid comprising probe nos. 8509, 8960 or 17545 from reference 40; (xv) nucleic acid comprising probe nos. 12376, 12685, 12194, 25151 or 25457 from reference 41; (xvi) nucleic acid comprising nucleic acid 4609 from reference 42; (xvii) nucleic acid comprising SEQ ID 3685, 12135 or 13658 from reference 43; (xviii) a nucleic acid known as of 7th Dec. 2001 (e.g. a nucleic acid whose sequence is available in a public database such as GenBank or GeneSeq before 7th Dec. 2001); or (xix) a nucleic acid known as of 10th Jun. 2002 (e.g. a nucleic acid whose sequence is available in a public database such as GenBank or GeneSeq before 10th Jun. 2002).

C—Detecting Polypeptide Expression Products

Where the method is based on polypeptide detection, it will involve detecting expression of a polypeptide encoded by a PCAV mRNA transcript. This will typically involve detecting one or more of the following polypeptides: gag (e.g. SEQ ID 57) or PCAP3/mORF (e.g. SEQ ID 87). Although some PCAV mRNAs encode all of these polypeptides (e.g. ERVK6 {44}), PCAV is an old virus and its prt, pol and env genes are highly fragmented.

The transcripts which encode HML-2 polypeptides are generated by alternative splicing of the full-length mRNA copy of the endogenous genome {e.g. FIG. 4 of ref. 45, FIG. 1 of ref. 54}. PCAV gag polypeptide is encoded by the first long ORF in the genome (nucleotides 2813-4683 of SEQ ID 1; SEQ ID 54). Full-length gag polypeptide is proteolytically cleaved. PCAV prt polypeptide is encoded by the second long ORF in the genome and is translated as a gag-prt fusion polypeptide which is proteolytically cleaved to give the protease. PCAV pol polypeptide is encoded by the third long ORF in the genome and is translated as a gag-prt-pol fusion polypeptide which is proteolytically cleaved to give three pol products—reverse transcriptase, endonuclease and integrase {46}. PCAV env polypeptide is encoded by the fourth long ORF in the genome. The translated polypeptide is proteolytically cleaved. PCAV cORF polypeptide is encoded by an ORF which shares the same 5′ region and start codon as env, but in which a splicing event removes env-coding sequences and shifts to a reading frame +1 relative to that of env {47, 48}. PCAP3 polypeptide is encoded by an ORF which shares the same 5′ region and start codon as env, but in which a splicing event removes env-coding sequences and shifts to a reading frame +2 relative to that of env (the third reading frame).

C.1—Direct Detection of HML-2 Polypeptides

Various techniques are available for detecting the presence or absence of a particular polypeptides in a sample. These are generally immunoassay techniques which are based on the specific interaction between an antibody and an antigenic amino acid sequence in the polypeptide. Suitable techniques include standard immunohistological methods, ELISA, RIA, FIA, immunoprecipitation, immunofluorescence, etc.

Polypeptides of the invention can also be detected by functional assays e.g. assays to detect binding activity or enzymatic activity. For instance, functional assays for cORF are disclosed in references 48 to 50, and a functional assay for the protease is disclosed in reference 51. PCAP3 has been found to cause apoptosis in primary prostate epithelial cells and, when apoptosis is suppressed, to enable cells to expand beyond their normal senescence point.

Another way of detecting polypeptides of the invention is to use standard proteomics techniques e.g. purify or separate polypeptides and then use peptide sequencing. For example, polypeptides can be separated using 2D-PAGE and polypeptide spots can be sequenced (e.g. by mass spectroscopy) in order to identify if a sequence is present in a target polypeptide.

Techniques may require the enrichment of target polypeptides prior to detection. However, immunofluorescence assays can be easily performed on cells without the need for such enrichment. Cells may first be fixed onto a solid support, such as a microscope slide or microtiter well. The membranes of the cells can then be permeablized in order to permit entry of antibody (NB: fixing and permeabilization can be achieved together). Next, the fixed cells can be exposed to fluorescently-labeled antibody which is specific for the polypeptide. The presence of this label identifies cells which express the target PCAV polypeptide. To increase the sensitivity of the assay, it is possible to use a second antibody to bind to the anti-PCAV antibody, with the label being carried by the second antibody. {52}

C.2—Indirect Detection of HML-2 Polypeptides

Rather than detect polypeptides directly, it may be preferred to detect molecules which are produced by the body in response to a polypeptide (i.e. indirect detection of a polypeptide). This will typically involve the detection of antibodies, so the patient sample will generally be a blood sample. Antibodies can be detected by conventional immunoassay techniques e.g. using PCAV polypeptides of the invention, which will typically be immobilized.

Antibodies against HERV-K polypeptides have been detected in humans {e.g. 45, 53, 54} e.g. in seminoma or teratocarcinoma tissue.

C.3-Polypeptide Materials

The invention provides polypeptides which can be used in detection methods of the invention, wherein the polypeptides are encoded by a human endogenous retrovirus located at megabase 20.428 on chromosome 22.

The invention provides a polypeptide comprising an amino acid sequence selected from the group consisting of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188. SEQ IDs 54, 55, 56, 87, 98 and 110 are preferred members of this group.

The invention also provides (a) a polypeptide comprising a fragment of at least dd amino acids of one or more of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188, and (b) a polypeptide comprising an amino acid sequence having at least ee % identity to one or more of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188. These polypeptides include variants (e.g. allelic variants, homologs, orthologs, mutants, etc.).

The fragment of (a) may comprise a T-cell or, preferably, a B-cell epitope of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188. T- and B-cell epitopes can be identified empirically (e.g. using PEPSCAN {55, 56} or similar methods), or they can be predicted (e.g. using the Jameson-Wolf antigenic index {57}, matrix-based approaches {58}, TEPITOPE {59}, neural networks {60}, OptiMer & EpiMer {61, 62}, ADEPT {63}, Tsites {64}, hydrophilicity {65}, antigenic index {66} or the methods disclosed in reference 67 etc.

Preferred fragments of (a) are SEQ IDs 55, 56 and 110, or are fragments of SEQ IDs 55, 56 or 110. SEQ IDs 55, 56 & 110 are found within the PCAV gag protein and are particularly useful for detecting PCAV expression above background expression of other HERV-Ks.

Within (b), the polypeptide may, compared to SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188, comprise one or more conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cystine, serine, threonine, tyrosine. Phenylalanine, tryptophan, and tyrosine are sometimes classified jointly as aromatic amino acids. In general, substitution of single amino acids within these families does not have a major effect on the biological activity.

The invention also provides a polypeptide having formula NH₂—XX—YY-ZZ-COOH, wherein: XX is a polypeptide sequence consisting of xx amino acids; ZZ is a polypeptide sequence consisting of zz amino acids; YY is a polypeptide sequence consisting of a fragment of yy amino acids of an amino acid sequence selected from the group consisting of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188; and said polypeptide NH₂—XX—YY-ZZ-COOH is not a fragment of a polypeptide sequence selected from SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188.

The sequence of —XX— preferably shares less than ff % sequence identity to the xx amino acids which are N-terminus to sequence —YY— in SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188. The sequence of -ZZ- preferably shares less than gg % sequence identity to the zz amino acids which are C-terminus to sequence —YY— in SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188.

Polypeptides of the invention can be prepared in various forms (e.g. native, fusions, glycosylated, non-glycosylated, myristoylated, non-myristoylated, lipdated, non-lipidated, monomeric, multimeric, particulate, denatured, etc.).

Polypeptides of the invention may be attached to a solid support.

Polypeptides of the invention may comprise a detectable label (e.g. a radioactive or fluorescent label, or a biotin label).

Polypeptides of the invention can be prepared in many ways e.g. by chemical synthesis (at least in part), by digesting longer polypeptides using proteases, by translation from RNA, by purification from cell culture (e.g. from recombinant expression), from the organism itself (e.g. isolation from prostate tissue), from a cell line source etc.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

In general, the polypeptides of the invention are provided in a non-naturally occurring environment e.g. they are separated from their naturally-occurring environment. In certain embodiments, the polypeptide is present in a composition that is enriched for the polypeptide as compared to a control. Polypeptides of the invention are thus preferably provided in isolated or substantially isolated form i.e. the polypeptide is present in a composition that is substantially free of other expressed polypeptides, where by substantially free is meant that less than 75% (by weight), preferably less than 50%, and more preferably less than 10% (e.g. 5%) of the composition is made up of other expressed polypeptides.

Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the polypeptide (e.g. a functional domain and/or, where the polypeptide is a member of a polypeptide family, a region associated with a consensus sequence). Selection of amino acid alterations for production of variants can be based upon the accessibility (interior vs. exterior) of the amino acid (e.g. ref 68), the thermostability of the variant polypeptide (e.g. ref. 69), desired glycosylation sites (e.g. ref. 70), desired disulfide bridges (e.g. refs. 71 & 72), desired metal binding sites (e.g. refs. 73 & 74), and desired substitutions with in proline loops (e.g. ref. 75). Cysteine-depleted muteins can be produced as disclosed in reference 76.

The percentage value of ee as used above may be 50, 60, 65, 70, 75, 80, 85, 90, 91, 92, 93, 94, 95, 96, 97, 98, 99, 99.5, 99.9 or 100.

The percentage values of ff and gg as used above are independently each preferably less than 60 (e.g. 50, 40, 30, 20, 10), or may even be 0. The values of ff and gg may be the same or different as each other.

The values of dd, xx, yy and zz as used above may each independently be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 75, 80, 90, 100 or more. The values of each of dd, xx, yy and zz may be the same or different as each other. The value of dd may be less than 2000 (e.g. less than 1000, 500, 100, or 50).

The value of xx+zz is at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100 etc.). It is preferred that the value of xx+yy+zz is at least 8 (e.g. at least 9, 10, 11, 12, 13, 14, 15, 16, 17, 18, 19, 20, 21, 22, 23, 24, 25, 30, 35, 40, 45, 50, 60, 70, 80, 90, 100 etc.). It is preferred that the value of xx+yy+zz is at most 500 (e.g. at most 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8).

Polypeptides of the invention are generally at least 7 amino acids in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 amino acids or longer).

For certain embodiments of the invention, polypeptides are preferably at most 500 amino acids in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 amino acids or shorter).

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of reference 28. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is taught in reference 77.

Preferred polypeptides of the invention comprise amino acid sequences which remain unmasked following application of a masking program for masking low complexity (e.g. XBLAST).

It is preferred that the invention does not encompass: (i) polypeptides comprising an amino acid sequence disclosed in reference 1; (ii) polypeptides comprising an amino acid sequence within SEQ IDs 1 to 225 in reference 1; (iii) a polypeptide comprising SEQ ID 592 from references 30, 30 or 32; (iv) a known polypeptide; (v) a polypeptide known as of 7th Dec. 2001 (e.g. a polypeptide whose sequence is available in a public database such as GenBank or GeneSeq before 7th Dec. 2001); or (vi) a polypeptide known as of 10th Jun. 2002 (e.g. a polypeptide whose sequence is available in a public database such as GenBank or GeneSeq before 10th Jun. 2002).

C.4—Antibody Materials

The invention provides antibody that binds to a polypeptide of the invention. The invention also provides antibody that binds to a polypeptide encoded by a nucleic acid of the invention.

Preferred antibodies of the invention recognize epitopes within SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188. More preferred antibodies of the invention recognize epitopes within SEQ IDs 54, 55, 56 or 110.

Other preferred antibodies of the invention recognize a HERV-K gag protein. The antibody may (a) recognize gag from PCAV and also from one or more further HERV-Ks, (b) recognize gag from PCAV but not from any other HERV-Ks, (c) recognize gag from PCAV and also from one or more old HERV-Ks, but not from new HERV-Ks, or (d) recognize gag from one or more HERV-Ks but not from PCAV. A preferred antibody in group (a) is 5G2; a preferred antibody in group (c) is 5A5.

Antibodies of the invention may be polyclonal or monoclonal.

Antibodies of the invention may be produced by any suitable means e.g. by recombinant expression, or by administering (e.g. injecting) a polypeptide of the invention to an appropriate animal (e.g. a rabbit, hamster, mouse or other rodent).

Antibodies of the invention may include a label. The label may be detectable directly, such as a radioactive or fluorescent label. Alternatively, the label may be detectable indirectly, such as an enzyme whose products are detectable (e.g. luciferase, β-galactosidase, peroxidase etc.).

Antibodies of the invention may be attached to a solid support.

In general, antibodies of the invention are provided in a non-naturally occurring environment e.g. they are separated from their naturally-occurring environment. In certain embodiments, the antibodies are present in a composition that is, enriched for them as compared to a control. Antibodies of the invention are thus preferably provided in isolated or substantially isolated form i.e. the antibody is present in a composition that is substantially free of other antibodies, where by substantially free is meant that less than 75% (by weight), preferably less than 50%, and more preferably less than 10% (e.g. 5%) of the composition is made up of other antibodies.

The term “antibody” includes any suitable natural or artificial immunoglobulin or derivative thereof. In general, the antibody will comprise a Fv region which possesses specific antigen-binding activity. This includes, but is not limited to: whole immunoglobulins, antigen-binding immunoglobulin fragments (e.g. Fv, Fab, F(ab′)₂ etc.), single-chain antibodies (e.g. scFv), oligobodies, chimeric antibodies, humanized antibodies, veneered antibodies, etc.

To increase compatibility with the human immune system, the antibodies may be chimeric or humanized {e.g. refs. 78 & 79}, or fully human antibodies may be used. Because humanized antibodies are far less immunogenic in humans than the original non-human monoclonal antibodies, they can be used for the treatment of humans with far less risk of anaphylaxis. Thus, these antibodies may be preferred in therapeutic applications that involve in vivo administration to a human such as, use as radiation sensitizers for the treatment of neoplastic disease or use in methods to reduce the side effects of cancer therapy.

Humanized antibodies may be achieved by a variety of methods including, for example: (1) grafting non-human complementarity determining regions (CDRs) onto a human framework and constant region (“humanizing”), with the optional transfer of one or more framework residues from the non-human antibody; (2) transplanting entire non-human variable domains, but “cloaking” them with a human-like surface by replacement of surface residues (“veneering”). In the present invention, humanized antibodies will include both “humanized” and “veneered” antibodies. {refs. 80 to 86}. CDRs are amino acid sequences which together define the binding affinity and specificity of a Fv region of a native immunoglobulin binding site {e.g. 87 & 88}.

The phrase “constant region” refers to the portion of the antibody molecule that confers effector functions. In chimeric antibodies, mouse constant regions are substituted by human constant regions. The constant regions of humanized antibodies are derived from human immunoglobulins. The heavy chain constant region can be selected from any of the 5 isotypes: alpha, delta, epsilon, gamma or mu, and thus antibody can be of any isotype (e.g. IgG, IgA, IgM, IgD, IgE). IgG is preferred, which may be of any subclass (e.g. IgG₁, IgG₂).

Humanized or fully-human antibodies can also be produced using transgenic animals that are engineered to contain human immunoglobulin loci. For example, ref 89 discloses transgenic animals having a human Ig locus wherein the animals do not produce functional endogenous immunoglobulins due to the inactivation of endogenous heavy and light chain loci. Ref. 90 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. Ref. 91 discloses the use of the Cre/Lox system to modify the immunoglobulin locus in a mammal, such as to replace all or a portion of the constant or variable region to form a modified antibody molecule. Ref. 92 discloses non-human mammalian hosts having inactivated endogenous Ig loci and functional human Ig loci. Ref. 93 discloses methods of making transgenic mice in which the mice lack endogenous heavy chains, and express an exogenous immunoglobulin locus comprising one or more xenogeneic constant regions.

Using a transgenic animal described above, an immune response can be produced to a PCAV polypeptide, and antibody-producing cells can be removed from the animal and used to produce hybridomas that secrete human monoclonal antibodies. Immunization protocols, adjuvants, and the like are known in the art, and are used in immunization of, for example, a transgenic mouse as described in ref. 94. The monoclonal antibodies can be tested for the ability to inhibit or neutralize the biological activity or physiological effect of the corresponding polypeptide.

It is preferred that the invention does not encompass: (i) antibodies which recognize a polypeptide disclosed in reference 1; (ii) antibodies which recognize a polypeptide comprising an amino acid sequence within SEQ IDs 1 to 225 in reference 1; (iii) known antibodies; (iv) an antibody known as of 7th Dec. 2001 (e.g. a polypeptide whose sequence is available in a public database such as GenBank or GeneSeq before 7th Dec. 2001); or (v) an antibody known as of 10th Jun. 2002 (e.g. a polypeptide whose sequence is available in a public database such as GenBank or GeneSeq before 10th Jun. 2002).

D—Patient Samples and Normal Samples

D.1—The Patient Sample

Where the diagnostic method of the invention is based on detecting mRNA expression, the patient sample will generally comprise cells (e.g. prostate cells, particularly those from the luminal epithelium). These may be present in a sample of tissue (e.g. prostate tissue), or may be cells which have escaped into circulation (e.g. during metastasis). Instead of or as well as comprising prostate cells, the sample may comprise virions which contain PCAV mRNA.

Where the diagnostic method of the invention is based on detecting polypeptide expression, the patient sample may comprise cells, preferably, prostate cells and/or virions (as described above for mRNA), or may comprise antibodies which recognize PCAV polypeptides. Such antibodies will typically be present in circulation.

In general, therefore, the patient sample is tissue sample, preferably, a prostate sample (e.g. a biopsy) or a blood sample. Other possible sources of patient samples include isolated cells, whole tissues, or bodily fluids (e.g. blood, plasma, serum, urine, pleural effusions, cerebro-spinal fluid, etc.). Another preferred patient sample is a semen sample.

The patient is generally a human, preferably a human male, and more preferably an adult human male.

Expression products may be detected in the patient sample itself, or may be detected in material derived from the sample (e.g. the supernatant of a cell lysate, a RNA extract, cDNA generated from a RNA extract, polypeptides translated from a RNA extract, cells derived from culturing cells extracted from a patient etc.). These are still considered to be “patient samples” within the meaning of the invention.

Detection methods of the invention can be conducted in vitro or in vivo.

D.2—Controls

PCAV transcripts are up-regulated in prostate tumors. To detect such up-regulation, a reference point is typically needed i.e. a control. Analysis of the control sample gives a standard level of mRNA and/or protein expression against which a patient sample can be compared. As PCAV transcription is negligible in normal cells and highly up-regulated in tumor cells, however, a reference point may not always be necessary—significant expression indicates disease. Even so, the use of controls is preferable, particularly for standardization or for quantitative assays.

A negative control gives a background or basal level of expression against which a patient sample can be compared. Higher levels of expression product relative to a negative control indicate that the patient from whom the sample was taken has a prostate tumor. Conversely, equivalent levels of expression product indicate that the patient does not have a PCAV-related cancer.

A negative control will generally comprise material from cells which are not tumor cells. The negative control could be a sample from the same patient as the patient sample, but from a tissue in which PCAV expression is not up-regulated e.g. a non-tumor non-prostate cell. The negative control could be a prostate cell from the same patient as the patient sample, but taken at an earlier stage in the patient's life (e.g. before the development of cancer, or from a BPH patient). The negative control could be a cell from a patient without a prostate tumor, and this cell may or may not be a prostate cell. The negative control could be a suitable cell line. Typically, the negative control will be the same tissue or cell type as the patient sample being tested (e.g. a prostate cell or a blood sample).

A positive control gives a level of expression against which a patient sample can be compared. Equivalent or higher levels of expression product relative to a positive control indicate that the patient from whom the sample was taken has a prostate tumor. Conversely, lower levels of expression product indicate that the patient does not have a PCAV-related tumor.

A positive control will generally comprise material from tumor cells or from a blood sample taken from a patient known to have a tumor. The positive control could be a prostate tumor cell from the same patient as the patient sample, but taken at an earlier stage in the patient's life (e.g. to monitor remission). The positive control could be a cell from another patient with a prostate tumor. The positive control could be a suitable prostate cell line.

Other suitable positive and negative controls will be apparent to the skilled person.

PCAV expression in the control can be assessed at the same time as expression in the patient sample. Alternatively, PCAV expression in the control can be assessed separately (earlier or later). Rather than actually compare two samples, however, the control may be an absolute value i.e. a level of expression which has been empirically determined from samples taken from prostate tumor patients (e.g. under standard conditions). Examples of such negative controls for prostate tumors include lifetime baseline levels of expression or the expression level e.g. as observed in pooled normals.

D.3—Degree of Up-Regulation

The up-regulation relative to the control (100%) will usually be at least 150% (e.g. 200%, 250%, 300%, 400%, 500%, 600% or more). A twenty- to forty-fold up-regulation is not uncommon.

E—Diagnostic Methods and Diagnosis

The invention provides a method for diagnosing prostate cancer, comprising the step of detecting in a patient sample the presence or absence of an expression product of a human endogenous retrovirus located at megabase 20.428 on chromosome 22.

E.1—Products for Use in Diagnosis

Preferred expression products for detection in diagnostic methods of the invention are described in sections B.1, B.3 and C.3 above.

Preferred reagents for use in diagnostic methods of the invention are described in sections B.4, C.3 and C.4 above.

Preferred kits for use in diagnostic methods of the invention are described in section B.5 above.

The invention provides nucleic acids, polypeptides and antibodies of the invention for use in diagnosis.

The invention also provides the use of nucleic acids, polypeptides and antibodies of the invention in the manufacture of diagnostic assays.

E.2—mRNA-Based Methods of the Invention

The invention provides a method for analyzing a patient sample, comprising the steps of: (a) contacting the patient sample with nucleic acid of the invention under hybridizing conditions; and (b) detecting the presence or absence of hybridization of nucleic acid of the invention to nucleic acid present in the patient sample. The presence of hybridization in step (b) indicates that the patient from whom the sample was taken has a prostate tumor.

The invention also provides a method for analyzing a patient sample, comprising the steps of: (a) enriching mRNA in the sample relative to DNA to give a mRNA-enriched sample; (b) contacting the mRNA-enriched sample with nucleic acid of the invention under hybridizing conditions; and (c) detecting the presence or absence of hybridization of nucleic acid of the invention to mRNA present in the mRNA-enriched sample. The presence of hybridization in step (c) indicates that the patient from whom the sample was taken has a prostate tumor. The enrichment in step (a) may take the form of extracting mRNA without extracting DNA, removing DNA without removing mRNA, or disrupting PCAV DNA without disrupting PCAV mRNA etc. (see section B.2 above).

The invention also provides a method for analyzing a patient sample, comprising the steps of: (a) preparing DNA copies of mRNA in the sample; (b) contacting the DNA copies with nucleic acid of the invention under hybridizing conditions; and (c) detecting the presence or absence of hybridization of nucleic acid of the invention to said DNA copies. The presence of hybridization in step (c) indicates that the patient from whom the sample was taken has a prostate tumor. Preparation of DNA in step (a) may be specific to PCAV (e.g. by using RT-PCR with appropriate primers) or may be non-specific (e.g. preparation of cellular cDNA).

In the above methods for analyzing a patient sample, the nucleic acid of the invention contacted with the sample may be a probe of the invention. As an alternative, it may comprise primers of the invention, in which case the relevant step of the method will generally involve two or more (e.g. 3, 4, 5, 6, 7, 8, 9, 10 or more) cycles of amplification. Where primers are used, the method may involve the use of a probe for detecting hybridization to amplified DNA.

The invention also provides a method for analyzing a patient sample, comprising the steps of: (a) amplifying any PCAV nucleic acid targets in the sample; and (b) detecting the presence or absence of amplified targets. The presence of amplified targets in step (b) indicates that the patient from whom the sample was taken has a prostate tumor.

These methods of the invention may be qualitative, quantitative, or semi-quantitative.

E.3—Polypeptide-Based Methods of the Invention

The invention provides an immunoassay method for diagnosing prostate cancer, comprising the step of contacting a patient sample with a polypeptide or antibody of the invention.

The invention also provides a method for analyzing a patient blood sample, comprising the steps of: (a) contacting the blood sample with a polypeptide of the invention; and (b) detecting the presence or absence of interaction between said polypeptide and antibodies in said sample. The presence of an interaction in step (b) indicates that the patient from whom the blood sample was taken has raised anti-PCAV antibodies, and thus that they have a prostate tumor. Step (a) may be preceded by a step wherein antibodies in the blood sample are enriched.

The invention also provides a method for analyzing a patient sample, comprising the steps of: (a) contacting the sample with antibody of the invention; and (b) detecting the presence or absence of interaction between said antibody and said sample. The presence of an interaction in step (b) indicates that the patient from whom the sample was taken is expressing PCAV polypeptides, and thus that they have a prostate tumor. Step (a) may be preceded by a step wherein cells in the sample are lysed or permeabilized and/or wherein polypeptides in the sample are enriched.

These methods of the invention may be qualitative, quantitative, or semi-quantitative.

The above methods may be adapted for use in vivo (e.g. to locate or identify sites where tumor cells are present). In these embodiments, an antibody specific for a target PCAV polypeptide is administered to an individual (e.g. by injection) and the antibody is located using standard imaging techniques (e.g. magnetic resonance imaging, computerized tomography scanning, etc.). Appropriate labels (e.g. spin labels etc.) will be used. Using these techniques, cancer cells are differentially labeled.

Other in vivo methods may detect PCAV polypeptides functionally. For instance, a construct comprising a PCAV LTR operatively linked to a reporter gene (e.g. a fluorescent protein such as GFP) will be expressed in parallel to native PCAV polypeptides.

To increase the sensitivity of immunoassays, it is possible to use a second antibody to bind to the anti-PCAV antibody, with a label being carried by the second antibody.

E.4—The Meaning of “Diagniosis”

The invention provides a method for diagnosing prostate cancer. It will be appreciated that “diagnosis” according to the invention can range from a definite clinical diagnosis of disease to an indication that the patient should undergo further testing which may lead to a definite diagnosis. For example, the method of the invention can be used as part of a screening process, with positive samples being subjected to further analysis.

Furthermore, diagnosis includes monitoring the progress of cancer in a patient already known to have the cancer. Cancer can also be staged by the methods of the invention. Preferably, the cancer is prostate cancer.

The efficacy of a treatment regimen (therametrics) of a cancer associated can also monitored by the method of the invention e.g. to determine its efficacy.

Susceptibility to a cancer can also be detected e.g. where up-regulation of expression has occurred, but before cancer has developed. Prognostic methods are also encompassed.

All of these techniques fall within the general meaning of “diagnosis” in the present invention.

F—Pharmaceutical Compositions

The invention provides a pharmaceutical composition comprising nucleic acid, polypeptide, or antibody of the invention. The invention also provides their use as medicaments, and their use in the manufacture of medicaments for treating prostate cancer. The invention also provides a method for raising an immune response, comprising administering an immunogenic dose of nucleic acid or polypeptide of the invention to an animal (e.g. to a patient).

Pharmaceutical compositions encompassed by the present invention include as active agent, the nucleic acids, polypeptides, or antibodies of the invention disclosed herein in a therapeutically effective amount. An “effective amount” is an amount sufficient to effect beneficial or desired results, including clinical results. An effective amount can be administered in one or more administrations. For purposes of this invention, an effective amount is an amount that is sufficient to palliate, ameliorate, stabilize, reverse, slow or delay the symptoms and/or progression of prostate cancer.

The compositions can be used to treat cancer as well as metastases of primary cancer. In addition, the pharmaceutical compositions can be used in conjunction with conventional methods of cancer treatment, e.g. to sensitize tumors to radiation or conventional chemotherapy. The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e. arresting its development; or (c) relieving the disease symptom, i.e. causing regression of the disease or symptom.

Where the pharmaceutical composition comprises an antibody that specifically binds to a gene product encoded by a differentially expressed nucleic acid, the antibody can be coupled to a drug for delivery to a treatment site or coupled to a detectable label to facilitate imaging of a site comprising cancer cells, such as prostate cancer cells. Methods for coupling antibodies to drugs and detectable labels are well known in the art, as are methods for imaging using detectable labels.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg of the compositions of the present invention in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g. mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in reference 95.

The composition is preferably sterile and/or pyrogen-free. It will typically be buffered at about pH 7.

Once formulated, the compositions contemplated by the invention can be (1) administered directly to the subject (e.g. as nucleic acid, polypeptides, small molecule agonists or antagonists, and the like); or (2) delivered ex vivo, to cells derived from the subject (e.g. as in ex vivo gene therapy). Direct delivery of the compositions will generally be accomplished by parenteral injection, e.g. subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art {e.g. ref. 96}. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the nucleic acid(s) in liposomes, and direct microinjection of the DNA into nuclei, all well known in the art.

Differential expression of PCAV nucleic acids has been found to correlate with prostate tumors. The tumor can be amenable to treatment by administration of a therapeutic agent based on the provided nucleic acid, corresponding polypeptide or other corresponding molecule (e.g. antisense, ribozyme, etc.). In other embodiments, the disorder can be amenable to treatment by administration of a small molecule drug that, for example, serves as an inhibitor (antagonist) of the function of the encoded gene product of a gene having increased expression in cancerous cells relative to normal cells or as an agonist for gene products that are decreased in expression in cancerous cells (e.g. to promote the activity of gene products that act as tumor suppressors).

The dose and the means of administration of the inventive pharmaceutical compositions are determined based on the specific qualities of the therapeutic composition, the condition, age, and weight of the patient, the progression of the disease, and other relevant factors. For example, administration of nucleic acid therapeutic compositions agents includes local or systemic administration, including injection, oral administration, particle gun or catheterized administration, and topical administration. Preferably, the therapeutic nucleic acid composition contains an expression construct comprising a promoter operably linked to a nucleic acid of the invention. Various methods can be used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. An antisense composition is directly administered to the surface of the tumor, for example, by topical application of the composition. X-ray imaging may be used to assist in certain of the above delivery methods.

Targeted delivery of therapeutic compositions containing an antisense nucleic acid, subgenomic nucleic acids, or antibodies to specific tissues can also be used. Receptor-mediated DNA delivery techniques are described in, for example, references 97 to 102. Therapeutic compositions containing a nucleic acid are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g. for enhancing or inhibiting levels of the encoded gene product) and efficacy of transformation and expression are considerations which will affect the dosage required for ultimate efficacy of the antisense subgenomic nucleic acids. Where greater expression is desired over a larger area of tissue, larger amounts of antisense subgenomic nucleic acids or the same amounts re-administered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect.

The therapeutic nucleic acids and polypeptides of the present invention can be delivered using gene delivery vehicles. The gene delivery vehicle can be of viral or non-viral origin (see generally references 103, 104, 105 and 106). Expression of such coding sequences can be induced using endogenous mammalian or heterologous promoters. Expression of the coding sequence can be either constitutive or regulated.

Viral-based vectors for delivery of a desired nucleic acid and expression in a desired cell are well known in the art. Exemplary viral-based vehicles include, but are not limited to, recombinant retroviruses (e.g. references 107 to 117), alphavirus-based vectors (e.g. Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR-1249; ATCC VR-532)), adenovirus vectors, and adeno-associated virus (AAV) vectors (e.g. see refs. 118 to 123). Administration of DNA linked to killed adenovirus {124} can also be employed.

Non-viral delivery vehicles and methods can also be employed, including, but not limited to, polycationic condensed DNA linked or unlinked to killed adenovirus alone {e.g. 124}, ligand-linked DNA {125}, eukaryotic cell delivery vehicles cells {e.g. refs. 126 to 130} and nucleic charge neutralization or fusion with cell membranes. Naked DNA can also be employed. Exemplary naked DNA introduction methods are described in refs. 131 and 132. Liposomes that can act as gene delivery vehicles are described in refs. 133 to 137. Additional approaches are described in refs. 138 & 139.

Further non-viral delivery suitable for use includes mechanical delivery systems such as the approach described in ref. 139. Moreover, the coding sequence and the product of expression of such can be delivered through deposition of photopolymerized hydrogel materials or use of ionizing radiation {e.g. refs. 140 & 141}. Other conventional methods for gene delivery that can be used for delivery of the coding sequence include, for example, use of hand-held gene transfer particle gun {142} or use of ionizing radiation for activating transferred genes {140 & 141}.

Vaccine Compositions

The pharmaceutical composition is preferably an immunogenic composition and is more preferably a vaccine composition. Such compositions can be used to raise antibodies in a mammal (e.g. a human).

The composition may additionally comprise an adjuvant. For example, the composition may comprise one or more of the following adjuvants: (1) oil-in-water emulsion formulations (with or without other specific immunostimulating agents such as muramyl peptides (see below) or bacterial cell wall components), such as for example (a) MF59™ {143; Chapter 10 in ref. 144}, containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (2) saponin adjuvants, such as QS21 or Stimulon™ (Cambridge Bioscience, Worcester, Mass.) may be used or particles generated therefrom such as ISCOMs (immunostimulating complexes), which ISCOMS may be devoid of additional detergent {145}; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) {e.g. 146, 147}; (6) combinations of 3dMPL with, for example, QS21 and/or oil-in-water emulsions {e.g. 148, 149, 150}; (7) oligonucleotides comprising CpG motifs i.e. containing at least one CG dinucleotide, with 5-methylcytosine optionally being used in place of cytosine; (8) a polyoxyethylene ether or a polyoxyethylene ester {151}; (9) a polyoxyethylene sorbitan ester surfactant in combination with an octoxynol {152} or a polyoxyethylene alkyl ether or ester surfactant in combination with at least one additional non-ionic surfactant such as an octoxynol {153}; (10) an immunostimulatory oligonucleotide (e.g. a CpG oligonucleotide) and a saponin {154}; (11) an immunostimulant and a particle of metal salt {155}; (12) a saponin and an oil-in-water emulsion {156}; (13) a saponin (e.g. QS21)+3dMPL+IL-12 (optionally+a sterol) {157}; (14) aluminium salts, preferably hydroxide or phosphate, but any other suitable salt may also be used (e.g. hydroxyphosphate, oxyhydroxide, orthophosphate, sulphate etc. {chapters 8 & 9 of ref. 144}). Mixtures of different aluminium salts may also be used. The salt may take any suitable form (e.g. gel, crystalline, amorphous etc.); (15) chitosan; (16) cholera toxin or E. coli heat labile toxin, or detoxified mutants thereof {158}; (17) microparticles of poly(a-hydroxy)acids, such as PLG; (18) other substances that act as immunostimulating agents to enhance the efficacy of the composition. Aluminium salts and/or MF59™ are preferred.

Vaccines of the invention may be prophylactic (i.e. to prevent disease) or therapeutic (i.e. to reduce or eliminate the symptoms of a disease).

Efficacy can be tested by monitoring expression of nucleic acids and/or polypeptides of the invention after administration of the composition of the invention.

G—Screening Methods and Drug Design

The invention provides methods of screening for compounds with activity against cancer, comprising: contacting a test compound with a tissue sample derived from a cell in which PCAV expression is up-regulated, or a cell line; and monitoring PCAV expression in the sample. A decrease in expression indicates potential anti-cancer efficacy of the test compound.

The invention also provides methods of screening for compounds with activity against prostate cancer, comprising: contacting a test compound with a nucleic acid or polypeptide of the invention; and detecting a binding interaction between the test compound and the nucleic acid/polypeptide. A binding interaction indicates potential anti-cancer efficacy of the test compound.

The invention also provides methods of screening for compounds with activity against prostate cancer, comprising: contacting a test compound with a polypeptide of the invention; and assaying the function of the polypeptide. Inhibition of the polypeptide's function (e.g. loss of protease activity, loss of RNA export, loss of reverse transcriptase activity, loss of endonuclease activity, loss of integrase activity etc.) indicates potential anti-cancer efficacy of the test compound.

Typical test compounds include, but are not restricted to, peptides, peptoids, proteins, lipids, metals, nucleotides, nucleosides, small organic molecules, antibiotics, polyamines, and combinations and derivatives thereof. Small organic molecules have a molecular weight of more than 50 and less than about 2,500 daltons, and most preferably between about 300 and about 800 daltons. Complex mixtures of substances, such as extracts containing natural products, or the products of mixed combinatorial syntheses, can also be tested and the component that binds to the target RNA can be purified from the mixture in a subsequent step.

Test compounds may be derived from large libraries of synthetic or natural compounds. For instance, synthetic compound libraries are commercially available from Maybridge Chemical Co. (Trevillet, Cornwall, UK) or Aldrich (Milwaukee, Wis.). Alternatively, libraries of natural compounds in the form of bacterial, fungal, plant and animal extracts may be used. Additionally, test compounds may be synthetically produced using combinatorial chemistry either as individual compounds or as mixtures.

Agonists or antagonists of the polypeptides of the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of an agonist or antagonist of a PCAV polypeptide. Such agonists and antagonists can be used to modulate, enhance, or inhibit PCAV expression and/or function. {159}

The present invention relates to methods of using the polypeptides of the invention to screen compounds for their ability to bind or otherwise modulate, such as, inhibit, the activity of PCAV polypeptides, and thus to identify compounds that can serve, for example, as agonists or antagonists of the PCAV polypeptides. In one screening assay, the PCAV polypeptide is incubated with cells susceptible to the growth stimulatory activity of PCAV, in the presence and absence of a test compound. The PCAV activity altering or binding potential of the test compound is measured. Growth of the cells is then determined. A reduction in cell growth in the test sample indicates that the test compound binds to and thereby inactivates the PCAV polypeptide, or otherwise inhibits the PCAV polypeptide activity.

Transgenic animals (e.g. rodents) that have been transformed to over-express PCAV genes can be used to screen compounds in vivo for the ability to inhibit development of tumors resulting from PCAV over-expression or to treat such tumors once developed. Transgenic animals that have prostate tumors of increased invasive or malignant potential can be used to screen compounds, including antibodies or peptides, for their ability to inhibit the effect of PCAV polypeptides. Such animals can be produced, for example, as described in the examples herein.

Screening procedures such as those described above are useful for identifying agents for their potential use in pharmacological intervention strategies in prostate cancer treatment. Additionally, nucleic acid sequences corresponding to PCAV, including LTRs, may be used to assay for inhibitors of elevated gene expression.

Antisense oligonucleotides complementary to PCAV mRNA can be used to selectively diminish or oblate the expression of the polypeptide. More specifically, antisense constructs or antisense oligonucleotides can be used to inhibit the production of PCAV polypeptide(s) in prostate tumor cells. Antisense mRNA can be produced by transfecting into target cancer cells an expression vector with a PCAV nucleic acid of the invention oriented in an antisense direction relative to the direction of PCAV-mRNA transcription. Appropriate vectors include viral vectors, including retroviral vectors, as well as non-viral vectors. Alternately, antisense oligonucleotides can be introduced directly into target cells to achieve the same goal. Oligonucleotides can be selected/designed to achieve the highest level of specificity and, for example, to bind to a PCAV-mRNA at the initiator ATG.

Monoclonal antibodies to PCAV polypeptides can be used to block the action of the polypeptides and thereby control growth of cancer cells. This can be accomplished by infusion of antibodies that bind to PCAV polypeptides and block their action.

The invention also provides high-throughput screening methods for identifying compounds that bind to a nucleic acid or polypeptide of the invention. Preferably, all the biochemical steps for this assay are performed in a single solution in, for instance, a test tube or microtitre plate, and the test compounds are analyzed initially at a single compound concentration. for the purposes of high throughput screening, the experimental conditions are adjusted to achieve a proportion of test compounds identified as “positive” compounds from amongst the total compounds screened. The assay is preferably set to identify compounds with an appreciable affinity towards the target e.g. when 0.1% to 1% of the total test compounds from a large compound library are shown to bind to a given target with a Kᵢ of 10 μM or less (e.g. 1 μM, 100 nM, 10 nM, or less).

H—Definitions

The term “comprising” means “including” as well as “consisting” e.g. a composition “comprising” X may consist exclusively of X or may include something additional e.g. X+Y.

The term “about” in relation to a numerical value x means, for example, x±10%.

The terms “neoplastic cells”, “neoplasia”, “tumor”, “tumor cells”, “cancer” and “cancer cells” (used interchangeably) refer to cells which exhibit relatively autonomous growth, so that they exhibit an aberrant growth phenotype characterized by a significant loss of control of cell proliferation (i.e. de-regulated cell division). Neoplastic cells can be malignant or benign and include prostate cancer derived tissue.

The word “substantially” does not exclude “completely” e.g. a composition which is “substantially free” from Y may be completely free from Y. Where necessary, the word “substantially” may be omitted from the definition of the invention.

### BRIEF DESCRIPTION OF DRAWINGS

FIG. 1 is a phylogenetic tree showing the relationship between various endogenous retroviral LTRs. “Old” and “new” HERV-K LTRs are highlighted.

FIG. 2 illustrates the arrangement the PCAV genome at its 5′ end.

FIG. 3 illustrates the arrangement the PCAV genome at its 3′ end.

FIG. 4 shows splicing events which take place in a prior art HERV-K (‘HTDV’ {45}) to produce env and cORF proteins.

FIG. 5 illustrates splicing events at the 5′ LTRs of PCAV.

FIG. 6 illustrates how splicing events at the tandem 5′ LTRs of PCAV (FIG. 6B) can be distinguished from those in other HERV-Ks (FIG. 6A).

FIG. 7 illustrates how primers can be used to specifically detect PCAV mRNA.

FIG. 8 illustrates how insertions at the 3′ end of PCAV can be exploited to distinguish it from other HERV-Ks.

FIG. 9 maps the location of positive array features to the PCAV genome.

FIG. 10 shows the results of RT-PCR analysis of the exon 1-2 splicing event in various tissues. Lanes are: (1) markers; (2) placenta; (3) & (4) brain; (5) testis; (6) prostate; (7) breast; (8) uterus; (9) thyroid; (10) cervix; and (11) lung.

FIG. 11 shows the results of RT-PCR analysis of the exon 1-2 splicing event in cell lines. Lanes are: (1) and (12) markers; (2) Teral; (3) colo360; (4) PC3; (5) DU145; (6) 22RV1; (7) PCA 2B; (8) LNCaP; (9) RWPE1; (10) RWPE2; and (11) PrEC.

FIG. 12 shows fluorescence results obtained using 5G2 monoclonal antibody against: (12B) MDA PCA 2b cells; (12C) PC3 cells; and (12D) NIH3T3 cells. FIG. 12A shows MDA PCA 2b cells without 5G2 antibody.

FIGS. 13 and 14 show staining of prostate tumor samples with (A) hematoxylin & eosin stained, (B) mAb 5G2 plus fluorescein-anti-mouse, or (C) fluorescein-anti-mouse only.

FIG. 15 shows expression of HERV-K gag proteins in yeast, with 15A being a stained protein gel and 15B being a western blot.

FIG. 16 shows western blots of gag proteins using eight monoclonal antibodies.

FIG. 17 is a not-to-scale schematic of certain SEQ IDs mapped against the genome.

FIG. 18 shows microarray analysis of PCAV expression in patient samples. In the expanded portion on the right, the headings indicate Gleason grades of the samples. Red identifies sequences up-regulated in cancer, green identifies those depressed in cancer, and black denotes unchanged spots. Individual sequences are arrayed vertically and patients are presented horizontally. The panel on the left shows all 6000 sequences assayed with RNA from 103 patients, and the region showing almost uniform up-regulation is expanded on the right.

FIG. 19 shows the sub-cellular localization of PCAP3 using immuno-staining.

FIG. 20 shows PIN staining using anti-gag immunofluorescence. A fresh frozen section of PIN tissue was used, and the assessment of PIN was made by a certified pathologist in an hemotoxylin and eosin stained serial section.

FIG. 21 shows TUNEL for cells transfected with PCAP3-encoding adenovirus at moi 100 (top left), 50 (top right), 25 (bottom left), or an untransfected control (bottom right).

FIG. 22 shows results from a cell division assay using bromo-deoxyuridine labeling.

FIG. 23 shows splicing within the PCAV genome, particularly for env, cORF & PCAP3.

FIG. 24 shows the adenovirus vector used in an expression assay to test for LTR activity, and FIG. 25 shows the results of GFP expression driven from this vector.

FIG. 26 shows the vector used to test the ability of PCAP3 to activate the PCAV LTR.

FIG. 27 shows immunofluorescence experiments using an anti-gag monoclonal antibody 5G2 to stain sections of tissue taken from a prostate cancer patient. FIG. 27A shows a normal prostate gland, 27B shows atrophied tissue, 27C shows a Gleason grade 3 cancer, and 27D shows a Gleason grade 4 cancer.

FIG. 28 shows the position of PCAV-specific primers (cf 5′ region of FIG. 2), and FIG. 29 shows the results of PCR using these primers. ‘P’ is prostate tissue and ‘B’ is breast tissue. FIG. 30 shows RT-PCR results using the primers. Pairs of matched normal (‘N’) or cancer (‘C’) prostate tissue was used, and the signal ratio is given above each pair.

FIG. 31 shows quantitative PCR results for various tissues. The y-axis shows PCAV levels normalized to HPRT. The tissues are, from left to right: placenta, fetal brain, fetal heart, fetal liver, brain, heart, liver, pancreas, stomach, small intestine, colon, rectum, testicle, prostate (47 year old man), ovary, adrenal, thyroid, kidney, bladder, breast, uterus, cervix, skeletal muscle, lung, spleen, thymus, skin.

FIG. 32 shows the age-related increase in PCAV mRNA expression in prostate tissue.

FIG. 33 shows the results of a RT-PCR scanning assay used to map the 5′ end of PCAV mRNAs.

FIG. 34 gives details of a RNase protection assay. Two antisense probes were used—a long probe (24B) and a short probe (24C). Both probes protected the region shown in 24A. In 24B, the position of the band expected based on the ‘usual’ 5′ end based on the position of the TATA signal is shown, plus the actual band achieved. The three lanes in 24B are: (1) Teral; (2) no RNA; (3) probe, no RNase. The two lanes in 24C are: (1) Teral; (2) probe, no RNase.

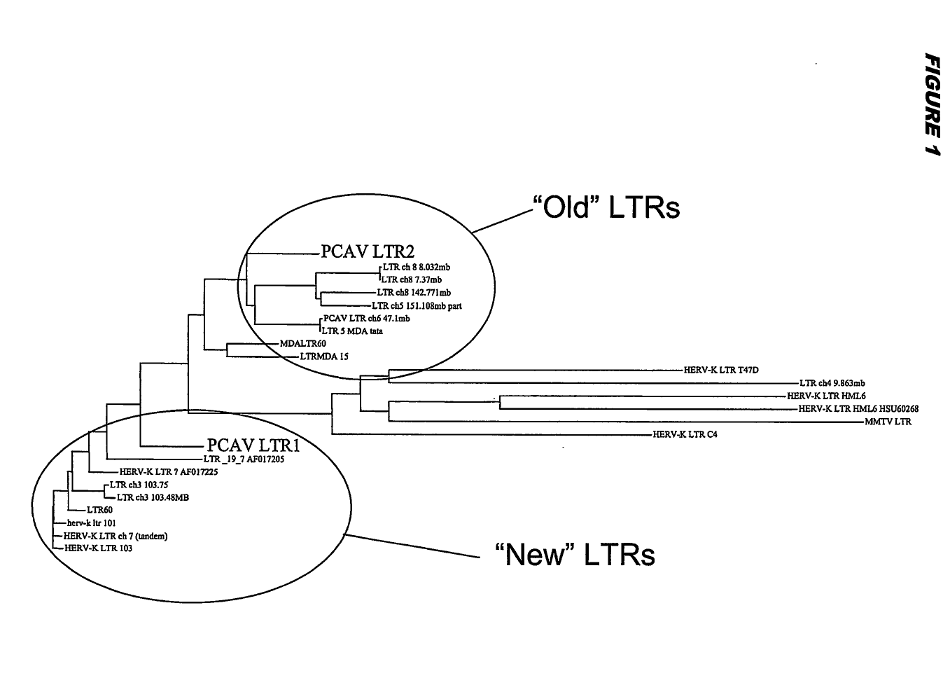

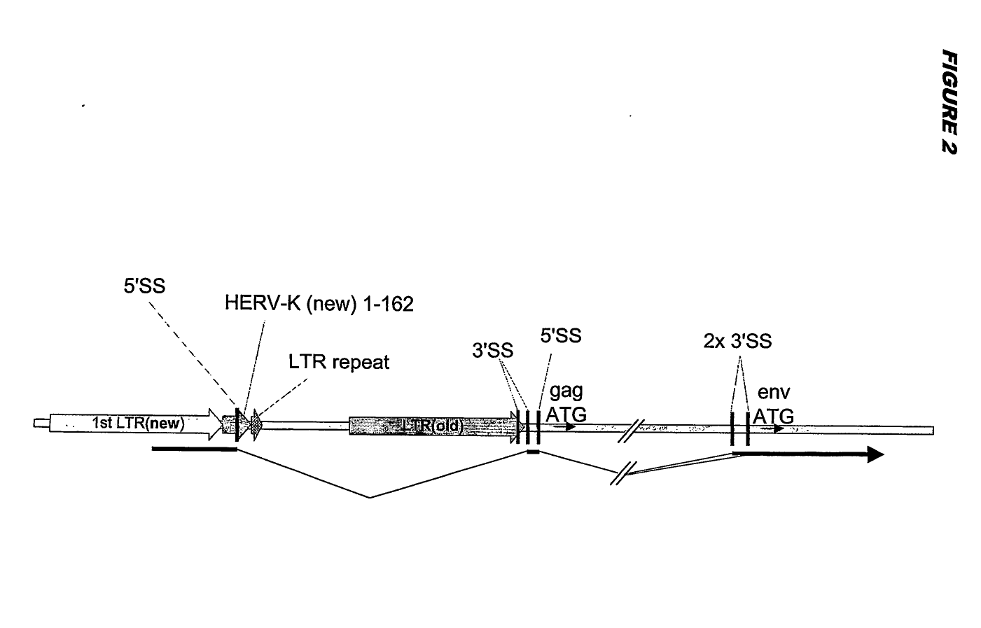

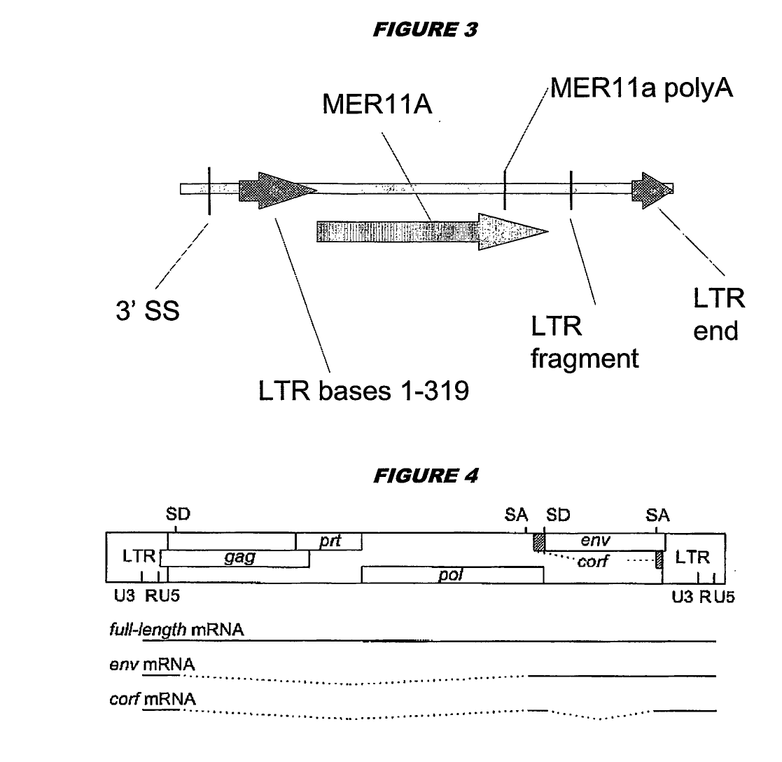

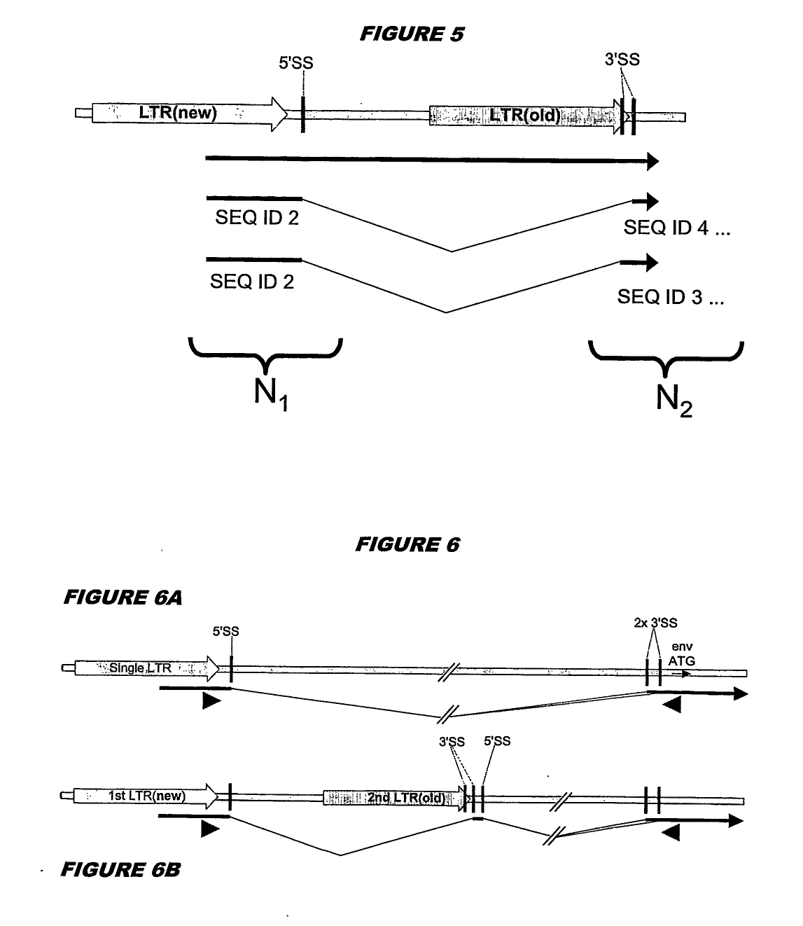

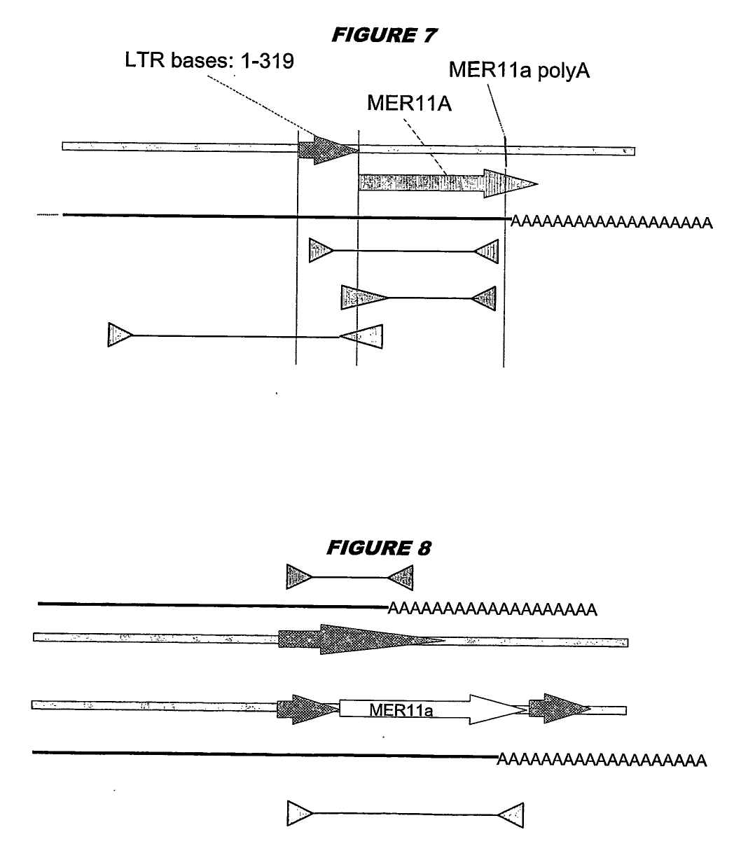

### MODES FOR CARRYING OUT THE INVENTION

Certain aspects of the present invention are described in greater detail in the non-limiting examples that follow. The examples are put forth so as to provide those of ordinary skill in the art with a disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

Source of Human Prostate Cell Samples and Isolation of Nucleic Acids Expressed by them

Candidate nucleic acids that may represent genes differentially expressed in cancer were obtained from both publicly-available sources and from cDNA libraries generated from selected cell lines and patient tissues. A normalized cDNA library was prepared from one patient tumor tissue and cloned nucleic acids for spotting on microarrays were isolated from the library. Normal and tumor tissues from 100 patients were processed to generate T7 RNA polymerase transcribed nucleic acids, which were, in turn, assessed for expression in the microarrays.

Normalization: The objective of normalization is to generate a cDNA library in which all transcripts expressed in a particular cell type or tissue are equally represented {refs. 160 & 161}, and therefore isolation of as few as 30,000 recombinant clones in an optimally normalized library may represent the entire gene expression repertoire of a cell, estimated to number 10,000 per cell. The source materials for generating the normalized prostate libraries were cryopreserved prostate tumor tissue from a patient with Gleason grade 3+3 adenocarcinoma and normal prostate biopsies from a pool of at-risk subjects under medical surveillance. Prostate epithelia were harvested directly from frozen sections of tissue by laser capture microdissection (LCM, Arcturus Engineering Inc., Mountain View, Calif.), carried out according to methods well known in the art (e.g. ref. 162), to provide substantially homogenous cell samples.

Total RNA was extracted from LCM-harvested cells using RNeasy™ Protect Kit (Qiagen, Valencia, Calif.), following manufacturer's recommended procedures. RNA was quantified using RiboGreen™ RNA quantification kit (Molecular Probes, Inc. Eugene, Oreg.). One μg of total RNA was reverse transcribed and PCR amplified using SMART™ PCR cDNA synthesis kit (ClonTech, Palo Alto, Calif.). The cDNA products were size-selected by agarose gel electrophoresis using standard procedures (ref. 21). The cDNA was extracted using Bio 101Geneclean® II kit (Qbiogene, Carlsbad, Calif.). Normalization of the cDNA was carried out using kinetics of hybridization principles: 1.0 μg of cDNA was denatured by heat at 100° C. for 10 minutes, then incubated at 42° C. for 42 hours in the presence of 120 mM NaCl, 10 mM Tris.HCl (pH=8.0), 5 mM EDTA.Na⁺ and 50% formamide. Single-stranded cDNA (“normalized” cDNA) was purified by hydroxyapatite chromatography (#130-0520, BioRad, Hercules, Calif.) following the manufacturer's recommended procedures, amplified and converted to double-stranded cDNA by three cycles of PCR amplification, and cloned into plasmid vectors using standard procedures (ref. 21). All primers/adaptors used in the normalization and cloning process are provided by the manufacturer in the SMART™ PCR cDNA synthesis kit (ClonTech, Palo Alto, Calif.). Supercompetent cells (XL-2 Blue Ultracompetent Cells, Stratagene, Calif.) were transfected with the normalized cDNA libraries, plated on plated on solid media and grown overnight at 36° C.

Characterization of normalized libraries: The sequences of 10,000 recombinants per library were analyzed by capillary sequencing using the ABI PRISM 3700 DNA Analyzer (Applied Biosystems, California). To determine the representation of transcripts in a library, BLAST analysis was performed on the clone sequences to assign transcript identity to each isolated clone, i.e. the sequences of the isolated nucleic acids were first masked to eliminate low complexity sequences using the XBLAST masking program (refs. 163, 164 and 165). Generally, masking does not influence the final search results, except to eliminate sequences of relative little interest due to their low complexity, and to eliminate multiple “hits” based on similarity to repetitive regions common to multiple sequences e.g. Alu repeats. The remaining sequences were then used in a BLASTN vs. GenBank search. The sequences were also used as query sequence in a BLASTX vs. NRP (non-redundant proteins) database search.

Automated sequencing reactions were performed using a Perkin-Elmer PRISM Dye Terminator Cycle Sequencing Ready Reaction Kit containing AmpliTaq DNA Polymerase, FS, according to the manufacturer's directions. The reactions were cycled on a GeneAmp PCR System 9600 as per manufacturer's instructions, except that they were annealed at 20° C. or 30° C. for one minute. Sequencing reactions were ethanol precipitated, pellets were resuspended in 8 microliters of loading buffer, 1.5 microliters was loaded on a sequencing gel, and the data was collected by an ABI PRISM 3700 DNA Sequencer. (Applied Biosystems, Foster City, Calif.).

The number of times a sequence is represented in a library is determined by performing sequence identity analysis on cloned cDNA sequences and assigning transcript identity to each isolated clone. First, each sequence was checked to see if it was a mitochondrial, bacterial or ribosomal contaminant. Such sequences were excluded from the subsequent analysis. Second, sequence artifacts (e.g. vector and repetitive elements) were masked and/or removed from each sequence.

The remaining sequences were compared via BLAST {166} to GenBank and EST databases for gene identification and were compared with each other via FastA {167} to calculate the frequency of cDNA appearance in the normalized cDNA library. The sequences were also searched against the GenBank and GeneSeq nucleotide databases using the BLASTN program (BLASTN 1.3 MP {166}). Fourth, the sequences were analyzed against a non-redundant protein (NRP) database with the BLASTX program (BLASTX 1.3 MP {166}). This protein database is a combination of the Swiss-Prot, PIR, and NCBI GenPept protein databases. The BLASTX program was run using the default BLOSUM-62 substitution matrix with the filter parameter: “xnu+seg”. The score cutoff utilized was 75.

Assembly of overlapping clones into contigs was done using the program Sequencher (Gene Codes Corp.; Ann Arbor, Mich.). The assembled contigs were analyzed using the programs in the GCG package (Genetic Computer Group, University Research Park, 575 Science Drive, Madison, Wis. 53711) Suite Version 10.1.

Detection of Elevated Levels of cDNA Associated with Prostate Cancer Using Arrays

cDNA sequences representing a variety of candidate genes to be screened for differential expression in prostate cancer were assayed by hybridization on nucleic acid arrays. The cDNA sequences included cDNA clones isolated from cell lines or tissues as described above. The cDNA sequences analyzed also included nucleic acids comprising sequence overlap with sequences in the Unigene database, and which encode a variety gene products of various origins, functionality, and levels of characterization. cDNAs were spotted onto reflective slides (Amersham) according to methods well known in the art at a density of 9,216 spots per slide representing 4608 sequences (including controls) spotted in duplicate, with approximately 0.8 μl of an approximately 200 ng/μl solution of cDNA.

PCR products of selected cDNA clones corresponding to the gene products of interest were prepared in a 50% DMSO solution. These PCR products were spotted onto Amersham aluminum microarray slides at a density of 9216 clones per array using a Molecular Dynamics Generation III spotting robot. Clones were spotted in duplicate, giving 4608 different sequences per array.

cDNA probes were prepared from total RNA obtained by laser capture microdissection (LCM, Arcturus Enginering Inc., Mountain View, Calif.) of tumor tissue samples and normal tissue samples isolated from the patients described above.

Total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (e.g. ref. 168), and the antisense RNA was then converted into cDNA. The second set of cDNAs were again transcribed in vitro, using the T7 promoter, to provide antisense RNA. This antisense RNA was then fluorescently labeled, or the RNA was again converted into cDNA, allowing for third round, of T7-mediated amplification to produce more antisense RNA. Thus the procedure provided for two or three rounds of in vitro transcription to produce the final RNA used for fluorescent labeling. Probes were labeled by making fluorescently labeled cDNA from the RNA starting material. Fluorescently-labeled cDNAs prepared from the tumor RNA sample were compared to fluorescently labeled cDNAs prepared from normal cell RNA sample. For example, the cDNA probes from the normal cells were labeled with Cy3 fluorescent dye (green) and cDNA probes prepared from the tumor cells were labeled with Cy5 fluorescent dye (red).

The differential expression assay was performed by mixing equal amounts of probes from tumor cells and normal cells of the same patient. The arrays were pre-hybridized by incubation for about 2 hrs at 60° C. in 5×SSC/0.2% SDS/1 mM EDTA, and then washed three times in water and twice in isopropanol. Following pre-hybridization of the array, the probe mixture was then hybridized to the array under conditions of high stringency (overnight at 42° C. in 50% formamide, 5×SSC, and 0.2% SDS. After hybridization, the array was washed at 55° C. three times as follows: 1) first wash in 1×SSC/0.2% SDS; 2) second wash in 0.1×SSC/0.2% SDS; and 3) third wash in 0.1×SSC.

The arrays were then scanned for green and red fluorescence using a Molecular Dynamics Generation III dual color laser-scanner/detector. The images were processed using BioDiscovery Autogene software, and the data from each scan set normalized. The experiment was repeated, this time labeling the two probes with the opposite color in order to perform the assay in both “color directions.” Each experiment was sometimes repeated with two more slides (one in each color direction). The data from each scan was normalized, and the level fluorescence for each sequence on the array expressed as a ratio of the geometric mean of 8 replicate spots/genes from the four arrays or 4 replicate spots/gene from 2 arrays or some other permutation.

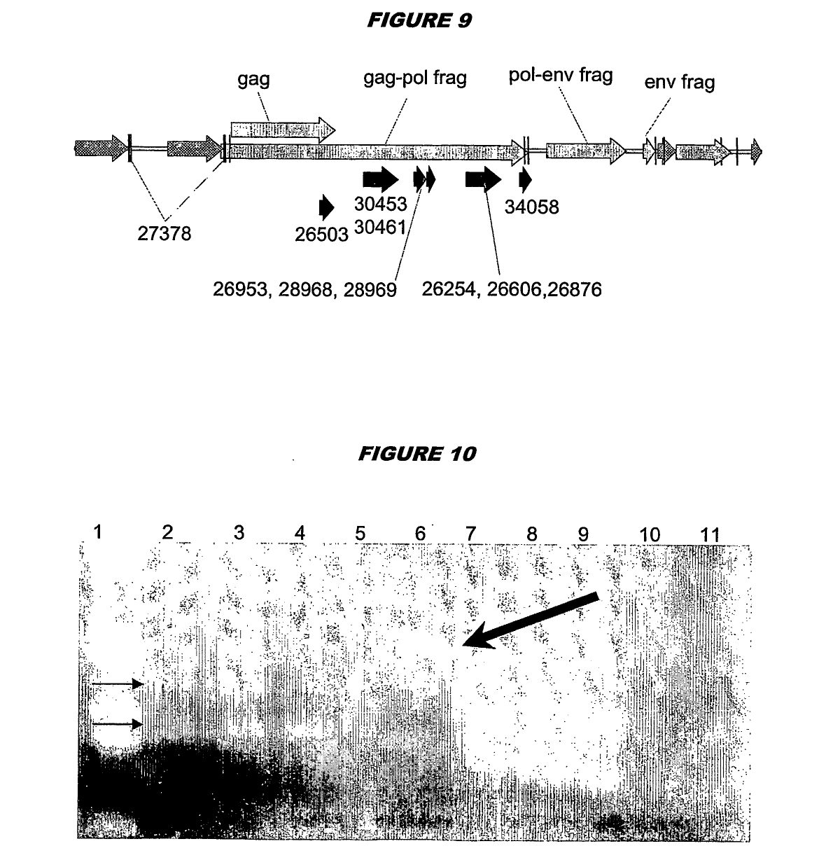

Array features which were found to give elevated signals using prostate tumor tissue were sequenced and mapped to the human genome sequence. The elevated array spots features span about 90% of PCAV and the locations of 11 such sequences on the PCAV genome are shown in FIG. 9, with five-digit numbers being the codes for individual array features.

Although some of the 11 elevated sequences come from regions in the genome which are highly conserved among the HERV-K HML2.0 family, and will thus not be specific for the virus at megabase 20.428 of chromosome 22, other spots are not.

Sequence 27378

27378 (SEQ ID 14) is present at elevated levels in prostate tumors. It aligns to two separate regions of the genomic DNA sequence on chromosome 22 (nucleotides 977-1075 & 2700-2777 of SEQ ID 1):

| | | | |
|---|---|---|---|
| PCAV ch22 20.428mb | + LTRs 27378 | (957) (1) | 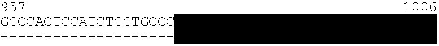 |
| PCAV ch22 20.428mb | + LTRs 27378 | (1007) (31) | 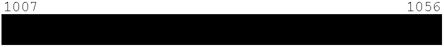 |
| PCAV ch22 20.428mb | + LTRs 27378 | (1057) (81) | 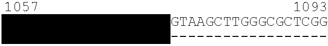 |
| | | | INTRON 1 |
| PCAV ch22 20.428mb | + LTRs 27378 | (2684) (100) | 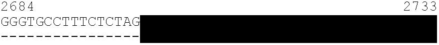 |
| PCAV ch22 20.428mb | + LTRs 27378 | (2734) (134) | 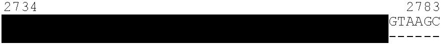 |
| | | | INTRON 2 |
| PCAV ch22 20.428mb | + LTRs 27378 | (8134) (178) | 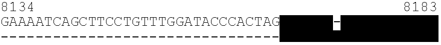 |
| PCAV ch22 20.428mb | + LTRs 27378 | (8183) (196) | 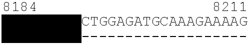 |

Within SEQ ID 1, nucleotides 1076-1077 are GT and nucleotides 2698-2699 are AG, these being consensus splice donor and acceptor sequences, respectively. Hybridization to 27378 thus verifies splicing in which the first 5′ LTR is joined to the splice acceptor site near the 3′ end of the second 5′ LTR (joins nucleotide 1075 of SEQ ID 1 to nucleotide 2700). Because the sequences in the two exons are from two different viruses (old and new), and these are significantly different from other family new and old family members, it is unlikely that the 27378 product was transcribed from a HERV-K other than PCAV.

Sequence 34058

Spot 34058 (SEQ ID 15) is highly elevated in prostate tumor tissue. Its sequence spans an alternative splice site that occurs in some “old” genomes and that connects the envelope ATG to a splice acceptor site near the 3′ LTR. The sequence matches PCAV more closely (single mismatch at 2443) than the related HERV-Ks found on chromosomes 3 and 6:

| | | |
|---|---|---|
| 34058 env genomic PCAV ch 22 20.4 mb env genomic PCAV ch 6 47.1 mb env genomic PCAV ch3 103 mb | (1) (1) (1) (1) | 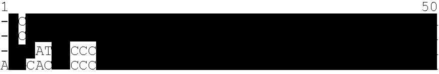 |
| 34058 env genomic PCAV ch22 20.4 mb env genomic PCAV ch 6 47.1 mb env genomic PCAV ch3 103 mb | (50) (50) (50) (51) | 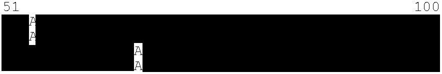 |
| 34058 env genomic PCAV ch 22 20.4 mb env genomic PCAV ch 6 47.1 mb env genomic PCAV ch3 103 mb | (100) (100) (100) (101) | 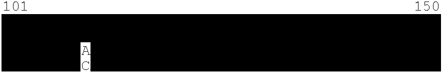 |
| 34058 env genomic PCAV ch 22 20.4 mb env genomic PCAV ch 6 47.1 mb env genomic PCAV ch3 103 mb | (150) (150) (150) (151) | 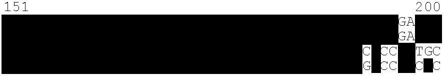 |
| 34058 env genomic PCAV ch 22 20.4 mb env genomic PCAV ch 6 47.1 mb env genomic PCAV ch3 103 mb | (200) (200) (200) (201) | 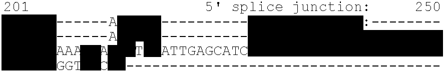 |
| | | <intron> |
| | 3′ | splice site: |
| 34058 env genomic PCAV ch 22 20.4 mb env genomic PCAV ch 6 47.1 mb env genomic PCAV ch3 103 mb Consensus | (225) (2106) (2135) (1835) (2201) | 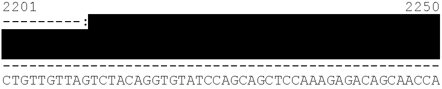 |
| 34058 env genomic PCAV ch 22 20.4 mb env genomic PCAV ch 6 47.1 mb env genomic PCAV ch3 103 mb Consensus | (265) (2156) (2185) (1835) (2251) | 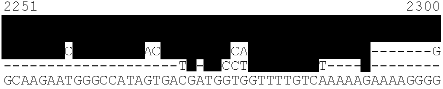 |
| 34058 env genomic PCAV ch 22 20.4 mb env genomic PCAV ch 6 47.1 mb env genomic PCAV ch3 103 mb Consensus | (315) (2206) (2228) (1852) (2301) | 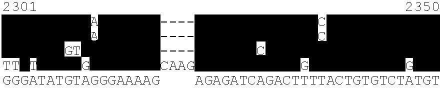 |
| 34058 env genomic PCAV ch 22 20.4 mb env genomic PCAV ch 6 47.1 mb env genomic PCAV ch3 103 mb Consensus | (361) (2252) (2274) (1902) (2351) | 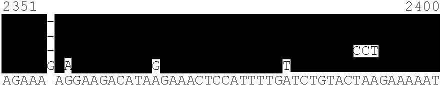 |
| 34058 env genomic PCAV ch 22 20.4 mb env genomic PCAV ch 6 47.1 mb env genomic PCAV ch3 103 mb Consensus | (410) (2301) (2323) (1952) (2401) | 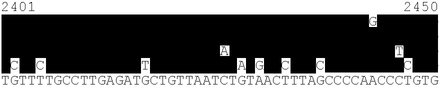 |
| 34058 env genomic PCAV ch 22 20.4 mb env genomic PCAV ch 6 47.1 mb env genomic PCAV ch3 103 mb Consensus | (460) (2351) (2373) (2002) (2451) | 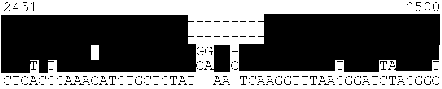 |
| 34058 env genomic PCAV ch 22 20.4 mb env genomic PCAV ch 6 47.1 mb env genomic PCAV ch3 103 mb Consensus | (501) (2392) (2422) (2052) (2501) | 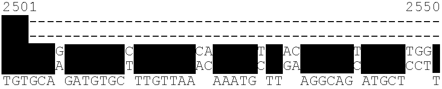 |

Sequence 26254

Signal from sequence 26254 on the array was elevated in prostate tumor tissue compared to normal tissue. The 26254 sequence (SEQ ID 16) aligns almost perfectly to chromosome 22 contigs AP000345 (SEQ ID 17=nucleotides 63683-64332 of AP000345) and AP000346 (SEQ ID 18=nucleotides 26271-26920 of AP000346) (nucleotides 7065-7701 of SEQ ID 1):

| | | |
|---|---|---|
| 26254 AP000346 AP000345 | (1) (1) (1) | 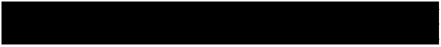 |
| 26254 AP000346 AP000345 | (51) (51) (51) | 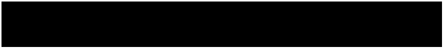 |
| 26254 AP000346 AP000345 | (101) (101) (101) | 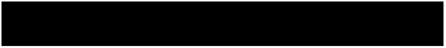 |
| 26254 AP000346 AP000345 | (151) (151) (151) | 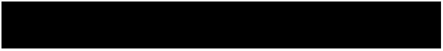 |
| 26254 AP000346 AP000345 | (201) (201) (201) | 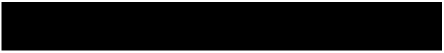 |
| 26254 AP000346 AP000345 | (251) (251) (251) | 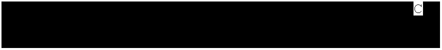 |
| 26254 AP000346 AP000345 | (301) (301) (301) | 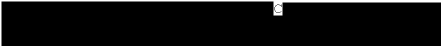 |
| 26254 AP000346 AP000345 | (351) (351) (351) | 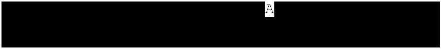 |
| 26254 AP000346 AP000345 | (401) (401) (401) | 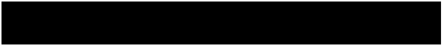 |
| 26254 AP000346 AP000345 | (451) (451) (451) | 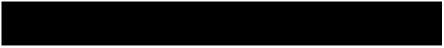 |
| 26254 AP000346 AP000345 | (501) (501) (501) | 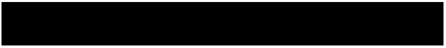 |
| 26254 AP000346 AP000345 | (551) (551) (551) | 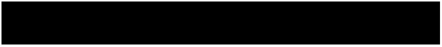 |
| 26254 AP000346 AP000345 | (601) (601) (601) | 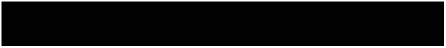 |

The four point mutations relative to the chromosome 22 sequence could represent sequencing errors (either for the chromosome or for 26254) or could, alternatively, be SNPs within the human genome.

PCAV is most closely related to HERV-Ks found on chromosomes 3 and 6. Alignment of the chromosome 3, 6 and 22 viruses in the region of 26254 shows that it is unlikely that 26254 is derived from chromosome 3 or 6 and that it is most likely derived from a chromosome 22 PCAV transcript:

| | | |
|---|---|---|
| ch22 AP000346 ch22 AP000345 ch3 103.75 ch6 47.1mb | (1) (1) (1) (1) | 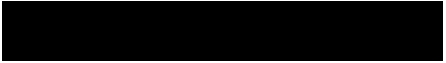 |
| ch22 AP000346 ch22 AP000345 ch3 103.75 ch6 47.1mb | (51) (51) (51) (51) | 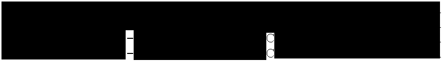 |
| ch22 AP000346 ch22 AP000345 ch3 103.75 ch6 47.1mb | (101) (101) (100) (100) | 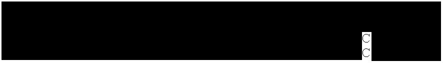 |
| ch22 AP000346 ch22 AP000345 ch3 103.75 ch6 47.1mb | (151) (151) (150) (150) | 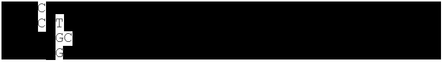 |
| ch22 AP000346 ch22 AP000345 ch3 103.75 ch6 47.1mb | (201) (201) (200) (200) | 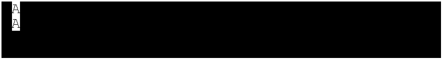 |
| ch22 AP000346 ch22 AP000345 ch3 103.75 ch6 47.1mb | (251) (251) (250) (250) | 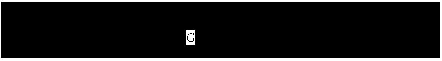 |
| ch22 AP000346 ch22 AP000345 ch3 103.75 ch6 47.1mb | (301) (301) (300) (300) | 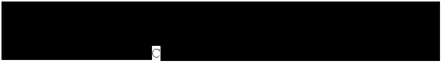 |
| ch22 AP000346 ch22 AP000345 ch3 103.75 ch6 47.1mb | (351) (351) (350) (350) | 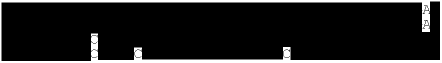 |
| ch22 AP000346 ch22 AP000345 ch3 103.75 ch6 47.1mb | (401) (401) (400) (400) | 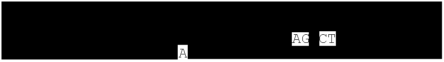 |
| ch22 AP000346 ch22 AP000345 ch3 103.75 ch6 47.1mb | (451) (451) (450) (450) | 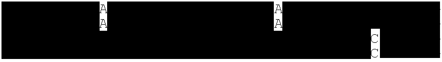 |
| ch22 AP000346 ch22 AP000345 ch3 103.75 ch6 47.1mb | (501) (501) (500) (500) | 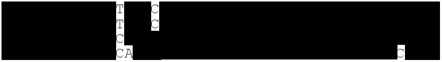 |
| ch22 AP000346 ch22 AP000345 ch3 103.75 ch6 47.1mb | (551) (551) (550) (550) | 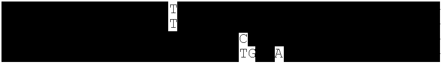 |
| ch22 AP000346 ch22 AP000345 ch3 103.75 ch6 47.1mb | (601) (601) (600) (600) | 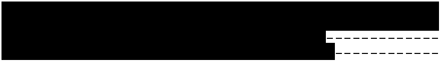 |

Although the HERVs on chromosomes 3, 6 and 22 are closely-related, therefore, they can be distinguished by hybridization.

Sequence 30453

Signal from sequence 30453 on the array was elevated in prostate tumor tissue compared to normal tissue. The 30453 sequence (SEQ ID 113) aligns with chromosome 22:

| Score = 1063 bits (536), Expect = 0.0 | | | |
|---|---|---|---|
| Identities = 635/654 (97%), Gaps = 11/654 (1%) | | | |
| Strand = Plus/Plus | | | |
| Query: | 51 | agggagatcaagtctaaatttgaagggagtccaaattcatactggggtaatttattcaga | 110 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 126730 | agggagatcaagtctaaatttgaagggagtccaaattcatactggggtaatttattcaga | 126789 |
| Query: | 111 | ttataaagggggaattcagttagtg-tcagctccactgttccccggagtgccaatccagg | 169 |
| | | \|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\| | |
| Sbjct: | 126790 | ttataaagggggaattcagttagtgatcagctccactgttccccggagtgccaatccagg | 126849 |
| Query: | 170 | tgatagaattgctcaattactgcttttgccttatgttaaaattggggaaaacaaaacgga | 229 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\| | |
| Sbjct: | 126850 | tgatagaattgctcaattactgcttttgccttatgttaaaattggggaaaacaaaaagga | 126909 |
| Query: | 230 | aagaacaggagggtttggaagtaccaaccctgcaggaaaagctgcttattgggctaatca | 289 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 126910 | aagaacaggagggtttggaagtaccaaccctgcaggaaaagctgcttattgggctaatca | 126969 |
| Query: | 290 | ggtctcagaagatagacccgtgtgtacagtcactattcagggaaagagtttgaaggatta | 349 |
| | | \|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 126970 | ggtctcagaggatagacccgtgtgtacagtcactattcagggaaagagtttgaaggatta | 127029 |
| Query: | 350 | gtggatacccaggctgat---tctatcatcggcataggtaccgcctcagaagtgtatcaa | 406 |
| | | \|\|\|\|\|\|\|\|\|\|\|\|\|\|\|\|\|\| \|\|\| \|\|\|\|\|\|\|\|\|\|\|\|\|\|\|\| \|\|\|\|\|\|\|\|\|\|\|\|\|\|\|\|\|\| | |
| Sbjct: | 127030 | gtggatacccaggctgatgtttctgtcatcggcataggtactgcctcagaagtgtatcaa | 127089 |
| Query: | 407 | agtgccatgattttacattgtctaggatctgataatcaagaaagtacggttcagcctgtg | 466 |
| | | \|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\| | |
| Sbjct: | 127090 | agtgccatgattttacattgtccaggatctgataatcaagaaagtacggttcagcctgtg | 127149 |
| Query: | 467 | atcacttcattccaatcaatttatggggccgagacttgttacaacaatggcatgcagaga | 526 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127150 | atcacttcattccaatcaatttatggggccgagacttgttacaacaatggcatgcagaga | 127209 |
| Query: | 527 | ttactatcccagcctccctatacagccccaggaatcaaaaaatcatgactaaaatgggat | 586 |
| | | \|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\| | |
| Sbjct: | 127210 | ttactatcccagcctccctatacagccccaggaataaaaaaatcatgactaaaatgggat | 127269 |
| Query: | 587 | agctccctaaaaagggactaggaaagaaagaagtcccaattgaggctg-aaaaaatcaaa | 645 |
| | | \|\|\|\|\|\|\|\|\|\|\|\|\|\|\|\|\|\|\|\|\| \|\|\|\|\|\|\|\|\|\|\|\|\|\|\|\|\|\|\|\|\|\|\| \|\|\|\|\|\|\|\|\|\|\| | |
| Sbjct: | 127270 | agctccctaaaaagggactag----gaaagaagtcccaattgaggctgaaaaaaatcaaa | 127325 |
| Query: | 646 | aaag-aaangaatagggcatcctttttaggagc-gtcactgtanagcctccaaa | 697 |
| | | \|\|\|\| \|\|\| \|\|\|\|\|\|\|\|\|\|\|\|\|\|\|\|\|\|\|\|\|\|\|\| \|\|\|\|\|\|\|\|\| \|\|\|\|\|\|\|\|\|\| | |
| Sbjct: | 127326 | aaagaaaaggaatagggcatcctttttaggagcggtcactgtagagcctccaaa | 127379 |

Sequence 26503

Signal from sequence 26503 on the array was elevated in prostate tumor tissue compared to normal tissue. The 26503 sequence (SEQ ID 116) aligns with chromosome 22:

| Score = 527 bits (266), Expect = e−147 | | | |
|---|---|---|---|
| Identities = 350/378 (92%) | | | |
| Strand = Plus/Plus | | | |
| Query: | 73 | tttcaccatgaaaatgttaaaagacataaaggaaggagctaaacaatatggacccaactc | 132 |
| | | \|\|\|\|\|\|\|\|\|\|\|\|\|\|\|\|\|\|\|\|\|\|\|\| \|\|\|\|\|\|\|\|\|\|\|\|\| \|\|\|\|\|\|\|\|\|\|\|\|\| \|\|\|\|\|\|\| | |
| Sbjct: | 125548 | tttcaccatgaaaatgttaaaagatataaaggaaggagttaaacaatatggatccaactc | 125607 |
| Query: | 133 | tccttatatgagaacgttattagattccattgctcatggaaatagacttattccttatga | 192 |
| | | \|\|\|\|\|\|\|\| \|\|\|\|\| \|\|\|\|\|\|\|\|\|\|\|\|\|\|\|\|\|\|\|\|\|\|\|\|\|\|\|\|\|\|\|\|\|\| \|\|\|\|\|\|\|\|\| | |
| Sbjct: | 125608 | cccttatataagaacattattagattccattgctcatggaaatagacttactccttatga | 125667 |
| Query: | 193 | ttgggaaattttacctaaatcttccctttcaccctctcagtatctacagtttaaaacctg | 252 |
| | | \|\|\|\|\|\|\|\|\|\|\| \| \|\|\|\|\|\|\|\|\|\|\|\|\|\|\| \|\|\|\|\|\|\|\|\|\|\|\|\|\|\|\|\|\|\|\|\|\|\|\|\|\|\|\| | |
| Sbjct: | 125668 | ctgggaaattttggccaaatcttccctttcatcctctcagtatctacagtttaaaacctg | 125727 |
| Query: | 253 | gtggattgatggagtacaagaacaggtacggaaaaatcaggctacttatcctgttgttaa | 312 |
| | | \|\|\|\|\|\|\|\|\|\|\|\|\|\|\|\|\|\|\|\|\|\|\|\|\|\|\|\|\|\| \|\|\|\|\|\|\|\|\|\|\|\|\|\|\| \| \|\| \|\|\|\|\|\| | |
| Sbjct: | 125728 | gtggattgatggagtacaagaacaggtacgaaaaaatcaggctactaagcccactgttaa | 125787 |
| Query: | 313 | tatagatgcagaccaattgctaggaacacgtccaaattggagcactattaaccaacaatc | 372 |
| | | \|\|\|\|\|\| \|\|\|\|\|\|\|\|\|\|\|\| \|\|\|\|\|\|\|\| \|\|\|\|\|\|\|\|\|\|\|\|\|\|\|\| \|\|\|\|\|\|\|\|\|\|\|\|\|\| | |
| Sbjct: | 125788 | tatagacgcagaccaattgttaggaacaggtccaaattggagcaccattaaccaacaatc | 125847 |
| Query: | 373 | agtaatgcaaaatgaggctattgaacaactaggggctatttgcctcagggcctgggaaaa | 432 |
| | | \|\|\| \|\|\|\|\| \|\|\|\|\|\|\|\|\|\|\|\|\|\|\|\|\|\| \|\| \|\|\|\|\|\|\|\|\|\|\|\|\|\|\|\|\|\|\|\|\|\|\|\| \|\|\| | |
| Sbjct: | 125848 | agtgatgcagaatgaggctattgaacaagtaagggctatttgcctcagggcctggggaaa | 125907 |
| Query: | 433 | gattcaggacccaggaac | 450 |
| | | \|\|\|\|\|\|\|\|\|\|\|\|\|\|\|\|\| | |
| Sbjct: | 125908 | aattcaggacccaggaac | 125925 |
| Score = 208 bits (105), Expect = 3e−51 | | | |
| Identities = 191/215 (88%), Gaps = 4/215 (1%) | | | |
| Strand = Plus/Plus | | | |
| Query: | 448 | aaccagttagagaca-gttttcagactgttatatcattcattatgttgatgatattttgt | 506 |
| | | \|\|\|\|\|\|\|\|\|\|\|\|\|\|\| \|\|\|\|\|\|\|\|\|\|\|\|\|\|\| \|\|\| \|\|\|\| \|\|\|\|\|\|\|\|\| \|\|\|\|\|\|\| | |
| Sbjct: | 127805 | aaccagttagagacaagttttcagactgttacatcgttcactatgttgat---attttgt | 127861 |
| Query: | 507 | gtgctgcagaaacaagagacaaattaattgacttttacatgtttctgcagacagaggttg | 566 |
| | | \|\|\|\|\|\|\|\|\|\|\|\|\| \|\|\|\|\|\|\|\|\|\|\|\|\|\|\|\|\|\| \|\|\|\|\| \|\|\|\|\|\|\|\|\|\|\|\|\|\|\|\|\|\|\| | |
| Sbjct: | 127862 | gtgctgcagaaacgagagacaaattaattgaccgttacacatttctgcagacagaggttg | 127921 |
| Query: | 567 | caaacacaggcctgacaatagcatctgataagattcagacctccactccttttaattatt | 626 |
| | | \| \|\|\| \| \|\| \|\|\|\|\|\|\|\|\| \|\|\|\|\|\|\|\|\|\|\|\|\|\|\|\| \|\|\|\|\| \|\|\|\|\|\|\|\| \|\|\| \| | |
| Sbjct: | 127922 | ccaacgcgggactgacaataacatctgataagattcaaacctctactcctttccgttact | 127981 |
| Query: | 627 | tgggaatgcaggtagaggaaagaaaaattaaacca | 661 |
| | | \|\|\|\|\|\|\|\|\|\|\|\|\|\|\|\|\|\|\|\|\|\| \|\|\|\|\|\|\|\|\|\|\|\| | |
| Sbjct: | 127982 | tgggaatgcaggtagaggaaaggaaaattaaacca | 128016 |

Patient Libraries

HERV-K HML2.0 cDNAs cloned from patient libraries align with PCAV. Clones from libraries derived from four patients align with >95% identity to PCAV.

SEQ ID 19 is from a cDNA which is present at elevated levels in prostate tumors. The first 463 of its 470 nucleotides align to four separate regions of the genomic DNA sequence on chromosome 22 (nucleotides 956-1075, 2700-2777, 8166-8244 & 10424-10609 of SEQ ID 1):

| SEQ ID 19 | AGATCTGATCATCTGGTGCCCAACGTGGAGGCTTTTCTCTAGGGTGAAGGGACTCTCGAG | 60 |
|---|---|---|
| | \|\| \| \|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\| | |
| SEQ ID 1 | AGGCCACTCCATCTGGTGCCCAACGTGGATGCTTTTCTCTAGGGTGAAGGGACTCTCGAG | 1015 |
| SEQ ID 19 | TGTGGTCATTGAGGACAAGTCAACGAGAGATTCCCGAGTACGTCTACAGTGAGCCTTGTG | 120 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SEQ ID 1 | TGTGGTCATTGAGGACAAGTCAACGAGAGATTCCCGAGTACGTCTACAGTGAGCCTTGTG | 1075 |
| <gap in SEQ ID 1> | | |
| SEQ ID 19 | GGTGAAGGTACTCTACAGTGTGGTCATTGAGGACAAGTTGACGAGAGAGTCCCAAGTACG | 180 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SEQ ID 1 | GGTGAAGGTACTCTACAGTGTGGTCATTGAGGACAAGTTGACGAGAGAGTCCCAAGTACG | 2759 |
| SEQ ID 19 | TCCACGGTCAGCCTTGCG | 198 |
| | \|\|\|\|\|\|\|\|\|\|\|\|\|\|\|\|\|\| | |
| SEQ ID 1 | TCCACGGTCAGCCTTGCG | 2777 |
| <gap in SEQ ID 1> | | |
| SEQ ID 19 | ACATTTAAAGTTCTACAATGAACTCACTGGAGATGCAAAGAAAAGTGTGGAGATGGAGAC | 258 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SEQ ID 1 | ACATTTAAAGTTCTACAATGAACTCACTGGAGATGCAAAGAAAAGTGTGGAGATGGAGAC | 8225 |
| SEQ ID 19 | ACCCCAATCGACTCGCCAG | 277 |
| | \|\|\|\|\|\|\|\|\|\|\|\|\|\|\|\|\|\|\| | |
| SEQ ID 1 | ACCCCAATCGACTCGCCAG | 8244 |
| <gap in SEQ ID 1> | | |
| SEQ ID 19 | TCTACAGGTGTATCCAGCAGCTCCAAAGAGACAGCAACCAGCAAGAATGGGCCATAGTGA | 337 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SEQ ID 1 | TCTACAGGTGTATCCAGCAGCTCCAAAGAGACAGCAACCAGCAAGAATGGGCCATAGTGA | 10483 |
| SEQ ID 19 | CGATGGTGGTTTTGTCAAAAAGAAAAGGGGGGGATATGTAAGGAAAAGAGAGATCAGACT | 397 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SEQ ID 1 | CGATGGTGGTTTTGTCAAAAAGAAAAGGGGGGGATATGTAAGGAAAAGAGAGATCAGACT | 10543 |
| SEQ ID 19 | TTCACTGTGTCTATGTAGAAAAGGAAGACATAAGAAACTCCATTTTGTTCTGTACTAAGA | 457 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\| | |
| SEQ ID 1 | TTCACTGTGTCTATGTAGAAAAGGAAGACATAAGAAACTCCATTTTGATCTGTACTAAGA | 10603 |
| SEQ ID 19 | ATTCGG | 463 |
| | \| | |
| SEQ ID 1 | AAAATT | 10609 |

The dinucleotide sequences before and after the “gaps” in SEQ ID 1 are as follows:

| SEQ | | SEQ | Preceding and following | |
|---|---|---|---|---|
| ID 19 | Exon | ID 1 | dinucleotide in SEQ ID 1 | |
| 1-120 | 1 | 956-1075 | — | 1076-1077: GT |
| 121-198 | 2 | 2700-2777 | 2698-2699: AG | 2778-2779: GT |
| 199-277 | 3 | 8166-8244 | 8164-8165: AG | 8245-8246: GT |
| 278-463 | 4 | 10424-10609 | 10422-10423: AG | — |

The “gaps” in SEQ ID 1 thus begin and end with consensus splice donor and acceptor sequences. The presence of SEQ ID 19 in a cDNA thus verifies splicing in which the first 5′ LTR is joined to the splice acceptor site near the 3′ end of the second 5′ LTR (nucleotide 1075 of SEQ ID 1 joined to nucleotide 2700), as well as other splicing events. Because the sequences in exons 1 and 2 are from two different viruses (old and new), and these are significantly different from other family new and old family members, it is unlikely that the SEQ ID 19 product was transcribed from a HERV-K other than PCAV.

SEQ ID 114 (035JN013.F03-FIS) aligns with available chromosome 22 sequence:

| Score = 1744 bits (880), Expect = 0.0 | | | |
|---|---|---|---|
| Identities = 907/913 (99%), Gaps = 1/913 (0%) | | | |
| Strand = Plus/Plus | | | |
| Query: | 152 | gattttgaaaaatttgctttcaccacaccagcctaaataataaagaaccagccaccaggt | 211 |
| | | \|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127680 | gattttgaaaaatttgcttttaccacaccagcctaaataataaagaaccagccaccaggt | 127739 |
| Query: | 212 | ttcagtggaaagtattgcctcagggaatgcttaatagttcaactatttgtcagctcaagc | 271 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127740 | ttcagtggaaagtattgcctcagggaatgcttaatagttcaactatttgtcagctcaagc | 127799 |
| Query: | 272 | tctgcaaccagttagagacaagttttcagactgttacatcgttcactatgttgatatttt | 331 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127800 | tctgcaaccagttagagacaagttttcagactgttacatcgttcactatgttgatatttt | 127859 |
| Query: | 332 | gtgtgctgcagaaacgagagacaaattaattgaccgttacacatttctgcagacagaggt | 391 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127860 | gtgtgctgcagaaacgagagacaaattaattgaccgttacacatttctgcagacagaggt | 127919 |
| Query: | 392 | tgccaacgcgggactgacaataacatctgataagattcaagcctctactcctttccgtta | 451 |
| | | \|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\| | |
| Sbjct: | 127920 | tgccaacgcgggactgacaataacatctgataagattcaaacctctactcctttccgtta | 127979 |
| Query: | 452 | cttgggaatgcaggtagaggaaaggaaaattaaaccacaaaaaaatagaaataagaaaag | 511 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\| | |
| Sbjct: | 127980 | cttgggaatgcaggtagaggaaaggaaaattaaaccacaaaaaa-tagaaataagaaaag | 128038 |
| Query: | 512 | acacattaaaagcattaaatgagtttcaaaagttgctaggagatactaattggatttgga | 571 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128039 | acacattaaaagcattaaatgagtttcaaaagttgctaggagatactaattggatttgga | 128098 |
| Query: | 572 | gatattaattggatttggccaactctaggcattcctacttatgccatgtcaaatttgttc | 631 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128099 | gatattaattggatttggccaactctaggcattcctacttatgccatgtcaaatttgttc | 128158 |
| Query: | 632 | tctttcttaagaggggactcggaattaaatagtgaaagaacgttaactccagaggcaact | 691 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128159 | tctttcttaagaggggactcggaattaaatagtgaaagaacgttaactccagaggcaact | 128218 |
| Query: | 692 | aaagaaattaaattaattgaagaaaaaattcggtcagcacaagtaaatagaatagatcac | 751 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128219 | aaagaaattaaattaattgaagaaaaaattcggtcagcacaagtaaatagaatagatcac | 128278 |
| Query: | 752 | ttggccccactccaaattttgatttttactactgcacattccctaacaggcatcattgtt | 811 |
| | | \|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\| | |
| Sbjct: | 128279 | ttggccccactccaaattttgatttttgctactgcacattccctaacaggcatcattgtt | 128338 |
| Query: | 812 | caaaacacagatcttgtggagtggtccttccttcctcacagtacaattaagacttttaca | 871 |
| | | \|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128339 | caaaatacagatcttgtggagtggtccttccttcctcacagtacaattaagacttttaca | 128398 |
| Query: | 872 | ttgtacttggatcaaatggctacattaattggtcagggaagattatgaataataacattg | 931 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128399 | ttgtacttggatcaaatggctacattaattggtcagggaagattatgaataataacattg | 128458 |
| Query: | 932 | tgtggaaatgacccagataaaatcactgttcctttcaacaagcaacaggttagacaagcc | 991 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128459 | tgtggaaatgacccagataaaatcactgttcctttcaacaagcaacaggttagacaagcc | 128518 |
| Query: | 992 | tttatcaattctggtgcatggcagattggtcttgccgattttgtgggaattattgacaat | 1051 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128519 | tttatcaattctggtgcatggcagattggtcttgccgattttgtgggaattattgacaat | 128578 |
| Query: | 1052 | cgttaccacaaaa | 1064 |
| | | \|\|\|\|\|\|\| \|\|\|\|\| | |
| Sbjct: | 128579 | cgttaccccaaaa | 128591 |

SEQ ID 115 (035JN015.H02-FIS) aligns with available chromosome 22 sequence:

| Score = 1618 bits (816), Expect = 0.0 | | | |
|---|---|---|---|
| Identities = 828/832 (99%) | | | |
| Strand = Plus/Plus | | | |
| Query: | 1 | ccaaaagaatgagtcatcaaaactcagtatcacttgactcaaagagcagagttggttgcc | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128720 | ccaaaagaatgagtcatcaaaactcagtatcacttgactcaaagagcagagttggttgcc | 128779 |
| Query: | 61 | gtcattacagtgttaacaagattttaatcagtctattaacattgtatcagattctgcata | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128780 | gtcattacagtgttaacaagattttaatcagtctattaacattgtatcagattctgcata | 128839 |
| Query: | 121 | tgtagtacaggctacaaaggatattgagagagccctaatcaaatacattatggatgatca | 180 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128840 | tgtagtacaggctacaaaggatattgagagagccctaatcaaatacattatggatgatca | 128899 |
| Query: | 181 | gttaaacccgctgtttaatttgttacaacaaaatgtaagaaaaagaaatttcccatttta | 240 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128900 | gttaaacccgctgtttaatttgttacaacaaaatgtaagaaaaagaaatttcccatttta | 128959 |
| Query: | 241 | tattactcatattcgagcacacactaatttaccagggcctttaactaaagcaaatgaaca | 300 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128960 | tattactcatattcgagcacacactaatttaccagggcctttaactaaagcaaatgaaca | 129019 |
| Query: | 301 | agctgactcgctagtatcatctgcattcatggaagcacaagaccttcatgccttgactca | 360 |
| | | \|\|\|\|\|\|\|\| \|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\| | |
| Sbjct: | 129020 | agctgacttgctagtatcatctgcattcatggaagcacaagaacttcatgccttgactca | 129079 |
| Query: | 361 | tgtaaatgcaataggattaaaaaataaatttaatatcacatggaaacagacaaaaaatat | 420 |
| | | \|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\| | |
| Sbjct: | 129080 | tgtaaatgcaataggattaaaaaataaatttgatatcacatggaaacagacaaaaaatat | 129139 |
| Query: | 421 | tgtacaacattgcacccagtgtcagattctacacctggccactcaggaggcaagagttaa | 480 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 129140 | tgtacaacattgcacccagtgtcagattctacacctggccactcaggaggcaagagttaa | 129199 |
| Query: | 481 | tcccagaggtctatgtcctaatgtgttatggcaaatggatgtcatgcacgtaccttcatt | 540 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 129200 | tcccagaggtctatgtcctaatgtgttatggcaaatggatgtcatgcacgtaccttcatt | 129259 |
| Query: | 541 | tggaaaattgtcatttgtccatgtgacagttgatacttattcacatttcatatgggcaac | 600 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 129260 | tggaaaattgtcatttgtccatgtgacagttgatacttattcacatttcatatgggcaac | 129319 |
| Query: | 601 | ctgccagacaggagaaagtacttcccatgttaagagacatttattatcttgttttcctgt | 660 |
| | | \|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\| | |
| Sbjct: | 129320 | ctgccagacaggagaaagtacttcccatgttaaaagacatttattatcttgttttcctgt | 129379 |
| Query: | 661 | catgggagttccagaaaaagttaaaacagacaatgggccaggttactgtagtaaagcagt | 720 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 129380 | catgggagttccagaaaaagttaaaacagacaatgggccaggttactgtagtaaagcagt | 129439 |
| Query: | 721 | tcaaaaattcttaaatcagtggaaaattacacatacaataggaattctctataattccca | 780 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 129440 | tcaaaaattcttaaatcagtggaaaattacacatacaatagg&attctctataattccca | 129499 |
| Query: | 781 | aggacaggccataattgaaagaactaatagaacactcaaagctcaattggtt | 832 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 129500 | aggacaggccataattgaaagaactaatagaacactcaaagctcaattggtt | 129551 |

SEQ ID 117 (035JN003.E06-FIS) aligns with available chromosome 22 sequence:

| Score = 1402 bits (707), Expect = 0.0 | | | |
|---|---|---|---|
| Identities = 710/711 (99%) | | | |
| Strand = Plus/Plus | | | |
| Query: | 1 | ctgaaaaaaatcaaaaaagaaaaggaatagggcatcctttttaggagcggtcactgtaga | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127311 | ctgaaaaaaatcaaaaaagaaaaggaatagggcatcctttttaggagcggtcactgtaga | 127370 |
| Query: | 61 | gcctccaaaacccattccattaacttgggggaaaaaaaaacaactgtatggtaaatcagc | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127371 | gcctccaaaacccattccattaacttgggggaaaaaaaaacaactgtatggtaaatcagc | 127430 |
| Query: | 121 | agcgcttccaaaacaaaaactggaggctttacatttattagcaaagaaacaattagaaaa | 180 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127431 | agcgcttccaaaacaaaaactggaggctttacatttattagcaaagaaacaattagaaaa | 127490 |
| Query: | 181 | aggacattgagccttcattttcgccttggaattctgtttgtaattcagaaaaaatccggc | 240 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127491 | aggacattgagccttcattttcgccttggaattctgtttgtaattcagaaaaaatccggc | 127550 |
| Query: | 241 | agatggcgtataatgccgtaattcaacccatgggggctctcccaccccggttgccctctc | 300 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127551 | agatggcgtataatgccgtaattcaacccatgggggctctcccaccccggttgccctctc | 127610 |
| Query: | 301 | cagccatggtcccctttaattataattgatctgaaggattgcttttttaccattcctctg | 360 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127611 | cagccatggtcccctttaattataattgatctgaaggattgcttttttaccattcctctg | 127670 |
| Query: | 361 | gcaaaacaggattttgagaaatttgcttttaccacaccagcctaaataataaagaaccag | 420 |
| | | \|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127671 | gcaaaacaggattttgaaaaatttgcttttaccacaccagcctaaataataaagaaccag | 127730 |
| Query: | 421 | ccaccaggtttcagtggaaagtattgcctcagggaatgcttaatagttcaactatttgtc | 480 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127731 | ccaccaggtttcagtggaaagtattgcctcagggaatgcttaatagttcaactatttgtc | 127790 |
| Query: | 481 | agctcaagctctgcaaccagttagagacaagttttcagactgttacatcgttcactatgt | 540 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127791 | agctcaagctctgcaaccagttagagacaagttttcagactgttacatcgttcactatgt | 127850 |
| Query: | 541 | tgatattttgtgtgctgcagaaacgagagacaaattaattgaccgttacacatttctgca | 600 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127851 | tgatattttgtgtgctgcagaaacgagagacaaattaattgaccgttacacatttctgca | 127910 |
| Query: | 601 | gacagaggttgccaacgcgggactgacaataacatctgataagattcaaacctctactcc | 660 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127911 | gacagaggttgccaacgcgggactgacaataacatctgataagattcaaacctctactcc | 127970 |
| Query: | 661 | tttccgttacttgggaatgcaggtagaggaaaggaaaattaaaccacaaaa | 711 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127971 | tttccgttacttgggaatgcaggtagaggaaaggaaaattaaaccacaaaa | 128021 |

SEQ ID 118 (035JN013.C11) aligns with available chromosome 22 sequence:

| Score = 894 bits (451), Expect = 0.0 | | | |
|---|---|---|---|
| Identities = 454/455 (99%) | | | |
| Strand = Plus/Plus | | | |
| Query: | 388 | taatgccgtaattcaacccatgggggctctcccaccccggttgccctctccagccatggt | 447 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127561 | taatgccgtaattcaacccatgggggctctcccaccccggttgccctctccagccatggt | 127620 |
| Query: | 448 | cccctttaattataattgatctgaaggattgcttttttaccattcctctggcaaaacagg | 507 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127621 | cccctttaattataattgatctgaaggattgcttttttaccattcctctggcaaaacagg | 127680 |
| Query: | 508 | attttgaaaaatttgcttttaccacaccagcctaaataataaagaaccagccaccaggtt | 567 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127681 | attttgaaaaatttgcttttaccacaccagcctaaataataaagaaccagccaccaggtt | 127740 |
| Query: | 568 | tcagtggaaagtattgcctcagggaatgcttaatagttcaactatttgtcagctcaagct | 627 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127741 | tcagtggaaagtattgcctcagggaatgcttaatagttcaactatttgtcagctcaagct | 127800 |
| Query: | 628 | ctgcaaccagttagagacaagttttcagactgttacatcgttcactatgttgatattttg | 687 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127801 | ctgcaaccagttagagacaagttttcagactgttacatcgttcactatgttgatattttg | 127860 |
| Query: | 688 | tgtgctgcagaaacgagagacaaattaattgaccgttacacatttctgcagacagaggtt | 747 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127861 | tgtgctgcagaaacgagagacaaattaattgaccgttacacatttctgcagacagaggtt | 127920 |
| Query: | 748 | gccaacgcggggctgacaataacatctgataagattcaaacctctactcctttccgttac | 807 |
| | | \|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127921 | gccaacgcgggactgacaataacatctgataagattcaaacctctactcctttccgttac | 127980 |
| Query: | 808 | ttgggaatgcaggtagaggaaaggaaaattaaacc | 842 |
| | | \|\|\|\|\|\|\|\|\|\|\|\|\|\|\|\|\|\|\|\|\|\|\|\|\|\|\|\|\|\|\|\|\|\|\| | |
| Sbjct: | 127981 | ttgggaatgcaggtagaggaaaggaaaattaaacc | 128015 |
| Score = 583 bits (294), Expect = e−164 | | | |
| Identities = 360/377 (95%), Gaps = 9/377 (2%) | | | |
| Strand = Plus/Plus | | | |
| Query: | 1 | acaacaatggcatgcagagattactatcccagcctccctatacagccccaggaatcaaaa | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\| | |
| Sbjct: | 127190 | acaacaatggcatgcagagattactatcccagcctccctatacagccccaggaataaaaa | 127249 |
| Query: | 61 | aatcatgactaaaatgggatagctccctaaaaagggactaggaaagaaagaagtcccaat | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\| | |
| Sbjct: | 127250 | aatcatgactaaaatgggatagctccctaaaaagggactaggaaagaa----gtcccaat | 127305 |
| Query: | 121 | tgaggctgaaaaaaattaaaaaagaaaaggaatagggcatcctttttaggagcggtcact | 180 |
| | | \|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127306 | tgaggctgaaaaaaatcaaaaaagaaaaggaatagggcatcctttttaggagcggtcact | 127365 |
| Query: | 181 | gtagagcctccaaaacccattccattaacttggg----aaaaaaaaaactgtatggtaaa | 236 |
| | | \|\|\|\|\|\|\|\|\|\|\|\|\|\|\|\|\|\|\|\|\|\|\|\|\|\|\|\|\|\|\|\|\|\| \|\|\|\|\|\|\| \|\|\|\|\|\|\|\|\|\|\|\|\|\| | |
| Sbjct: | 127366 | gtagagcctccaaaacccattccattaacttgggggaaaaaaaaacaactgtatggtaaa | 127425 |
| Query: | 237 | tcagcagccgcttccaaaacaaaagctggaggccttacacttattagcaaagaaaccatt | 296 |
| | | \|\|\|\|\|\|\|\| \|\|\|\|\|\|\|\|\|\|\|\|\|\|\| \|\|\|\|\|\|\|\| \|\|\|\|\| \|\|\|\|\|\|\|\|\|\|\|\|\|\|\|\| \|\|\| | |
| Sbjct: | 127426 | tcagcagc-gcttccaaaacaaaaactggaggctttacatttattagcaaagaaacaatt | 127484 |
| Query: | 297 | agaaaaaggacattgagccttcattttcgccttggaattctgtttgtgattcagaaaaaa | 356 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\| | |
| Sbjct: | 127485 | agaaaaaggacattgagccttcattttcgccttggaattctgtttgtaattcagaaaaaa | 127544 |
| Query: | 357 | tccggcagatggcgtat | 373 |
| | | \|\|\|\|\|\|\|\|\|\|\|\|\|\|\|\|\| | |
| Sbjct: | 127545 | tccggcagatggcgtat | 127561 |

SEQ ID 119 (035JN001.F06) aligns with available chromosome 22 sequence:

| Score = 1310 bits (661), Expect = 0.0 | | | |
|---|---|---|---|
| Identities = 664/665 (99%) | | | |
| Strand = Plus/Plus | | | |
| Query: | 96 | taatgccgtaattcaacccatgggggctctcccaccccggttgccctctccagccatggt | 155 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127561 | taatgccgtaattcaacccatgggggctctcccaccccggttgccctctccagccatggt | 127620 |
| Query: | 156 | cccctttaattataattgatctgaaggattgcttttttaccattcctctggcaaaacagg | 215 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127621 | cccctttaattataattgatctgaaggattgcttttttaccattcctctggcaaaacagg | 127680 |
| Query: | 216 | attttgaaaaatttgcttttaccacaccagcctaaataataaagaaccagccaccaggtt | 275 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127681 | attttgaaaaatttgcttttaccacaccagcctaaataataaagaaccagccaccaggtt | 127740 |
| Query: | 276 | tcagtggaaagtattgcctcagggaatgcttaatagttcaactatttgtcagctcaagct | 335 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127741 | tcagtggaaagtattgcctcagggaatgcttaatagttcaactatttgtcagctcaagct | 127800 |
| Query: | 336 | ctgcaaccagttagagacaagttttcagactgttacatcgttcactatgttgatattttg | 395 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127801 | ctgcaaccagttagagacaagttttcagactgttacatcgttcactatgttgatattttg | 127860 |
| Query: | 396 | tgtgctgcagaaacgagagacaaattaattgaccgttacacatttctgcagacagaggtt | 455 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127861 | tgtgctgcagaaacgagagacaaattaattgaccgttacacatttctgcagacagaggtt | 127920 |
| Query: | 456 | gccaacgcgggactgacaataacatctgataagattcaaacctctactcctttccgttac | 515 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127921 | gccaacgcgggactgacaataacatctgataagattcaaacctctactcctttccgttac | 127980 |
| Query: | 516 | ttgggaatgcaggtagaggaaaggaaaattaaaccacaaaaaatagaaataagaaaagac | 575 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127981 | ttgggaatgcaggtagaggaaaggaaaattaaaccacaaaaaatagaaataagaaaagac | 128040 |
| Query: | 576 | acattaaaagcattaaatgagtttcaaaagttgctaggagatactaattggatttggaga | 635 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128041 | acattaaaagcattaaatgagtttcaaaagttgctaggagatactaattggatttggaga | 128100 |
| Query: | 636 | tattaattggatttggccaactctaggcattcctacttatgccatgtcaaatttgtactc | 695 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\| | |
| Sbjct: | 128101 | tattaattggatttggccaactctaggcattcctacttatgccatgtcaaatttgttctc | 128160 |
| Query: | 696 | tttcttaagaggggactcggaattaaatagtgaaagaacgttaactccagaggcaactaa | 755 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 128161 | tttcttaagaggggactcggaattaaatagtgaaagaacgttaactccagaggcaactaa | 128220 |
| Query: | 756 | agaaa | 760 |
| | | \|\|\|\|\| | |
| Sbjct: | 128221 | agaaa | 128225 |
| Score = 159 bits (80), Expect = 3e−36 | | | |
| Identities = 80/80 (100%) | | | |
| Strand = Plus/Plus | | | |
| Query: | 2 | attagaaaaaggacattgagccttcattttcgccttggaattctgtttgtaattcagaaa | 61 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 127482 | attagaaaaaggacattgagccttcattttcgccttggaattctgtttgtaattcagaaa | 127541 |
| Query: | 62 | aaatccggcagatggcgtat | 81 |
| | | \|\|\|\|\|\|\|\|\|\|\|\|\|\|\|\|\|\|\|\| | |
| Sbjct: | 127542 | aaatccggcagatggcgtat | 127561 |

Patient Tumor Samples

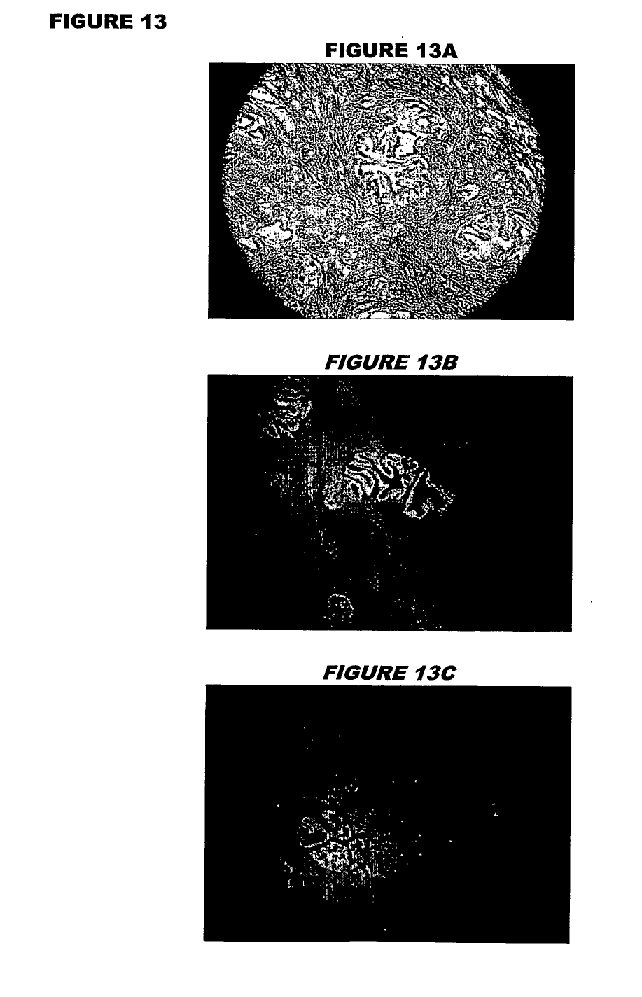

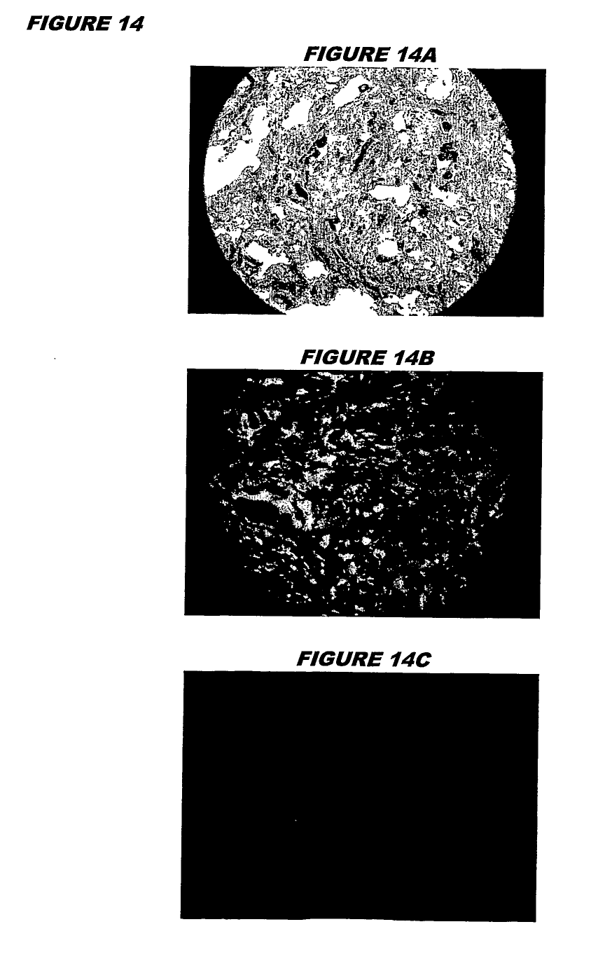

Fresh frozen prostate cancer tissue from two patients was cut in 10 micron sections, mounted on glass slides, and stained with murine monoclonal antibody 5G2. The staining was visualized with a second antibody (fluorescein-coupled goat anti-mouse). Staining was found to be specific for cancerous tissue. The samples were also analyzed by hybridization to 26254 and signal was 3540 times stronger than in control samples from the same patient:

| patient ID# | Gleason grade | 5G2 staining | spot 26254 ratio |
|---|---|---|---|
| 101 | 3 + 3 | +(FIG. 13) | 35 |
| 153 | 3 + 3 | +(FIG. 14) | 40 |

RT-PCR

RNA extracts from various tissues were analyzed by RT-PCR. In particular, the splicing event between exons 1 and 2 was investigated using primers as shown in FIG. 6. Results are shown in FIG. 10. All lanes show background levels of HERV-K HML2.0 (i.e. new virus) expression (thin lines) but prostate tissue (lane 6) shows a longer product (thick line), indicating expression of a HERV-K with a longer sequence between the 5′ LTR and the start of ENV. The difference in length between the long lane 6 product and the background product seen in other tissues (˜80 bp) corresponds in length to the length of exon 2 illustrated in FIG. 6B.

Extracts from cell lines were also tested (FIG. 11). Again, background levels of “ubiquitous” HERV-K expression were evident in most cell lines. Prostate cell lines MDA PCA 2b (lane 7) and, to a lesser extent, 22RV1 (lane 6), clearly showed longer RT-PCR products.

MDA PCA 2b Cell Line

RNA was extracted from MDA PCA 2b cell lines. Spliced mRNAs were cloned and sequenced which confirm that splice acceptor sites near the 3′ end of the second 5′ LTR are used. These mRNAs have four exons with sequences exactly matching PCAV. They have exons adjacent to LTRs 1 and 2 followed by an exon containing the envelope ATG and a very short open reading frame and finally terminating in the final fragmentary 3′ LTR.

The use of a splice acceptor site near the 3′ end of the second 5′ LTR was also seen in a cDNA present in a private prostate cancer library (Chiron clone ID 035JN024.B09).

The 3′ end of MDA PCA 2b RNA was mapped by RACE. The forward PCR primer was SEQ ID 21, which matches PCAV and new HERV-Ks. The reverse PCR primer was SEQ ID 22. The primer for reverse transcription was SEQ ID 20. Using mRNA targets from MDA PCA 2b gave a major band at 1.3 kb. The bands were cloned and sequenced (using either T7 or SP6 sequencing primers) and an alignment is shown below:

| | | 1 ∠ 40 | |
|---|---|---|---|
| PCAV ch22 Mer11a | (1) | TGTTGTGGGAAGTCAGGGACCCCGAATGGAGGGACCAGCT | |
| MDARU3#1 × T7 rev | (1) | ---------------------------------------- | |
| MDARU3#2 × SP6 REV | (1) | ---------------------------------------- | |
| MDARU3#4 × SP6 rev | (1) | ---------------------------------------- | |
| MDARU3#5 × T7 rev | (1) | ---------------------------------------- | |
| MDARU3#6 × T7 rev | (1) | ---------------------------------------- | |
| Consensus | (1) | | |
| | | 41 80 | |
| PCAV ch22 Mer11a | (41) | GGTGCTGCATCAGGAAACATAAATTGTGAAGATTTCTTGG | |
| MDARU3#1 × T7 rev | (1) | ---------------------------------------- | |
| mdaru3#2 × SP6 REV | (1) | ---------------------------------------- | |
| MDARU3#4 × SP6 rev | (1) | ---------------------------------------- | |
| MDARU3#5 × T7 rev | (1) | ---------------------------------------- | |
| MDARU3#6 × T7 rev | (1) | ---------------------------------------- | |
| Consensus | (41) | | |
| | | 81 120 | |
| PCAV ch22 Mer11a | (81) | ACATTTATCAGTTTCCAAAATTAATACTTTTATAATTTCT | |
| MDARU3#1 × T7 rev | (1) | ---------------------------------------- | |
| MDARU3#2 × SP6 REV | (1) | ---------------------------------------- | |
| MDARU3#4 × SP6 rev | (1) | ---------------------------------------- | |
| MDARU3#5 × T7 rev | (1) | ---------------------------------------- | |
| MDARU3#6 × T7 rev | (1) | ---------------------------------------- | |
| Consensus | (81) | | |
| | | 121 160 | |
| PCAV ch22 Mer11a | (121) | TACACCTGTCTTACTTTAATCTCTTAATCCTGTTATCTTT | |
| MDARU3#1 × T7 rev | (1) | ---------------------------------------- | |
| MDARU3#2 × SP6 REV | (1) | ---------------------------------------- | |
| MDARU3#4 × SP6 rev | (1) | ---------------------------------------- | |
| MDARU3#5 × T7 rev | (1) | ---------------------------------------- | |
| MDARU3#6 × T7 rev | (1) | ---------------------------------------- | |
| Consensus | (121) | | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (161) (1) (1) (1) (1) (1) (161) | 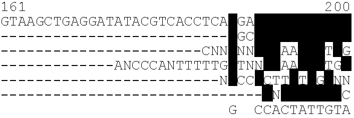 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (201) (15) (18) (28) (16) (11) (201) | 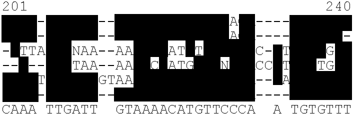 | |
| PCAV ch22 Mer11a MDARU3#1 × T7 rev MDARU3#2 > SP6 REV MDARU3#4 × SP6 rev MDARU3#5 × T7 rev MDARU3#6 × T7 rev Consensus | (234) (47) (53) (62) (54) (44) (241) | 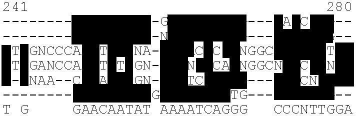 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (260) (73) (92) (101) (87) (71) (281) | 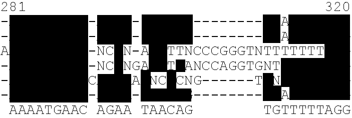 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (289) (102) (130) (139) 9120) (100) (321) | 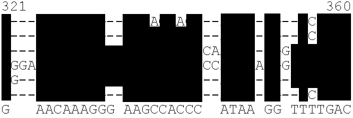 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (319) (132) (166) (179) (154) (130) (361) | 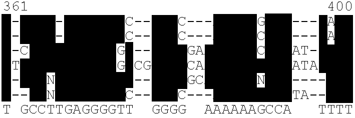 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (349) (162) (202) (219) (187) (161) (401) | 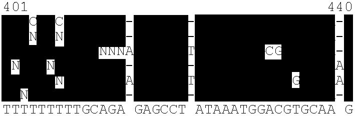 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (386) (199) (241) 9257) (227) (198) (441) | 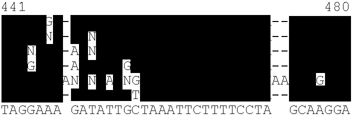 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (423) (236) (278) (294) (267) (235) (481) | 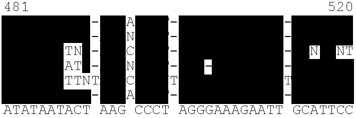 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (460) (273) (315) (330) (307) (272) (521) | 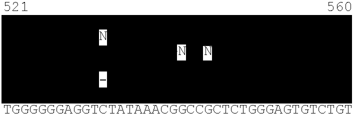 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (500) (313) (355) (370) (346) (312) (561) | 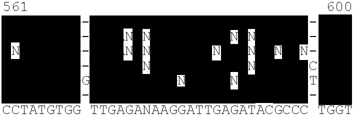 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (538) (351) (393) (409( (386) (350) (601) | 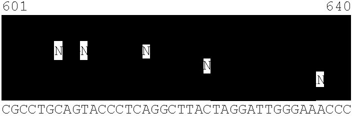 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (578) (391) (433) (449) (426) (390) (641) | 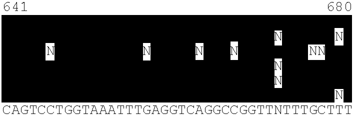 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (618) (431) (473) (489) (466) (430) (681) | 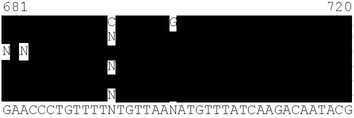 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (658) (471) (513) (529) (506) (470) (721) | 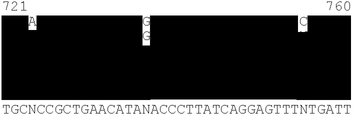 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (698) (511) (553) (569) (546) (510) (761) | 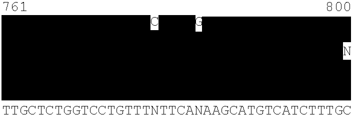 | |
| PCAV ch22 Mer11a MDARU3#1 T7 rev MDARU3#2 × SP6 REV MDARU3#4 × SP6 rev MDARU3#5 × T7 rev MDARU3#6 × T7 rev Consensus | (738) (551) (593) (609) (586) (550) (801) | 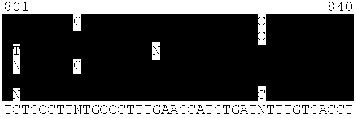 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (778) (591) (633) (649) (626) (590) (841) | 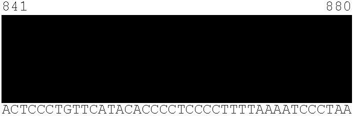 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (818) (631) (673) (689) (666) (630) (881) | 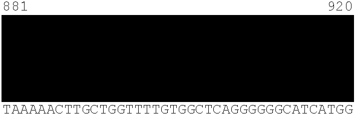 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (858) (671) (713) (729) (706) (670) (921) | 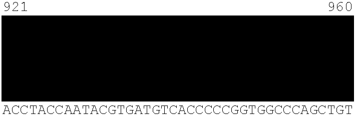 | |
| PCAV ch22 Mer11a MDARU3#1 × T7 rev MDARU3#2 × SP6 REV MDARU3#4 × SP6 rev MDARU3#5 × T7 rev MDARU3#6 × T7 rev Consensus | (898) (711) (753) (769) (746) (710) (961) | 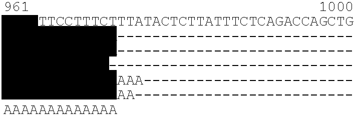 | |
| | | 1001 1040 | |
| PCAV ch22 Mer11a | (938) | ACACTTAGGGAAAATAGAAAGAACCTATGTTGAAATATTG | |
| MDARU3#1 × T7 rev | (724) | ---------------------------------------- | |
| MDARU3#2 × SP6 REV | (766) | ---------------------------------------- | |
| MDARU3#4 × SP6 rev | (781) | ---------------------------------------- | |
| MDARU3#5 × T7 rev | (762) | ---------------------------------------- | |
| MDARU3#6 × T7 rev | (725) | ---------------------------------------- | |
| Consensus | (1001) | | |
| | | 1041 1059 | |
| PCAV ch22 Mer11a | (978) | GAGGCGGGTTCCCCCGATA | |
| MDARU3#1 × T7 rev | (724) | ------------------- | <SEQ ID 89> |
| MDARU3#2 × SP6 REV | (766) | ------------------- | <SEQ ID 90> |
| MDARU3#4 × SP6 rev | (781) | ------------------- | <SEQ ID 91> |
| MDARU3#5 × T7 rev | (762) | ------------------- | |
| MDARU3#6 × T7 rev | (725) | ------------------- | |
| Consensus | (1041) | | |

Sequencing of these amplification products shows that transcripts terminate using a polyA signal within a MER11a insertion (see row beginning with nucleotide 961). Again, this is a perfect match for PCAV.

Anti-Gag Monoclonal Antibodies

PCAV is an “old” HERV-K. Low-level expression of “new” HERV-Ks can also be detected. The gag open reading frames from PCAV and the “new” HERV-Ks are homologous at the primary sequence level, but with significant divergence. Gag protein was expressed in yeast and purified for both PCAV and “new” HERV-K, and mouse monoclonal antibodies were raised.

The “new” HERV-K gag sequence used for expression was isolated from the prostate cancer cell line LnCap and the PCAV gag sequence was isolated from the prostate cancer cell line MDA PCA 2b. These sequences were genetically engineered for expression in Saccharomyces cerevisiae AD3 strain, using the yeast expression vector pBS24.1. This vector contains the 2μ sequence for autonomous replication in yeast and the yeast genes leu2d and URA3 as selectable markers. The β-lactamase gene and the ColE1 origin of replication, required for plasmid replication in bacteria, are also present in this expression vector, as well as the a-factor terminator. Expression of the recombinant proteins is under the control of the hybrid ADH2/GAPDH promoter.

The coding sequences for “new” HERV-K and PCAV gag were cloned as HindIII-SalI fragments of 2012 bp and 2168 bp respectively. Each gag was subcloned in two parts:

1. The “new” HERV-K gag was subcloned into pSP72. A 143 bp synthetic oligonucleotide from the HindIII site adjoined the ADH/GAPDH promoter to a NcoI site within the gag coding sequence. The remaining 1869 bp of “new” HERV-K gag sequence, from NcoI to SalI, was derived by PCR using a cDNA clone obtained from LnCaP cells named orf-99 as the template.

2. PCR was used to create a 1715 bp HindIII-Ava3 fragment PCAV gag, using a cDNA clone obtained from MDA PCa 2b cells named 2B11.12-44 as the template. The resulting PCR product was subcloned into pGEM7-Z. The Ava3-SalI fragment encoding the 3′ end of this construct was isolated from the “new” HERV-K gag clone above, since the 3′ end of the gag protein was missing in the 2B11.12-44 clone.

After sequence confirmation the respective fragments were ligated with the ADH2/GAPDH promoter into the yeast expression vector to create pd.LnCap.gag (encoding the “new” HERV-K gag) and pd.MDA.gag (encoding the hybrid PCAV/“new” HERV-K gag) yeast expression plasmids.

The “new” expression construct is SEQ ID 1185 and encodes SEQ ID 1186:

| \|_{————}\|_{————}\|_{——}\|_{——————}\|_\|_{————————————————————}\|\|_{——}\|\|_{—} | |
|---|---|
| HIND3 NCOI XMNI NAEI | |
| AHA3 BGL2 ALWN1 ALWN1 | |
| ECORV | |
| BSMI | |
| \|_{———}\|_{————}\|_{————————}\|_{——————}\|_{——}\|_{———————————}\|_{——}\|_{——————}\| | |
| KAS1 BGL2 BAMHI BALI MST2 ASE1 | |
| DRA3 AHA3 ALWN1 | |
| ASE1 | |
| NARI | |
| _{—————————}\|_{———————————}\|_{———————\|}\|_{——}\|_{————}\|_{——}\|_{—} | |
| AVA3 PFLM1 MST2 PVU2 | |
| BSTXI SALI | |
| MST2 | |
| | MetGlyGlnThrGluSerLysTyrAlaSerTyrLeuSerPheIle |
| 2 | AGCTTACAAAACAAAATGGGGCAAACTGAAAGTAAATATGCCTCTTATCTCAGCTTTATT |
| | TCGAATGTTTTGTTTTACCCCGTTTGACTTTCATTTATACGGAGAATAGAGTCGAAATAA |
| | \hat{ } |
| | 1 HIND3, |
| | LysIleLeuLeuLysArgGlyGlyValArgValSerThrLysAsnLeuIleLysLeuPhe |
| 62 | AAAATTCTTTTAAAAAGAGGGGGAGTTAGAGTATCTACAAAAAATCTAATCAAGCTATTT |
| | TTTTAAGAAAATTTTTCTCCCCCTCAATCTCATAGATGTTTTTTAGATTAGTTCGATAAA |
| | \hat{ } |
| | 70 AHA3, |
| | GlnIleIleGluGlnPheCysProTrpPheProGluGlnGlyThrLeuAspLeuLysAsp |
| 122 | CAAATAATAGAACAATTTTGCCCATGGTTTCCAGAACAAGGAACTTTAGATCTAAAAGAT |
| | GTTTATTATCTTGTTAAAACGGGTACCAAAGGTCTTGTTCCTTGAAATCTAGATTTTCTA |
| | \hat{ } \hat{ } |
| | 143 NCOI, 169 BGL2, |
| | TrpLysArgIleGlyGluGluLeuLysGlnAlaGlyArgLysGlyAsnIleIleProLeu |
| 182 | TGGAAAAGAATTGGCGAGGAACTAAAACAAGCAGGTAGAAAGGGTAATATCATTCCACTT |
| | ACCTTTTCTTAACCGCTCCTTGATTTTGTTCGTCCATCTTTCCCATTATAGTAAGGTGAA |
| | ThrValTrpAsnAspTrpAlaIleIleLysAlaAlaLeuGluProPheGlnThrLysGlu |
| 242 | ACAGTATGGAATGATTGGGCCATTATTAAAGCAGCTTTAGAACCATTTCAAACAAAAGAA |
| | TGTCATACCTTACTAACGCGGTAATAATTTCGTCGAAATCTTGGTAAAGTTTGTTTTCTT |
| | \hat{ } |
| | 281 XMNI, |
| | AspSerValSerValSerAspAlaProGlySerCysValIleAspCysAsnGluLysThr |
| 302 | GATAGCGTTTCAGTTTCTGATGCCCCTGGAAGCTGTGTAATAGATTGTAATGAAAAGACA |
| | CTATCGCAAAGTCAAAGACTACGGGGAGCTTCGACACATTATCTAACATTACTTTTCTGT |
| | \hat{ } |
| | 312 ALWN1, |
| | GlyArgLysSerGlnLysGluThrGluSerLeuHisCysGluTyrValThrGluProVal |
| 362 | GGGAGAAAATCCCAGAAAGAAACAGAAAGTTTACATTGCGAATATGTAACAGAGCCAGTA |
| | CCCTCTTTTAGGGTCTTTCTTTGTCTTTCAAATGTAACGCTTATACATTGTCTCGGTCAT |
| | MetAlaGlnSerThrGlnAsnValAspTyrAsnGlnLeuGlnGlyValIleTyrProGlu |
| 422 | ATGGCTCAGTCAACGCAAAATGTTGACTATAATCAATTACAGGGGGTGATATATCCTGAA |
| | TACCGAGTCAGTTGCGTTTTACPACTGATATTAGTTAATGTCCCCCACTATATAGGACTT |
| | ThrLeuLysLeuGluGlyLysGlyProGluLeuValGlyProSerGluSerLysProArg |
| 482 | ACGTTAAAATTAGAAGGAAAAGGTCCAGAATTAGTGGGGCCATCAGAGTCTAAACCACGA |
| | TGCAATTTTAATCTTCCTTTTCCAGGTGTTAATCACCCCGGTAGTCTCAGATTTGGTGCT |
| | GlyProSerProLeuProAlaGlyGlnValProValThrLeuGlnProGlnThrGlnVal |
| 542 | GGGCCAAGTCCTCTTCCAGCAGGTCAGGTGCCCGTAACATTACAACCTCAAACGCAGGTT |
| | CCCGGTTCAGGAGAAGGTCGTCCAGTCCACGGGCATTGTAATGTTGGAGTTTGCGTCCAA |
| | LysGluAsnLysThrGlnProProValAlaTyrGlnTyrTrpProProAlaGluLeuGln |
| 602 | AAAGAAAATAAGACCCAACCGCCAGTAGCTTATCAATACTGGCCGCCGGCTGAACTTCAG |
| | TTTCTTTTATTCTGGGTTGGCGGTCATCGAATAGTTATGACCGGCGGCCGACTTGAAGTC |
| | \hat{ } \hat{ } |
| | 646 NAEI, 659 ALWN1, |
| | TyrLeuProProProGluSerGlnTyrGlyTyrProGlyMetProProAlaLeuGlnGly |
| 662 | TATCTGCCACCCCCAGAAAGTCAGTATGGATATCCAGGAATGCCCCCAGCACTACAGGGC |
| | ATAGACGGTGGGGGTCTTTCAGTCATACCTATAGGTCCTTACGGGGGTCGTGATGTCCCG |
| | \hat{ } \hat{ } |
| | 690 ECORV, 699 BSMI, |
| | ArgAlaProTyrProGlnProProThrValArgLeuAsnProThrAlaSerArgSerGly |
| 722 | AGGGCGCCATATCCTCAGCCGCCCACTGTGAGACTTAATCCTACAGCATCACGTAGTGGA |
| | TCCCGCGGTATAGGAGTCGGCGGGTGACACTCTGAATTAGGATGTCGTAGTGCATCACCT |
| | \hat{ } \hat{ } |
| | 724 KAS1 NARI, 771 DRA3, |
| | GlnGlyGlyThrLeuHisAlaValIleAspGluAlaArgLysGlnGlyAspLeuGluAla |
| 782 | CAAGGTGGTACACTGCACGCAGTCATTGATGAAGCCAGAAAACAGGGAGATCTTGAGGCA |
| | GTTCCACCATGTGACGTGCGTCAGTAACTACTTCGGTCTTTTGTCCCTCTAGAACTCCGT |
| | \hat{ } |
| | 829 BGL2, |
| | TrpArgPheLeuValIleLeuGlnLeuValGlnAlaGLyGluGluThrGlnValGlyAla |
| 842 | TGGCGGTTCCTGGTAATTTTACAACTGGTACAGGCCGGGGAAGAGACTCAAGTAGGAGCG |
| | ACCGCCAAGGACCATTAAAATGTTGACCATGTCCGCCCCCTTCTCTGAGTTCATCCTCGC |
| | ProAlaArgAlaGluThrArgCysGluProPheThrMetLysMetLeuLysAspIleLys |
| 902 | CCTGCCCGAGCTGAGACTAGATGTGAACCTTTCACCATGAAAATGTTAAAAGATATAAAG |
| | GGACGGGCTCGACTCTGATCTACACTTGGAAAGTGGTACTTTTACAATTTTCTATATTTC |
| | GluGlyValLysGlnTyrGlySerAsnSerProTyrIleArgThrLeuLeuAspSerIle |
| 962 | GAAGGAGTTAAACAATATGGATCCAACTCCCCTTATATAAGAACATTATTAGATTCCATT |
| | CTTCCTCAATTTGTTATACCTAGGTTGAGGGGAATATATTCTTGTAATAATCTAAGGTAA |
| | \hat{ } |
| | 980 BAMHI, |
| | AlaHisGlyAsnArgLeuThrProTyrAspTrpGluSerLeuAlaLysSerSerLeuSer |
| 1022 | GCTCATGGAAATAGACTTACTCCTTATGACTGGGAAAGTTTGGCCAAATCTTCCCTTTCA |
| | CGAGTACCTTTATCTGAATGAGGAATACTGACCCTTTCAAACCGGTTTAGAAGGGAAAGT |
| | \hat{ } |
| | 1062 BALI, |
| | SerSerGlnTyrLeuGlnPheLysThrTrpTrpIleAspGlyValGlnGluGlnValArg |
| 1082 | TCCTCTCAGTATCTACAGTTTAAAACCTGGTGGATTGATGGAGTACAAGAACAGGTACGA |
| | AGGAGAGTCATAGATGTCAAATTTTGGACCACCTAACTACCTCATGTTCTTGTCCATGCT |
| | \hat{ } |
| | 1100 AHA3, |
| | LysAsnGlnAlaThrLysProThrValAsnIleAspAlaAspGlnLeuLeuGlyThrGly |
| 1142 | AAAAATCAGGCTACTAAGCCCACTGTTAATATAGACGCAGACCAATTGTTAGGAACAGGT |
| | TTTTTAGTCCGATGATTCGGGTGACAATTATATCTGCGTCTGGTTAACAATCCTTGTCCA |
| | ProAsnTrpSerThrIleAsnGlnGlnSerValMetGlnAsnGluAlaIleGluGlnVal |
| 1202 | CCAAATTGGAGCACCATTAACCAACAATCAGTGATGCAGAATGAGGCTATTGAACAAGTA |
| | GGTTTAACCTCGTGGTAATTGGTTGTTAGTCACTACGTCTTACTCCGATAACTTGTTCAT |
| | ArgAlaIleCysLeuArgAlaTrpGlyLysIleGlnAspProGlyThrAlaPheProIle |
| 1262 | AGGGCTATTTGCCTCAGGGCCTGGGGAAAAATTCAGGACCCAGGAACAGCTTTCCCTATT |
| | TCCCGATAAACGGAGTCCCGGACCCCTTTTTAAGTCCTGGGTCCTTGTCGAAAGGGATAA |
| | \hat{ } \hat{ } \hat{ } |
| | 1273 MST2, 1276 ALWN1, 1319 ASE1, |
| | AsnSerIleArgGlnGlySerLysGluProTyrProAspPheValAlaArgLeuGlnAsp |
| 1322 | AATTCAATTAGACAAGGCTCTAAAGAGCCATATCCTGACTTTGTGGCAAGATTACAAGAT |
| | TTAAGTTAATCTGTTCCGAGATTTCTCGGTATAGGACTGAAACACCGTTCTAATGTTCTA |
| | AlaAlaGlnLysSerIleThrAspAspAsnAlaArgLysValIleValGluLeuMetAla |
| 1382 | GCTGCTCAAAAGTCTATTACAGATGACAATGCCCGAAAAGTTATTGTAGAATTAATGGCC |
| | CGACGAGTTTTCAGATAATGTCTACTGTTACGGGCTTTTCAATAACATCTTAATTACCGG |
| | \hat{ } |
| | 1432 ASE1, |
| | TyrGluAsnAlaAsnProGluCysGlnSerAlaIleLysProLeuLysGlyLysValPro |
| 1442 | TATGAAAATGCAAATCCAGAATGTCAGTCGGCCATAAAGCCATTAAAAGGAAAAGTTCCA |
| | ATACTTTTACGTTTAGGTCTTACAGTCAGCCGGTATTTCGGTAATTTTCCTTTTCAAGGT |
| | AlaGlyValAspValIleThrGluTyrValLysAlaCysAspGlyIleGlyGlyAlaMet |
| 1502 | GCAGGAGTTGATGTAATTACAGAATATGTGAAGGCTTGTGATGGGATTGGAGGAGCTATG |
| | CGTCCTCAACTACATTAATGTCTTATACACTTCCGAACACTACCCTAACCTCCTCGATAC |
| | \hat{ } |
| | 1559 AVA3, |
| | HisLysAlaMetLeuMetAlaGlnAlaMetArgGlyLeuThrLeuGlyGlyGlnValArg |
| 1562 | CATAAGGCAATGCTAATGGCTCAAGCAATGAGGGGGCTCACTCTAGGAGGACAAGTTAGA |
| | GTATTCCGTTACGATTACCGAGTTCGTTACTCCCCCGAGTGAGATCCTCCTGTTCAATCT |
| | ThrPheGlyLysLysCysTyrAsnCysGlyGlnIleGlyHisLeuLysArgSerCysPro |
| 1622 | ACATTTGGGAAAAAATGTTATAATTGTGGTCAAATCGGTCATCTGAAAAGGAGTTGCCCA |
| | TGTAAACCCTTTTTTACAATATTAACACCAGTTTAGCCAGTAGACTTTTCCTCAACGGGT |
| | ValLeuAsnLysGlnAsnIleIleAsnGlnAlaIleThrAlaLysAsnLysLysProSer |
| 1682 | GTCTTAAATAAACAGAATATAATAAATCAAGCTATTACAGCAAAAAATAAAAAGCCATCT |
| | CAGAATTTATTTGTCTTATATTATTTAGTTCGATAATGTCGTTTTTTATTTTTCGGTAGA |
| | GlyLeuCysProLysCysGlyLysGlyLysHisTrpAlaAsnGlnCysHisSerLysPhe |
| 1742 | GGCCTGTGTCCAAAATGTGGAAAAGGAAAACATTGGGCCAATCAATGTCATTCTAAATTT |
| | CCGGACACAGGTTTTACACCTTTTCCTTTTGTAACCCGGTTAGTTACAGTAAGATTTAAA |
| | \hat{ } |
| | 1751 PFLM1, |
| | AspLysAspGlyGlnProLeuSerGlyAsnArgLysArgGlyGlnProGlnAlaProGln |
| 1802 | GATAAGGATGGGCAACCATTGTCGGGAAACAGGAAGAGGGGCCAGCCTCAGGCCCCCCAA |
| | CTATTCCTACCCGTTGGTAACAGCCCTTTGTCCTTCTCCCCGGTCGGAGTCCGGGGGGTT |
| | \hat{ } \hat{ } |
| | 1847 MST2, 1858 BSTXI, |
| | GlnThrGlyAlaPheProValGlnLeuPheValProGlnGlyPheGlnGlyGlnGlnPro |
| 1862 | CAAACTGGGGCATTCCCAGTTCAACTGTTTGTTCCTCAGGGTTTTCAAGGACAACAACCC |
| | GTTTGACCCCGTAAGGGTCAAGTTGACAAACAAGGAGTCCCAAAAGTTCCTGTTGTTGGG |
| | \hat{ } |
| | 1895 MST2, |
| | LeuGlnLysIleProProLeuGlnGlyValSerGlnLeuGlnGlnSerAsnSerCysPro |
| 1922 | CTACAGAAAATACCACCACTTCAGGGAGTCAGCCAATTACAACAATCCAACAGCTGTCCC |
| | GATGTCTTTTATGGTGGTGAAGTCCCTCAGTCGGTTAATGTTGTTAGGTTGTCGACAGGG |
| | \hat{ } |
| | 1972 PVU2, |
| | AlaProGlnGlnAlaAlaProGlnAM OC |
| 1982 | GCGCCACAGCAGGCAGCACCGCAGTAGTAAGTCGAC |
| | CGCGGTGTCGTCCGTCGTGGCGTCATCATTCAGCTG |
| | \hat{ } |
| | 2012 SALI, |

The hybrid construct is SEQ ID 1187 and encodes SEQ ID 1188:

| \|_{———————}\|_\|_{——————}\|_\|_{—————}\|_{———————}\|_{—————}\|_{————}\|_{————} | |
|---|---|
| HIND3 NCOI XMNI ALWN1 PVUI TTH3I-I | |
| BGL2 ALWN1 RSPI PFLM1 | |
| BSAB1 | |
| \|_{————}\|_{—————}\|\|\|_{————————}\|_{—————}\|_{——}\|_{————————————}\|\|_{——}\|_{—————} | |
| HGIE2 DRA3 BAMHI AHA3 MST2 | |
| BSTXI APAL1 BALI ALWN1 | |
| SPHI ASE1 | |
| _{——}\|_{————————}\|_{——————————}\|_{——————}\|_{——}\|_{—————}\|_{——}\|_{—} | |
| ASE1 AVA3 PFLM1 MST2 PVU2 | |
| BSTXI SALI | |
| MST2 | |
| | MetGlyGlnThrGluSerLysTyrAlaSerTyrLeuSerPheIle |
| 2 | AGCTTACAAAACAAAATGGGGCAAACTGAAAGTAAATATGCCTCTTATCTCAGCTTTATT |
| | TCGAATGTTTTGTTTTACCCCGTTTGACTTTCATTTATACGGAGAATAGAGTCGAAATAA |
| | \hat{ } |
| | 1 HIND3, |
| | LysIleLeuLeuArgArgGlyGlyValArgAlaSerThrGluAsnLeuIleThrLeuPhe |
| 62 | AAAATTCTTTTAAGAAGAGGGGGAGTTAGAGCTTCTACAGAAAATCTAATTACGCTATTT |
| | TTTTAAGAAAATTCTTCTCCCCCTCAATCTCGAAGATGTCTTTTAGATTAATGCGATAAA |
| | GlnThrIleGluGlnPheCysProTrpPheProGluGlnGlyThrLeuAspLeuLysAsp |
| 122 | CAAACAATAGAACAATTCTGCCCATGGTTTCCAGAACAGGGAACTTTAGATCTAAAAGAT |
| | GTTTGTTATCTTGTTAAGACGGGTACCAAAGGTCTTGTCCCTTGAAATCTAGATTTTCTA |
| | \hat{ } \hat{ } |
| | 143 NCOI, 169 BGL2, |
| | TrpGluLysIleGlyLysGluLeuLysGlnAlaAsnArgGluGlyLysIleIleProLeu |
| 182 | TGGGAAAAAATTGGCAAAGAATTAAAACAAGCAAATAGGGAAGGTAAAATCATCCCACTT |
| | ACCCTTTTTTAAGCGTTTCTTAATTTTGTTCGTTTATCCCTTCCATTTTAGTAGGGTGAA |
| | ThrValTrpAsnAspTrpAlaIleIleLysAlaThrLeuGluProPheGlnThrGlyGlu |
| 242 | ACAGTATGGAATGATTGGGCCATTATTAAAGCAACTTTAGAACCATTTCAAACAGGAGAA |
| | TGTCATACCTTACTAACCGGGTAATAATTTCGTTGAAATCTTGGTAAAGTTTGTCCTCTT |
| | \hat{ } |
| | 281 XMNI, |
| | AspIleValSerValSerAspAlaProLysSerCysValThrAspCysGluGluGluAla |
| 302 | GATATTGTTTGAGTTTCTGATGCCCCTAAAAGCTGTGTAACAGATTGTGAAGAAGAGGCA |
| | CTATAACAAAGTCAAAGACTACGGGGATTTTCGACACATTGTCTAACACTTCTTCTCCGT |
| | \hat{ } |
| | 312 ALWN1, |
| | GlyThrGluSerGlnGlnGlyThrGluSerSerHisCysLysTyrValAlaGluSerVal |
| 362 | GGGACAGAATCCCAGCAAGGAACGGAAAGTTCACATTGTAAATATGTAGCAGAGTCTGTA |
| | CCCTGTCTTAGGGTCGTTCCTTGCCTTTCAAGTGTAACATTTATACATCGTCTCAGACAT |
| | \hat{ } |
| | 411 ALWN1, |
| | MetAlaGlnSerThrGlnAsnValAspTyrSerGlnLeuGlnGluIleIleTyrProGlu |
| 422 | ATGGCTCAGTCAACGCAAAATGTTGACTACAGTCAATTACAGGAGATAATATACCCTGAA |
| | TACCGAGTCAGTTGCGTTTTACAACTGATGTCAGTTAATGTCCTCTATTATATGGGACTT |
| | SerSerLysLeuGlyGluGlyGlyProGluSerLeuGlyProSerGluProLysProArg |
| 482 | TCATCAAAATTGGGGGAAGGAGGTCCAGAATCATTGGGGCCATCAGAGCCTAAACCACGA |
| | AGTAGTTTTAACCCCCTTCCTCCAGGTCTTAGTAACCCCGGTAGTCTCGGATTTGGTGCT |
| | \hat{ }\hat{ } |
| | 539 PVUI RSPI, 540 BSAB1, |
| | SerProSerThrProProProValValGlnMetProValThrLeuGlnProGlnThrGln |
| 542 | TCGCCATCAACTCCTCCTCCCGTGGTTCAGATGCCTGTAACATTACAACCTCAAACGCAG |
| | AGCGGTAGTTGAGGAGGAGGGCACCAAGTCTACGGACATTGTAATGTTGGAGTTTGCGTC |
| | ValArgGlnAlaGlnThrProArgGluAsnGlnValGluArgAspArgValSerIlePro |
| 602 | GTTAGACAAGCAGAAACCCCAAGAGAAAATCAAGTAGAAAGGGACAGAGTCTCTATCCCG |
| | CAATCTGTTCGTGTTTGGGGTTCTCTTTTAGTTCATCTTTCCCTGTCTCACAGATAGGGC |
| | \hat{ } |
| | 644 TTH3I, |
| | AlaMetProThrGlnIleGlnTyrProGlnTyrGlnProValGluAsnLysThrGlnPro |
| 662 | GCAATGCCAACTCAGATACAGTATCCACAATATCAGCCGGTAGAAAATAAGACCCAACCG |
| | CGTTACGGTTGAGTCTATGTCATAGGTGTTATAGTCGGCCATCTTTTATTCTGGGTTGGC |
| | \hat{ } |
| | 715 PFLM1, |
| | LeuValValTyrGlnTyrArgLeuProThrGluLeuGlnTyrArgProProSerGluVal |
| 722 | CTGGTAGTTTATCAATACCGGCTGCCAACCGAGCTTCAGTATCGGCCTCCTTCAGAGGTT |
| | GACCATCAAATAGTTATGGCCGACGGTTGGCTCGAAGTCATAGCCGGAGGAAGTCTCCAA |
| | GlnTyrArgProGlnAlaValCysProValProAsnSerThrAlaProTyrGlnGlnPro |
| 782 | CAATACAGACCTCAAGCGGTGTGTCCTGTGCCAAATAGCACGGCACCATACCAGCAACCC |
| | GTTATGTCTGGAGTTCGCCACACAGGACACGGTTTATCCTGCCGTGGTATGGTCGTTGGG |
| | \hat{ } \hat{ } |
| | 790 HGIE2, 840 BSTXI, |
| | ThrAlaMetAlaSerAsnSerProAlaThrGlnAspAlaAlaLeuTyrProGlnProPro |
| 842 | ACAGCGATGGCGTCTAATTCACCAGCAACACAGGACGCGGCGCTGTATCCTCAGCGGCCC |
| | TGTCGCTACCGCAGATTAAGTGGTCGTTGTGTCCTGCGCCGCGACATAGGAGTCGGCGGG |
| | ThrValArgLeuAsnProThrAlaSerArgSerGlyGlnGlyGlyAlaLeuHisAlaVal |
| 902 | ACTGTGAGACTTAATCCTACAGCATCACGTAGTGGACAGGGTGGTGCACTGCATGCAGTC |
| | TGACACTCTGAATTAGGATGTCGTAGTGCATCACCTGTCCCACCACGTGACGTACGTCAG |
| | \hat{ } \hat{ } \hat{ } |
| | 927 DRA3, 945 APAL1, 952 SPHI, |
| | IleAspGluAlaArgLysGlnGlyAspLeuGluAlaTrpArgPheLeuValIleLeuGln |
| 962 | ATTGATGAAGCCAGAAAACAGGGCGATCTTGAGGCATGGCGGTTCCTGGTAATTTTACAA |
| | TAACTACTTCGGTCTTTTGTCCCGCTAGAACTCCGTACCGCCAAGGACCATTAAAATGTT |
| | LeuValGlnAlaGlyGluGluThrGlnValGlyAlaProAlaArgAlaGluThrArgCys |
| 1022 | CTGGTACAGGCCGGGGAAGAGACTCAAGTAGGAGCGCCTGCCCGAGCTGAGACTAGATGT |
| | GACCATGTCCGGCCCCTTCTCTGAGTTCATCCTCGCGGACGGGCTCGACTCTGATCTACA |
| | GluProPheThrMetLysMetLeuLysAspIleLysGluGlyValLysGlnTyrGlySer |
| 1082 | GAACCTTTCACCATGAAAATGTTAAAAGATATAAAGGAAGGAGTTAAACAATATGGATCC |
| | CTTGGAAAGTGGTACTTTTACAATTTTCTATATTTCCTTCGTCAATTTGTTATACCTAGG |
| | \hat{ } |
| | 1136 BAMHI, |
| | AsnSerProTyrIleArgThrLeuLeuAspSerIleAlaHisGlyAsnArgLeuThrPro |
| 1142 | AACTCCCCTTATATAAGAACATTATTAGATTCCATTGCTCATGGAAATAGACTTACTCCT |
| | TTGAGGGGAATATATTCTTGTAATAATCTAAGGTAACGAGTACCTTTATCTGAATGAGGA |
| | TyrAspTrpGluIleLeuAlaLysSerSerLeuSerSerSerGlnTyrLeuGlnPheLys |
| 1202 | TATGACTGGGAAATTTTGGCCAAATCTTCCCTTTCATCCTCTCAGTATCTACAGTTTAAA |
| | ATACTGACCCTTTAAAACCGGTTTAGAAGGGAAAGTAGGAGAGTCATAGATGTCAAATTT |
| | \hat{ } \hat{ } |
| | 1218 BALI, 1256 AHA3, |
| | ThrTrpTrpIleAspGlyValGlnGluGlnValArgLysAsnGlnAlaThrLysProThr |
| 1262 | ACCTGGTGGATTGATGGAGTACAAGAACAGGTACGAAAAAATCAGGCTACTAAGCCCACT |
| | TGGACCACCTAACTACCTCATGTTCTTGTCCATGCTTTTTTAGTCCGATGATTCGGGTGA |
| | ValAsnIleAspAlaAspGlnLeuLeuGlyThrGlyProAsnTrpSerThrIleAsnGln |
| 1322 | GTTAATATAGACGCAGACCAATTGTTAGGAACAGGTCCAAATTGGAGCACCATTAACCAA |
| | CAATTATATCTGCGTCTGGTTAACAATCCTTGTCCAGGTTTAAGCTCGTGGTAATTGGTT |
| | GlnSerValMetGlnAsnGluAlaIleGluGlnValArgAlaIleCysLeuArgAlaTrp |
| 1382 | CAATCAGTGATGCAGAATGAGGCTATTGAACAAGTAAGGGCTATTTGCCTCAGGGCCTGG |
| | GTTAGTCACTACGTCTTACTCCGATAACTTGTTCATTCCCGATAAACGGAGTCCCGGACC |
| | \hat{ } \hat{ } |
| | 1429 MST2, 1432 ALWN1, |
| | GlyLysIleGlnAspProGlyThrAlaPheProIleAsnSerIleArgGlnGlySerLys |
| 1442 | GGAAAAATTCAGGACCCAGGAACAGCTTTCCCTATTAATTCAATTAGACAAGGCTCTAAA |
| | CCTTTTTAAGTCCTGGGTCCTTGTCGAAAGGGATAATTAAGTTAATCTGTTCCGAGATTT |
| | \hat{ } |
| | 1475 ASE1, |
| | GluProTyrProAspPheValAlaArgLeuGlnAspAlaAlaGlnLysSerIleThrAsp |
| 1502 | GAGCCATATCCTGACTTTGTGGCAAGATTACAAGATGCTGCTCAAAAGTCTATTACAGAT |
| | CTCGGTATAGGACTGAAACACCGTTCTAATGTTCTACGACGAGTTTTCAGATAATGTCTA |
| | AspAsnAlaArgLysValIleValGluLeuMetAlaTyrGluAsnAlaAsnProGluCys |
| 1562 | GACAATGCCCGAAAAGTTATTGTAGAATTAATGGCCTATGAAAATGCAAATCCAGAATGT |
| | CTGTTACGGGCTTTTCAATAACATCTTAATTACCGGATACTTTTACGTTTAGGTCTTACA |
| | \hat{ } |
| | 1588 ASE1, |
| | GlnSerAlaIleLysProLeuLysGlyLysValProAlaGlyValAspValIleThrGlu |
| 1622 | CAGTCGGCCATAAAGCCATTAAAAGGAAAAGTTCCAGCAGGAGTTGATGTAATTACAGAA |
| | GTCAGCCGGTATTTCGGTAATTTTCCTTTTCAAGGTCGTCCTCAACTACATTAATGTCTT |
| | TyrValLysAlaCysAspGlyIleGlyGlyAlaMetHisLysAlaMetLeuMetAlaGln |
| 1682 | TATGTGAaGGCTTGTGATGGGATTGGAGGAGCTATGCATAAGGCAATGCTAATGGCTCAA |
| | ATACACTTCCGAACACTACCCTAACCTCCTCGATACGTATTCCGTTACGATTACCGAGTT |
| | \hat{ } |
| | 1715 AVA3, |
| | AlaMetArgGlyLeuThrLeuGlyGlyGlnValArgThrPheGlyLysLysCysTyrAsn |
| 1742 | GCAATGAGGGGGCTCACTCTAGGAGGACAAGTTAGAACATTTGGGAAAAAATGTTATAAT |
| | CGTTACTCCCCCGAGTGAGATCCTCCTGTTCAATCTTGTAAACCCTTTTTTACAATATTA |
| | CysGlyGlnIleGlyHisLeuLysArgSerCysProValLeuAsnLysGlnAsnIleIle |
| 1802 | TGTGGTGAAATCGGTCATCTGAAAAGGAGTTGCCCAGTCTTAAATAAACAGAATATAATA |
| | ACACCAGTTTAGCCAGTAGACTTTTCCTCAACGGGTCAGAATTTATTTGTCTTATATTAT |
| | AsnGlnAlaIleThrAlaLysAsnLysLysProSerGlyLeuCysProLysCysGlyLys |
| 1862 | AATCAAGCTATTACAGCAAAAAATAAAAAGCCATCTGGCCTGTGTCCAAAATGTGGAAAA |
| | TTAGTTCGATAATGTCGTTTTTTATTTTTCGGTAGACCGGACACAGGTTTTACACCTTTT |
| | \hat{ } |
| | 1907 PFLM1, |
| | GlyLysHisTrpAlaAsnGlnCysHisSerLysPheAspLysAspGlyGlnProLeuSer |
| 1922 | GGAAAACATTGGGCCAATCAATGTCATTCTAAATTTGATAAGGATGGGCAACCATTGTCG |
| | CCTTTTGTAACCCGGTTAGTTACAGTAAGATTTAAACTATTCCTACCCGTTGGTAAGAGC |
| | GlyAsnArgLysArgGlyGlnProGlnAlaProGlnGlnThrGlyAlaPheProValGln |
| 1982 | GGAAACAGGAAGAGGGGCCAGCCTCAGGCCCCCCAACAAACTGGGGCATTCCCAGTTCAA |
| | CCTTTGTCCTTGTCCCCGGTCGGAGTCCGGGGGGTTGTTTGACCCCGTAAGGGTCAAGTT |
| | \hat{ } \hat{ } |
| | 2003 MST2, 2014 BSTXI, |
| | LeuPheValProGlnGlyPheGlnGlyGlnGlnProLeuGlnLysIleProProLeuGln |
| 2042 | CTGTTTGTTCCTCAGGGTTTTCAAGGACAACAACCCCTACAGAAAATACCACCACTTCAG |
| | GACAAACAAGGAGTCCCAAAAGTTCCTGTTGTTGGGGATGTCTTTTATGGTGGTGAAGTC |
| | \hat{ } |
| | 2051 MST2, |
| | GlyValSerGlnLeuGlnGlnSerAsnSerCysProAlaProGlnGlnAlaAlaProGln |
| 2102 | GGAGTCAGCCAATTACAACAATCCAACAGCTGTCCCGCGCCACAGCAGGCAGCACCGCAG |
| | CCTCAGTCGGTTAATGTTGTTAGGTTGTCGACAGGGCGCGGTGTCGTCCGTCGTGGCGTC |
| | \hat{ } |
| | 2128 PVU2, |
| | AM OC |
| 2162 | TAGTAAGTCGAC |
| | ATCATTCAGCTG |
| | \hat{ } |
| | 2168 SALI, |

An alignment of the encoded proteins is below:

| #1: y.MDA.2b1112.44.aa 715 78.60% | |
|---|---|
| #2: y.orf99.aa (LNCap) 663 84.77% | |
| ALIGNMENT MAP | - showing sequences and aligned repeats {in brackets} |
| | - in each given alphabet |

| In alphabet in which alignment was found: | |
|---|---|
| 0 | {MGQTESKYASYLSFIKILL} r {RGGVR} aste {NLI} t {LFQ} t {IEQFC |
| 0 | {MGQTESKYASYLSFIKILL} k {RGGVR} vstk {NLI} k {LFQ} i {IEQFC |
| 42 | PWFPEQGTLDLKDW} ekigk {ELKQA} nregk {IIPLTVWNDWAIIKA} t |
| 42 | PWFPEQGTLDLKDW} krige {ELKQA} grkgn {IIPLTVWNDWAIIKA} a |
| 90 | {LEPFQT} gedi {VSVSDAP} k {SCV} tdceeeagtesqqg {TES} shckyvaes |
| 90 | {LEPFQT} keds {VSVSDAP} g {SCV} idcnektgrksqke {TES} lhceyvtep |
| 134 | {VMAQSTQNVDY} s {QLQ} ei {IYPE} ssklgeg {GPE} sl {GPSE} p |
| 134 | {VMAQSTQNVDY} n {QLQ} gv {IYPE} tlklegk {GPE} lv {GPSE} s |
| 174 | {KPR} spstpppvvqm {PVTLQPQTQV} rqaqtprenqverdrvsipamptqiqypqyqp |
| 174 | {KPR} gpsplpagqv. {PVTLQPQTQV} k............................... |
| 228 | v {ENKTQP} lvy {YQY} rlpt {ELQY} rppsevqyrpqavcpvpnstapyqqpt |
| 196 | . {ENKTQP} pva {YQY} wppa {ELQY} lpppesqygypgmppalqgrap..... |
| 276 | amasnspatqdaal {YPQPPTVRLNPTASRSGQGG} a {LHAVIDEARKQGDLEAWRF |
| 238 | .............. {YPQPPTVRLNPTASRSGQGG} t {LHAVIDEARKQGDLEAWRF |
| 330 | LVILQLVQAGEETQVGAPARAETRCEPFTMKMLKDIKEGVKQYGSNSPYIRTLLDSIAHG |
| 278 | LVILQLVQAGEETQVGAPARAETRCEPFTMKMLKDIKEGVKQYGSNSPYIRTLLDSIAHG |
| 390 | NRLTPYDWE} i {LAKSSLSSSQYLQFKTWWIDGVQEQVRKNQATKPTVNIDADQLLGT |
| 338 | NRLTPYDWE} s {LAKSSLSSSQYLQFKTWWIDGVQEQVRKNQATKPTVNIDADQLLGT |
| 446 | GPNWSTINQQSVMQNEAIEQVRAICLRAWGKIQDPGTAFPINSIRQGSKEPYPDFVARLQ |
| 394 | GPNWSTINQQSVMQNEAIEQVRAICLRAWGKIQDPGTAFPINSIRQGSKEPYPDFVARLQ |
| 506 | DAAQKSITDDNARKVIVELMAYENANPECQSAIKPKLGKVPAGVDVITEYVKACDGIGGA |
| 454 | DAAQKSITDDNARKVIVELMAYENANPECQSAIKPLKGKVPAGVDVITEYVKSCDGIGGA |
| 566 | MHKAMLMAQAMRGLTLGGQVRTFGKKCYNCGQIGHLKRSCPVLNKQNIINQAITAKNKKP |
| 514 | MHKAMLMAQAMRGLTLGGQVRTFGKKCYNCGQIGHLKRSCPVLNKQNIINQAITAKNKKP |
| 626 | SGLCPKCGKGKHWANQCHSKFDKDGQPLSGNRKRGQPQAPQQTGAFPVQLFVPQGFQGQQ |
| 574 | SGLCPKCGKGKHWANQCHSKFDKDGQPLSGNRKRGQPQAPQQTGAFPVQLFVPQGFQGQQ |
| 686 | PLQKIPPLQGVSQLQQSNSCPAPQQAAPQ} |
| 634 | PLQKIPPLQGVSQLQQSNSCPAPQQAAPQ} |

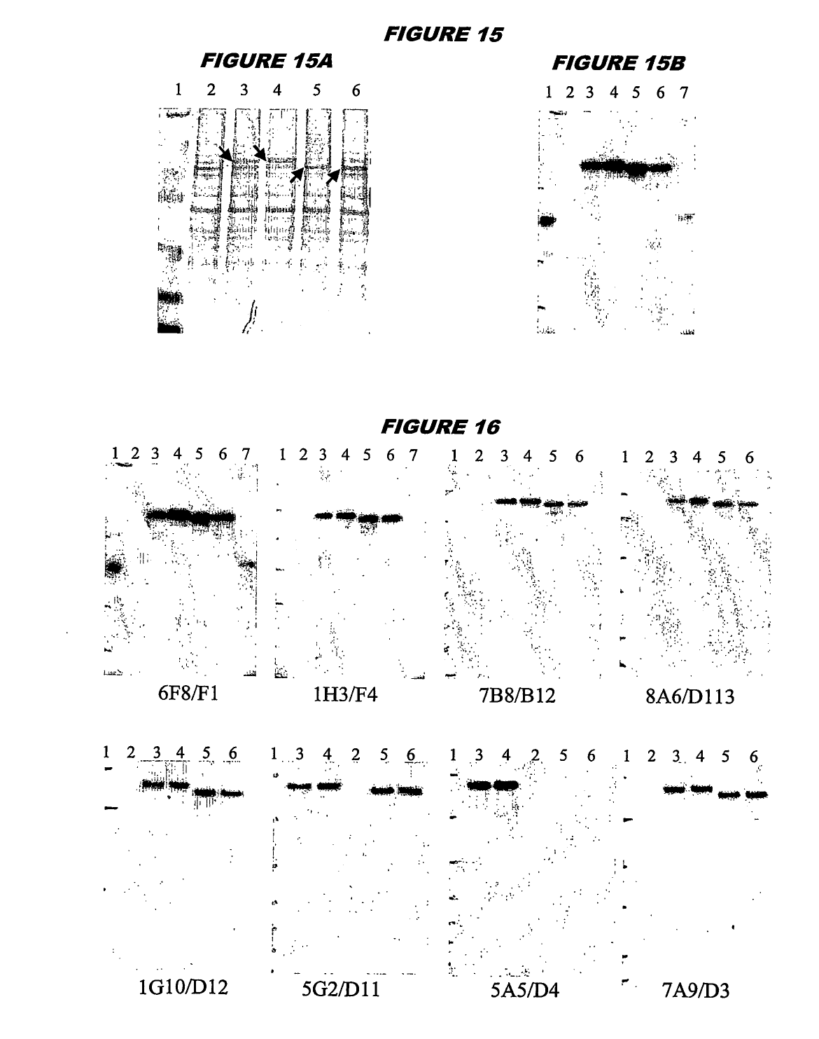

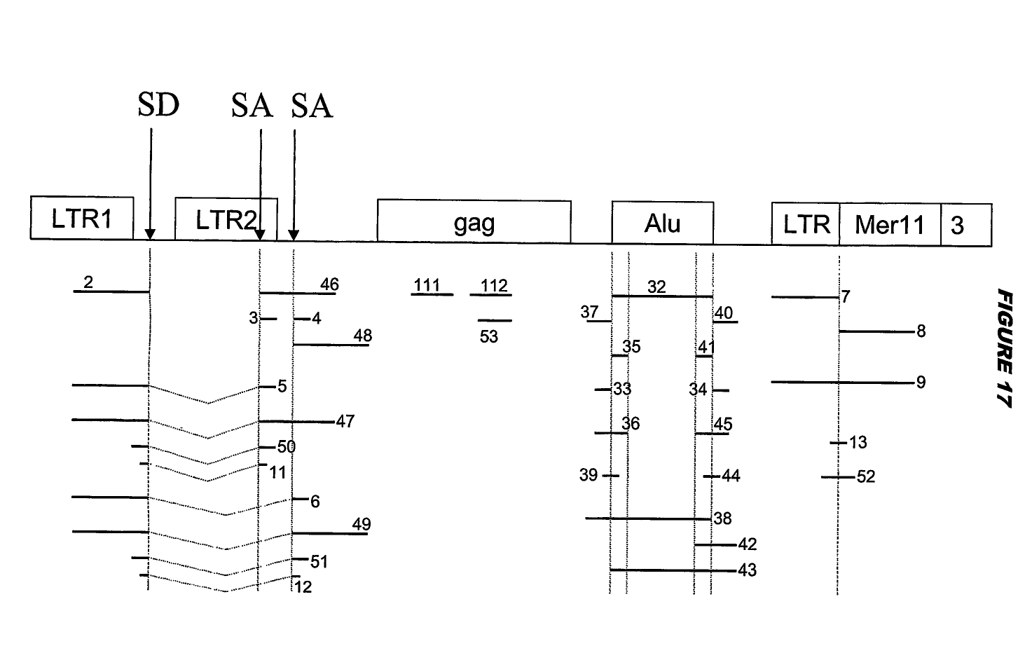

S. cerevisiae AD3 strain (mata,leu2,trp1,ura3-52,prb-1122,pep-4-3,prc1-407,cir^{o},trp+: DM15[GAP/ADR]) was transformed and single transformants were checked for expression after depletion of glucose in the medium. The recombinant proteins were expressed at high level in yeast, as detected in total yeast extracts by Coomassie blue staining (FIG. 15A). The expressed proteins were easily observed in a total yeast extract (arrows), with “new” gag in lanes 5 & 6 and the hybrid gag in lanes 3 & 4. Un-transformed control cells are shown in lane 2.

After a large-scale fermentation, proteins were purified and used for monoclonal antibody production. Eight mAbs were obtained in large quantities and they were tested for their ability to recognize both gag proteins in Western blots (FIG. 16). Of the 8 mAbs, 7 recognize both of the recombinant proteins and one (5A5/D4) recognizes only the PCAV/HERV-K hybrid gag protein. Antibody 5G2 cross-reacts with both old and new gag antigens:

| | | | PCAV/ |
|---|---|---|---|
| | | “New” | HERV-K |
| mAb | Antigen | HERV-K gag | hybrid gag |
| 5G2/D11 | “New” HERV-K gag | POSITIVE | POSITIVE |
| 7B8/B12 | “New” HERV-K gag | POSITIVE | POSITIVE |
| 8A6/D113 | “New” HERV-K gag | POSITIVE | POSITIVE |
| 7A9/D3 | “New” HERV-K gag | POSITIVE | POSITIVE |
| 1G10/D12 | “New” HERV-K gag | POSITIVE | POSITIVE |
| 1H3/F4 | “New” HERV-K gag | POSITIVE | POSITIVE |
| 5A5/D4 | PCAV/HERV-K hybrid gag | NEGATIVE | POSITIVE |
| 6F8/F1 | PCAV/HERV-K hybrid gag | POSITIVE | POSITIVE |

mAb 6F8/F1 was used in a Western blot (FIG. 15B) of a gel containing the yeast extracts in the same order and in FIG. 15A. To reduce signal intensity, the samples containing the gag recombinant proteins were diluted 50-fold relative to the samples shown in FIG. 15A using the yeast extract containing no recombinant protein.

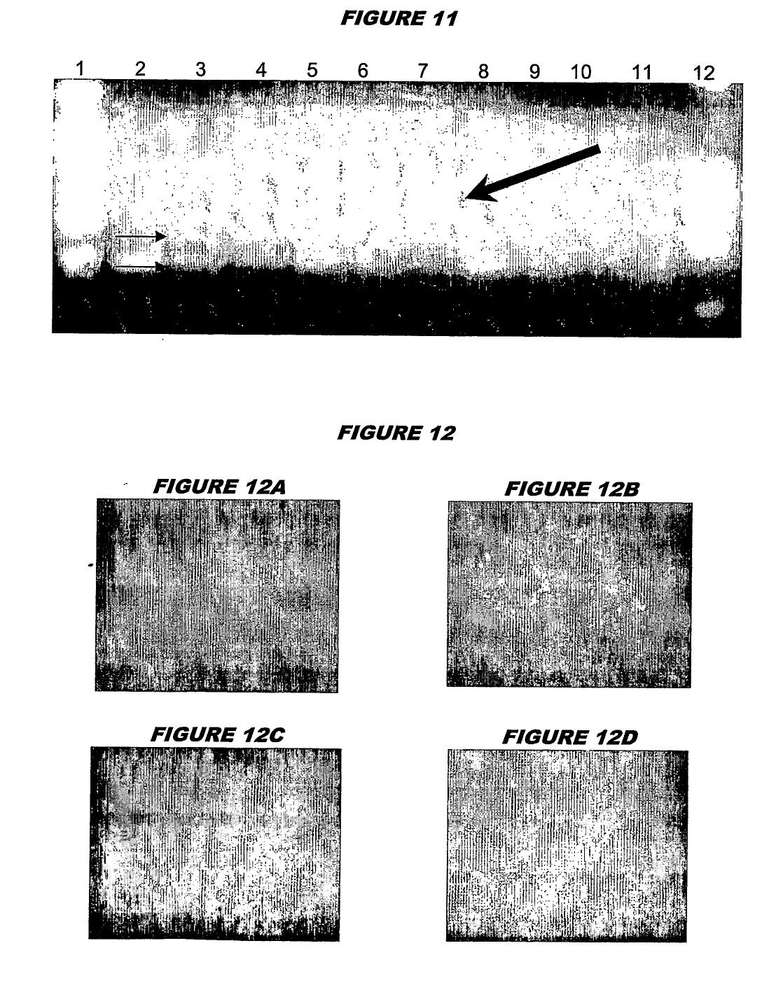

5G2 antibody binds to MDA PCA 2b cells (FIG. 12B). The cells did not fluoresce in the absence of the antibody (FIG. 12A). Prostate cell line PC3 was also reactive (FIG. 12C), but less so than MDA PCA 2b. A transformed fibroblast cell line (NIH3T3) was not reactive with anti-HERV-K-gag antibody (FIG. 12D).

The gag mRNA structure found in MDA PCA 2b cells begins in the first 5′ LTR and splices out the second 5′ LTR. Such an arrangement is necessary in order for the RNA to be translationally competent because the second 5′ LTR contains many stop codons which, in unspliced mRNA, would prevent gag translation.

PCAV Sequence Analysis

The genomic sequence of PCAV from chromosome 22 is given as SEQ ID 1. This sequence extends from the start of the first 5′ LTR in the genome to the end of the final fragment of the 3′ LTR. It is 12366 bp in total.

Within SEQ ID 1, the first 5′ LTR (new) is nucleotides 1-968. This is followed by HERV-K sequence up to nucleotide 1126. Nucleotides 1127-1678 are non-viral, including TG repeats at 1464-1487. The second 5′ LTR (old) is from nucleotides 1679-2668. The 3′ LTR is fragmented as nucleotides 10520-10838 and 11929-12366. The MER11a insertion is at nucleotides 10839-11834, with its polyA signal located between 11654-11659. The polyA addition site is located between 11736 and 11739, but it is not possible to say precisely where, because these four nucleotides are already As.

Basic coding regions within SEQ ID 1 are:

| | Product | | | | | |
|---|---|---|---|---|---|---|
| | Gag-pol | | | | | |
| | frag | PCAP6 | Gag | Prt | Pol-Env frag | Env frag |
| Start (5′) | 2669 | 2680 | 2813 | 4762 | 8513 | 10244 |
| End (3′) | 8227 | 2777 | 4960 | 5688 | 9946 | 10463 |

Splice donor (5′SS) sites are located at nucleotides 999-1004, 1076-1081, 2778-2783, 8243-8249, 8372-8378, 8429-8436, 8634-8641, 8701-8708 and 8753-8760. Splice acceptor (3′SS) sites are located at nucleotides 2593-2611, 2680-2699, 8112-8131, 8143-8165 and 10408-10423.

After the first transcribed region, there are three main downstream exons located at nucleotides 2700-2777, 8166-8244 and 10424-11739.

The gag gene (nucleotides 2813-4960 of SEQ ID 1; SEQ ID 57) encodes a 715aa polypeptide (SEQ ID 54).

The protease gene (nucleotides 4762-5688 of SEQ ID 1; SEQ ID 58) is interrupted by three stop codons:

WATIVWKQEEGPASGPPTNWGIPS*TVCSSGFSRTTTPTENTTTSGSQPITTIQQLS
RATAGSTAVDLCSTQMVFLLPGKPPQKIPRGVYGPLPEGRVGL*GRSSLNLKGVQIH
TGVIYSDYKGGIQLVISSTVPRSANPGDRIAQLLLLPYVKIGENKKERTGGFGSTNP
AGKAAYWANQVSEDRPVCTVTIQGKSLKDVDTQADVSVIGIGTASEVYQSAMILHCP
GSDNQESTVQPVITSFIPINLWGRDLLQQWHAEITIPASLYSPRNKKIMTKMG*LPK
KGLGKKEVPIEAEKNQKRKGIGHPF

The four amino acid sequences between stop codons are SEQ IDs 59 to 62.

The pol gene (SEQ ID 86) is also interrupted. Alignment with known pol sequences reveals various fragments of amino acid sequences (SEQ IDs 92 to 97):

ESSKLSIT*LKEQSWLPSLQC*QDFNQSINIVSDSAYVVQATKDIERALIKYIMDDQ
LNPLFNLLQQNVRKRNFPFYITHIRAHTNLPGPLTKANEQADLLVSSAFMEAQELHA
LTHVNAIGLKNKFDITWKQTKNIVQHCTQCQILHLATQEARVNPRGLCPNVLWQMDV
MHVPSFGKLSFVHVTVDTYSHFIWATCQTGESTSHVKRHLLSCFPVMGVPEKVKTDN
GPGYCSKAVQKFLNQWKITHTIGILYNSQGQAIIERTNRTLKAQLVKQKKGKDRSIT
LPRCNLI
MSNLFSFLRGDSELNSERTLTPEATKEIKLIEEKIRSAQVNRIDHLAPLQILIFATA
HSLTGIIVQNTDLVEWSFLPHSTIKTFTLYLDQMATLIGQGRL*IITLCGNDPDKIT
VPFNKQQVRQAFINSGAWQIGLADFVGIIDNRYPKTKIFQFLKLTTWILPKVTKHKP
LKNALAVFTDGSSNGKVAYTGPKE*
*TKKRKRQEYNTPQMQLNLALYTLNVLNIYRNQTTTSAEQHLTGKRNSPHEGKLIWW
KDNKNKTWEMGKVITWGRGFACVSPGENQLPVWIPTRHLKFYNELTGDAKKSVEMET
PQSTRQVNKMVISEEQKKLPSIKEAELPI

The env gene (nucleotides 9165-9816 of SEQ ID 1; SEQ ID 63) is interrupted by stop codons. The longest uninterrupted sequence encodes amino acid sequence SEQ ID 64. The reading frame +1 to SEQ ID 63 contains several short amino acid sequences (SEQ IDs 65 to 80) between stop codons:

HPELGSLLWPHTTLEFVLEIKL*EQEIVSHIILST*IPV*QFLCKIV*NSLILLVVG
KT*LLNLIPKP*SVKIVECLLALI*LLIGSTVFY*EEQERVCGSLCPWTDHGRLRYP
SIF*RKY*KEF*LDPKDSFLL*WQ*LWASLQSQLLLRLLELLYTPLFKLQNT*MIGK
RIPQNCGILRSK*IKNWQTKLMILDKLSFGWERLMSLEYLFQLRC

Nucleotides 8916-9155 of SEQ ID 1 (SEQ ID 81) are also interrupted to give several short amino acid sequences (SEQ IDs 82 to 85):

VQNNEF*TMIDWVP*GQLYHNCTGQTHSCSQAPSIWPINPAYDGDVTERLDQVYRRL
ESLCPRKWGEKGISSP*PKLVLLLVL

A polypeptide product called ‘morf’ or ‘PCAP3’ (SEQ ID 87) is roughly equivalent to the ‘cORF’ product previously seen for HERV-Ks. Its coding sequence begins at nucleotide 8183 of SEQ ID 1, with splicing occurring after nucleotide 8244 and joining to nucleotide 10424. The splice junction forms a AGT serine codon within SEQ ID 88 (FIG. 23):

| | |
|---|---|
| ATGAACTCACTGGAGATGCAAAGAAAAGTGTGGAGATGGAGACACCCCAATCGACTCGCCAGgtaaacaaa | 8253 |
| M N S L E M Q R K V W R W R H P N R L A r * | |
| ...cctgttctgtctgttgttagTCTACAGGTGTATCCAGCAGCTCCAAAGAGACAGCAACCAGCAAGAATGGGCCATAG | 10480 |
| L Q V Y P A A P K R Q Q P A R M G H S | |
| TGACGATGGTGGTTTTGTCAAAAAGAAAAGGGGGGGATATGTAAGGAAAAGAGAGATCAGACTTTCACTGTGTCTATGTA | 10560 |
| D D G G F V K K K R G G Y V R K R E I R L S L C L C R | |
| GAAAAGGAAGACATAAGAAACTCCATTTTGATCTGTACTAA | 10601 |
| K G R H K K L H F D L Y * | |

Further details about PCAP3 are given below.

Unique DNA Sequence within PCAV gag

PCAV gag contains a 48 nucleotide sequence (SEQ ID 53) which is not found in the closely-related HERV-Ks on chromosomes 3, 6 and 16. The 48mer encodes 16mer SEQ ID 110, which is not found in new or in other old HERV-Ks. The top 5 hits in BLAST analysis of a 99mer (3614 to 3712 from SEQ ID 1) comprising SEQ ID 53 shows:

| Query = PCAV ch22 gag specific | | | |
|---|---|---|---|
| (99 letters) | | | |
| Database: NCBI Contigs | | | |
| 13,079 sequences; 2,842,562,037 total letters | | | |
| >NT_011520S13.7 Genomic Viewer | | | |
| Homo sapiens chromosome 22 working draft sequence segment | | | |
| Length = 276008 | | | |
| Score = 196 bits (99), Expect = 1e−48 | | | |
| Identities = 99/99 (100%) | | | |
| Strand = Plus/Plus | | | |
| Query: | 1 | agcacggcaccataccagcaacccacagcgatggcgtctaattcaccagcaacacaggac | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 125279 | agcacggcaccataccagcaacccacagcgatggcgtctaattcaccagcaacacaggac | 125338 |
| Query: | 61 | gcggcgctgtatcctcagccgcccactgtgagacttaat | 99 |
| | | \|\|\|\|\|\|\|\|\|\|\|\|\|\|\|\|\|\|\|\|\|\|\|\|\|\|\|\|\|\|\|\|\|\|\|\|\|\|\| | |
| Sbjct: | 125339 | gcggcgctgtatcctcagccgcccactgtgagacttaat | 125377 |
| >NT_015360S4.5 Genomic Viewer | | | |
| Homo sapiens chromosome 16 working draft sequence segment | | | |
| Length = 244218 | | | |
| Score = 75.8 bits (38), Expect = 3e−12 | | | |
| Identities = 83/98 (84%) | | | |
| Strand = Plus/Plus | | | |
| Query: | 2 | gcacggcaccataccagcaacccacagcgatggcgtctaattcaccagcaacacaggacg | 61 |
| | | \|\|\|\|\|\|\|\| \| \|\|\| \|\|\|\|\|\|\|\|\| \|\|\| \|\|\| \|\| \|\|\|\| \| \|\|\|\|\| \|\|\|\|\|\| \|\| | |
| Sbjct: | 15122 | gcacggcatcgtacaagcaacccatggcggtggtgtttaatacgtcagcaccacagggcg | 15181 |
| Query: | 62 | cggcgctgtatcctcagccgcccactgtgagacttaat | 99 |
| | | \|\|\|\|\|\|\|\|\| \|\|\|\|\|\|\|\|\|\|\|\|\|\|\|\| \|\|\|\|\|\|\|\|\|\|\| | |
| Sbjct: | 15182 | cggcgctgtgtcctcagccgcccactatgagacttaat | 15219 |
| >NT_005863S5.5 Genomic Viewer | | | |
| Homo sapiens chromosome 3 working draft sequence segment | | | |
| Length = 278948 | | | |
| Score = 60.0 bits (30), Expect = 2e−07 | | | |
| Identities = 30/30 (100%) | | | |
| Strand = Plus/Plus | | | |
| Query: | 70 | tatcctcagccgcccactgtgagacttaat | 99 |
| | | \|\|\|\|\|\|\|\|\|\|\|\|\|\|\|\|\|\|\|\|\|\|\|\|\|\|\|\|\|\| | |
| Sbjct: | 116212 | tatcctcagccgcccactgtgagacttaat | 116241 |
| >NT_023409S14.5 Genomic Viewer | | | |
| Homo sapiens chromosome 6 working draft sequence segment | | | |
| Length = 238047 | | | |
| Score = 52.0 bits (26), Expect = 5e−05 | | | |
| Identities = 26/26 (100%) | | | |
| Strand = Plus/Minus | | | |
| Query: | 1 | agcacggcaccataccagcaacccac | 26 |
| | | \|\|\|\|\|\|\|\|\|\|\|\|\|\|\|\|\|\|\|\|\|\|\|\|\|\| | |
| Sbjct: | 63402 | agcacggcaccataccagcaacccac | 63377 |
| >NT_007592S47.5 Genomic Viewer | | | |
| Homo sapiens chromosome 6 working draft sequence segment | | | |
| Length = 250001 | | | |
| Score = 50.1 bits (25), Expect = 2e−04 | | | |
| Identities = 28/29 (96%) | | | |
| Strand = Plus/Minus | | | |
| Query: | 71 | atcctcagccgcccactgtgagacttaat | 99 |
| | | \|\|\|\|\|\|\|\|\|\|\| \|\|\|\|\|\|\|\|\|\|\|\|\|\|\|\|\| | |
| Sbjct: | 81143 | atcctcagccgtccactgtgagacttaat | 81115 |

Epitopes within PCAV gag

An alignment of the N-termini of various HERV-Ks is shown below:

| | | |
|---|---|---|
| HERV-K gag tandem PCAV gag CH8 8.032mb PCAV gag CH8 7.37mb PCAV gag CH6 47.1 mb PCAV ch22 20.428mb + LTRsPCAV gag ch6 30.9Mb PCAV gag CH3 103.75mb PCAV gag ch5 151.108mb PCAV gag ch8 142.771mb PCAV gag ch11 57.875mb | (1) (1) (1) (1) (1)(1) (1) (1) (1) (1) | 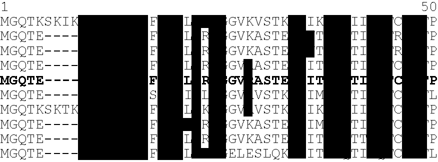 |
| HERV-K gag tandem PCAV gag CH8 8.032mb PCAV gag CH8 7.37mb PCAV gag CH6 47.1 mb PCAV ch22 20.428mb + LTRsPCAV gag ch6 30.9Mb PCAV gag CH3 103.75mb PCAV gag ch5 151.108mb PCAV gag ch8 142.771mb PCAV gag ch11 57.875mb | (51) (47) (47) (47) (47)(47) (51) (47) (47) (47) | 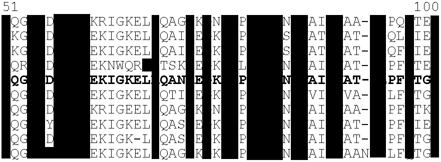 |
| HERV-K gag tandem PCAV gag CH8 8.032mb PCAV gag CH8 7.37mb PCAV gag CH6 47.1 mb PCAV ch22 20.428mb + LTRsPCAV gag ch6 30.9Mb PCAV gag CH3 103.75mb PCAV gag ch5 151.108mb PCAV gag ch8 142.771mb PCAV gag ch11 57.875mb | (100) (96) (96) (96) (96)(96) (100) (96) (95) (97) | 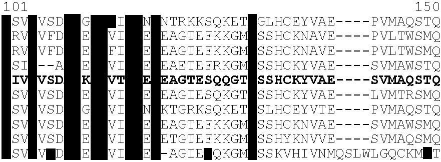 |
| HERV-K gag tandem PCAV gag CH8 8.032mb PCAV gag CH8 7.37mb PCAV gag CH6 47.1 mb PCAV ch22 20.428mb + LTRsPCAV gag ch6 30.9Mb PCAV gag CH3 103.75mb PCAV gag ch5 151.108mb PCAV gag ch8 142.771mb PCAV gag ch11 57.875mb | (146) (142) (142) (140) (142)(142) (146) (142) (141) (146) | 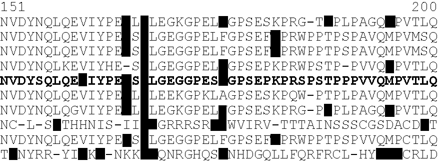 |
| HERV-K gag tandem PCAV gag CH8 8.032mb PCAV gag CH8 7.37mb PCAV gag CH6 47.1 mb PCAV ch22 20.428mb + LTRsPCAV gag ch6 30.9Mb PCAV gag CH3 103.75mb PCAV gag ch5 151.108mb PCAV gag ch8 142.771mb PCAV gag ch11 57.875mb | (195) (192) (192) (188) (192)(191) (195) (188) (191) (193) | 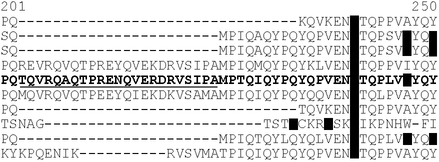 |
| HERV-K gag tandem PCAV gag CH8 8.032mb PCAV gag CH8 7.37mb PCAV gag CH6 47.1 mb PCAV ch22 20.428mb + LTRsPCAV gag ch6 30.9Mb PCAV gag CH3 103.75mb PCAV gag ch5 151.108mb PCAV gag ch8 142.771mb PCAV gag ch11 57.875mb | (213) (219) (219) (238) (242)(241) (213) (212) (218) (233) | 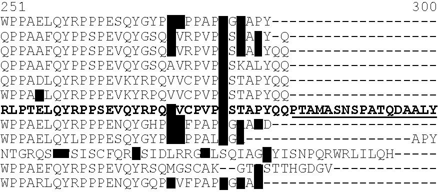 |
| HERV-K gag tandem PCAV gag CH8 8.032mb PCAV gag CH8 7.37mb PCAV gag CH6 47.1 mb PCAV ch22 20.428mb + LTRsPCAV gag ch6 30.9Mb PCAV gag CH3 103.75mb PCAV gag ch5 151.108mb PCAV gag ch8 142.771mb PCAV gag ch11 57.875mb | (244) (251) (252) (271) (292)(272) (244) (257) (254) (263) | 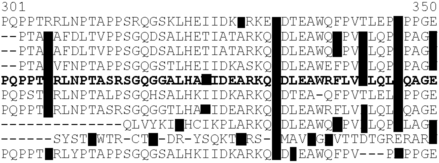 |
| HERV-K gag tandem PCAV gag CH8 8.032mb PCAV gag CH8 7.37mb PCAV gag CH6 47.1 mb PCAV ch22 20.428mb + LTRsPCAV gag ch6 30.9Mb PCAV gag CH3 103.75mb PCAV gag ch5 151.108mb PCAV gag ch8 142.771mb PCAV gag ch11 57.875mb | (294) (299) (300) (319) (342)(321) (294) (293) (294) (310) | 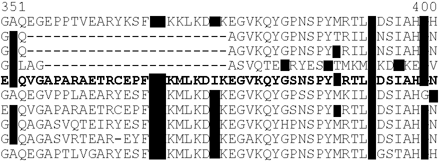 |

Two regions are particularly useful for generating PCAV-specific detection reagents. The first is from amino acid 203 to 225 in the alignment (SEQ ID 55; encoded by SEQ ID 111). Although this region is present in two other HERV-Ks on chromosome 6, those two viruses are in the old HERV-K group. Background (“ubiquitous”) expression of new HERV-Ks is seen in many tissues (e.g. FIG. 10), but not of old HERV-Ks. Detection of SEQ ID 55 therefore distinguishes over background expression of new HERV-Ks and can be used to detect PCAV expression.

The second region is found from amino acids 284-300 (SEQ ID 56; encoded by SEQ ID 112), as this sequence is unique to PCAV. SEQ ID 110 (SEQ ID 53) is a single amino acid truncation fragment of SEQ ID 56.

TBLASTN analysis of SEQ ID 110 against the human genome sequence reveals 100% matches in clones KB208E9 and KB1572G7 at chromosome 22q11.2 but nowhere else. BLASTP analysis fails to identify any matches.

BLASTN analysis of SEQ ID 53 against the human genome sequence reveals a 100% match at nucleotides 3180761 to 3180808 of the Homo sapiens chromosome 22 working draft sequence, and no further hits.

The top five BLASTP hits using SEQ ID 110 against the non-redundant GenBank CDS database are shown below:

>gi|21230944|ref|NP_636861.1| (NC_003902) con-
served hypothetical protein
{Xanthomonas campestris pv. campestris str. ATCC
33913}
Length = 515
Score = 27.8 bits (58), Expect = 12
Identities = 10/16 (62%), Positives = 12/16 (74%),
Gaps = 2/16 (12%)

| | | | |
|---|---|---|---|
| Query: | 1 | TAMASNSPATQ--DAA | 14 |
| | | T MAS++ ATQ DAA | |
| Sbjct: | 483 | TGMASDASATQEDDAA | 498 |

>gi|12852148|dbj|BAB29293.1| (AK014354) data
source:SPTR, source key:Q92524,
evidence:ISS-homolog to 26S PROTEASE REGULATORY
SUBUNIT
S10B (PROTEASOME SUBUNIT P42) ˜putative {Mus
musculus}
Length = 389
Score = 27.4 bits (57), Expect = 16
Identities = 9/13 (69%), positives = 10/13 (76%)

| | | | |
|---|---|---|---|
| Query: | 3 | MASNSPATQDAAL | 15 |
| | | MA+NSP T D AL | |
| Sbjct: | 277 | MATNSPDTLDPAL | 289 |

>gi|7105525|gb|AAF35993.1|AC005836_5 (AC005836)
26S Protease Regulatory Subunit
{Leishmnnia major}
Length = 396
Score = 26.9 bits (56), Expect = 22
Identities = 9/13 (69%), Positives = 10/13 (76%)

| | | | |
|---|---|---|---|
| Query: | 3 | MASNSPATQDAAL | 15 |
| | | MA+N P T DAAL | |
| Sbjct: | 283 | MATNRPDTLDAAL | 295 |

>gi|15233182|ref|NP_191727.1| (NM_116033)
putative protein {Arabidopsis thaliana}
Length = 658
Score = 26.1 bits (54), Expect = 39
Identities = 8/9 (88%), Positives = 8/9 (88%)

| | | | |
|---|---|---|---|
| Query: | 1 | TAMASNSPA | 9 |
| | | TAMAS SPA | |
| Sbjct: | 5 | TAMASTSPA | 13 |

>gi|21243749|ref|NP_643331.1| (NC_003919)
hypothetical protein {Xanthomonas
axonopodis pv. Citri str. 306}
Length = 206
Score = 25.7 bits (53), Expect = 52
Identities = 8/12 (66%), Positives = 10/12 (82%)

| | | | |
|---|---|---|---|
| Query: | 2 | AMASNSPATQDA | 13 |
| | | AMA+ SPAT +A | |
| Sbjct: | 189 | AMAATSPATPNA | 200 |

SEQ ID 110 is therefore unique to PCAV.

Prediction of cDNA Sequences

On the basis of splice donor and acceptor sites, SEQ IDs 99 to 109 were constructed. SEQ ID 109 begins in the second 5′ LTR.

SEQ IDs 99 to 108 align: to SEQ ID 10 as follows:

| SEQ ID 10 | GAGATAGGAGAAAACTGCCTTAGGGCTGGAGGTGGGACATGCTGGCGGCAATACTGCTCTTTAAGGCATT |
|---|---|
| SEQ ID 106 | GAGATAGGAGAAAACTGCCTTAGGGCTGGAGGTGGGACATGCTGGCGGCAATACTGCTCTTTAAGGCATT |
| SEQ ID 105 | GAGATAGGAGAAAACTGCCTTAGGGCTGGAGGTGGGACATGCTGGCGGCAATACTGCTCTTTAAGGCATT |
| SEQ ID 99 | GAGATAGGAGAAAACTGCCTTAGGGCTGGAGGTGGGACATGCTGGCGGCAATACTGCTCTTTAAGGCATT |
| SEQ ID 100 | GAGATAGGAGAAAACTGCCTTAGGGCTGGAGGTGGGACATGCTGGCGGCAATACTGCTCTTTAAGGCATT |
| SEQ ID 104 | GAGATAGGAGAAAACTGCCTTAGGGCTGGAGGTGGGACATGCTGGCGGCAATACTGCTCTTTAAGGCATT |
| SEQ ID 103 | GAGATAGGAGAAAACTGCCTTAGGGCTGGAGGTGGGACATGCTGGCGGCAATACTGCTCTTTAAGGCATT |
| SEQ ID 101 | GAGATAGGAGAAAACTGCCTTAGGGCTGGAGGTGGGACATGCTGGCGGCAATACTGCTCTTTAAGGCATT |
| SEQ ID 102 | GAGATAGGAGAAAACTGCCTTAGGGCTGGAGGTGGGACATGCTGGCGGCAATACTGCTCTTTAAGGCATT |
| SEQ ID 108 | ---------------------------------------------------------------------- |
| SEQ ID 107 | ---------------------------------------------------------------------- |
| SEQ ID 10 | GAGATGTTTATGTATATGCACATCAAAAGCACAGCACTTTTTTCTTTACCTTGTTTATGATGCAGAGACA |
| SEQ ID 106 | GAGATGTTTATGTATATGCACATCAAAAGCACAGCACTTTTTTCTTTACCTTGTTTATGATGCAGAGACA |
| SEQ ID 105 | GAGATGTTTATGTATATGCACATCAAAAGCACAGCACTTTTTTCTTTACCTTGTTTATGATGCAGAGACA |
| SEQ ID 99 | GAGATGTTTATGTATATGCACATCAAAAGCACAGCACTTTTTTCTTTACCTTGTTTATGATGCAGAGACA |
| SEQ ID 100 | GAGATGTTTATGTATATGCACATCAAAAGCACAGCACTTTTTTCTTTACCTTGTTTATGATGCAGAGACA |
| SEQ ID 104 | GAGATGTTTATGTATATGCACATCAAAAGCACAGCACTTTTTTCTTTACCTTGTTTATGATGCAGAGACA |
| SEQ ID 103 | GAGATGTTTATGTATATGCACATCAAAAGCACAGCACTTTTTTCTTTACCTTGTTTATGATGCAGAGACA |
| SEQ ID 101 | GAGATGTTTATGTATATGCACATCAAAAGCACAGCACTTTTTTCTTTACCTTGTTTATGATGCAGAGACA |
| SEQ ID 102 | GAGATGTTTATGTATATGCACATCAAAAGCACAGCACTTTTTTCTTTACCTTGTTTATGATGCAGAGACA |
| SEQ ID 108 | ---------------------------------------------------------------------- |
| SEQ ID 107 | ---------------------------------------------------------------------- |
| SEQ ID 10 | TTTGTTCACATGTTTTCCTGCTGGCCCTCTCCCCACTATTACCCTATTGTCCTGCCACATCCCCCTCTCC |
| SEQ ID 106 | TTTGTTCACATGTTTTCCTGCTGGCCCTCTCCCCACTATTACCCTATTGTCCTGCCACATCCCCCTCTCC |
| SEQ ID 105 | TTTGTTCACATGTTTTCCTGCTGGCCCTCTCCCCACTATTACCCTATTGTCCTGCCACATCCCCCTCTCC |
| SEQ ID 99 | TTTGTTCACATGTTTTCCTGCTGGCCCTCTCCCCACTATTACCCTATTGTCCTGCCACATCCCCCTCTCC |
| SEQ ID 100 | TTTGTTCACATGTTTTCCTGCTGGCCCTCTCCCCACTATTACCCTATTGTCCTGCCACATCCCCCTCTCC |
| SEQ ID 104 | TTTGTTCACATGTTTTCCTGCTGGCCCTCTCCCCACTATTACCCTATTGTCCTGCCACATCCCCCTCTCC |
| SEQ ID 103 | TTTGTTCACATGTTTTCCTGCTGGCCCTCTCCCCACTATTACCCTATTGTCCTGCCACATCCCCCTCTCC |
| SEQ ID 101 | TTTGTTCACATGTTTTCCTGCTGGCCCTCTCCCCACTATTACCCTATTGTCCTGCCACATCCCCCTCTCC |
| SEQ ID 102 | TTTGTTCACATGTTTTCCTGCTGGCCCTCTCCCCACTATTACCCTATTGTCCTGCCACATCCCCCTCTCC |
| SEQ ID 108 | ---------------------------------------------------------------------- |
| SEQ ID 107 | ---------------------------------------------------------------------- |
| SEQ ID 10 | GAGATGGTAGAGATAATGATCAATAAATACTGAGGGAACTCAGAGACCGGTGCGGCGCGGGTCCTCCATA |
| SEQ ID 106 | GAGATGGTAGAGATAATGATCAATAAATACTGAGGGAACTCAGAGACCGGTGCGGCGCGGGTCCTCCATA |
| SEQ ID 105 | GAGATGGTAGAGATAATGATCAATAAATACTGAGGGAACTCAGAGACCGGTGCGGCGCGGGTCCTCCATA |
| SEQ ID 99 | GAGATGGTAGAGATAATGATCAATAAATACTGAGGGAACTCAGAGACCGGTGCGGCGCGGGTCCTCCATA |
| SEQ ID 100 | GAGATGGTAGAGATAATGATCAATAAATACTGAGGGAACTCAGAGACCGGTGCGGCGCGGGTCCTCCATA |
| SEQ ID 104 | GAGATGGTAGAGATAATGATCAATAAATACTGAGGGAACTCAGAGACCGGTGCGGCGCGGGTCCTCCATA |
| SEQ ID 103 | GAGATGGTAGAGATAATGATCAATAAATACTGAGGGAACTCAGAGACCGGTGCGGCGCGGGTCCTCCATA |
| SEQ ID 101 | GAGATGGTAGAGATAATGATCAATAAATACTGAGGGAACTCAGAGACCGGTGCGGCGCGGGTCCTCCATA |
| SEQ ID 102 | GAGATGGTAGAGATAATGATCAATAAATACTGAGGGAACTCAGAGACCGGTGCGGCGCGGGTCCTCCATA |
| SEQ ID 108 | ---------------------------------------------------------------------- |
| SEQ ID 107 | ---------------------------------------------------------------------- |
| SEQ ID 10 | TGCTGAGCGCCGGTCCCCTGGGCCCACTTTTCTTTCTCTATACTTTGTCTCTGTTGTCTTTCTTTTCTCA |
| SEQ ID 106 | TGCTGAGCGCCGGTCCCCTGGGCCCACTTTTCTTTCTCTATACTTTGTCTCTGTTGTCTTTCTTTTCTCA |
| SEQ ID 105 | TGCTGAGCGCCGGTCCCCTGGGCCCACTTTTCTTTCTCTATACTTTGTCTCTGTTGTCTTTCTTTTCTCA |
| SEQ ID 99 | TGCTGAGCGCCGGTCCCCTGGGCCCACTTTTCTTTCTCTATACTTTGTCTCTGTTGTCTTTCTTTTCTCA |
| SEQ ID 100 | TGCTGAGCGCCGGTCCCCTGGGCCCACTTTTCTTTCTCTATACTTTGTCTCTGTTGTCTTTCTTTTCTCA |
| SEQ ID 104 | TGCTGAGCGCCGGTCCCCTGGGCCCACTTTTCTTTCTCTATACTTTGTCTCTGTTGTCTTTCTTTTCTCA |
| SEQ ID 103 | TGCTGAGCGCCGGTCCCCTGGGCCCACTTTTCTTTCTCTATACTTTGTCTCTGTTGTCTTTCTTTTCTCA |
| SEQ ID 101 | TGCTGAGCGCCGGTCCCCTGGGCCCACTTTTCTTTCTCTATACTTTGTCTCTGTTGTCTTTCTTTTCTCA |
| SEQ ID 102 | TGCTGAGCGCCGGTCCCCTGGGCCCACTTTTCTTTCTCTATACTTTGTCTCTGTTGTCTTTCTTTTCTCA |
| SEQ ID 108 | ---------------------------------------------------------------------- |
| SEQ ID 107 | ---------------------------------------------------------------------- |
| SEQ ID 10 | AGTCTCTCGTTCCACCTGAGGAGAAATGCCCACAGCTGTGGAGGCGCAGGCCACTCCATCTGGTGCCCAA |
| SEQ ID 106 | AGTCTCTCGTTCCACCTGAGGAGAAATGCCCACAGCTGTGGAGGCGCAGGCCACTCCATCTGGTGCCCAA |
| SEQ ID 105 | AGTCTCTCGTTCCACCTGAGGAGAAATGCCCACAGCTGTGGAGGCGCAGGCCACTCCATCTGGTGCCCAA |
| SEQ ID 99 | AGTCTCTCGTTCCACCTGAGGAGAAATGCCCACAGCTGTGGAGGCGCAGGCCACTCCATCTGGTGCCCAA |
| SEQ ID 100 | AGTCTCTCGTTCCACCTGAGGAGAAATGCCCACAGCTGTGGAGGCGCAGGCCACTCCATCTGGTGCCCAA |
| SEQ ID 104 | AGTCTCTCGTTCCACCTGAGGAGAAATGCCCACAGCTGTGGAGGCGCAGGCCACTCCATCTGGTGCCCAA |
| SEQ ID 103 | AGTCTCTCGTTCCACCTGAGGAGAAATGCCCACAGCTGTGGAGGCGCAGGCCACTCCATCTGGTGCCCAA |
| SEQ ID 101 | AGTCTCTCGTTCCACCTGAGGAGAAATGCCCACAGCTGTGGAGGCGCAGGCCACTCCATCTGGTGCCCAA |
| SEQ ID 102 | AGTCTCTCGTTCCACCTGAGGAGAAATGCCCACAGCTGTGGAGGCGCAGGCCACTCCATCTGGTGCCCAA |
| SEQ ID 108 | ---------------------------------------------------------------------- |
| SEQ ID 107 | ---------------------------------------------------------------------- |
| SEQ ID 10 | CGTGGATGCTTTTCTCTAGGGTGAAGGGACTCTCGAGTGTGGTCATTGAGGACAAGTCAACGAGAGATTC |
| SEQ ID 106 | CGTGGATGCTTTTCTCTAGGGTGAAGGGACTCTCGAGTGTGGTCATTGAGGACAAGTCAACGAGAGATTC |
| SEQ ID 105 | CGTGGATGCTTTTCTCTAGGGTGAAGGGACTCTCGAGTGTGGTCATTGAGGACAAGTCAACGAGAGATTC |
| SEQ ID 99 | CGTGGATGCTTTTCTCTAGGGTGAAGGGACTCTCGAGTGTGGTCATTGAGGACAAGTCAACGAGAGATTC |
| SEQ ID 100 | CGTGGATGCTTTTCTCTAGGGTGAAGGGACTCTCGAGTGTGGTCATTGAGGACAAGTCAACGAGAGATTC |
| SEQ ID 104 | CGTGGATGCTTTTCTCTAGGGTGAAGGGACTCTCGAGTGTGGTCATTGAGGACAAGTCAACGAGAGATTC |
| SEQ ID 103 | CGTGGATGCTTTTCTCTAGGGTGAAGGGACTCTCGAGTGTGGTCATTGAGGACAAGTCAACGAGAGATTC |
| SEQ ID 101 | CGTGGATGCTTTTCTCTAGGGTGAAGGGACTCTCGAGTGTGGTCATTGAGGACAAGTCAACGAGAGATTC |
| SEQ ID 102 | CGTGGATGCTTTTCTCTAGGGTGAAGGGACTCTCGAGTGTGGTCATTGAGGACAAGTCAACGAGAGATTC |
| SEQ ID 108 | ---------------------------------------------------------------------- |
| SEQ ID 107 | ---------------------------------------------------------------------- |
| SEQ ID 10 | CCGAGTACGTCTACAGTGAGCCTTGTGGTAAGCTTGGGCGCTCGGAAGAAGCCAGGGTTAATGGGGCAAA |
| SEQ ID 106 | CCGAGTACGTCTACAGTGAGCCTTGTGG------------------------------------------ |
| SEQ ID 105 | CCGAGTACGTCTACAGTGAGCCTTGTGG------------------------------------------ |
| SEQ ID 99 | CCGAGTACGTCTACAGTGAGCCTTGTG------------------------------------------- |
| SEQ ID 100 | CCGAGTACGTCTACAGTGAGCCTTGTG------------------------------------------- |
| SEQ ID 104 | CCGAGTACGTCTACAGTGAGCCTTGTGG------------------------------------------ |
| SEQ ID 103 | CCGAGTACGTCTACAGTGAGCCTTGTGG------------------------------------------ |
| SEQ ID 101 | CCGAGTACGTCTACAGTGAGCCTTGTG------------------------------------------- |
| SEQ ID 102 | CCGAGTACGTCTACAGTGAGCCTTGTGG------------------------------------------ |
| SEQ ID 108 | ---------------------------------------------------------------------- |
| SEQ ID 107 | ---------------------------------------------------------------------- |
| <break> | |
| SEQ ID 10 | CTGTGTCTTATTTCTTTCCTCAGTCTCTCATCCCTCCTGACGAGAAATACCCACAGGTGTGGAGGGGCTG |
| SEQ ID 106 | ---------------------------------------------------------------------- |
| SEQ ID 105 | ---------------------------------------------------------------------- |
| SEQ ID 99 | -----------------------TCTCTCATCCCTCCTGACGAGAAATACCCACAGGTGTGGAGGGGCTG |
| SEQ ID 100 | -----------------------TCTCTCATCCCTCCTGACGAGAAATACCCACAGGTGTGGAGGGGCTG |
| SEQ ID 104 | ---------------------------------------------------------------------- |
| SEQ ID 103 | ---------------------------------------------------------------------- |
| SEQ ID 101 | -----------------------TCTCTCATCCCTCCTGACGAGAAATACCCACAGGTGTGGAGGGGCTG |
| SEQ ID 102 | ---------------------------------------------------------------------- |
| SEQ ID 108 | ---------------------------------------------------------------------- |
| SEQ ID 107 | ---------------------------------------------------------------------- |
| SEQ ID 10 | GCCCCCTTCATCTGATGCCCAATGTGGGTGCCTTTCTCTAGGGTGAAGGTACTCTACAGTGTGGTCATTG |
| SEQ ID 106 | ------------------------------------------GTGAAGGTACTCTACAGTGTGGTCATTG |
| SEQ ID 105 | ------------------------------------------GTGAAGGTACTCTACAGTGTGGTCATTG |
| SEQ ID 99 | GCCCCCTTCATCTGATGCCCAATGTGGGTGCCTTTCTCTAGGGTGAAGGTACTCTACAGTGTGGTCATTG |
| SEQ ID 100 | GCCCCCTTCATCTGATGCCCAATGTGGGTGCCTTTCTCTAGGGTGAAGGTACTCTACAGTGTGGTCATTG |
| SEQ ID 104 | ------------------------------------------GTGAAGGTACTCTACAGTGTGGTCATTG |
| SEQ ID 103 | ------------------------------------------GTGAAGGTACTCTACAGTGTGGTCATTG |
| SEQ ID 101 | GCCCCCTTCATCTGATGCCCAATGTGGGTGCCTTTCTCTAGGGTGAAGGTACTCTACAGTGTGGTCATTG |
| SEQ ID 102 | ------------------------------------------GTGAAGGTACTCTACAGTGTGGTCATTG |
| SEQ ID 108 | ---------------------------------------------------------------------- |
| SEQ ID 107 | ---------------------------------------------------------------------- |
| SEQ ID 10 | AGGACAAGTTGACGAGAGAGTCCCAAGTACGTCCACGGTCAGCCTTGCGGTAAGCTTGTGTGCTTAGAGG |
| SEQ ID 106 | AGGACAAGTTGACGAGAGAGTCCCAAGTACGTCCACGGTCAGCCTTGCGG-------------------- |
| SEQ ID 105 | AGGACAAGTTGACGAGAGAGTCCCAAGTACGTCCACGGTCAGCCTTGCG--------------------- |
| SEQ ID 99 | AGGACAAGTTGACGAGAGAGTCCCAAGTACGTCCACGGTCAGCCTTGCG--------------------- |
| SEQ ID 100 | AGGACAAGTTGACGAGAGAGTCCCAAGTACGTCCACGGTCAGCCTTGCGG-------------------- |
| SEQ ID 104 | AGGACAAGTTGACGAGAGAGTCCCAAGTACGTCCACGGTCAGCCTTGCGG-------------------- |
| SEQ ID 103 | AGGACAAGTTGACGAGAGAGTCCCAAGTACGTCCACGGTCAGCCTTGCG--------------------- |
| SEQ ID 101 | AGGACAAGTTGACGAGAGAGTCCCAAGTACGTCCACGGTCAGCCTTGCG--------------------- |
| SEQ ID 102 | AGGACAAGTTGACGAGAGAGTCCCAAGTACGTCCACGGTCAGCCTTGCG--------------------- |
| SEQ ID 108 | ---------------------------------------------------------------------- |
| SEQ ID 107 | ---------------------------------------------------------------------- |
| <break> | |
| SEQ ID 10 | TTGGTGGAAAGATAATAAAAATAAAACATGGGAAATGGGGAAGGTGATAACGTGGGGGAGAGGTTTTGCT |
| SEQ ID 106 | ---------------------------------------------------------------------- |
| SEQ ID 105 | ---------------------------------------------------------------------- |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | ---------------------------------------------------------------TTTTGCT |
| SEQ ID 107 | ---------------------------------------------------------------TTTTGCT |
| SEQ ID 10 | TGTGTTTCACCAGGAGAAAATCAGCTTCCTGTTTGGATACCCACTAGACATTTAAAGTTCTACAATGAAC |
| SEQ ID 106 | --------------AGAAAATCAGCTTCCTGTTTGGATACCCACTAGACATTTAAAGTTCTACAATGAAC |
| SEQ ID 105 | -----------------------------------------------ACATTTAAAGTTCTACAATGAAC |
| SEQ ID 99 | -----------------------------------------------ACATTTAAAGTTCTACAATGAAC |
| SEQ ID 100 | --------------AGAAAATCAGCTTCCTGTTTGGATACCCACTAGACATTTAAAGTTCTACAATGAAC |
| SEQ ID 104 | --------------AGAAAATCAGCTTCCTGTTTGGATACCCACTAGACATTTAAAGTTCTACAATGAAC |
| SEQ ID 103 | -----------------------------------------------ACATTTAAAGTTCTACAATGAAC |
| SEQ ID 101 | ---------------------------------------------------------------------- |
| SEQ ID 102 | ---------------------------------------------------------------------- |
| SEQ ID 108 | TGTGTTTCACCAGGAGAAAATCAGCTTCCTGTTTGGATACCCACTAGACATTTAAAGTTCTACAATGAAC |
| SEQ ID 107 | TGTGTTTCACCAGGAGAAAATCAGCTTCCTGTTTGGATACCCACTAGACATTTAAAGTTCTACAATGAAC |
| SEQ ID 10 | TCACTGGAGATGCAAAGAAAAGTGTGGAGATGGAGACACCCCAATCGACTCGCCAGGTAAACAAAATGGT |
| SEQ ID 106 | TCACTGGAGATGCAAAGAAAAGTGTGGAGATGGAGACACCCCAATCGACTCGCCAGGTAAACAAAATGGT |
| SEQ ID 105 | TCACTGGAGATGCAAAGAAAAGTGTGGAGATGGAGACACCCCAATCGACTCGCCAGGTAAACAAAATGGT |
| SEQ ID 99 | TCACTGGAGATGCAAAGAAAAGTGTGGAGATGGAGACACCCCAATCGACTCGCCAG-------------- |
| SEQ ID 100 | TCACTGGAGATGCAAAGAAAAGTGTGGAGATGGAGACACCCCAATCGACTCGCCAG-------------- |
| SEQ ID 104 | TCACTGGAGATGCAAAGAAAAGTGTGGAGATGGAGACACCCCAATCGACTCGCCAG-------------- |
| SEQ ID 103 | TCACTGGAGATGCAAAGAAAAGTGTGGAGATGGAGACACCCCAATCGACTCGCCAG-------------- |
| SEQ ID 101 | ---------------------------------------------------------------------- |
| SEQ ID 102 | ---------------------------------------------------------------------- |
| SEQ ID 108 | TCACTGGAGATGCAAAGAAAAGTGTGGAGATGGAGACACCCCAATCGACTCGCCAGGTAAACAAAATGGT |
| SEQ ID 107 | TCACTGGAGATGCAAAGAAAAGTGTGGAGATGGAGACACCCCAATCGACTCGCCAGGTAAACAAAATGGT |
| SEQ ID 10 | GATATCAGAAGAACAGAAAAAGTTGCCTTCCATCAAGGAAGCAGAGTTGCCAATATAGGCACAATTAAAG |
| SEQ ID 106 | GATATCAGAAGAACAGAAAAAGTTGCCTTCCATCAAGGAAGCAGAGTTGCCAATATAGGCACAATTAAAG |
| SEQ ID 105 | GATATCAGAAGAACAGAAAAAGTTGCCTTCCATCAAGGAAGCAGAGTTGCCAATATAGGCACAATTAAAG |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | GATATCAGAAGAACAGAAAAAGTTGCCTTCCATCAAGGAAGCAGAGTTGCCAATATAGGCACAATTAAAG |
| SEQ ID 107 | GATATCAGAAGAACAGAAAAAGTTGCCTTCCATCAAGGAAGCAGAGTTGCCAATATAGGCACAATTAAAG |
| SEQ ID 10 | AAGCTGACACAGTTAGCTAAAAAAAAAAGCCTAGAGAATACAAAGGTGACACCAACTCCAGAGAATATGC |
| SEQ ID 106 | AAGCTGACACAGTTAGCTAAAAAAAAAAGCCTAGAGAATACAAAGGTGACACCAACTCCAGAGAATATGC |
| SEQ ID 105 | AAGCTGACACAGTTAGCTAAAAAAAAAAGCCTAGAGAATACAAAGGTGACACCAACTCCAGAGAATATGC |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | AAGCTGACACAGTTAGCTAAAAAAAAAAGCCTAGAGAATACAAAGGTGACACCAACTCCAGAGAATATGC |
| SEQ ID 107 | AAGCTGACACAGTTAGCTAAAAAAAAAAGCCTAGAGAATACAAAGGTGACACCAACTCCAGAGAATATGC |
| SEQ ID 10 | TGCTTGCAGCTCTGATGATTGTATCAACGGTGGTAAGTCTTCCCAAGTCTGCAGGAGCAGCTGCAGCTAA |
| SEQ ID 106 | TGCTTGCAGCTCTGATGATTGTATCAACGGTG-------------------------------------- |
| SEQ ID 105 | TGCTTGCAGCTCTGATGATTGTATCAACGGTG-------------------------------------- |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | TGCTTGCAGCTCTGATGATTGTATCAACGGTGGTAAGTCTTCCCAAGTCTGCAGGAGCAGCTGCAGCTAA |
| SEQ ID 107 | TGCTTGCAGCTCTGATGATTGTATCAACGGTGGTAAGTCTTCCCAAGTCTGCAGGAGCAGCTGCAGCTAA |
| SEQ ID 10 | TTATACTTACTGGGCCTATGTGCCTTTCCCACCCTTAATTCGGGCAGTTACATAGATGGATAATCCTATT |
| SEQ ID 106 | ---------------------------------------------------------------------- |
| SEQ ID 105 | ---------------------------------------------------------------------- |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | TTATACTTACTGGGCCTATGTGCCTTTCCCACCCTTAATTCGGGCAGTTACATAGATGGATAATCCTATT |
| SEQ ID 107 | TTATACTTACTGGGCCTATGTGCCTTTCCCACCCTTAATTCGGGCAGTTACATAGATGGATAATCCTATT |
| SEQ ID 10 | GAAGTAGATGTTAATAATAGTGCATGGGTGCCTGGCCCCACAGATGACTGTTGCCCTGCCCAACCTGAAG |
| SEQ ID 106 | ---------------------------------------------------------------------- |
| SEQ ID 105 | ---------------------------------------------------------------------- |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | GAAGTAGATGTTAATAATAGTGCATGGGTGCCTGGCCCCACAGATGACTGTTGCCCTGCCCAACCTGAAG |
| SEQ ID 107 | GAAGTAGATGTTAATAATAGTGCATGGGTGCCTGGCCCCACAGATGACTGTTGCCCTGCCCAACCTGAAG |
| SEQ ID 10 | AAGGAATGATGATGAATATTTCCATTGGGTATCCTTATCCTCCTGTTTGCCTAGGGAAGGCACCAGGATG |
| SEQ ID 106 | ---------------------------------------------------------------------- |
| SEQ ID 105 | ---------------------------------------------------------------------- |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | AAGGAATGATGATGAATATTTCCATTGGGTATCCTTATCCTCCTGTTTGCCTAGGGAAGGCACCAGGATG |
| SEQ ID 107 | AAGGAATGATGATGAATATTTCCATTGGGTATCCTTATCCTCCTGTTTGCCTAGGGAAGGCACCAGGATG |
| | 8130 8140 8150 8160 8170 8180 8190 |
| | \| \| \| \| \| \| \| |
| SEQ ID 10 | CTTAATGCCTACAACCCAAAATTGGTTGGTAGAAGTACCTACAGTCAGTGCTACCAGTAGATTTACTTAT |
| SEQ ID 106 | ---------------------------------------------------------------------- |
| SEQ ID 105 | ---------------------------------------------------------------------- |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | CTTAATGCCTACAACCCAAAATTGGTTGGTAGAAGTACCTACAGTCAGTGCTACCAGTAGATTTACTTAT |
| SEQ ID 107 | CTTAATGCCTACAACCCAAAATTG---------------------------------------------- |
| SEQ ID 10 | CACATGGTAAGTGGAATGTCACAGATAAATAATTTACAGGACCCTTCTTATCAAAGATCATTACAATGTA |
| SEQ ID 106 | ---------------------------------------------------------------------- |
| SEQ ID 105 | ---------------------------------------------------------------------- |
| SEQ ID 99 | ---------------------------------------------------------------------- |
| SEQ ID 100 | ---------------------------------------------------------------------- |
| SEQ ID 104 | ---------------------------------------------------------------------- |
| SEQ ID 103 | ---------------------------------------------------------------------- |
| SEQ ID 101 | ---------------------------------------------------------------------- |
| SEQ ID 102 | ---------------------------------------------------------------------- |
| SEQ ID 108 | CACATG---------------------------------------------------------------- |
| SEQ ID 107 | ---------------------------------------------------------------------- |
| <break> | |
| SEQ ID 10 | CATCAGAAGTTTCACTATTGTAAATTTCATATTAATCCTTGTATGCCTGTTCTGTCTGTTGTTAGTCTAC |
| SEQ ID 106 | -----------------------------------------------------------------TCTAC |
| SEQ ID 105 | -----------------------------------------------------------------TCTAC |
| SEQ ID 99 | -----------------------------------------------------------------TCTAC |
| SEQ ID 100 | -----------------------------------------------------------------TCTAC |
| SEQ ID 104 | -----------------------------------------------------------------TCTAC |
| SEQ ID 103 | -----------------------------------------------------------------TCTAC |
| SEQ ID 101 | -----------------------------------------------------------------TCTAC |
| SEQ ID 102 | -----------------------------------------------------------------TCTAC |
| SEQ ID 108 | -----------------------------------------------------------------TCTAC |
| SEQ ID 107 | -----------------------------------------------------------------TCTAC |
| SEQ ID 10 | AGGTGTATCCAGCAGCTCCAAAGAGACAGCAACCAGCAAGAATGGGCCATAGTGACGATGGTGGTTTTGT |
| SEQ ID 106 | AGGTGTATCCAGCAGCTCCAAAGAGACAGCAACCAGCAAGAATGGGCCATAGTGACGATGGTGGTTTTGT |
| SEQ ID 105 | AGGTGTATCCAGCAGCTCCAAAGAGACAGCAACCAGCAAGAATGGGCCATAGTGACGATGGTGGTTTTGT |
| SEQ ID 99 | AGGTGTATCCAGCAGCTCCAAAGAGACAGCAACCAGCAAGAATGGGCCATAGTGACGATGGTGGTTTTGT |
| SEQ ID 100 | AGGTGTATCCAGCAGCTCCAAAGAGACAGCAACCAGCAAGAATGGGCCATAGTGACGATGGTGGTTTTGT |
| SEQ ID 104 | AGGTGTATCCAGCAGCTCCAAAGAGACAGCAACCAGCAAGAATGGGCCATAGTGACGATGGTGGTTTTGT |
| SEQ ID 103 | AGGTGTATCCAGCAGCTCCAGAAAGACAGCAACCAGCAAGAATGGGCCATAGTGACGATGGTGGTTTTGT |
| SEQ ID 101 | AGGTGTATCCAGCAGCTCCAAAGAGACAGCAACCAGCAAGAATGGGCCATAGTGACGATGGTGGTTTTGT |
| SEQ ID 102 | AGGTGTATCCAGCAGCTCCAAAGAGACAGCAACCAGCAAGAATGGGCCATAGTGACGATGGTGGTTTTGT |
| SEQ ID 108 | AGGTGTATCCAGCAGCTCCAAAGAGACAGCAACCAGCAAGAATGGGCCATAGTGACGATGGTGGTTTTGT |
| SEQ ID 107 | AGGTGTATCCAGCAGCTCCAAAGAGACAGCAACCAGCAAGAATGGGCCATAGTGACGATGGTGGTTTTGT |
| SEQ ID 10 | CAAAAAGAAAAGGGGGGGATATGTAAGGAAAAGAGAGATCAGACTTTCACTGTGTCTATGTAGAAAAGGA |
| SEQ ID 106 | CAAAAAGAAAAGGGGGGGATATGTAAGGAAAAGAGAGATCAGACTTTCACTGTGTCTATGTAGAAAAGGA |
| SEQ ID 105 | CAAAAAGAAAAGGGGGGGATATGTAAGGAAAAGAGAGATCAGACTTTCACTGTGTCTATGTAGAAAAGGA |
| SEQ ID 99 | CAAAAAGAAAAGGGGGGGATATGTAAGGAAAAGAGAGATCAGACTTTCACTGTGTCTATGTAGAAAAGGA |
| SEQ ID 100 | CAAAAAGAAAAGGGGGGGATATGTAAGGAAAAGAGAGATCAGACTTTCACTGTGTCTATGTAGAAAAGGA |
| SEQ ID 104 | CAAAAAGAAAAGGGGGGGATATGTAAGGAAAAGAGAGATCAGACTTTCACTGTGTCTATGTAGAAAAGGA |
| SEQ ID 103 | CAAAAAGAAAAGGGGGGGATATGTAAGGAAAAGAGAGATCAGACTTTCACTGTGTCTATGTAGAAAAGGA |
| SEQ ID 101 | CAAAAAGAAAAGGGGGGGATATGTAAGGAAAAGAGAGATCAGACTTTCACTGTGTCTATGTAGAAAAGGA |
| SEQ ID 102 | CAAAAAGAAAAGGGGGGGATATGTAAGGAAAAGAGAGATCAGACTTTCACTGTGTCTATGTAGAAAAGGA |
| SEQ ID 108 | CAAAAAGAAAAGGGGGGGATATGTAAGGAAAAGAGAGATCAGACTTTCACTGTGTCTATGTAGAAAAGGA |
| SEQ ID 107 | CAAAAAGAAAAGGGGGGGATATGTAAGGAAAAGAGAGATCAGACTTTCACTGTGTCTATGTAGAAAAGGA |
| SEQ ID 10 | AGACATAAGAAACTCCATTTTGATCTGTACTAAGAAAAATTGTTTTGCCTTGAGATGCTGTTAATCTGTA |
| SEQ ID 106 | AGACATAAGAAACTCCATTTTGATCTGTACTAAGAAAAATTGTTTTGCCTTGAGATGCTGTTAATCTGTA |
| SEQ ID 105 | AGACATAAGAAACTCCATTTTGATCTGTACTAAGAAAAATTGTTTTGCCTTGAGATGCTGTTAATCTGTA |
| SEQ ID 99 | AGACATAAGAAACTCCATTTTGATCTGTACTAAGAAAAATTGTTTTGCCTTGAGATGCTGTTAATCTGTA |
| SEQ ID 100 | AGACATAAGAAACTCCATTTTGATCTGTACTAAGAAAAATTGTTTTGCCTTGAGATGCTGTTAATCTGTA |
| SEQ ID 104 | AGACATAAGAAACTCCATTTTGATCTGTACTAAGAAAAATTGTTTTGCCTTGAGATGCTGTTAATCTGTA |
| SEQ ID 103 | AGACATAAGAAACTCCATTTTGATCTGTACTAAGAAAAATTGTTTTGCCTTGAGATGCTGTTAATCTGTA |
| SEQ ID 101 | AGACATAAGAAACTCCATTTTGATCTGTACTAAGAAAAATTGTTTTGCCTTGAGATGCTGTTAATCTGTA |
| SEQ ID 102 | AGACATAAGAAACTCCATTTTGATCTGTACTAAGAAAAATTGTTTTGCCTTGAGATGCTGTTAATCTGTA |
| SEQ ID 108 | AGACATAAGAAACTCCATTTTGATCTGTACTAAGAAAAATTGTTTTGCCTTGAGATGCTGTTAATCTGTA |
| SEQ ID 107 | AGACATAAGAAACTCCATTTTGATCTGTACTAAGAAAAATTGTTTTGCCTTGAGATGCTGTTAATCTGTA |
| SEQ ID 10 | ACTTTAGCCCCAACCCTGTGCTCACGGAAACATGTGCTGTAAGGTTTAAGGGATCTAGGGCTGTGCAGGA |
| SEQ ID 106 | ACTTTAGCCCCAACCCTGTGCTCACGGAAACATGTGCTGTAAGGTTTAAGGGATCTAGGGCTGTGCAGGA |
| SEQ ID 105 | ACTTTAGCCCCAACCCTGTGCTCACGGAAACATGTGCTGTAAGGTTTAAGGGATCTAGGGCTGTGCAGGA |
| SEQ ID 99 | ACTTTAGCCCCAACCCTGTGCTCACGGAAACATGTGCTGTAAGGTTTAAGGGATCTAGGGCTGTGCAGGA |
| SEQ ID 100 | ACTTTAGCCCCAACCCTGTGCTCACGGAAACATGTGCTGTAAGGTTTAAGGGATCTAGGGCTGTGCAGGA |
| SEQ ID 104 | ACTTTAGCCCCAACCCTGTGCTCACGGAAACATGTGCTGTAAGGTTTAAGGGATCTAGGGCTGTGCAGGA |
| SEQ ID 103 | ACTTTAGCCCCAACCCTGTGCTCACGGAAACATGTGCTGTAAGGTTTAAGGGATCTAGGGCTGTGCAGGA |
| SEQ ID 101 | ACTTTAGCCCCAACCCTGTGCTCACGGAAACATGTGCTGTAAGGTTTAAGGGATCTAGGGCTGTGCAGGA |
| SEQ ID 102 | ACTTTAGCCCCAACCCTGTGCTCACGGAAACATGTGCTGTAAGGTTTAAGGGATCTAGGGCTGTGCAGGA |
| SEQ ID 108 | ACTTTAGCCCCAACCCTGTGCTCACGGAAACATGTGCTGTAAGGTTTAAGGGATCTAGGGCTGTGCAGGA |
| SEQ ID 107 | ACTTTAGCCCCAACCCTGTGCTCACGGAAACATGTGCTGTAAGGTTTAAGGGATCTAGGGCTGTGCAGGA |
| SEQ ID 10 | TGTACCTTGTTAACAATATGTTTGCAGGCAGTATGTTTGGTAAAAGTCATCGCCATTCTCCATTCTCGAT |
| SEQ ID 106 | TGTACCTTGTTAACAATATGTTTGCAGGCAGTATGTTTGGTAAAAGTCATCGCCATTCTCCATTCTCGAT |
| SEQ ID 105 | TGTACCTTGTTAACAATATGTTTGCAGGCAGTATGTTTGGTAAAAGTCATCGCCATTCTCCATTCTCGAT |
| SEQ ID 99 | TGTACCTTGTTAACAATATGTTTGCAGGCAGTATGTTTGGTAAAAGTCATCGCCATTCTCCATTCTCGAT |
| SEQ ID 100 | TGTACCTTGTTAACAATATGTTTGCAGGCAGTATGTTTGGTAAAAGTCATCGCCATTCTCCATTCTCGAT |
| SEQ ID 104 | TGTACCTTGTTAACAATATGTTTGCAGGCAGTATGTTTGGTAAAAGTCATCGCCATTCTCCATTCTCGAT |
| SEQ ID 103 | TGTACCTTGTTAACAATATGTTTGCAGGCAGTATGTTTGGTAAAAGTCATCGCCATTCTCCATTCTCGAT |
| SEQ ID 101 | TGTACCTTGTTAACAATATGTTTGCAGGCAGTATGTTTGGTAAAAGTCATCGCCATTCTCCATTCTCGAT |
| SEQ ID 102 | TGTACCTTGTTAACAATATGTTTGCAGGCAGTATGTTTGGTAAAAGTCATCGCCATTCTCCATTCTCGAT |
| SEQ ID 108 | TGTACCTTGTTAACAATATGTTTGCAGGCAGTATGTTTGGTAAAAGTCATCGCCATTCTCCATTCTCGAT |
| SEQ ID 107 | TGTACCTTGTTAACAATATGTTTGCAGGCAGTATGTTTGGTAAAAGTCATCGCCATTCTCCATTCTCGAT |
| SEQ ID 10 | TAACCAGGGGCTCAATGCACTGTGGAAAGCCACAGGAACCTCTGCCCAAGAAAGCCTGGCTGTTGTGGGA |
| SEQ ID 106 | TAACCAGGGGCTCAATGCACTGTGGAAAGCCACAGGAACCTCTGCCCAAGAAAGCCTGGCTGTTGTGGGA |
| SEQ ID 105 | TAACCAGGGGCTCAATGCACTGTGGAAAGCCACAGGAACCTCTGCCCAAGAAAGCCTGGCTGTTGTGGGA |
| SEQ ID 99 | TAACCAGGGGCTCAATGCACTGTGGAAAGCCACAGGAACCTCTGCCCAAGAAAGCCTGGCTGTTGTGGGA |
| SEQ ID 100 | TAACCAGGGGCTCAATGCACTGTGGAAAGCCACAGGAACCTCTGCCCAAGAAAGCCTGGCTGTTGTGGGA |
| SEQ ID 104 | TAACCAGGGGCTCAATGCACTGTGGAAAGCCACAGGAACCTCTGCCCAAGAAAGCCTGGCTGTTGTGGGA |
| SEQ ID 103 | TAACCAGGGGCTCAATGCACTGTGGAAAGCCACAGGAACCTCTGCCCAAGAAAGCCTGGCTGTTGTGGGA |
| SEQ ID 101 | TAACCAGGGGCTCAATGCACTGTGGAAAGCCACAGGAACCTCTGCCCAAGAAAGCCTGGCTGTTGTGGGA |
| SEQ ID 102 | TAACCAGGGGCTCAATGCACTGTGGAAAGCCACAGGAACCTCTGCCCAAGAAAGCCTGGCTGTTGTGGGA |
| SEQ ID 108 | TAACCAGGGGCTCAATGCACTGTGGAAAGCCACAGGAACCTCTGCCCAAGAAAGCCTGGCTGTTGTGGGA |
| SEQ ID 107 | TAACCAGGGGCTCAATGCACTGTGGAAAGCCACAGGAACCTCTGCCCAAGAAAGCCTGGCTGTTGTGGGA |
| SEQ ID 10 | AGTCAGGGACCCCGAATGGAGGGACCAGCTGGTGCTGCATCAGGAAACATAAATTGTGAAGATTTCTTGG |
| SEQ ID 106 | AGTCAGGGACCCCGAATGGAGGGACCAGCTGGTGCTGCATCAGGAAACATAAATTGTGAAGATTTCTTGG |
| SEQ ID 105 | AGTCAGGGACCCCGAATGGAGGGACCAGCTGGTGCTGCATCAGGAAACATAAATTGTGAAGATTTCTTGG |
| SEQ ID 99 | AGTCAGGGACCCCGAATGGAGGGACCAGCTGGTGCTGCATCAGGAAACATAAATTGTGAAGATTTCTTGG |
| SEQ ID 100 | AGTCAGGGACCCCGAATGGAGGGACCAGCTGGTGCTGCATCAGGAAACATAAATTGTGAAGATTTCTTGG |
| SEQ ID 104 | AGTCAGGGACCCCGAATGGAGGGACCAGCTGGTGCTGCATCAGGAAACATAAATTGTGAAGATTTCTTGG |
| SEQ ID 103 | AGTCAGGGACCCCGAATGGAGGGACCAGCTGGTGCTGCATCAGGAAACATAAATTGTGAAGATTTCTTGG |
| SEQ ID 101 | AGTCAGGGACCCCGAATGGAGGGACCAGCTGGTGCTGCATCAGGAAACATAAATTGTGAAGATTTCTTGG |
| SEQ ID 102 | AGTCAGGGACCCCGAATGGAGGGACCAGCTGGTGCTGCATCAGGAAACATAAATTGTGAAGATTTCTTGG |
| SEQ ID 108 | AGTCAGGGACCCCGAATGGAGGGACCAGCTGGTGCTGCATCAGGAAACATAAATTGTGAAGATTTCTTGG |
| SEQ ID 107 | AGTCAGGGACCCCGAATGGAGGGACCAGCTGGTGCTGCATCAGGAAACATAAATTGTGAAGATTTCTTGG |
| SEQ ID 10 | ACATTTATCAGTTTCCAAAATTAATACTTTTATAATTTCTTACACCTGTCTTACTTTAATCTCTTAATCC |
| SEQ ID 106 | ACATTTATCAGTTTCCAAAATTAATACTTTTATAATTTCTTACACCTGTCTTACTTTAATCTCTTAATCC |
| SEQ ID 105 | ACATTTATCAGTTTCCAAAATTAATACTTTTATAATTTCTTACACCTGTCTTACTTTAATCTCTTAATCC |
| SEQ ID 99 | ACATTTATCAGTTTCCAAAATTAATACTTTTATAATTTCTTACACCTGTCTTACTTTAATCTCTTAATCC |
| SEQ ID 100 | ACATTTATCAGTTTCCAAAATTAATACTTTTATAATTTCTTACACCTGTCTTACTTTAATCTCTTAATCC |
| SEQ ID 104 | ACATTTATCAGTTTCCAAAATTAATACTTTTATAATTTCTTACACCTGTCTTACTTTAATCTCTTAATCC |
| SEQ ID 103 | ACATTTATCAGTTTCCAAAATTAATACTTTTATAATTTCTTACACCTGTCTTACTTTAATCTCTTAATCC |
| SEQ ID 101 | ACATTTATCAGTTTCCAAAATTAATACTTTTATAATTTCTTACACCTGTCTTACTTTAATCTCTTAATCC |
| SEQ ID 102 | ACATTTATCAGTTTCCAAAATTAATACTTTTATAATTTCTTACACCTGTCTTACTTTAATCTCTTAATCC |
| SEQ ID 108 | ACATTTATCAGTTTCCAAAATTAATACTTTTATAATTTCTTACACCTGTCTTACTTTAATCTCTTAATCC |
| SEQ ID 107 | ACATTTATCAGTTTCCAAAATTAATACTTTTATAATTTCTTACACCTGTCTTACTTTAATCTCTTAATCC |
| SEQ ID 10 | TGTTATCTTTGTAAGCTGAGGATATACGTCACCTCAGGACCACTATTGTACAAATTGATTGTAAAACATG |
| SEQ ID 106 | TGTTATCTTTGTAAGCTGAGGATATACGTCACCTCAGGACCACTATTGTACAAATTGATTGTAAAACATG |
| SEQ ID 105 | TGTTATCTTTGTAAGCTGAGGATATACGTCACCTCAGGACCACTATTGTACAAATTGATTGTAAAACATG |
| SEQ ID 99 | TGTTATCTTTGTAAGCTGAGGATATACGTCACCTCAGGACCACTATTGTACAAATTGATTGTAAAACATG |
| SEQ ID 100 | TGTTATCTTTGTAAGCTGAGGATATACGTCACCTCAGGACCACTATTGTACAAATTGATTGTAAAACATG |
| SEQ ID 104 | TGTTATCTTTGTAAGCTGAGGATATACGTCACCTCAGGACCACTATTGTACAAATTGATTGTAAAACATG |
| SEQ ID 103 | TGTTATCTTTGTAAGCTGAGGATATACGTCACCTCAGGACCACTATTGTACAAATTGATTGTAAAACATG |
| SEQ ID 101 | TGTTATCTTTGTAAGCTGAGGATATACGTCACCTCAGGACCACTATTGTACAAATTGATTGTAAAACATG |
| SEQ ID 102 | TGTTATCTTTGTAAGCTGAGGATATACGTCACCTCAGGACCACTATTGTACAAATTGATTGTAAAACATG |
| SEQ ID 108 | TGTTATCTTTGTAAGCTGAGGATATACGTCACCTCAGGACCACTATTGTACAAATTGATTGTAAAACATG |
| SEQ ID 107 | TGTTATCTTTGTAAGCTGAGGATATACGTCACCTCAGGACCACTATTGTACAAATTGATTGTAAAACATG |
| SEQ ID 10 | TTCACATGTGTTTGAACAATATGAAATCAGTGCACCTTGAAAATGAACAGAATAACAGTGATTTTAGGGA |
| SEQ ID 106 | TTCACATGTGTTTGAACAATATGAAATCAGTGCACCTTGAAAATGAACAGAATAACAGTGATTTTAGGGA |
| SEQ ID 105 | TTCACATGTGTTTGAACAATATGAAATCAGTGCACCTTGAAAATGAACAGAATAACAGTGATTTTAGGGA |
| SEQ ID 99 | TTCACATGTGTTTGAACAATATGAAATCAGTGCACCTTGAAAATGAACAGAATAACAGTGATTTTAGGGA |
| SEQ ID 100 | TTCACATGTGTTTGAACAATATGAAATCAGTGCACCTTGAAAATGAACAGAATAACAGTGATTTTAGGGA |
| SEQ ID 104 | TTCACATGTGTTTGAACAATATGAAATCAGTGCACCTTGAAAATGAACAGAATAACAGTGATTTTAGGGA |
| SEQ ID 103 | TTCACATGTGTTTGAACAATATGAAATCAGTGCACCTTGAAAATGAACAGAATAACAGTGATTTTAGGGA |
| SEQ ID 101 | TTCACATGTGTTTGAACAATATGAAATCAGTGCACCTTGAAAATGAACAGAATAACAGTGATTTTAGGGA |
| SEQ ID 102 | TTCACATGTGTTTGAACAATATGAAATCAGTGCACCTTGAAAATGAACAGAATAACAGTGATTTTAGGGA |
| SEQ ID 108 | TTCACATGTGTTTGAACAATATGAAATCAGTGCACCTTGAAAATGAACAGAATAACAGTGATTTTAGGGA |
| SEQ ID 107 | TTCACATGTGTTTGAACAATATGAAATCAGTGCACCTTGAAAATGAACAGAATAACAGTGATTTTAGGGA |
| SEQ ID 10 | ACAAAGGAAGACAACCATAAGGTCTGACTGCCTGAGGGGTCGGGCAAAAAGCCATATTTTTCTTCTTGCA |
| SEQ ID 106 | ACAAAGGAAGACAACCATAAGGTCTGACTGCCTGAGGGGTCGGGCAAAAAGCCATATTTTTCTTCTTGCA |
| SEQ ID 105 | ACAAAGGAAGACAACCATAAGGTCTGACTGCCTGAGGGGTCGGGCAAAAAGCCATATTTTTCTTCTTGCA |
| SEQ ID 99 | ACAAAGGAAGACAACCATAAGGTCTGACTGCCTGAGGGGTCGGGCAAAAAGCCATATTTTTCTTCTTGCA |
| SEQ ID 100 | ACAAAGGAAGACAACCATAAGGTCTGACTGCCTGAGGGGTCGGGCAAAAAGCCATATTTTTCTTCTTGCA |
| SEQ ID 104 | ACAAAGGAAGACAACCATAAGGTCTGACTGCCTGAGGGGTCGGGCAAAAAGCCATATTTTTCTTCTTGCA |
| SEQ ID 103 | ACAAAGGAAGACAACCATAAGGTCTGACTGCCTGAGGGGTCGGGCAAAAAGCCATATTTTTCTTCTTGCA |
| SEQ ID 101 | ACAAAGGAAGACAACCATAAGGTCTGACTGCCTGAGGGGTCGGGCAAAAAGCCATATTTTTCTTCTTGCA |
| SEQ ID 102 | ACAAAGGAAGACAACCATAAGGTCTGACTGCCTGAGGGGTCGGGCAAAAAGCCATATTTTTCTTCTTGCA |
| SEQ ID 108 | ACAAAGGAAGACAACCATAAGGTCTGACTGCCTGAGGGGTCGGGCAAAAAGCCATATTTTTCTTCTTGCA |
| SEQ ID 107 | ACAAAGGAAGACAACCATAAGGTCTGACTGCCTGAGGGGTCGGGCAAAAAGCCATATTTTTCTTCTTGCA |
| SEQ ID 10 | GAGAGCCTATAAATGGACGTGCAAGTAGGAGAGATATTGCTAAATTCTTTTCCTAGCAAGGAATATAATA |
| SEQ ID 106 | GAGAGCCTATAAATGGACGTGCAAGTAGGAGAGATATTGCTAAATTCTTTTCCTAGCAAGGAATATAATA |
| SEQ ID 105 | GAGAGCCTATAAATGGACGTGCAAGTAGGAGAGATATTGCTAAATTCTTTTCCTAGCAAGGAATATAATA |
| SEQ ID 99 | GAGAGCCTATAAATGGACGTGCAAGTAGGAGAGATATTGCTAAATTCTTTTCCTAGCAAGGAATATAATA |
| SEQ ID 100 | GAGAGCCTATAAATGGACGTGCAAGTAGGAGAGATATTGCTAAATTCTTTTCCTAGCAAGGAATATAATA |
| SEQ ID 104 | GAGAGCCTATAAATGGACGTGCAAGTAGGAGAGATATTGCTAAATTCTTTTCCTAGCAAGGAATATAATA |
| SEQ ID 103 | GAGAGCCTATAAATGGACGTGCAAGTAGGAGAGATATTGCTAAATTCTTTTCCTAGCAAGGAATATAATA |
| SEQ ID 101 | GAGAGCCTATAAATGGACGTGCAAGTAGGAGAGATATTGCTAAATTCTTTTCCTAGCAAGGAATATAATA |
| SEQ ID 102 | GAGAGCCTATAAATGGACGTGCAAGTAGGAGAGATATTGCTAAATTCTTTTCCTAGCAAGGAATATAATA |
| SEQ ID 108 | GAGAGCCTATAAATGGACGTGCAAGTAGGAGAGATATTGCTAAATTCTTTTCCTAGCAAGGAATATAATA |
| SEQ ID 107 | GAGAGCCTATAAATGGACGTGCAAGTAGGAGAGATATTGCTAAATTCTTTTCCTAGCAAGGAATATAATA |
| SEQ ID 10 | CTAAGACCCTAGGGAAAGAATTGCATTCCTGGGGGGAGGTCTATAAACGGCCGCTCTGGGAGTGTCTGTC |
| SEQ ID 106 | CTAAGACCCTAGGGAAAGAATTGCATTCCTGGGGGGAGGTCTATAAACGGCCGCTCTGGGAGTGTCTGTC |
| SEQ ID 105 | CTAAGACCCTAGGGAAAGAATTGCATTCCTGGGGGGAGGTCTATAAACGGCCGCTCTGGGAGTGTCTGTC |
| SEQ ID 99 | CTAAGACCCTAGGGAAAGAATTGCATTCCTGGGGGGAGGTCTATAAACGGCCGCTCTGGGAGTGTCTGTC |
| SEQ ID 100 | CTAAGACCCTAGGGAAAGAATTGCATTCCTGGGGGGAGGTCTATAAACGGCCGCTCTGGGAGTGTCTGTC |
| SEQ ID 104 | CTAAGACCCTAGGGAAAGAATTGCATTCCTGGGGGGAGGTCTATAAACGGCCGCTCTGGGAGTGTCTGTC |
| SEQ ID 103 | CTAAGACCCTAGGGAAAGAATTGCATTCCTGGGGGGAGGTCTATAAACGGCCGCTCTGGGAGTGTCTGTC |
| SEQ ID 101 | CTAAGACCCTAGGGAAAGAATTGCATTCCTGGGGGGAGGTCTATAAACGGCCGCTCTGGGAGTGTCTGTC |
| SEQ ID 102 | CTAAGACCCTAGGGAAAGAATTGCATTCCTGGGGGGAGGTCTATAAACGGCCGCTCTGGGAGTGTCTGTC |
| SEQ ID 108 | CTAAGACCCTAGGGAAAGAATTGCATTCCTGGGGGGAGGTCTATAAACGGCCGCTCTGGGAGTGTCTGTC |
| SEQ ID 107 | CTAAGACCCTAGGGAAAGAATTGCATTCCTGGGGGGAGGTCTATAAACGGCCGCTCTGGGAGTGTCTGTC |
| SEQ ID 10 | CTATGTGGTTGAGATAAGGACTGAGATACGCCCTGGTCTCCTGCAGTACCCTCAGGCTTACTAGGATTGG |
| SEQ ID 106 | CTATGTGGTTGAGATAAGGACTGAGATACGCCCTGGTCTCCTGCAGTACCCTCAGGCTTACTAGGATTGG |
| SEQ ID 105 | CTATGTGGTTGAGATAAGGACTGAGATACGCCCTGGTCTCCTGCAGTACCCTCAGGCTTACTAGGATTGG |
| SEQ ID 99 | CTATGTGGTTGAGATAAGGACTGAGATACGCCCTGGTCTCCTGCAGTACCCTCAGGCTTACTAGGATTGG |
| SEQ ID 100 | CTATGTGGTTGAGATAAGGACTGAGATACGCCCTGGTCTCCTGCAGTACCCTCAGGCTTACTAGGATTGG |
| SEQ ID 104 | CTATGTGGTTGAGATAAGGACTGAGATACGCCCTGGTCTCCTGCAGTACCCTCAGGCTTACTAGGATTGG |
| SEQ ID 103 | CTATGTGGTTGAGATAAGGACTGAGATACGCCCTGGTCTCCTGCAGTACCCTCAGGCTTACTAGGATTGG |
| SEQ ID 101 | CTATGTGGTTGAGATAAGGACTGAGATACGCCCTGGTCTCCTGCAGTACCCTCAGGCTTACTAGGATTGG |
| SEQ ID 102 | CTATGTGGTTGAGATAAGGACTGAGATACGCCCTGGTCTCCTGCAGTACCCTCAGGCTTACTAGGATTGG |
| SEQ ID 108 | CTATGTGGTTGAGATAAGGACTGAGATACGCCCTGGTCTCCTGCAGTACCCTCAGGCTTACTAGGATTGG |
| SEQ ID 107 | CTATGTGGTTGAGATAAGGACTGAGATACGCCCTGGTCTCCTGCAGTACCCTCAGGCTTACTAGGATTGG |
| SEQ ID 10 | GAAACCCCAGTCCTGGTAAATTTGAGGTCAGGCCGGTTCTTTGCTCTGAACCCTGTTTTCTGTTAAGATG |
| SEQ ID 106 | GAAACCCCAGTCCTGGTAAATTTGAGGTCAGGCCGGTTCTTTGCTCTGAACCCTGTTTTCTGTTAAGATG |
| SEQ ID 105 | GAAACCCCAGTCCTGGTAAATTTGAGGTCAGGCCGGTTCTTTGCTCTGAACCCTGTTTTCTGTTAAGATG |
| SEQ ID 99 | GAAACCCCAGTCCTGGTAAATTTGAGGTCAGGCCGGTTCTTTGCTCTGAACCCTGTTTTCTGTTAAGATG |
| SEQ ID 100 | GAAACCCCAGTCCTGGTAAATTTGAGGTCAGGCCGGTTCTTTGCTCTGAACCCTGTTTTCTGTTAAGATG |
| SEQ ID 104 | GAAACCCCAGTCCTGGTAAATTTGAGGTCAGGCCGGTTCTTTGCTCTGAACCCTGTTTTCTGTTAAGATG |
| SEQ ID 103 | GAAACCCCAGTCCTGGTAAATTTGAGGTCAGGCCGGTTCTTTGCTCTGAACCCTGTTTTCTGTTAAGATG |
| SEQ ID 101 | GAAACCCCAGTCCTGGTAAATTTGAGGTCAGGCCGGTTCTTTGCTCTGAACCCTGTTTTCTGTTAAGATG |
| SEQ ID 102 | GAAACCCCAGTCCTGGTAAATTTGAGGTCAGGCCGGTTCTTTGCTCTGAACCCTGTTTTCTGTTAAGATG |
| SEQ ID 108 | GAAACCCCAGTCCTGGTAAATTTGAGGTCAGGCCGGTTCTTTGCTCTGAACCCTGTTTTCTGTTAAGATG |
| SEQ ID 107 | GAAACCCCAGTCCTGGTAAATTTGAGGTCAGGCCGGTTCTTTGCTCTGAACCCTGTTTTCTGTTAAGATG |
| SEQ ID 10 | TTTATCAAGACAATACATGCACCGCTGAACATAGACCCTTATCAGGAGTTTCTGATTTTGCTCTGGTCCT |
| SEQ ID 106 | TTTATCAAGACAATACATGCACCGCTGAACATAGACCCTTATCAGGAGTTTCTGATTTTGCTCTGGTCCT |
| SEQ ID 105 | TTTATCAAGACAATACATGCACCGCTGAACATAGACCCTTATCAGGAGTTTCTGATTTTGCTCTGGTCCT |
| SEQ ID 99 | TTTATCAAGACAATACATGCACCGCTGAACATAGACCCTTATCAGGAGTTTCTGATTTTGCTCTGGTCCT |
| SEQ ID 100 | TTTATCAAGACAATACATGCACCGCTGAACATAGACCCTTATCAGGAGTTTCTGATTTTGCTCTGGTCCT |
| SEQ ID 104 | TTTATCAAGACAATACATGCACCGCTGAACATAGACCCTTATCAGGAGTTTCTGATTTTGCTCTGGTCCT |
| SEQ ID 103 | TTTATCAAGACAATACATGCACCGCTGAACATAGACCCTTATCAGGAGTTTCTGATTTTGCTCTGGTCCT |
| SEQ ID 101 | TTTATCAAGACAATACATGCACCGCTGAACATAGACCCTTATCAGGAGTTTCTGATTTTGCTCTGGTCCT |
| SEQ ID 102 | TTTATCAAGACAATACATGCACCGCTGAACATAGACCCTTATCAGGAGTTTCTGATTTTGCTCTGGTCCT |
| SEQ ID 108 | TTTATCAAGACAATACATGCACCGCTGAACATAGACCCTTATCAGGAGTTTCTGATTTTGCTCTGGTCCT |
| SEQ ID 107 | TTTATCAAGACAATACATGCACCGCTGAACATAGACCCTTATCAGGAGTTTCTGATTTTGCTCTGGTCCT |
| SEQ ID 10 | GTTTCTTCAGAAGCATGTCATCTTTGCTCTGCCTTCTGCCCTTTGAAGCATGTGATCTTTGTGACCTACT |
| SEQ ID 106 | GTTTCTTCAGAAGCATGTCATCTTTGCTCTGCCTTCTGCCCTTTGAAGCATGTGATCTTTGTGACCTACT |
| SEQ ID 105 | GTTTCTTCAGAAGCATGTCATCTTTGCTCTGCCTTCTGCCCTTTGAAGCATGTGATCTTTGTGACCTACT |
| SEQ ID 99 | GTTTCTTCAGAAGCATGTCATCTTTGCTCTGCCTTCTGCCCTTTGAAGCATGTGATCTTTGTGACCTACT |
| SEQ ID 100 | GTTTCTTCAGAAGCATGTCATCTTTGCTCTGCCTTCTGCCCTTTGAAGCATGTGATCTTTGTGACCTACT |
| SEQ ID 104 | GTTTCTTCAGAAGCATGTCATCTTTGCTCTGCCTTCTGCCCTTTGAAGCATGTGATCTTTGTGACCTACT |
| SEQ ID 103 | GTTTCTTCAGAAGCATGTCATCTTTGCTCTGCCTTCTGCCCTTTGAAGCATGTGATCTTTGTGACCTACT |
| SEQ ID 101 | GTTTCTTCAGAAGCATGTCATCTTTGCTCTGCCTTCTGCCCTTTGAAGCATGTGATCTTTGTGACCTACT |
| SEQ ID 102 | GTTTCTTCAGAAGCATGTCATCTTTGCTCTGCCTTCTGCCCTTTGAAGCATGTGATCTTTGTGACCTACT |
| SEQ ID 108 | GTTTCTTCAGAAGCATGTCATCTTTGCTCTGCCTTCTGCCCTTTGAAGCATGTGATCTTTGTGACCTACT |
| SEQ ID 107 | GTTTCTTCAGAAGCATGTCATCTTTGCTCTGCCTTCTGCCCTTTGAAGCATGTGATCTTTGTGACCTACT |
| SEQ ID 10 | CCCTGTTCATACACCCCTCCCCTTTTAAAATCCCTAATAAAAACTTGCTGGTTTTGTGGCTCAGGGGGGC |
| SEQ ID 106 | CCCTGTTCATACACCCCTCCCCTTTTAAAATCCCTAATAAAAACTTGCTGGTTTTGTGGCTCAGGGGGGC |
| SEQ ID 105 | CCCTGTTCATACACCCCTCCCCTTTTAAAATCCCTAATAAAAACTTGCTGGTTTTGTGGCTCAGGGGGGC |
| SEQ ID 99 | CCCTGTTCATACACCCCTCCCCTTTTAAAATCCCTAATAAAAACTTGCTGGTTTTGTGGCTCAGGGGGGC |
| SEQ ID 100 | CCCTGTTCATACACCCCTCCCCTTTTAAAATCCCTAATAAAAACTTGCTGGTTTTGTGGCTCAGGGGGGC |
| SEQ ID 104 | CCCTGTTCATACACCCCTCCCCTTTTAAAATCCCTAATAAAAACTTGCTGGTTTTGTGGCTCAGGGGGGC |
| SEQ ID 103 | CCCTGTTCATACACCCCTCCCCTTTTAAAATCCCTAATAAAAACTTGCTGGTTTTGTGGCTCAGGGGGGc |
| SEQ ID 101 | CCCTGTTCATACACCCCTCCCCTTTTAAAATCCCTAATAAAAACTTGCTGGTTTTGTGGCTCAGGGGGGC |
| SEQ ID 102 | CCCTGTTCATACACCCCTCCCCTTTTAAAATCCCTAATAAAAACTTGCTGGTTTTGTGGCTCAGGGGGGC |
| SEQ ID 108 | CCCTGTTCATACACCCCTCCCCTTTTAAAATCCCTAATAAAAACTTGCTGGTTTTGTGGCTCAGGGGGGC |
| SEQ ID 107 | CCCTGTTCATACACCCCTCCCCTTTTAAAATCCCTAATAAAAACTTGCTGGTTTTGTGGCTCAGGGGGGC |
| SEQ ID 10 | ATCATGGACCTACCAATACGTGATGTCACCCCCGGTGGCCCAGCTGT---- |
| SEQ ID 106 | ATCATGGACCTACCAATACGTGATGTCACCCCCGGTGGCCCAGCTGTAAAA |
| SEQ ID 105 | ATCATGGACCTACCAATACGTGATGTCACCCCCGGTGGCCCAGCTGTAAAA |
| SEQ ID 99 | ATCATGGACCTACCAATACGTGATGTCACCCCCGGTGGCCCAGCTGTAAAA |
| SEQ ID 100 | ATCATGGACCTACCAATACGTGATGTCACCCCCGGTGGCCCAGCTGTAAAA |
| SEQ ID 104 | ATCATGGACCTACCAATACGTGATGTCACCCCCGGTGGCCCAGCTGTAAAA |
| SEQ ID 103 | ATCATGGACCTACCAATACGTGATGTCACCCCCGGTGGCCCAGCTGTAAAA |
| SEQ ID 101 | ATCATGGACCTACCAATACGTGATGTCACCCCCGGTGGCCCAGCTGTAAAA |
| SEQ ID 102 | ATCATGGACCTACCAATACGTGATGTCACCCCCGGTGGCCCAGCTGTAAAA |
| SEQ ID 108 | ATCATGGACCTACCAATACGTGATGTCACCCCCGGTGGCCCAGCTGTAAAA |
| SEQ ID 107 | ATCATGGACCTACCAATACGTGATGTCACCCCCGGTGGCCCAGCTGTAAAA |

The Transcription Start Site of PCAV

By homology to other retroviruses, the 5′ end of PCAV-mRNA (i.e. the transcription start site within the PCAV genome) should fall 30 bases downstream of the canonical TATA sequence, at nucleotide 559 in SEQ ID 1.

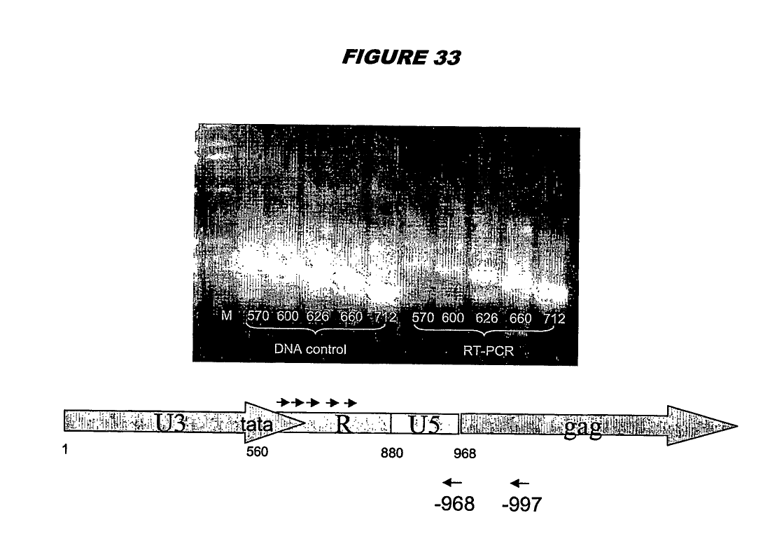

However, empirical work suggests that the 5′ end of PCAV-mRNA is further downstream. FIG. 33 shows the results of a RT-PCR scanning assay used to map the 5′ end. cDNA of the 5′ LTR was prepared by priming total Teral RNA with an antisense oligonucleotide spanning 997 to 972 in the proviral genome (SEQ ID 1202). This cDNA was then divided and run in PCR analyses with an antisense primer from 968 to 950 (SEQ ID 1203) combined with a sense primer from a set of primers designed to cover the likely 5′ ends: 1) 571<SEQ ID 1204>, 2) 600<SEQ ID 1205>, 3) 626<SEQ ID 1206>, 4) 660<SEQ ID 1207>, 5) 712<SEQ ID 1208>. Duplicate PCR reactions on 1 μg genomic HeLa DNA were used as a positive control, and these reactions showed all primer pairs were effective. The reactions primed with cDNA showed a marked difference between primers 600 and 626, suggesting that the 5′ end lies near position 626 in the proviral genome.

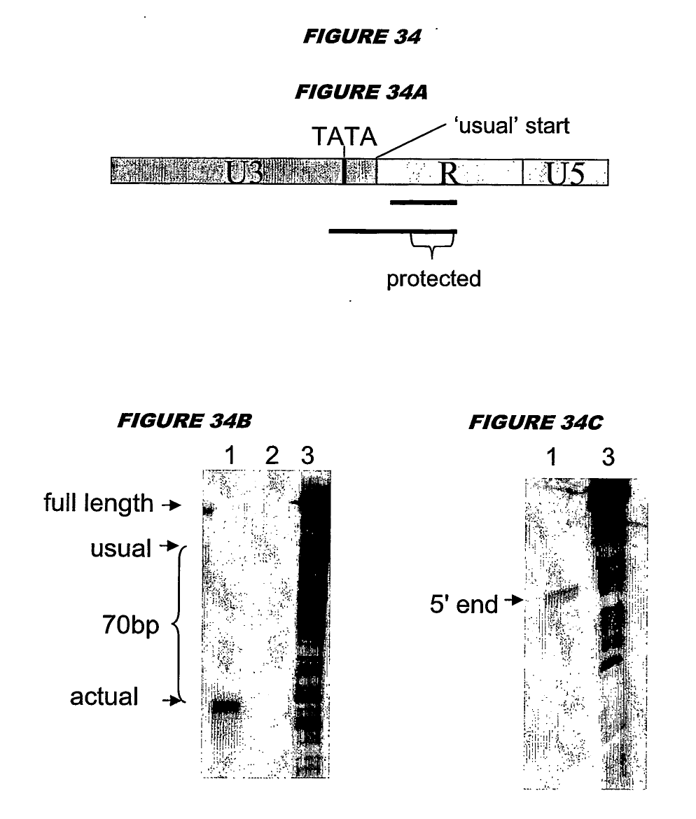

This result was confirmed using RNase protection assays (FIG. 34). Labeled antisense RNA probes covering bases (34B) 509-735 and (34C) 600-735 in the proviral genome were hybridized to total RNA from Teral cells and digested with RNase under standard conditions. After processing and detection by urea-containing PAGE, both probes gave 100 base products. These two results agree and show that 5′ end of HERV-K RNA is around base 635 in the proviral genome i.e. around 100 bp downstream of the TATA signal, rather than the 30 bp which is usual for TATA-dependent genes.

PCAP3

Within the final exon in the env region of PCAV, reading frames 1 and 2 encode env and cORF, respectively (FIG. 23). SEQ ID 87 is PCAP3, which shares the same 5′ region and start codon as env, but in which a splicing event removes env-coding sequences and shifts to a reading frame +2 relative to that of env (SEQ IDs 88 & 1191):

| | |
|---|---|
| ATGAACTCACTGGAGATGCAAAGAAAAGTGTGGAGATGGAGACACCCCAATCGACTCGCCAGgtaaacaaa | 8253 |
| M N S L E M Q R K V W R W R H P N R L A | |
| ...cctgttctgtctgttgttagTCTACAGGTGTATCCAGCAGCTCCAAAGAGACAGCAACCAGCAAGAATGGGCCATAG | 10480 |
| L Q V Y P A A P K R Q Q P A R M G H S | |
| TGACGATGGTGGTTTTGTCAAAAAGAAAAGGGGGGGATATGTAAGGAAAAGAGAGATCAGACTTTCACTGTGTCTATGTA | 10560 |
| D D G G F V K K K R G G Y V R K R E I R L S L C L C R | |
| GAAAAGGAAGACATAAGAAACTCCATTTTGATCTGTACTAA | 10601 |
| K G R H K K L H F D L Y * | |

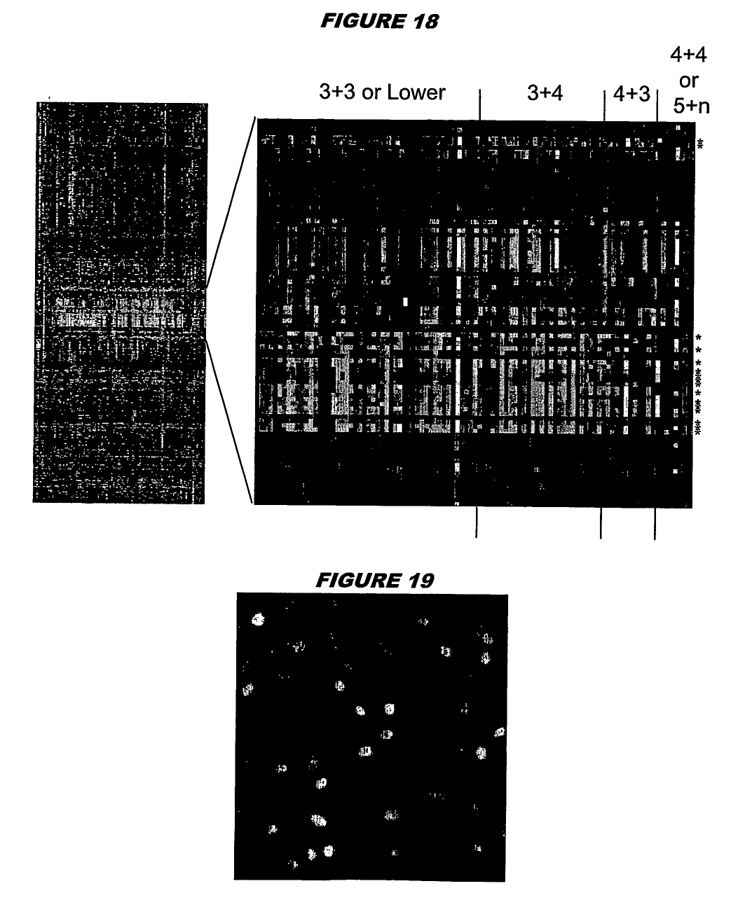

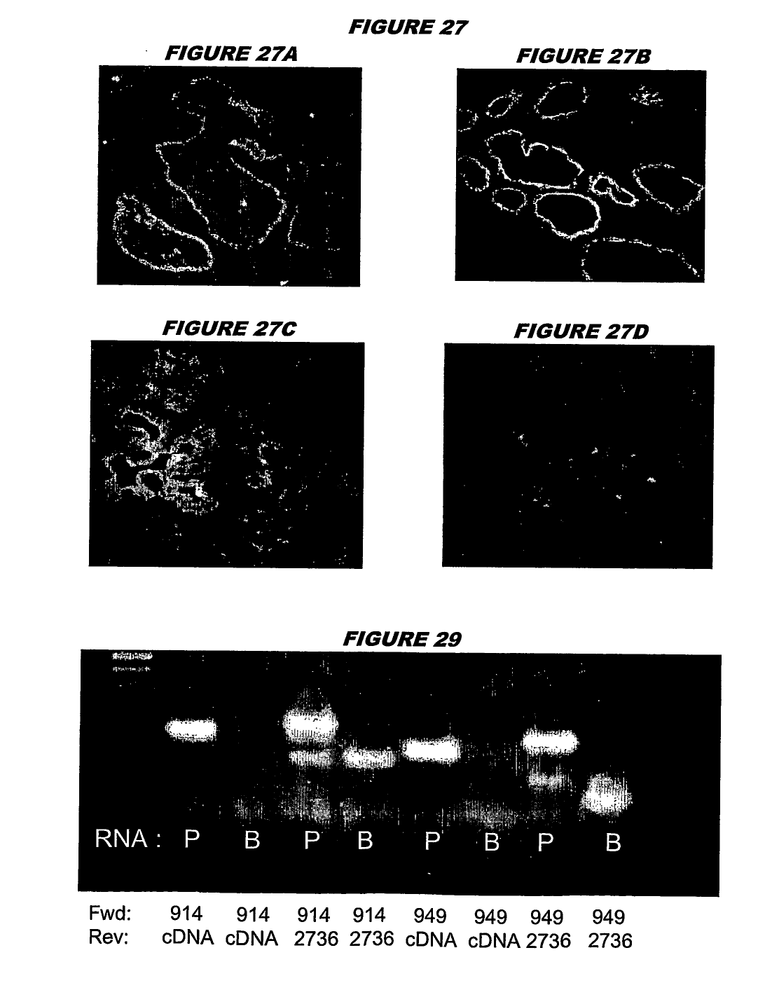

The majority of the coding sequence is thus located after the splice, within the exon which contains the 3′ LTR. Although the +2 reading frame has no known function in HERV-K, cDNA prepared from prostate cancer cell line MDA Pca-2b included these transcripts, as did prostate cancer mRNA. For example, spot 34058 (see above) encodes PCAP3 and was up-regulated more than 2-fold in 79% of patient samples and more than 5-fold in 53%. These figures support the view that PCAP3 is involved in many prostate cancers. Furthermore, the figures do not reflect the whole relationship between cancer and PCAP3 expression—if patients are grouped according to Gleason grades, grade 3 tumors show high up-regulation of PCAP3 whereas more developed grade 4 tumors seem to show PCAP3 suppression. FIG. 18 shows microarray analysis of prostate cancer employing 6000 random ESTs from a normalized prostate library. RNA levels prepared from laser-captured, micro-dissected tumor is compared to peri-tumor normal tissue RNA. The sequences tagged with asterisks in FIG. 18 are up-regulated and are all from a single 12 kb site in chromosome 22. These sequences span all portions of PCAV. Relative PCAV expression is very high in grade 3 tumors, with many of the patients having tumor/normal ratios in the 10 to 50 fold range. In Gleason grade 4 and above, however, the ratios return to 1 and in some cases the virus expression is suppressed. A similar pattern is seen with gag expression (FIG. 27), suggestion that PCAV expression is involved in the early stages of prostate cancer.

PCAP3 is similar to the cORF protein, and the two ORFs share a start codon, but two small deletions in PCAV introduce both a frameshift and an ‘old virus’ 5′ splice site (splice acceptor), thereby permitting the PCAP3-specific splice event. Inspection of various aligned HERV-K genomes gives further evidence that PCAP3 is a mutated form of an original protein. The protein is thus unlikely to be functioning in its original capacity, and oncogenic activity could arise through retention of a functional domain. The coding exon common to env, cORF and PCAP3 contains a RNA-binding domain that also functions as a nuclear localization signal (NLS).

To study the subcellular localization of PCAP3, in order to better understand its role, an adenovirus expressing PCAP3 with a C-terminal V5 tag (SEQ ID 1189) was used to infect primary prostate epithelial cells. The protein was relatively stable and was labeled in the nucleoplasm by anti-V5 (FIG. 19). The concentration of this small protein in this cellular location shows that it is specifically interacting with something within the nucleus.

A functional expression assay was also designed. The first component of the assay is an adenovirus vector with a PCAV LTR (SEQ ID 1190) driving GFP expression (FIG. 24). A variety of human cell lines were infected with this virus and fluorescence was measured either by fluorescent microscopy or by FACS. As a positive control, a vector was used in which GFP expression was driven by the EF-a promoter, which should be active in all eukaryotic cells.

GFP expression was minimal in ovarian, colon and liver cancer cells. It was also minimal in 293 cells, an immortalized kidney cell line, and in primary prostate epithelium cells. GFP was easily detected in various prostate cancer cell lines (PC3, LNCaP, MDA2B PCA, DU145). Representative data are shown in FIG. 25. The GFP expression pattern exactly matches genomics results from patient samples. These data indicate that expression driven from a PCAV-mRNA LTR is a marker for prostate cancer.

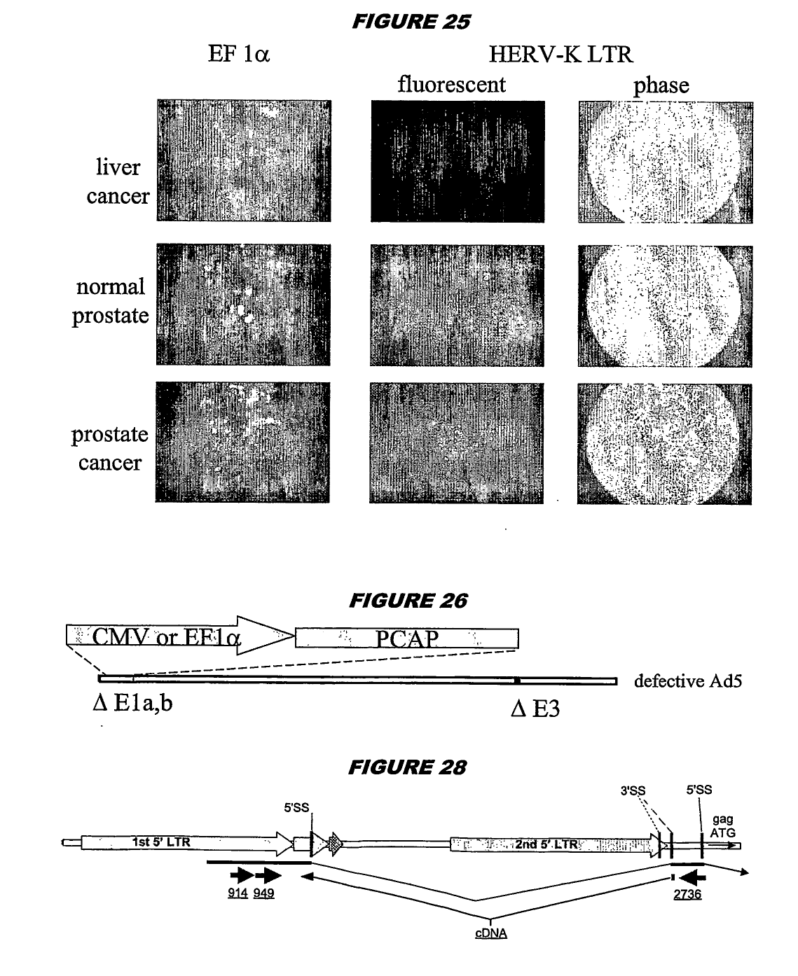

As GFP expression from the LTR appeared to be silent in primary prostate cells, but active in prostate cancer tissue, PCAP3 was tested for its ability to activate expression in primary prostate cells. The coding sequence was inserted into an expression cassette and incorporated into an adenovirus vector (FIG. 26). The vector was co-infected with the GFP vector into primary prostate epithelial cells, and PCAP3 weakly activated GFP expression.

In a separate experiment, high passage PrECs (approaching senescence) were co-infected with an adenovirus vector expressing GFP from an old-type HERV-K LTR (‘MDALTR’: SEQ ID 1196), and a second vector expressing PCAP3 at moi of about 20. After 3 days, the fluorescent intensity was measured by FACs and activation by PCAP3 was seen. In a similar experiment with LTR60, however, there was no activation.

PCAP3 and Senescence

Prostate cancer is believed to arise in the luminal epithelial layer, but normal luminal epithelial cells are capable of very few cell divisions. In contrast, NIH3T3 and RWPE1 cells (see FIGS. 11 & 12) are immortal. Because PCAV seems to be involved in early stages of cancer, the effects of PCAP3 on primary prostate epithelial cells (PrEC), which normally senesce rapidly, were tested.

Primary human epithelial cells have a very limited division potential. After a certain number of divisions the cells will enter senescence. Senescence is distinct from quiescence (immortal or pre-senescent cells enter quiescence when a positive growth signal is withdrawn, or when an inhibitory signal such as cell-cell contact is received, but can be induced to divide again by adding growth factors or by re-plating the cells at lower density) and is a permanent arrest in division, although senescent cells can live for many months without dividing if growth medium is regularly renewed.

Certain genes, particularly viral oncogenes (e.g. SV40 T-antigen) force cells to ignore senescence signals. T-antigen stimulates cells to continue division up to a further expansion barrier termed ‘replicative crisis’. Two processes occur in crisis: cells continue to divide, but cells die in parallel at a very high rate from accumulated genetic damage. When cell death exceeds division then virtually all cells die in a short period. The rare cells which grow out after crisis have become immortal and yield cell lines. Cell lines typically have obvious genetic rearrangements: they are frequently close to tetraploid, there are frequent non-reciprocal chromosomal translocations, and many chromosomes have deletions and amplifications of multiple loci {169, 170, 171}.

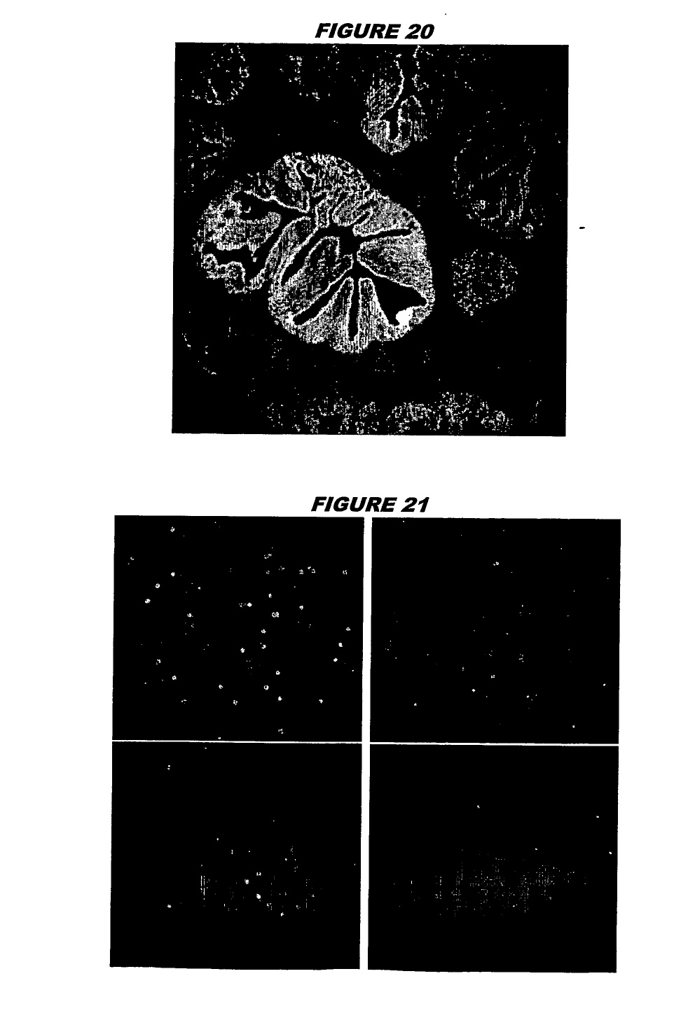

Gene products that lead to crisis are particularly interesting because prostate cancers exhibit high genomic instability, which could be caused by post-senescence replication. Current theory holds that prostate cancer arises from lesions termed prostatic intraepithelial neoplasia (PIN) {172}. Genetic analyses of PIN show that many of the genetic rearrangements characteristic of prostate cancer have already occurred at this stage {173}. PIN cells were thus tested for PCAV expression to determine if the virus could play a role in the earliest stages of prostate cancer. PCAV gag was found to be abundantly expressed (FIG. 20), indicating that PCAV expression is high at the time when the genetic changes associated with prostate cancer occur. As PCAP3 was seen to be expressed in prostate cancer, its role was investigated by seeing if it is capable of inducing cell division in PrEC after senescence.

Initial attempts to select drug-resistant PrECs after transfection with PCAP expression plasmids failed. Analysis of PrEC after infection with adenovirus vectors expressing either GFP or PCAP3 revealed abundant cell death on day 4 post-infection in the PCAP3 cells. A dose-dependent increase in terminal deoxytransferase end labeling (TUNEL), to mark nuclei with nicked DNA, confirmed that the cells were undergoing apoptosis (FIG. 21). This apoptosis may explain the failure to isolate drug-resistant PrECs, and is consistent with engagement of cell division machinery by PCAP3, as an unbalanced growth signal is an inducer of apoptosis.

These results suggested that apoptosis would have to be blocked before the effect of PCAP3 expression in PrECs could be assessed. Plasmids encoding PCAP3 plus a neomycin marker were thus co-transfected with an expression plasmid encoding bcl-2 (anti-apoptosis) and lacZ (marker). As controls, cells were transfected with plasmids expressing neomycin and either lacZ, bcl-2, bcl-X_{L}, or PCAP3. After two weeks under selection, the lacZ, bcl-2 and bcl-XL dishes all had numerous resistant cells that grew to fill in a fraction of the dish. When these cell were split they failed to divide further, but were viable and resembled senescent parental cells. In contrast, the cells which expressed PCAP3 and bcl-2 yielded some colonies made up of small cells which divided to fill the initial plate and continued to divide when split.

In parallel to the above drug selections, the growth potential of cells was assessed. The parental PrECs went through seven population doublings before reaching senescence. In contrast, drug-resistant cells co-transfected with an anti-apoptotic gene plus PCAP3 expanded well beyond the senescence point before ceasing to grow, going through sixteen doublings. After rapid growth for around two weeks, expansion of the cells slowed and finally ceased. Concomitantly, the number of floating and dead cells increased and the appearance of the cells changed—they no longer had the regular “cobblestone” appearance of epithelial cells, but instead had several morphologies, and there were many multinucleate cells. Cells died two weeks later, while the cells transfected with lacZ or lacZ+bcl-2 were still alive one month later.

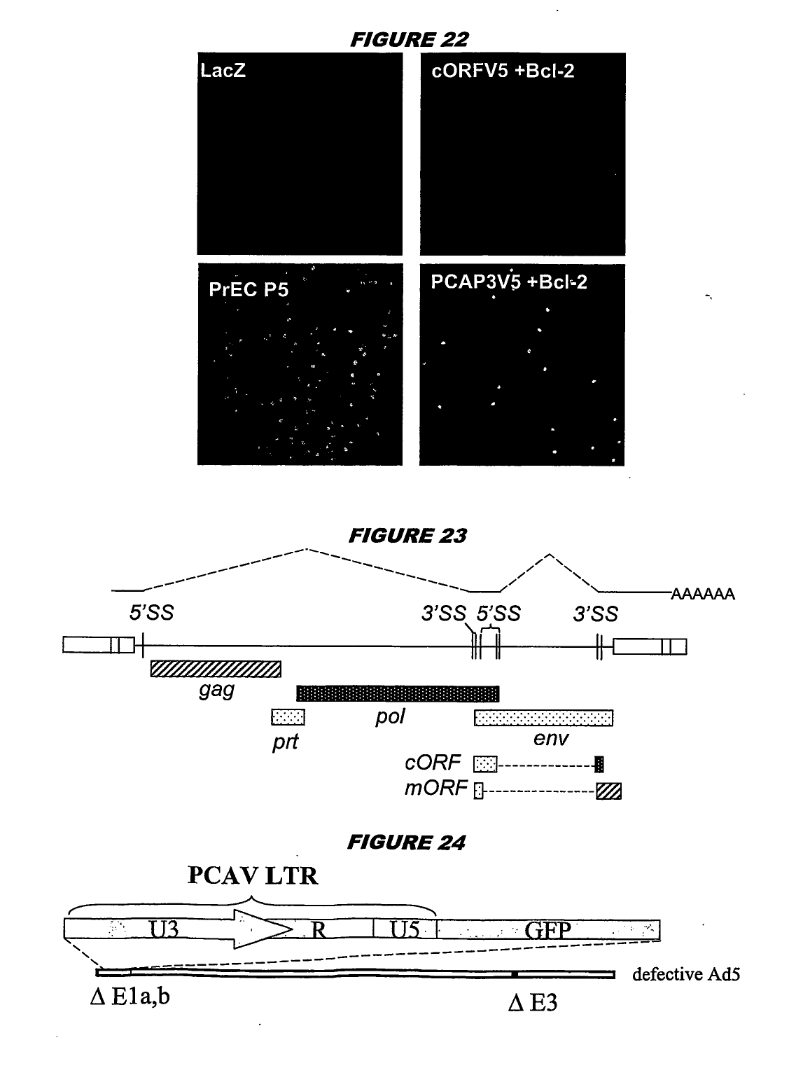

Neither senescent cells nor cells approaching crisis expand in number. One difference between them, however, is that cells approaching crisis are dividing and dying at an appreciable rate, and so cell division can distinguish between the two states. After labeling with bromo-deoxyuridine, 30% of pre-senescent PrECs were labeled, as were 10% of PrEC transfected with PCAP3+bcl-2, but none of the senescent lacZ or cORF+bcl-2 controls were labeled (FIG. 22).

These results show that PCAP3 is capable of inducing growth in prostate epithelial cells, and this growth could be an underlying cause of prostate cancer.

PCAV Detection by PCR

Primer pairs were tested to determine those which produced the expected PCAV product on prostate samples (P) and little or no product on breast sample (B). The primers are shown on the map of the 5′ LTRs of PCAV in FIG. 28. Forward primers were ‘914’ (SEQ ID 1192) or ‘949’ (SEQ ID 1193); reverse primers were ‘2736’ (SEQ ID 1194) or ‘cDNA’ (SEQ ID 1195). The cDNA primer spans the splice junction. Each reaction was run for 30 cycles on dT-primed cDNA prepared from total RNA extracted from either MCF7 (B) or MDA PCA 2b (P) cells.

Results are shown in FIG. 29. The primers clearly show preferential amplification in the prostate cells, and the primer bridging the splice junction (‘cDNA’) is highly specific.

Semi-quantitative RT-PCR experiments were also performed. Amplified RNA from LCM-derived prostate tissue from 10 patients was reverse transcribed using the 2736 primer, followed by PCR amplification either with the ‘914’ and ‘cDNA’ primer pairs (28 cycles), or with standard primers for human β-actin (25 cycles). Results are shown in FIG. 30. Matched samples of normal (N) or cancer (C) were amplified. The signal ratio in cancer tissue compared to normal tissue for each pair is shown above the PCAV PCR products.

Primers ‘914’ and ‘cDNA’ were also tested in quantitative PCR against dT-primed cDNA from a variety of tissues. As shown in FIG. 31, only prostate tissue from a 47 year old patient gave a significant signal.

RT-PCR was also performed on prostate tissue from patients of various ages. Expression levels were compared to gusB (β-glucuronidase). Results were as follows:

| | PCAV | GusB | Normalized | Normalized |
|---|---|---|---|---|
| Age | RT-PCR | RT-PCR | PCAV | GusB |
| 22 | 546 | 1105 | 1.60 | 340 |
| 47 | 430 | 729 | 1.06 | 406 |
| 67 | 848 | 689 | 1 | 848 |

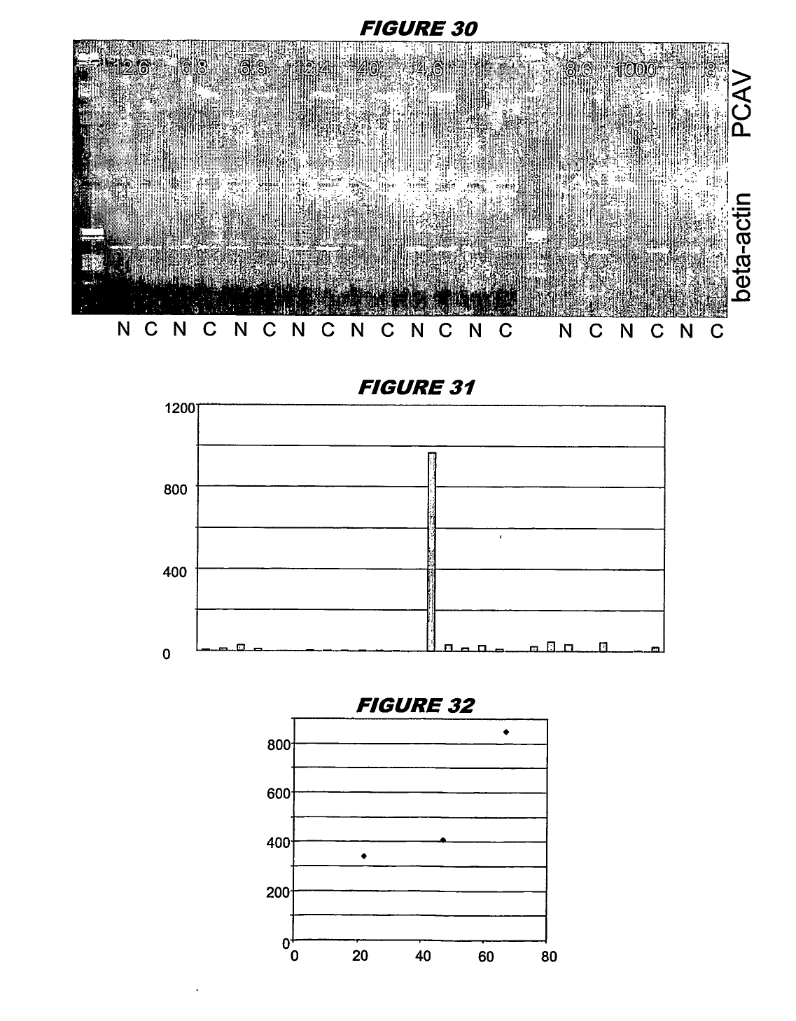

The normalized PCAV figures are also shown in FIG. 32.

The above description of preferred embodiments of the invention has been presented by way of illustration and example for purposes of clarity and understanding. It is not intended to be exhaustive or to limit the invention to the precise forms disclosed. It will be readily apparent to those of ordinary skill in the art in light of the teachings of this invention that many changes and modifications may be made thereto without departing from the spirit of the invention. It is intended that the scope of the invention be defined by the appended claims and their equivalents.

All patents, applications and references cited herein are incorporated by reference in their entirety.

SEQUENCE LISTING INDEX

| SEQ ID | DESCRIPTION |
|---|---|
| 1 | PCAV, from the beginning of its first 5′ LTR to |
| | the end of its fragmented 3′ LTR |
| 2 | Fragment of SEQ ID 1, from predicted transcription |
| | start site (559) to conserved splice donor site (1075) |
| 3 | Fragment of SEQ ID 1, following a splice acceptor |
| | site within second 5′ LTR (2611-2620) |
| 4 | Fragment of SEQ ID 1, following a splice acceptor |
| | site downstream of second 5′ LTR (2700-2709) |
| 5 | SEQ ID 2 + SEQ ID 3 |
| 6 | SEQ ID 2 + SEQ ID 4 |
| 7 | Fragment of SEQ ID 1: 5′ end of 3′ LTR (10520-10838) |
| 8 | Fragment of SEQ ID 1: MER11a insertion within 3′ |
| | LTR, up to polyA site (10839-11736) |
| 9 | SEQ ID 7 + SEQ ID 8 |
| 10 | Fragment of SEQ ID 1, from transcription start site |
| | to poly-A signal |
| 11 | Four 3′ nucleotides of SEQ ID 2 + four 5′ |
| | nucleotides of SEQ ID 3 |
| 12 | Four 3′ nucleotides of SEQ ID 2 + four 5′ |
| | nucleotides of SEQ ID 4 |
| 13 | Four 3′ nucleotides of SEQ ID 7 + four 5′ |
| | nucleotides of SEQ ID 8 |
| 14 | 27378 |
| 15 | 34058 |
| 16 | 26254 |
| 17 | Contig AP000345 |
| 18 | Contig AP000346 |
| 19 | cDNA sequence SP MDA#6 × SP6 rev |
| 20-22 | RACE primers |
| 23 | mRNA form of SEQ ID 10 |
| 24 | mRNA form of SEQ ID 5 |
| 25 | mRNA form of SEQ ID 6 |
| 26 | mRNA form of SEQ ID 2 |
| 27 | mRNA form of SEQ ID 3 |
| 28 | mRNA form of SEQ ID 4 |
| 29 | mRNA form of SEQ ID 9 |
| 30 | mRNA form of SEQ ID 7 |
| 31 | mRNA form of SEQ ID 8 |
| 32 | The alu interruption of env (9938-10244 of SEQ ID 1) |
| 33 | The 10 nucleotides upstream of SEQ ID 32 in SEQ ID 1 |
| 34 | The 10 nucleotides downstream of SEQ ID 32 in SEQ ID 1 |
| 35 | First 10 nucleotides of SEQ ID 32 |
| 36 | SEQ ID 33 + SEQ ID 35 |
| 37 | The 100 nucleotides upstream of SEQ ID 32 in SEQ ID 1 |
| 38 | SEQ ID 37 + SEQ ID 32 |
| 39 | Four 3′ nucleotides of SEQ ID 37 + four 5′ |
| | nucleotides of SEQ ID 32 |
| 40 | The 100 nucleotides downstream of SEQ ID 32 in SEQ |
| | ID 1 |
| 41 | Last 10 nucleotides of SEQ ID 32 |
| 42 | SEQ ID 41 + SEQ ID 40 |
| 43 | SEQ ID 32 + SEQ ID 40 |
| 44 | Four 3′ nucleotides of SEQ ID 32 + four 5′ |
| | nucleotides of SEQ ID 40 |
| 45 | Ten 3′ nucleotides of SEQ ID 32 + ten 5′ |
| | nucleotides of SEQ ID 40 |
| 46 | Fragment of SEQ ID 1, following a splice acceptor |
| | site within second 5′ LTR (2611-2710) |
| 47 | SEQ ID 2 + SEQ ID 46 |
| 48 | Fragment of SEQ ID 1, following a splice acceptor |
| | site downstream of second 5′ LTR (2700-2799) |
| 49 | SEQ ID 2 + SEQ ID 48 |
| 50 | Ten 3′ nucleotides of SEQ ID 2 + SEQ ID 3 |
| 51 | Ten 3′ nucleotides of SEQ ID 2 + SEQ ID 4 |
| 52 | Ten 3′ nucleotides of SEQ ID 7 + ten 5′ |
| | nucleotides of SEQ ID 8 |
| 53 | Gag nucleotide sequence unique to PCAV |
| 54 | PCAV gag |
| 55 | Gag fragment of SEQ ID 54 |
| 56 | Gag fragment of SEQ ID 54 |
| 57 | Gag (encodes SEQ ID 54) |
| 58 | Prt |
| 59-62 | Prt amino acid fragments |
| 63 | Env |
| 64-80 | Env amino acid fragments |
| 81 | Env |
| 82-85 | Env amino acid fragments |
| 86 | Pol |
| 87 | PCAP3 amino acid sequence |
| 88 | PCAP3 gene (spliced) |
| 89 | MDARU3#1 × T7rev |
| 90 | MDARU3#2 × SP6REV |
| 91 | MDARU3#4 × SP6rev |
| 92-97 | Pol amino acid fragment |
| 98 | Variant of SEQ ID 87 |
| 99-109 | Sequences of spliced cDNAs |
| 110 | Amino acids encoded by SEQ ID 53 |
| 111 | Nucleotides encoding SEQ ID 55 |
| 112 | Nucleotides encoding SEQ ID 56 |
| 113-119 | Hybridizing sequences with homology to chromosome 22 |
| 120-599 | 25mer PCAV fragments |
| 600-1184 | 25mer PCAV fragments with good predicted Tm values |
| 1185 | “New” gag construct |
| 1186 | “New” gag protein |
| 1187 | “Hybrid” gag construct |
| 1188 | “Hybrid” gag protein |
| 1189 | V5 tag |
| 1190 | HML-2 LTR |
| 1191 | cDNA sequence encoding PCAP3 |
| 1192-95 | PCAV-specific primers |
| 1196 | MDALTR |
| 1197 | SEQ ID 23 excluding its 77 5′ nucleotides |
| 1198 | SEQ ID 23 excluding its 100 5′ nucleotides |
| 1199 | SEQ ID 24 excluding its 77 5′ nucleotides |
| 1200 | SEQ ID 25 excluding its 77 5′ nucleotides |
| 1201 | SEQ ID 26 excluding its 77 5′ nucleotides |
| 1202-08 | Oligonucleotides used during RT-PCR mapping of |
| | transcription start site |

### REFERENCES (THE CONTENTS OF WHICH ARE HEREBY INCORPORATED IN FULL BY REFERENCE)

{1} International patent application WO02/46477 (PCT/US01/47824. filed Dec. 7, 2001).
{2} U.S. patent application Ser. No. 10/016,604 (filed Dec. 7, 2001).
{3} Reus et al. (2001) J. Virol. 75:8917-8926.
{4} Dunham et al. (1999) Nature 402:489-495.
{5} Prediger (2001) Methods Mol Biol 160:49-63.
{6} Bustin (2000) J. Mol. Endocrinol. 25:169-193.
{7} Gene Cloning and Analysis by RT-PCR (eds. Siebert et al.) ISBN: 1881299147.
{8} RT-PCR Protocols (ed. O'Connell) ISBN: 0896038750.
{9} The PCR Technique: RT-PCR (ed. Siebert) ISBN: 1881299139.
{10} Thaker (1999) Methods Mol Biol 115:379-402.
{11} Seiden & Sklar (1996) Important Adv Oncol 191-204.
{12} Hagen-Mann & Mann (1995) Exp Clin Endocrinol Diabetes 103:150-155.
{13} Clementi et al. (1993) PCR Methods Appl 2:191-196.
{14} Robbins et al. (1997) Clin Lab Sci 10(5):265-71.
{15} de la Taille (1999) Prog Urol 9:1084-1089.
{16} Ylikoski et al. (1999) Clin Chem 45(9):1397-1407.
{17} Yao et al. (1996) Cancer Treat Res 88:77-91.
{18} Ylikoski et al. (2001) Biotechniques 30:832-840
{19} Shirahata & Pegg (1986) J. Biol. Chem. 261(29):13833-7.
{20} RNA Methodologies (Farrell, 1998) (Academic Press; ISBN 0-12-249695-7).
{21} Sambrook et al. (1989) Molecular Cloning: A Laboratory Manual. NY, Cold Spring Harbor Laboratory
{22} Yang et al. (1999) Proc Natl Acad Sci USA 96(23):13404-8
{23} Short protocols in molecular biology (4th edition, 1999) Ausubel et al. eds. ISBN 0-471-32938-X.
{24} U.S. Pat. No. 5,707,829
{25} Fille et al. (1997) Biotechniques 23:34-36.
{26} EP-B-0509612
{27} EP-B-0505012
{28} Current Protocols in Molecular Biology (F. M. Ausubel et al. eds., 1987) Supplement 30.
{29} International patent application WO00/73801
{30} International patent application WO01/51633
{31} International patent application WO01/73032
{32} US patent application 20020022248.
{33} International patent application WO01/57270.
{34} International patent application WO01/75067.
{35} International patent application WO01/57182.
{36} International patent application WO01/57277.
{37} International patent application WO01/57274.
{38} International patent application WO01/57275.
{39} International patent application WO01/57276.
{40} International patent application WO01/57278.
{41} International patent application WO01/57272.
{42} International patent application WO01/42467.
{43} European patent application EP-A-1074617.
{44} Mayer et al. (1999) Nat. Genet. 21 (3), 257-258
{45} Löwer et al. (1996) Proc. Natl. Acad. Sci USA 93:5177
{46} Berkhout et al. (1999) J. Virol. 73:2365-2375.
{47} Löwer et al. (1995) J. Virol. 69:141-149.
{48} Magin et al. (1999) J. Virol. 73:9496-9507.
{49} Magin et al. (2000) Virology 274:11-16.
{50} Boese et al. (2001) FEBS Lett 493(2-3):117-21.
{51} Mueller-Lantzsch et al. AIDS Research and Human Retroviruses 9:343-350 (1993)
{52} Hashido et al. (1992) Biochem. Biophys. Res. Comm. 187:1241-1248.
{53} Vogetseder et al. (1995) Exp Clin Immunogenet. 12:96-102.
{54} Sauter et al. (1995) J. Virol. 69:414-421.
{55} Geysen et al. (1984) PNAS USA 81:3998-4002.
{56} Carter (1994) Methods Mol Biol 36:207-23.
{57} Jameson, B A et al. 1988, CABIOS 4(1):181-186.
{58} Raddrizzani & Hammer (2000) Brief Bioinform 1(2):179-89.
{59} De Lalla et al. (1999) J. Immunol. 163:1725-29.
{60} Brusic et al. (1998) Bioinformatics 14(2):121-30
{61} Meister et al. (1995) Vaccine 13(6):581-91.
{62} Roberts et al. (1996) AIDS Res Hum Retroviruses 12(7):593-610.
{63} Maksyutov & Zagrebelnaya (1993) Comput Appl Biosci 9(3):291-7.
{64} Feller & de la Cruz (1991) Nature 349(6311):720-1.
{65} Hopp (1993) Peptide Research 6:183-190.
{66} Welling et al. (1985) FEBS Lett. 188:215-218.
{67} Davenport et al. (1995) Immunogenetics 42:392-297.
{68} Go et al. (1980) Int. J. Peptide Protein Res. 15:211
{69} Querol et al. (1996) Prot. Eng. 9:265
{70} Olsen & Thomsen (1991) J. Gen. Microbiol. 137:579
{71} Clarke et al. (1993) Biochemistry 32:4322
{72} Wakarchuk et al. (1994) Protein Eng. 7:1379
{73} Toma et al. (1991) Biochemistry 30:97
{74} Haezerbrouck et al. (1993) Protein Eng. 6:643
{75} Masul et al. (1994) Appl. Env. Microbiol. (1994) 60:3579
{76} U.S. Pat. No. 4,959,314
{77} Smith & Waterman (1981) Adv. Appl. Math. 2: 482-489.
{78} Breedveld (2000) Lancet 355(9205):735-740.
{79} Gorman & Clark (1990) Semin. Immunol. 2:457-466
{80} Jones et al. (1986) Nature 321:522-525.
{81} Morrison et al. (1984) Proc. Natl. Acad. Sci, U.S.A., 81:6851-6855.
{82} Morrison & Oi (1988) Adv. Immunol., 44:65-92.
{83} Verhoeyer et al. (1988) Science 239:1534-1536.
{84} Padlan (1991) Molec. Immun. 28:489-498.
{85} Padlan (1994) Molec. Immunol. 31(3):169-217.
{86} Kettleborough et al. (1991) Protein Eng. 4(7):773-83
{87} Chothia et al. (1987) J. Mol. Biol. 196:901-917.
{88} Kabat et al. U.S. Dept. of Health and Human Services NIH Publication No. 91-3242 (1991)
{89} WO 98/24893
{90} WO 91/10741
{91} WO 96/30498
{92} WO 94/02602
{93} U.S. Pat. No. 5,939,598.
{94} WO 96/33735
{95} Gennaro (2000) Remington: The Science and Practice of Pharmacy. 20th edition, ISBN: 0683306472.
{96} WO 93/14778
{97} Findeis et al. (1993) Trends Biotechnol. 11:202
{98} Chiou et al. (1994) Gene Therapeutics: Methods And Applications Of Direct Gene Transfer. ed. Wolff
{99} Wu et al. (1988), J. Biol. Chem. 263:621
{100} Wu et al. (1994) J. Biol. Chem. 269:542
{101} Zenke et al. (1998) Proc. Natl. Acad. Sci. (USA) 87:3655
{102} Wu et al. (1991) J. Biol. Chem. 266:338.
{103} Jolly (1994) Cancer Gene Therapy 1:51.
{104} Kimura (1994) Human Gene Therapy 5:845
{105} Connelly (1995) Human Gene Therapy 1:185
{106} Kaplitt (1994) Nature Genetics 6:148
{107} WO 90/07936
{108} WO 94/03622
{109} WO 93/25698
{110} WO 93/25234
{111} U.S. Pat. No. 5,219,740
{112} WO 93/11230
{113} WO 93/10218
{114} U.S. Pat. No. 4,777,127
{115} GB Patent No. 2,200,651
{116} EP-A-0 345 242
{117} WO 91/02805
{118} WO 94/12649
{119} WO 93/03769
{120} WO 93/19191
{121} WO 94/28938
{122} WO 95/11984
{123} WO 95/00655
{124} Curiel (1992) Hum. Gene Ther. 3:147
{125} Wu, (1989) J. Biol. Chem. 264:16985
{126} U.S. Pat. No. 5,814,482
{127} WO 95/07994
{128} WO 96/17072
{129} WO 95/30763
{130} WO 97/42338
{131} WO 90/11092
{132} U.S. Pat. No. 5,580,859
{133} U.S. Pat. No. 5,422,120
{134} WO 95/13796
{135} WO 94/23697
{136} WO 91/14445
{137} EP 0524968
{138} Philip (1994) Mol. Cell Biol. 14:2411
{139} Woffendin (1994) Proc. Natl. Acad. Sci. USA 91:11581
{140} U.S. Pat. No. 5,206,152
{141} WO 92/11033
{142} U.S. Pat. No. 5,149,655
{143} WO90/14837
{144} Vaccine Design—the subunit and adjuvant approach (1995) eds. Powell & Newman. ASIN: 030644867X
{145} WO00/07621
{146} GB-2220221
{147} EP-A-0689454
{148} EP-A-0835318
{149} EP-A-0735898
{150} EP-A-0761231
{151} WO99/52549
{152} WO01/21207
{153} WO01/21152
{154} WO00/62800
{155} WO00/23105
{156} WO99/11241
{157} WO98/57659
{158} WO93/13202.
{159} McSharry (1999) Antiviral Res 43(1):1-21.
{160} Weissman (1987) Mol Biol. Med. 4(3):133-143
{161} Patanjali et al. (1991) Proc. Natl. Acad. Sci. USA 88: 1943-1947
{162} Simone et al. (2000) Am J Pathol. 156(2):445-52.
{163} Claverie (1996) Meth. Enzymol. 266:212-227.
{164} Chapter 36 (page 267ff) of Automated DNA Sequencing and Analysis Techniques (eds. Adams et al.) ISBN: 0127170103.
{165} Claverie et al. (1993) Comput. Chem. 17:191
{166} Altschul et al. (1990), J. Mol. Biol. 215:403-410.
{167} Pearson & Lipman (1988) PNAS USA, 85:2444.
{168} Luo et al. (1999) Nature Med 5:117-122.
{169} Sedivy (1998) Proc Natl Acad Sci USA 95:9078-9081.
{170} Hahn et al. (2002) Mol Cell Biol. 22(7):2111-2123.
{171} Hahn et al. (1999) Nature 400(6743):464-468.
{172} De Marzo et al. (1998) J Urol. 160:2381-2392.
{173} Sakr & Partin (2001) Urology 57(4 Suppl 1):115-120.

1. A method for diagnosing cancer, especially prostate cancer, the method comprising the step of detecting in a patient sample the presence or absence of an expression product of a human endogenous retrovirus (PCAV) located at megabase 20.428 on chromosome 22.

2. The method of claim 1, wherein the expression product which is detected is a mRNA transcript or a polypeptide.

3. The method of claim 1 or claim 2, wherein a mRNA transcript is detected by hybridization, by sequencing, or by a reverse transcriptase polymerase chain reaction.

4. The method of any preceding claim, wherein the method comprises an initial step of: (a) extracting mRNA from the patient sample; (b) removing DNA from the patient sample without removing mRNA; and/or (c) removing or disrupting PCAV DNA, but not PCAV mRNA, in the patient sample.

5. The method of any preceding claim, wherein the expression product is a mRNA transcript selected from the group consisting of:
(a) a mRNA transcript transcribed from a human endogenous retrovirus located at megabase 20.428 on chromosome 22;
(b) a mRNA transcript comprising a nucleotide sequence with 70% or more sequence identity to SEQ ID 23, to SEQ ID 1197 and/or to SEQ ID 1198;
(c) a mRNA transcript comprising the sequence —N₁—N₂—, where: N₁ is a nucleotide sequence from (1) the 5′ end of a mRNA transcribed from the first 5′ LTR of a human endogenous retrovirus located at megabase 20.428 on chromosome 22, to (2) a first splice donor site downstream of the U₅ region of said mRNA transcribed from the first 5′ LTR; and N₂ is a nucleotide sequence immediately downstream of a splice acceptor site located (1) downstream of said first splice donor site and (2) upstream of a second splice donor site, the second splice donor site being downstream of the second 5′ LTR of said endogenous retrovirus;
(d) a mRNA transcript comprising the sequence —N₁—N₂—, where: N₁ is a nucleotide sequence with 70% or more sequence identity to SEQ ID 26 and/or SEQ ID 1201 and N₂ is a nucleotide sequence with 70% or more sequence identity to SEQ ID 27 or SEQ ID 28;
(e) a mRNA transcript comprising a nucleotide sequence with 70% or more sequence identity to SEQ ID 24, SEQ ID 25, SEQ ID 1199 or SEQ ID 1200;
(f) a mRNA transcript comprising the sequence —N₃—N₄—, where: N₃ is a nucleotide sequence from the 3′ end of the 5′ fragment of the 3′ LTR of a human endogenous retrovirus located at megabase 20.428 on chromosome 22, and N₄ is a nucleotide sequence from 5′ end of the MER11a insertion in a human endogenous retrovirus located at megabase 20.428 on chromosome 22;
(g) a mRNA transcript comprising the sequence —N₃—N₄—, where: N₃ is a nucleotide sequence with 70% or more sequence identity to SEQ ID 30 and N₄ is a nucleotide sequence with 70% or more sequence identity to SEQ ID 31;
(h) a mRNA transcript comprising a nucleotide sequence with 70% or more sequence identity to SEQ ID 29;
(i) a mRNA transcript comprising the sequence —N₇—N₈—, where: N₇ is a nucleotide sequence preceding the alu insertion within the env gene of a human endogenous retrovirus located at megabase 20.428 on chromosome 22, and N₈ is a nucleotide sequence beginning at the 5′ end of said alu insertion;
(j) a mRNA transcript comprising the sequence —N₇—N₈—, where: N₇ is a nucleotide sequence with 70% or more sequence identity to SEQ ID 37 and N₈ is a nucleotide sequence with 70% or more sequence identity to SEQ ID 32;
(k) a mRNA transcript comprising a nucleotide sequence with 70% or more sequence identity to SEQ ID 38;
(l) a mRNA transcript comprising the sequence —N₉—N₁₀—, where: N₉ is a nucleotide sequence at the end of the alu insertion within the env gene of a human endogenous retrovirus located at megabase 20.428 on chromosome 22, and N₁₀ is a nucleotide sequence immediately downstream of said alu insertion;
(m) a mRNA transcript comprising the sequence —N₉—N₁₀—, where: N₉ is a nucleotide sequence with 70% or more sequence identity to SEQ ID 41 and N₁₀ is a nucleotide sequence with 70% or more sequence identity to SEQ ID 40;
(n) a mRNA transcript comprising a nucleotide sequence with 70% or more sequence identity to SEQ ID 42;
(o) a mRNA transcript comprising a nucleotide sequence with 70% or more sequence identity to SEQ ID 41;
(p) a mRNA transcript comprising a nucleotide sequence with 70% or more sequence identity to SEQ ID 53;
(q) a mRNA transcript comprising a nucleotide sequence with 70% or more sequence identity to SEQ ID 111;
(r) a mRNA transcript comprising a nucleotide sequence with 70% or more sequence identity to SEQ ID 1191; and
(s) a mRNA transcript which encodes a polypeptide having at least 70% sequence identity to SEQ ID 98.

6. The method of claim 5, wherein the mRNA transcript comprises one or more of SEQ IDs 24, 25, 26, 27, 28, 29, 30, 31, 32, 37, 38, 40, 41, 42, 43, 53, 111 and/or 1191.

7. The method of any preceding claim, comprising the steps of: (a) contacting the patient sample with nucleic acid primers and/or probe(s) under hybridizing conditions; and (b) detecting the presence or absence of hybridization in the patient sample.

8. The method of any preceding claim, comprising the steps of: (a) enriching mRNA in the sample relative to DNA to give a mRNA-enriched sample; (b) contacting the mRNA-enriched sample with nucleic acid primers and/or probe(s) under hybridizing conditions; and (c) detecting the presence or absence of hybridization to mRNA present in the mRNA-enriched sample.

9. The method of any preceding claim, comprising the steps of: (a) preparing DNA copies of mRNA in the sample; (b) contacting the DNA copies with nucleic acid primers and/or probe(s) under hybridizing conditions; and (c) detecting the presence or absence of hybridization to said DNA copies.

10. The method of claim 2, comprising the step of contacting the patient sample with an antibody which recognizes an expressed polypeptide from the retrovirus.

11. The method of any preceding claim, wherein the patient sample comprises prostate cells.

12. The method of any preceding claim, wherein the patient is an adult human male.

13. Nucleic acid selected from the group consisting of:
(a) nucleic acid comprising the nucleotide sequence of a mRNA transcript transcribed from a human endogenous retrovirus located at megabase 20.428 on chromosome 22;
(b) nucleic acid comprising a nucleotide sequence with 90% or more sequence identity to SEQ ID 10, SEQ ID 1197 and/or SEQ ID 1198;
(c) nucleic acid comprising a nucleotide sequence —N₁—N₂—;
(d) nucleic acid comprising a nucleotide sequence with 70% or more sequence identity to SEQ ID 5, SEQ ID 6, SEQ ID 1199 or SEQ ID 1200;
(e) nucleic acid comprising a nucleotide sequence —N₃—N₄—;
(f) nucleic acid comprising a nucleotide sequence with 70% or more sequence identity to SEQ ID 9;
(g) nucleic acid comprising a nucleotide sequence —N₇—N₈—;
(h) nucleic acid comprising a nucleotide sequence with 70% or more sequence identity to SEQ ID 38;
(i) nucleic acid comprising a nucleotide sequence —N₉—N₁₀—;
(j) nucleic acid comprising nucleotide sequence SEQ ID 42;
(k) nucleic acid comprising a nucleotide sequence with 70% or more sequence identity to SEQ ID 42;
(l) nucleic acid comprising nucleotide sequence SEQ ID 53;
(m) nucleic acid comprising a nucleotide sequence with 70% or more sequence identity to SEQ ID 53;
(n) nucleic acid comprising nucleotide sequence SEQ ID 111;
(o) nucleic acid comprising a nucleotide sequence with 70% or more sequence identity to SEQ ID 111;
(p) nucleic acid comprising nucleotide sequence SEQ ID 1191;
(q) nucleic acid comprising one or more of SEQ IDs 120 to 1184;
(r) nucleic acid which can hybridize under stringent conditions to a mRNA transcript as defined in (a) to (s) of claim 5; and
(s) the complement of (a), (b), (c), (d), (e), (f), (g), (h), (i), (j), (k), (l), (m), (n), (o), (p), (q), or (r),
wherein N₁ to N₁₀ are as defined in claim 5.

14. Nucleic acid of claim 13, comprising one or more of SEQ IDs 5, 6, 9, 38, 42, 53, 99, 100, 101, 102, 103, 104, 105, 106, 107, 108, 109, 111, 337-599, and 600-1184.

15. A nucleic acid probe selected from the group consisting of:
(a) a probe which can hybridize to sequence —N₁—N₂— (or the complement thereof) within a PCAV nucleic acid target, but which does not hybridize to sequences N₁ or N₂ alone (or to their complements alone);
(b) a probe which can hybridize to sequence —N₃—N₄— (or the complement thereof) within a PCAV nucleic acid target, but which does not hybridize to sequences N₃ or N₄ alone (or to their complements alone);
(c) a probe which can hybridize to sequence —N₇—N₈— (or the complement thereof) within a PCAV nucleic acid target, but which does not hybridize to sequences N₇ or N₈ alone (or to their complements alone);
(d) a probe which can hybridize to sequence —N₉—N₁₀— (or the complement thereof) within a PCAV nucleic acid target, but which does not hybridize to sequences N₉ or N₁₀ alone (or to their complements alone);
(e) a probe comprising a nucleotide sequence with 70% or more sequence identity to a fragment of SEQ ID 10, SEQ ID 1197 or SEQ ID 1198, or to the complement of a fragment of SEQ ID 10, SEQ ID 1197 or SEQ ID 1198;
(f) a probe comprising a nucleotide sequence with 70% or more sequence identity to a fragment of SEQ ID 5 and/or SEQ ID 1199 or to the complement of a fragment of SEQ ID 5 and/or SEQ ID 1199;
(g) a probe comprising a nucleotide sequence with 70% or more sequence identity to a fragment of SEQ ID 6 and/or SEQ ID 1200 or to the complement of a fragment of SEQ ID 6 and/or SEQ ID 1200;
(h) a probe comprising a nucleotide sequence with 70% or more sequence identity to a fragment of SEQ ID 9 or to the complement of a fragment of SEQ ID 9;
(i) a probe comprising a nucleotide sequence with 70% or more sequence identity to a fragment of SEQ ID 53 or to the complement of a fragment of SEQ ID 53;
(j) a probe comprising a nucleotide sequence with 70% or more sequence identity to a fragment of SEQ ID 1191 or to the complement of a fragment of SEQ ID 1191;
(k) a probe comprising a fragment of at least 10 contiguous nucleotides of SEQ ID 10 and/or SEQ ID 1198 or of the complement of SEQ ID 10 and/or SEQ ID 1198;
(l) a probe comprising a fragment of at least 10 contiguous nucleotides of SEQ ID 47 or of the complement of SEQ ID 47;
(m) a probe comprising nucleotide sequence B₁ₐ-B₂ₐ (or its complement), wherein B₁ₐ comprises 6 or more nucleotides from the 3′ end of SEQ ID 2 and B₂ₐ comprises 6 or more nucleotides from the 5′ end of SEQ ID 46;
(n) a probe comprising a fragment of at least 10 contiguous nucleotides of SEQ ID 49 or of the complement of SEQ ID 49;
(o) a probe comprising nucleotide sequence B_{1b}-B_{2b} (or its complement), wherein B_{1b} comprises 6 or more nucleotides from the 3′ end of SEQ ID 2 and B_{2b} comprises 6 or more nucleotides from the 5′ end of SEQ ID 48;
(p) a probe comprising a fragment of at least 10 contiguous nucleotides of SEQ ID 9 or of the complement of SEQ ID 9;
(q) a probe comprising nucleotide sequence B₃-B₄ (or its complement), wherein B₃ comprises 6 or more nucleotides from the 3′ end of SEQ ID 7 and B₄ comprises 6 or more nucleotides from the 5′ end of SEQ ID 8;
(r) a probe comprising a fragment of at least 10 contiguous nucleotides of SEQ ID 38 or of the complement of SEQ ID 38;
(s) a probe comprising nucleotide sequence B₇-B₈ (or its complement), wherein B₇ comprises 6 or more nucleotides from the 3′ end of SEQ ID 37 and B₄ comprises 6 or more nucleotides from the 5′ end of SEQ ID 32;
(t) a probe comprising a fragment of at least 10 contiguous nucleotides of SEQ ID 43 or of the complement of SEQ ID 43;
(u) a probe comprising nucleotide sequence B₉-B₁₀ (or its complement) wherein B₉ comprises 6 or more nucleotides from the 3′ end of SEQ ID 32 and B₁₀ comprises 6 or more nucleotides from the 5′ end of SEQ ID 40;
(v) a probe comprising a fragment of at least 10 contiguous nucleotides of SEQ ID 53 or of the complement of SEQ ID 53;
(w) a probe comprising a fragment of at least 10 contiguous nucleotides of SEQ ID 111 or of the complement of SEQ ID 111;
(x) a probe comprising a fragment of at least 10 contiguous nucleotides of SEQ ID 112 or of the complement of SEQ ID 112; and
(y) a probe comprising a fragment of at least 10 contiguous nucleotides of SEQ ID 1191 or of the complement of SEQ ID 1191;
wherein N₁ to N₁₀ are as defined in claim 5, and wherein ‘PCAV’ is the endogenous retrovirus located at megabase 20.428 on human chromosome 22.

16. The probe of claim 15, comprising one or more of SEQ IDs 11, 12, 13, 36, 39, 44, 45, 50, 51, 52, (or their complements).

17. Nucleic acid of formula 5′-X-Y-Z-3′, wherein:
—X— is a nucleotide sequence consisting of x nucleotides;
-Z- is a nucleotide sequence consisting of z nucleotides;
—Y— is a nucleotide sequence consisting of either (a) a fragment of y nucleotides of any of SEQ IDs 1-13, 20-53, 57, 58, 63, 81, 86, 88-91, 99-109, 111, or 112 or 1191, or (b) the complement of (a);
said nucleic acid 5′-X-Y-Z-3′ is neither (i) a fragment of SEQ IDs 1-13, 20-53, 57, 58, 63, 81, 86, 88-91, 99-109, 111, or 112 or 1191 or (ii) the complement of (i);
the value of x+z is at least 1; and
the value of x+y+z is at least 8.

18. The nucleic acid of claim 17, wherein the —X— and/or -Z- moieties comprises a promoter sequence (or its complement).

19. A kit comprising primers for amplifying a template sequence contained within the endogenous retrovirus located at megabase 20.428 on human chromosome 22, the kit comprising a first primer and a second primer, wherein the first primer comprises a sequence substantially complementary to a portion of said template sequence and the second primer comprises a sequence substantially complementary to a portion of the complement of said template sequence, wherein the sequences within said primers which have substantial complementarity define the termini of the template sequence to be amplified.

20. The kit of claim 19, further comprising a probe which is substantially complementary to the template sequence and/or to its complement and which can hybridize thereto.

21. The kit of claim 19 or claim 20, wherein the template sequence is located within a transcript of a HERV-K located at megabase 20.428 of chromosome 22

22. The kit of claim 21, wherein the template sequence is a fragment of SEQ ID 10 or of SEQ ID 23 or of SEQ ID 1197 or of SEQ ID 1198, and/or wherein the template comprises SEQ ID 53 and/or SEQ ID 111.

23. The kit of any one of claims 19 to 22, wherein the first and second primers are located in different exons of the template sequence.

24. The kit of any one of claims 19 to 23, wherein one of the primers comprises nucleotide sequence SEQ IDs 120 to 336.

25. The kit of any one of claims 19 to 24, wherein:
(a) the first primer comprises a sequence which is substantially identical to a portion of N₁ and the second primer comprises a sequence which is substantially complementary to a portion of N₂;
(b) the first primer comprises a sequence which is substantially identical to a portion of the complement of N₁ and the second primer comprises a sequence which is substantially complementary to a portion of the complement of N₂;
(c) the first primer comprises a sequence which is substantially identical to a portion of N₁ and the second primer comprises a sequence which is substantially complementary to a portion of PCAV sequence downstream of a splice donor which is itself downstream of the splice acceptors near the 3′ end of the second PCAV 5′ LTR
(d) the first primer comprises a sequence which is substantially identical to a portion of the complement of N₁ and the second primer comprises a sequence which is substantially complementary to a portion of the complement of a PCAV sequence downstream of a splice donor which is itself downstream of the splice acceptors near the 3′ end of the second PCAV 5′ LTR;
(e) the first primer comprises a sequence which is substantially identical to the splice junction site in N₁—N₂ and the second primer comprises a sequence which is substantially complementary to a portion of a PCAV sequence upstream or downstream of the splice junction site;
(f) the first primer comprises a sequence which is substantially identical to the complement of the splice junction site in N₁—N₂ and the second primer comprises a sequence which is substantially complementary to a portion of a PCAV upstream or sequence downstream of the splice junction site;
(g) the first primer comprises a sequence which is substantially identical to a portion of N₃ and the second primer comprises a sequence which is substantially complementary to a portion of N₄;
(h) the first primer comprises a sequence which is substantially identical to a portion of the complement of N₃ and the second primer comprises a sequence which is substantially complementary to a portion of the complement of N₄;
(i) the first primer comprises a first sequence which is substantially identical to a portion of N₃ and a second sequence which is substantially identical to a portion of N₄, and the second primer comprises a sequence which is substantially complementary to a ortion of an upstream or downstream PCAV sequence;
(j) the first primer comprises a first sequence which is substantially identical to a portion of the complement of N₃ and a second sequence which is substantially identical to a portion of the complement of N₄, and the second primer comprises a sequence which is substantially complementary to a portion of the complement of an upstream or downstream PCAV sequence;
(k) the first primer comprises a sequence which is substantially identical to a portion of N₃ and the second primer comprises a sequence which is substantially complementary to a portion of a polyA tail;
(l) the first primer comprises a sequence which is substantially identical to a portion of the complement of N₃ and the second primer comprises a sequence which is substantially complementary to a portion of the complement of polyA tail;
(m) the first primer comprises a sequence which is substantially identical to a portion of N₇ and the second primer comprises a sequence which is substantially complementary to a portion of N₈;
(n) the first primer comprises a sequence which is substantially identical to a portion of the complement of N₇ and the second primer comprises a sequence which is substantially complementary to a portion of the complement of N₈;
(o) the first primer comprises a first sequence which is substantially identical to a portion of N₇ and a second sequence which is substantially identical to a portion of N₈, and the second primer comprises a sequence which is substantially complementary to a portion of an upstream or downstream PCAV sequence;
(p) the first primer comprises a first sequence which is substantially identical to a portion of the complement of N₇ and a second sequence which is substantially identical to a portion of the complement of N₈, and the second primer comprises a sequence which is substantially complementary to a portion of the complement of an upstream or downstream PCAV sequence;
(q) the first primer comprises a sequence which is substantially identical to a portion of N₉ and the second primer comprises a sequence which is substantially complementary to a portion of N₁₀;
(r) the first primer comprises a sequence which is substantially identical to a portion of the complement of N₉ and the second primer comprises a sequence which is substantially complementary to a portion of the complement of N₁₀;
(s) the first primer comprises a first sequence which is substantially identical to a portion of N₉ and a second sequence which is substantially identical to a portion of N₁₀, and the second primer comprises a sequence which is substantially complementary to a portion of an upstream or downstream PCAV sequence;
(t) the first primer comprises a first sequence which is substantially identical to a portion of the complement of N₉ and a second sequence which is substantially identical to a portion of the complement of N₁₀, and the second primer comprises a sequence which is substantially complementary to the complement of an upstream or downstream PCAV sequence;
(u) the first primer comprises a sequence which is substantially identical to a first portion of SEQ ID 111, 112 or 53 and the second primer comprises a sequence which is substantially complementary to a second portion of SEQ ID 111, 112 or 53, such that the primer pair defines a template sequence within, consisting of or comprising SEQ ID 111, 112 or 53;
(v) the first primer comprises a sequence which is substantially identical to a first portion of the complement of SEQ ID 111, 112 or 53 and the second primer comprises a sequence which is substantially complementary to a second portion of the complement of SEQ ID 111, 112 or 53, such that the primer pair defines a template sequence within, consisting of or comprising SEQ ID 111, 112 or 53,
wherein N₁ to N₁₀ are as defined in claim 5, and wherein ‘PCAV’ is the endogenous retrovirus located at megabase 20.428 on human chromosome 22.

26. A polypeptide selected from the group consisting of:
(a) a polypeptide encoded by a human endogenous retrovirus located at megabase 20.428 on chromosome 22;
(b) a polypeptide comprising an amino acid sequence selected from the group consisting of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188;
(c) a polypeptide comprising a fragment of at least 7 amino acids of one or more of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188;
(d) a polypeptide comprising an amino acid sequence having at least 70% identity to one or more of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188;
(e) a polypeptide comprising a T-cell or a B-cell epitope of SEQ ID 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 or 1188; and
(f) a polypeptide having formula NH₂—XX—YY-ZZ-COOH, wherein:
XX is a polypeptide sequence consisting of xx amino acids;
ZZ is a polypeptide sequence consisting of zz amino acids;
YY is a polypeptide sequence consisting of a fragment of yy amino acids of an amino acid sequence selected from the group consisting of SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188;
said polypeptide NH₂—XX—YY-ZZ-COOH is not a fragment of a polypeptide sequence selected from SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and 1188;
xx+zz is at least 1; and
xx+yy+zz is at most 100.

27. An antibody that binds to a polypeptide of claim 26.

28. The antibody of claim 27, which recognize an epitope within SEQ IDs 54, 55, 56, 59, 60, 61, 62, 64, 65, 66, 67, 68, 69, 70, 71, 72, 73, 74, 75, 76, 77, 78, 79, 80, 82, 83, 84, 85, 87, 92, 93, 94, 95, 96, 97, 98, 110, 1186 and/or 1188.

29. The antibody of claim 27 or claim 28, which recognizes a HERV-K gag protein.

30. The antibody of claim 29, which recognizes gag from the human endogenous retrovirus located at megabase 20.428 on chromosome 22, but not the gag from other HERVs.

31. The antibody of any one of claims 28 to 30, wherein the antibody is monoclonal.

32. The nucleic acid, polypeptide or antibody of any one of claims 13 to 31, for use in diagnosis.

33. A pharmaceutical composition comprising the nucleic acid, polypeptide or antibody of any one of claims 13 to 31, and a pharmaceutically acceptable carrier.

34. A method for raising an immune response in a patient, comprising administering an immunogenic dose of the composition of claim 33.

35. The pharmaceutical composition is preferably an immunogenic composition and is more preferably a vaccine composition. Such compositions can be used to raise antibodies in a mammal (e.g. a human).

36. The composition of claim 35, further comprising a vaccine adjuvant.

37. A method of screening for compounds with activity against cancer, comprising: contacting a test compound with a tissue sample derived from a cell in which expression of the human endogenous retrovirus located at megabase 20.428 on chromosome 22 is up-regulated, or a cell line; and monitoring expression of the retrovirus in the sample, wherein a decrease in expression indicates anti-cancer efficacy of the test compound.

38. A method of screening for compounds with activity against prostate cancer, comprising:
contacting a test compound with a nucleic acid or polypeptide according to any of claims 13 to 26; and detecting a binding interaction between the test compound and the nucleic acid or polypeptide, wherein a binding interaction indicates potential anti-cancer efficacy of the test compound.


SEQUENCE LISTING (1208 sequences)

SEQ ID NO: 1 - DNA, 12366 bp, HERV-K
tgtggggaaaagaaagagagatcagactgttactgtgtctatgtagaaagaaatagacataagagactccattttgttctgtactaagaaaaattcttctgctttgagatgctgttaatctgtaaccctagccccaaccctgtgctcacagaaacaggtgctgtgttgactcaaggtttaatggattcagggctgtgcaggatgtgctttgttaaacaaatgcttgaaggcagcaagcttgttaagagtcatcaccactccctaatctcaagtaagcagggacacaaacactgcggaaggccgcagggacctctgcctaggaaagccaggtgttgtccaaggtttctccccatgtgacagtctgaaatatggcctcttgggaagggaaagacctgactgtcccctggcccgacacccgtaaagggtctgtgctgaggattagtaaaagaggaaggaaggcctctttgcagttgagataagaggaaggcatctgtctcctgctcatccctgggcaatggaatgtcttggtgtaaagcctgattgtatatgccatctactgagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtggtaagcttgggcgctcggaagaagccagggttaatggggcaaactaaaagtaaagtctctcattccacctgatgagaaacacccagaggtgtggaggggcaggccaccccttcagggtagggtcccctccatgcagaccatagagcacaggtgtgccccaaagaggagcagagagaaggagggagagggcccacgagagacttggaaatgaatggcaggattttaggcgctggacttgggttcggggcacctggcctttccttgtgtatttctcctactgtctgcctaactatttaatacaataaaagaaaaccagcccctggttcttgtggtgtttccaccctcccgggtccccgctggctgcctggcttcctcccgcagctcctgctgtgtgtgtatgtgtgtgtgtgtgcacatctgtggggcgtatgtgtgttcgtctttgtaattgaggctgcagagtggagagagcaggggttttctctggggacccagagagaaggaggcgttttcaccacagccgaacagggcaggaccccagcacccgggacccagcgggactttgccaaggggatggacctggctgggccacgcggctgtttgtgtagggaaaagaaagagagatcacactgttactgtgtctatgtagaaaaggaagacataaactccattttgagctgtactaagaaaaattattttgccttgacctgctgttaacctgtaactgtagccccaaccctgtgctcaaagaaacatgtgctgtatggaatcaaggtttaagggatcaagggctgtacaggatgtgccttgttaacaatgtgtttacaggcagtatgcttggtaaaagtcatcgccattctccattctccattaatcaggggcacgatgcactgcggaaagccacagggacctctgcccgagaaagcctgggtattgtccaaggcttccccccactgagacagcctgagatacggcctcgtgggaagggaaagacctgaccgtcccccagcccgacacccgtaaagggtctgtgctgaggaggattagtaaaaggggaaggcctcttgcagttgagataagaggaaggcctccgtctcctgcatgtccttgggaatggaatgtcttggtgtaaaacccgatagtacattccttctattctgagagaagaaaaccaccctgtggctggaggtgagatatgctagcggcaatgctgctctgttactctttgctacactgagatgtttgggtggagagaagcataaatctggcctatgtgcacatctgggcacagaacctccccttgaacttgtgacacagattcctttgttcacatgttttcctgctgaccttctccccactatcgccctgttctcccaccgcattccccttgctgagatagtgaaaatagtaatctgtagataccaagggaactcagagaccatggccggtgcacatcctccgtacgctgagcgctggtcccctgggcccattgttctttctctatactttgtctctgtgtcttatttctttcctcagtctctcatccctcctgacgagaaatacccacaggtgtggaggggctggcccccttcatctgatgcccaatgtgggtgcctttctctagggtgaaggtactctacagtgtggtcattgaggacaagttgacgagagagtcccaagtacgtccacggtcagccttgcggtaagcttgtgtgcttagaggaacccagggtaacgatggggcaaactgaaagtaaatatgcctcttatctcagctttattaaaattcttttaagaagagggggagttagagcttctacagaaaatctaattacgctatttcaaacaatagaacaattctgcccatggtttccagaacagggaactttagatctaaaagattgggaaaaaattggcaaagaattaaaacaagcaaatagggaaggtaaaatcatcccacttacagtatggaatgattgggccattattaaagcaactttagaaccatttcaaacaggagaagatattgtttcagtttctgatgcccctaaaagctgtgtaacagattgtgaagaagaggcagggacagaatcccagcaaggaacggaaagttcacattgtaaatatgtagcagagtctgtaatggctcagtcaacgcaaaatgttgactacagtcaattacaggagataatataccctgaatcatcaaaattgggggaaggaggtccagaatcattggggccatcagagcctaaaccacgatcgccatcaactcctcctcccgtggttcagatgcctgtaacattacaacctcaaacgcaggttagacaagcacaaaccccaagagaaaatcaagtagaaagggacagagtctctatcccggcaatgccaactcagatacagtatccacaatatcagccggtagaaaataagacccaaccgctggtagtttatcaataccggctgccaaccgagcttcagtatcggcctccttcagaggttcaatacagacctcaagcggtgtgtcctgtgccaaatagcacggcaccataccagcaacccacagcgatggcgtctaattcaccagcaacacaggacgcggcgctgtatcctcagccgcccactgtgagacttaatcctacagcatcacgtagtggacagggtggtgcactgcatgcagtcattgatgaagccagaaaacagggcgatcttgaggcatggcggttcctggtaattttacaactggtacaggccggggaagagactcaagtaggagcgcctgcccgagctgagactagatgtgaacctttcaccatgaaaatgttaaaagatataaaggaaggagttaaacaatatggatccaactccccttatataagaacattattagattccattgctcatggaaatagacttactccttatgactgggaaattttggccaaatcttccctttcatcctctcagtatctacagtttaaaacctggtggattgatggagtacaagaacaggtacgaaaaaatcaggctactaagcccactgttaatatagacgcagaccaattgttaggaacaggtccaaattggagcaccattaaccaacaatcagtgatgcagaatgaggctattgaacaagtaagggctatttgcctcagggcctggggaaaaattcaggacccaggaacagctttccctattaattcaattagacaaggctctaaagagccatatcctgactttgtggcaagattacaagatgctgctcaaaagtctattacagatgacaatgcccgaaaagttattgtagaattaatggcctatgaaaatgcaaatccagaatgtcagtcggccataaagccattaaaaggaaaagttccagcaggagttgatgtaattacagaatatgtgaaggcttgtgatgggattggaggagctatgcataaggcaatgctaatggctcaagcaatgagggggctcactctaggaggacaagttagaacatttgggaaaaaatgttataattgtggtcaaatcggtcatctgaaaaggagttgcccaggcttaaataaacagaatataataaatcaagctattacagcaaaaaataaaaagccatctggcctgtgtccaaaatgtggaaaagcaaaacattgggccaatcaatgtcattctaaatttgataaagatgggcaaccattgtctggaaacaggaagaggggccagcctcaggccccccaacaaactggggcattcccagttaaactgtttgttcctcagggttttcaaggacaacaacccctacagaaaataccaccacttcagggagtcagccaattacaacaatccaacagctgtcccgcgccacagcaggcagcaccgcagtagatttatgttccacccaaatggtctttttactccctggaaagcccccacaaaagattcctagaggggtatatggcccgctgccagaagggagggtaggcctttgagggagatcaagtctaaatttgaagggagtccaaattcatactggggtaatttattcagattataaagggggaattcagttagtgatcagctccactgttccccggagtgccaatccaggtgatagaattgctcaattactgcttttgccttatgttaaaattggggaaaacaaaaaggaaagaacaggagggtttggaagtaccaaccctgcaggaaaagctgcttattgggctaatcaggtctcagaggatagacccgtgtgtacagtcactattcagggaaagagtttgaaggattagtggatacccaggctgatgtttctgtcatcggcataggtactgcctcagaagtgtatcaaagtgccatgattttacattgtccaggatctgataatcaagaaagtacggttcagcctgtgatcacttcattccaatcaatttatggggccgagacttgttacaacaatggcatgcagagattactatcccagcctccctatacagccccaggaataaaaaaatcatgactaaaatgggatagctccctaaaaagggactaggaaagaagtcccaattgaggctgaaaaaaatcaaaaaagaaaaggaatagggcatcctttttaggagcggtcactgtagagcctccaaaacccattccattaacttgggggaaaaaaaaacaactgtatggtaaatcagcagcgcttccaaaacaaaaactggaggctttacatttattagcaaagaaacaattagaaaaaggacattgagccttcattttcgccttggaattctgtttgtaattcagaaaaaatccggcagatggcgtataatgccgtaattcaacccatgggggctctcccaccccggttgccctctccagccatggtcccctttaattataattgatctgaaggattgcttttttaccattcctctggcaaaacaggattttgaaaaatttgcttttaccacaccagcctaaataataaagaaccagccaccaggtttcagtggaaagtattgcctcagggaatgcttaatagttcaactatttgtcagctcaagctctgcaaccagttagagacaagttttcagactgttacatcgttcactatgttgatattttgtgtgctgcagaaacgagagacaaattaattgaccgttacacatttctgcagacagaggttgccaacgcgggactgacaataacatctgataagattcaaacctctactcctttccgttacttgggaatgcaggtagaggaaaggaaaattaaaccacaaaaaatagaaataagaaaagacacattaaaagcattaaatgagtttcaaaagttgctaggagatactaattggatttggagatattaattggatttggccaactctaggcattcctacttatgccatgtcaaatttgttctctttcttaagaggggactcggaattaaatagtgaaagaacgttaactccagaggcaactaaagaaattaaattaattgaagaaaaaattcggtcagcacaagtaaatagaatagatcacttggccccactccaaattttgatttttgctactgcacattccctaacaggcatcattgttcaaaatacagatcttgtggagtggtccttccttcctcacagtacaattaagacttttacattgtacttggatcaaatggctacattaattggtcagggaagattatgaataataacattgtgtggaaatgacccagataaaatcactgttcctttcaacaagcaacaggttagacaagcctttatcaattctggtgcatggcagattggtcttgccgattttgtgggaattattgacaatcgttaccccaaaacaaaaatcttccagtttttaaaattgactacttggattttacctaaagttaccaaacataagcctttaaaaaatgctctggcagtgtttactgatggttccagcaatggaaaagtggcttacaccgggccaaaagaatgagtcatcaaaactcagtatcacttgactcaaagagcagagttggttgccgtcattacagtgttaacaagattttaatcagtctattaacattgtatcagattctgcatatgtagtacaggctacaaaggatattgagagagccctaatcaaatacattatggatgatcagttaaacccgctgtttaatttgttacaacaaaatgtaagaaaaagaaatttcccattttatattactcatattcgagcacacactaatttaccagggcctttaactaaagcaaatgaacaagctgacttgctagtatcatctgcattcatggaagcacaagaacttcatgccttgactcatgtaaatgcaataggattaaaaaataaatttgatatcacatggaaacagacaaaaaatattgtacaacattgcacccagtgtcagattctacacctggccactcaggaggcaagagttaatcccagaggtctatgtcctaatgtgttatggcaaatggatgtcatgcacgtaccttcatttggaaaattgtcatttgtccatgtgacagttgatacttattcacatttcatatgggcaacctgccagacaggagaaagtacttcccatgttaaaagacatttattatcttgttttcctgtcatgggagttccagaaaaagttaaaacagacaatgggccaggttactgtagtaaagcagttcaaaaattcttaaatcagtggaaaattacacatacaataggaattctctataattcccaaggacaggccataattgaaagaactaatagaacactcaaagctcaattggttaaacaaaaaaaaggaaaagacaggagtataacactccccagatgcaacttaatctagcactctatactttaaatgttttaaacatttatagaaatcagaccactacctctgcagaacaacatcttactggtaaaaggaacagcccacatgaaggaaaactgatttggtggaaagataataaaaataaaacatgggaaatggggaaggtgataacgtgggggagaggttttgcttgtgtttcaccaggagaaaatcagcttcctgtttggatacccactagacatttaaagttctacaatgaactcactggagatgcaaagaaaagtgtggagatggagacaccccaatcgactcgccaggtaaacaaaatggtgatatcagaagaacagaaaaagttgccttccatcaaggaagcagagttgccaatataggcacaattaaagaagctgacacagttagctaaaaaaaaaagcctagagaatacaaaggtgacaccaactccagagaatatgctgcttgcagctctgatgattgtatcaacggtggtaagtcttcccaagtctgcaggagcagctgcagctaattatacttactgggcctatgtgcctttcccacccttaattcgggcagttacatagatggataatcctattgaagtagatgttaataatagtgcatgggtgcctggccccacagatgactgttgccctgcccaacctgaagaaggaatgatgatgaatatttccattgggtatccttatcctcctgtttgcctagggaaggcaccaggatgcttaatgcctacaacccaaaattggttggtagaagtacctacagtcagtgctaccagtagatttacttatcacatggtaagtggaatgtcacagataaataatttacaggacccttcttatcaaagatcattacaatgtaggcctaaggggaaggcttgccccaaggaaattcccaaagaatcaaaaagcccagaagtcttagtctgcggagaatgtgtggctgatactgcagtgtagtacaaaacaatgaattttgaactatgatagactgggtcccttgaggccaattatatcataactgtacaggccagactcattcatgttcacaggccccatccatctggcccattaatccagcctatgacggtgatgtaactgaaaggctggaccaggtttatagaaggttagaatcactctgtccaaggaaatggggtgaaaagggaatttcatcaccttgaccaaagttagtcctgttactggtcctgaacatccagaattaggaagcttactgtggcctcacaccacattagaatttgttctggaaatcaagctataggaacaagagatcgtaagtcatattatactatcaacctaaattccagtctgacaattcctttgcaaaattgtgtaaaactcccttatattgctagttgtaggaaaaacatagttattaaacctgattcccaaaccataatctgtgaaaattgtggaatgtttacttgcattgatttgacttttaattggcagcaccgtattctactaggaagagcaagagagggtgtgtggatccttgtgtccatggaccgaccatgggaggcttcgctatccatccatattttaacggaagtattaaaaggaattctaactagatccaaaagattcatttttactttgatggcagtgattatgggcctcattgcagtcacagctactgctgcggctgctggaattgctttacactcctctgttcaaactgcagaatacgtaaatgattggcaaaagaattcctcaaaattgtggaattctcagatccaaatagatcaaaaattggcaaaccaaattaatgatcttagacaaactgtcatttggatgggagaggctcatgagcttggaatatctttttcagttacgatgtgactggaatacatcagatttttgtgttacaccacaagcctataatgagtctgagcatcactgggacatggttagatgccatctgcaaggaggagaagataatcttactttagacatttcaaaattaaaagaattttttttttctttgagacagagtctcgctctgtcgcccaggctggagtgcagtggcgtgatctcagctcactgcaagttccgcctcctgggtttacaccattctcctgcctcagcctcccaagtagttgggactacaggagcccaccaccatgcctggctaattttttttgggtttttaatagagatggagtttcaccgtgttagccaggatggtctcgatctcctgaccttgtgatctgcccaccttggcctcccaaagtgctgggattacagtcgtgagccaccgtgcccagccaagaaaaaatttttgaggcatcaaaagcccatttaaatttggtgccaggaacggagacaatcgtgaaagctgctgatagcctcacaaatcttaagccagtcacttgggttaaaagcatcagaagtttcactattgtaaatttcatattaatccttgtatgcctgttctgtctgttgttagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaattcctttctttatactcttatttctcagaccagctgacacttagggaaaatagaaagaacctatgttgaaatattggaggcgggttcccccgatacctgggtattgtccaaggtttcctttgctgaggaggattagtaaaaggaatgcctccatctcctgcatgtccctgggaacagaatgttcccaccaaccaccctgtggctggaggcgggatatgctggcagcaatgctgctctattactctttgctacactgagatgtttgggtggagagaagcataaatctggcctatgtgcacatctgggcacagcaccttcctttgaacttatttgtgacacagattcctttgctcacgttttcctgttgactttctcaccactcaccctattctcctgtggcattcgccttgcggagatagtgaaaatagtaataaatactgagggaactcagactgagggaactcagactgggcagaccggggccagtgtgggtcctccatatgctgagcgccggttccctgggcccactgttctttctctatactttgtctctgtgccttattttctcagtctctcattccacctgatgagaaatacccacaggtgtggaggggctggcccccttca

SEQ ID NO: 2 - DNA, 517 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtg

SEQ ID NO: 3 - DNA, 10 bp, HERV-K
tctctcatcc

SEQ ID NO: 4 - DNA, 10 bp, HERV-K
ggtgaaggta

SEQ ID NO: 5 - DNA, 527 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgtctctcatcc

SEQ ID NO: 6 - DNA, 527 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgggtgaaggta

SEQ ID NO: 7 - DNA, 319 bp, HERV-K
tgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggc

SEQ ID NO: 8 - DNA, 897 bp, HERV-K
tgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgt

SEQ ID NO: 9 - DNA, 1216 bp, HERV-K
tgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgt

SEQ ID NO: 10 - DNA, 11177 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtggtaagcttgggcgctcggaagaagccagggttaatggggcaaactaaaagtaaagtctctcattccacctgatgagaaacacccagaggtgtggaggggcaggccaccccttcagggtagggtcccctccatgcagaccatagagcacaggtgtgccccaaagaggagcagagagaaggagggagagggcccacgagagacttggaaatgaatggcaggattttaggcgctggacttgggttcggggcacctggcctttccttgtgtatttctcctactgtctgcctaactatttaatacaataaaagaaaaccagcccctggttcttgtggtgtttccaccctcccgggtccccgctggctgcctggcttcctcccgcagctcctgctgtgtgtgtatgtgtgtgtgtgtgcacatctgtggggcgtatgtgtgttcgtctttgtaattgaggctgcagagtggagagagcaggggttttctctggggacccagagagaaggaggcgttttcaccacagccgaacagggcaggaccccagcacccgggacccagcgggactttgccaaggggatggacctggctgggccacgcggctgtttgtgtagggaaaagaaagagagatcacactgttactgtgtctatgtagaaaaggaagacataaactccattttgagctgtactaagaaaaattattttgccttgacctgctgttaacctgtaactgtagccccaaccctgtgctcaaagaaacatgtgctgtatggaatcaaggtttaagggatcaagggctgtacaggatgtgccttgttaacaatgtgtttacaggcagtatgcttggtaaaagtcatcgccattctccattctccattaatcaggggcacgatgcactgcggaaagccacagggacctctgcccgagaaagcctgggtattgtccaaggcttccccccactgagacagcctgagatacggcctcgtgggaagggaaagacctgaccgtcccccagcccgacacccgtaaagggtctgtgctgaggaggattagtaaaaggggaaggcctcttgcagttgagataagaggaaggcctccgtctcctgcatgtccttgggaatggaatgtcttggtgtaaaacccgatagtacattccttctattctgagagaagaaaaccaccctgtggctggaggtgagatatgctagcggcaatgctgctctgttactctttgctacactgagatgtttgggtggagagaagcataaatctggcctatgtgcacatctgggcacagaacctccccttgaacttgtgacacagattcctttgttcacatgttttcctgctgaccttctccccactatcgccctgttctcccaccgcattccccttgctgagatagtgaaaatagtaatctgtagataccaagggaactcagagaccatggccggtgcacatcctccgtacgctgagcgctggtcccctgggcccattgttctttctctatactttgtctctgtgtcttatttctttcctcagtctctcatccctcctgacgagaaatacccacaggtgtggaggggctggcccccttcatctgatgcccaatgtgggtgcctttctctagggtgaaggtactctacagtgtggtcattgaggacaagttgacgagagagtcccaagtacgtccacggtcagccttgcggtaagcttgtgtgcttagaggaacccagggtaacgatggggcaaactgaaagtaaatatgcctcttatctcagctttattaaaattcttttaagaagagggggagttagagcttctacagaaaatctaattacgctatttcaaacaatagaacaattctgcccatggtttccagaacagggaactttagatctaaaagattgggaaaaaattggcaaagaattaaaacaagcaaatagggaaggtaaaatcatcccacttacagtatggaatgattgggccattattaaagcaactttagaaccatttcaaacaggagaagatattgtttcagtttctgatgcccctaaaagctgtgtaacagattgtgaagaagaggcagggacagaatcccagcaaggaacggaaagttcacattgtaaatatgtagcagagtctgtaatggctcagtcaacgcaaaatgttgactacagtcaattacaggagataatataccctgaatcatcaaaattgggggaaggaggtccagaatcattggggccatcagagcctaaaccacgatcgccatcaactcctcctcccgtggttcagatgcctgtaacattacaacctcaaacgcaggttagacaagcacaaaccccaagagaaaatcaagtagaaagggacagagtctctatcccggcaatgccaactcagatacagtatccacaatatcagccggtagaaaataagacccaaccgctggtagtttatcaataccggctgccaaccgagcttcagtatcggcctccttcagaggttcaatacagacctcaagcggtgtgtcctgtgccaaatagcacggcaccataccagcaacccacagcgatggcgtctaattcaccagcaacacaggacgcggcgctgtatcctcagccgcccactgtgagacttaatcctacagcatcacgtagtggacagggtggtgcactgcatgcagtcattgatgaagccagaaaacagggcgatcttgaggcatggcggttcctggtaattttacaactggtacaggccggggaagagactcaagtaggagcgcctgcccgagctgagactagatgtgaacctttcaccatgaaaatgttaaaagatataaaggaaggagttaaacaatatggatccaactccccttatataagaacattattagattccattgctcatggaaatagacttactccttatgactgggaaattttggccaaatcttccctttcatcctctcagtatctacagtttaaaacctggtggattgatggagtacaagaacaggtacgaaaaaatcaggctactaagcccactgttaatatagacgcagaccaattgttaggaacaggtccaaattggagcaccattaaccaacaatcagtgatgcagaatgaggctattgaacaagtaagggctatttgcctcagggcctggggaaaaattcaggacccaggaacagctttccctattaattcaattagacaaggctctaaagagccatatcctgactttgtggcaagattacaagatgctgctcaaaagtctattacagatgacaatgcccgaaaagttattgtagaattaatggcctatgaaaatgcaaatccagaatgtcagtcggccataaagccattaaaaggaaaagttccagcaggagttgatgtaattacagaatatgtgaaggcttgtgatgggattggaggagctatgcataaggcaatgctaatggctcaagcaatgagggggctcactctaggaggacaagttagaacatttgggaaaaaatgttataattgtggtcaaatcggtcatctgaaaaggagttgcccaggcttaaataaacagaatataataaatcaagctattacagcaaaaaataaaaagccatctggcctgtgtccaaaatgtggaaaagcaaaacattgggccaatcaatgtcattctaaatttgataaagatgggcaaccattgtctggaaacaggaagaggggccagcctcaggccccccaacaaactggggcattcccagttaaactgtttgttcctcagggttttcaaggacaacaacccctacagaaaataccaccacttcagggagtcagccaattacaacaatccaacagctgtcccgcgccacagcaggcagcaccgcagtagatttatgttccacccaaatggtctttttactccctggaaagcccccacaaaagattcctagaggggtatatggcccgctgccagaagggagggtaggcctttgagggagatcaagtctaaatttgaagggagtccaaattcatactggggtaatttattcagattataaagggggaattcagttagtgatcagctccactgttccccggagtgccaatccaggtgatagaattgctcaattactgcttttgccttatgttaaaattggggaaaacaaaaaggaaagaacaggagggtttggaagtaccaaccctgcaggaaaagctgcttattgggctaatcaggtctcagaggatagacccgtgtgtacagtcactattcagggaaagagtttgaaggattagtggatacccaggctgatgtttctgtcatcggcataggtactgcctcagaagtgtatcaaagtgccatgattttacattgtccaggatctgataatcaagaaagtacggttcagcctgtgatcacttcattccaatcaatttatggggccgagacttgttacaacaatggcatgcagagattactatcccagcctccctatacagccccaggaataaaaaaatcatgactaaaatgggatagctccctaaaaagggactaggaaagaagtcccaattgaggctgaaaaaaatcaaaaaagaaaaggaatagggcatcctttttaggagcggtcactgtagagcctccaaaacccattccattaacttgggggaaaaaaaaacaactgtatggtaaatcagcagcgcttccaaaacaaaaactggaggctttacatttattagcaaagaaacaattagaaaaaggacattgagccttcattttcgccttggaattctgtttgtaattcagaaaaaatccggcagatggcgtataatgccgtaattcaacccatgggggctctcccaccccggttgccctctccagccatggtcccctttaattataattgatctgaaggattgcttttttaccattcctctggcaaaacaggattttgaaaaatttgcttttaccacaccagcctaaataataaagaaccagccaccaggtttcagtggaaagtattgcctcagggaatgcttaatagttcaactatttgtcagctcaagctctgcaaccagttagagacaagttttcagactgttacatcgttcactatgttgatattttgtgtgctgcagaaacgagagacaaattaattgaccgttacacatttctgcagacagaggttgccaacgcgggactgacaataacatctgataagattcaaacctctactcctttccgttacttgggaatgcaggtagaggaaaggaaaattaaaccacaaaaaatagaaataagaaaagacacattaaaagcattaaatgagtttcaaaagttgctaggagatactaattggatttggagatattaattggatttggccaactctaggcattcctacttatgccatgtcaaatttgttctctttcttaagaggggactcggaattaaatagtgaaagaacgttaactccagaggcaactaaagaaattaaattaattgaagaaaaaattcggtcagcacaagtaaatagaatagatcacttggccccactccaaattttgatttttgctactgcacattccctaacaggcatcattgttcaaaatacagatcttgtggagtggtccttccttcctcacagtacaattaagacttttacattgtacttggatcaaatggctacattaattggtcagggaagattatgaataataacattgtgtggaaatgacccagataaaatcactgttcctttcaacaagcaacaggttagacaagcctttatcaattctggtgcatggcagattggtcttgccgattttgtgggaattattgacaatcgttaccccaaaacaaaaatcttccagtttttaaaattgactacttggattttacctaaagttaccaaacataagcctttaaaaaatgctctggcagtgtttactgatggttccagcaatggaaaagtggcttacaccgggccaaaagaatgagtcatcaaaactcagtatcacttgactcaaagagcagagttggttgccgtcattacagtgttaacaagattttaatcagtctattaacattgtatcagattctgcatatgtagtacaggctacaaaggatattgagagagccctaatcaaatacattatggatgatcagttaaacccgctgtttaatttgttacaacaaaatgtaagaaaaagaaatttcccattttatattactcatattcgagcacacactaatttaccagggcctttaactaaagcaaatgaacaagctgacttgctagtatcatctgcattcatggaagcacaagaacttcatgccttgactcatgtaaatgcaataggattaaaaaataaatttgatatcacatggaaacagacaaaaaatattgtacaacattgcacccagtgtcagattctacacctggccactcaggaggcaagagttaatcccagaggtctatgtcctaatgtgttatggcaaatggatgtcatgcacgtaccttcatttggaaaattgtcatttgtccatgtgacagttgatacttattcacatttcatatgggcaacctgccagacaggagaaagtacttcccatgttaaaagacatttattatcttgttttcctgtcatgggagttccagaaaaagttaaaacagacaatgggccaggttactgtagtaaagcagttcaaaaattcttaaatcagtggaaaattacacatacaataggaattctctataattcccaaggacaggccataattgaaagaactaatagaacactcaaagctcaattggttaaacaaaaaaaaggaaaagacaggagtataacactccccagatgcaacttaatctagcactctatactttaaatgttttaaacatttatagaaatcagaccactacctctgcagaacaacatcttactggtaaaaggaacagcccacatgaaggaaaactgatttggtggaaagataataaaaataaaacatgggaaatggggaaggtgataacgtgggggagaggttttgcttgtgtttcaccaggagaaaatcagcttcctgtttggatacccactagacatttaaagttctacaatgaactcactggagatgcaaagaaaagtgtggagatggagacaccccaatcgactcgccaggtaaacaaaatggtgatatcagaagaacagaaaaagttgccttccatcaaggaagcagagttgccaatataggcacaattaaagaagctgacacagttagctaaaaaaaaaagcctagagaatacaaaggtgacaccaactccagagaatatgctgcttgcagctctgatgattgtatcaacggtggtaagtcttcccaagtctgcaggagcagctgcagctaattatacttactgggcctatgtgcctttcccacccttaattcgggcagttacatagatggataatcctattgaagtagatgttaataatagtgcatgggtgcctggccccacagatgactgttgccctgcccaacctgaagaaggaatgatgatgaatatttccattgggtatccttatcctcctgtttgcctagggaaggcaccaggatgcttaatgcctacaacccaaaattggttggtagaagtacctacagtcagtgctaccagtagatttacttatcacatggtaagtggaatgtcacagataaataatttacaggacccttcttatcaaagatcattacaatgtaggcctaaggggaaggcttgccccaaggaaattcccaaagaatcaaaaagcccagaagtcttagtctgcggagaatgtgtggctgatactgcagtgtagtacaaaacaatgaattttgaactatgatagactgggtcccttgaggccaattatatcataactgtacaggccagactcattcatgttcacaggccccatccatctggcccattaatccagcctatgacggtgatgtaactgaaaggctggaccaggtttatagaaggttagaatcactctgtccaaggaaatggggtgaaaagggaatttcatcaccttgaccaaagttagtcctgttactggtcctgaacatccagaattaggaagcttactgtggcctcacaccacattagaatttgttctggaaatcaagctataggaacaagagatcgtaagtcatattatactatcaacctaaattccagtctgacaattcctttgcaaaattgtgtaaaactcccttatattgctagttgtaggaaaaacatagttattaaacctgattcccaaaccataatctgtgaaaattgtggaatgtttacttgcattgatttgacttttaattggcagcaccgtattctactaggaagagcaagagagggtgtgtggatccttgtgtccatggaccgaccatgggaggcttcgctatccatccatattttaacggaagtattaaaaggaattctaactagatccaaaagattcatttttactttgatggcagtgattatgggcctcattgcagtcacagctactgctgcggctgctggaattgctttacactcctctgttcaaactgcagaatacgtaaatgattggcaaaagaattcctcaaaattgtggaattctcagatccaaatagatcaaaaattggcaaaccaaattaatgatcttagacaaactgtcatttggatgggagaggctcatgagcttggaatatctttttcagttacgatgtgactggaatacatcagatttttgtgttacaccacaagcctataatgagtctgagcatcactgggacatggttagatgccatctgcaaggaggagaagataatcttactttagacatttcaaaattaaaagaattttttttttctttgagacagagtctcgctctgtcgcccaggctggagtgcagtggcgtgatctcagctcactgcaagttccgcctcctgggtttacaccattctcctgcctcagcctcccaagtagttgggactacaggagcccaccaccatgcctggctaattttttttgggtttttaatagagatggagtttcaccgtgttagccaggatggtctcgatctcctgaccttgtgatctgcccaccttggcctcccaaagtgctgggattacagtcgtgagccaccgtgcccagccaagaaaaaatttttgaggcatcaaaagcccatttaaatttggtgccaggaacggagacaatcgtgaaagctgctgatagcctcacaaatcttaagccagtcacttgggttaaaagcatcagaagtttcactattgtaaatttcatattaatccttgtatgcctgttctgtctgttgttagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgt

SEQ ID NO: 11 - DNA, 8 bp, HERV-K
taggtctc

SEQ ID NO: 12 - DNA, 8 bp, HERV-K
taggggtg

SEQ ID NO: 13 - DNA, 8 bp, HERV-K
tggctgtt

SEQ ID NO: 14 - DNA, 204 bp, HERV-K
aacgtggatgcttttctctagggtgaagggactctcgagtgtggtcattgaggacaagtcaacgagagattcccgagtacgtctacagtgagccttgtgggtgaaggtactctacagtgtggtcattgaggacaagttgacgagagagtcccaagtacgtccacggtcagccttgcgacattttaaagttctacaatgaactca

SEQ ID NO: 15 - DNA, 503 bp, HERV-K
acagaagggtacatgaaggaaaactgatttggtggaaagataataaaaataaaacatgggaaatggggaaggtgataacgtgggggagaggttttgcttgtgtttcaccaggagaaaatcagcttcctgtttggatacccactagacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccagccctgtgctcacggaaacatgtgctgtaaggtttaagggatctagggctgt

SEQ ID NO: 16 - DNA, 637 bp, HERV-K
agtcatcaaaactcagtatcacttgactcaaagagcagagttggttgccgtcattacagtgttaacaagattttaatcagtctattaacattgtatcagattctgcatatgtagtacaggctacaaaggatattgagagagccctaatcaaatacattatggatgatcagttaaacccgctgtttaatttgttacaacaaaatgtaagaaaaagaaatttcccattttatattactcatattcgagcacacactaatttaccagggcctttaactaaagcaaatgaacaagctgactcgctagtatcatctgcattcatggaagcacaagaccttcatgccttgactcatgtaaatgcaataggattaaaaaataaatttaatatcacatggaaacagacaaaaaatattgtacaacattgcacccagtgtcagattctacacctggccactcaggaggcaagagttaatcccagaggtctatgtcctaatgtgttatggcaaatggatgtcatgcacgtaccttcatttggaaaattgtcatttgtccatgtgacagttgatacttattcacatttcatatgggcaacctgccagacaggagaaagtacttcccatgttaagagacatttattatc

SEQ ID NO: 17 - DNA, 650 bp, HERV-K
agtcatcaaaactcagtatcacttgactcaaagagcagagttggttgccgtcattacagtgttaacaagattttaatcagtctattaacattgtatcagattctgcatatgtagtacaggctacaaaggatattgagagagccctaatcaaatacattatggatgatcagttaaacccgctgtttaatttgttacaacaaaatgtaagaaaaagaaatttcccattttatattactcatattcgagcacacactaatttaccagggcctttaactaaagcaaatgaacaagctgacttgctagtatcatctgcattcatggaagcacaagaacttcatgccttgactcatgtaaatgcaataggattaaaaaataaatttgatatcacatggaaacagacaaaaaatattgtacaacattgcacccagtgtcagattctacacctggccactcaggaggcaagagttaatcccagaggtctatgtcctaatgtgttatggcaaatggatgtcatgcacgtaccttcatttggaaaattgtcatttgtccatgtgacagttgatacttattcacatttcatatgggcaacctgccagacaggagaaagtacttcccatgttaaaagacatttattatcttgttttcctgtc

SEQ ID NO: 18 - DNA, 650 bp, HERV-K; identical to SEQ ID NO: 17

SEQ ID NO: 19 - DNA, 200 bp, HERV-K
agatctgatcatctggtgcccaacgtggaggcttttctctagggtgaagggactctcgagtgtggtcattgaggacaagtcaacgagagattcccgagtacgtctacagtgagccttgtgggtgaaggtactctacagtgtggtcattgaggacaagttgacgagagagtcccaagtacgtccacggtcagccttgcgac

SEQ ID NO: 20 - DNA, 53 bp, Artificial Sequence
  RACE primer
aactggaagaattcgcggccgcatttttttttttttttttttttttttttttt

SEQ ID NO: 21 - DNA, 20 bp, Artificial Sequence
  RACE primer
caggtgtayccarcagctcc

SEQ ID NO: 22 - DNA, 23 bp, Artificial Sequence
  RACE primer
aactggaagaattcgcggccgca

SEQ ID NO: 23 - RNA, 11177 nt, HERV-K
gagauaggagaaaacugccuuagggcuggaggugggacaugcuggcggcaauacugcucuuuaaggcauugagauguuuauguauaugcacaucaaaagcacagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugugguaagcuugggcgcucggaagaagccaggguuaauggggcaaacuaaaaguaaagucucucauuccaccugaugagaaacacccagagguguggaggggcaggccaccccuucaggguaggguccccuccaugcagaccauagagcacaggugugccccaaagaggagcagagagaaggagggagagggcccacgagagacuuggaaaugaauggcaggauuuuaggcgcuggacuuggguucggggcaccuggccuuuccuuguguauuucuccuacugucugccuaacuauuuaauacaauaaaagaaaaccagccccugguucuugugguguuuccacccucccggguccccgcuggcugccuggcuuccucccgcagcuccugcuguguguguaugugugugugugugcacaucuguggggcguauguguguucgucuuuguaauugaggcugcagaguggagagagcagggguuuucucuggggacccagagagaaggaggcguuuucaccacagccgaacagggcaggaccccagcacccgggacccagcgggacuuugccaaggggauggaccuggcugggccacgcggcuguuuguguagggaaaagaaagagagaucacacuguuacugugucuauguagaaaaggaagacauaaacuccauuuugagcuguacuaagaaaaauuauuuugccuugaccugcuguuaaccuguaacuguagccccaacccugugcucaaagaaacaugugcuguauggaaucaagguuuaagggaucaagggcuguacaggaugugccuuguuaacaauguguuuacaggcaguaugcuugguaaaagucaucgccauucuccauucuccauuaaucaggggcacgaugcacugcggaaagccacagggaccucugcccgagaaagccuggguauuguccaaggcuuccccccacugagacagccugagauacggccucgugggaagggaaagaccugaccgucccccagcccgacacccguaaagggucugugcugaggaggauuaguaaaaggggaaggccucuugcaguugagauaagaggaaggccuccgucuccugcauguccuugggaauggaaugucuugguguaaaacccgauaguacauuccuucuauucugagagaagaaaaccacccuguggcuggaggugagauaugcuagcggcaaugcugcucuguuacucuuugcuacacugagauguuuggguggagagaagcauaaaucuggccuaugugcacaucugggcacagaaccuccccuugaacuugugacacagauuccuuuguucacauguuuuccugcugaccuucuccccacuaucgcccuguucucccaccgcauuccccuugcugagauagugaaaauaguaaucuguagauaccaagggaacucagagaccauggccggugcacauccuccguacgcugagcgcugguccccugggcccauuguucuuucucuauacuuugucucugugucuuauuucuuuccucagucucucaucccuccugacgagaaauacccacagguguggaggggcuggcccccuucaucugaugcccaaugugggugccuuucucuagggugaagguacucuacaguguggucauugaggacaaguugacgagagagucccaaguacguccacggucagccuugcgguaagcuugugugcuuagaggaacccaggguaacgauggggcaaacugaaaguaaauaugccucuuaucucagcuuuauuaaaauucuuuuaagaagagggggaguuagagcuucuacagaaaaucuaauuacgcuauuucaaacaauagaacaauucugcccaugguuuccagaacagggaacuuuagaucuaaaagauugggaaaaaauuggcaaagaauuaaaacaagcaaauagggaagguaaaaucaucccacuuacaguauggaaugauugggccauuauuaaagcaacuuuagaaccauuucaaacaggagaagauauuguuucaguuucugaugccccuaaaagcuguguaacagauugugaagaagaggcagggacagaaucccagcaaggaacggaaaguucacauuguaaauauguagcagagucuguaauggcucagucaacgcaaaauguugacuacagucaauuacaggagauaauauacccugaaucaucaaaauugggggaaggagguccagaaucauuggggccaucagagccuaaaccacgaucgccaucaacuccuccucccgugguucagaugccuguaacauuacaaccucaaacgcagguuagacaagcacaaaccccaagagaaaaucaaguagaaagggacagagucucuaucccggcaaugccaacucagauacaguauccacaauaucagccgguagaaaauaagacccaaccgcugguaguuuaucaauaccggcugccaaccgagcuucaguaucggccuccuucagagguucaauacagaccucaagcgguguguccugugccaaauagcacggcaccauaccagcaacccacagcgauggcgucuaauucaccagcaacacaggacgcggcgcuguauccucagccgcccacugugagacuuaauccuacagcaucacguaguggacaggguggugcacugcaugcagucauugaugaagccagaaaacagggcgaucuugaggcauggcgguuccugguaauuuuacaacugguacaggccggggaagagacucaaguaggagcgccugcccgagcugagacuagaugugaaccuuucaccaugaaaauguuaaaagauauaaaggaaggaguuaaacaauauggauccaacuccccuuauauaagaacauuauuagauuccauugcucauggaaauagacuuacuccuuaugacugggaaauuuuggccaaaucuucccuuucauccucucaguaucuacaguuuaaaaccugguggauugauggaguacaagaacagguacgaaaaaaucaggcuacuaagcccacuguuaauauagacgcagaccaauuguuaggaacagguccaaauuggagcaccauuaaccaacaaucagugaugcagaaugaggcuauugaacaaguaagggcuauuugccucagggccuggggaaaaauucaggacccaggaacagcuuucccuauuaauucaauuagacaaggcucuaaagagccauauccugacuuuguggcaagauuacaagaugcugcucaaaagucuauuacagaugacaaugcccgaaaaguuauuguagaauuaauggccuaugaaaaugcaaauccagaaugucagucggccauaaagccauuaaaaggaaaaguuccagcaggaguugauguaauuacagaauaugugaaggcuugugaugggauuggaggagcuaugcauaaggcaaugcuaauggcucaagcaaugagggggcucacucuaggaggacaaguuagaacauuugggaaaaaauguuauaauuguggucaaaucggucaucugaaaaggaguugcccaggcuuaaauaaacagaauauaauaaaucaagcuauuacagcaaaaaauaaaaagccaucuggccuguguccaaaauguggaaaagcaaaacauugggccaaucaaugucauucuaaauuugauaaagaugggcaaccauugucuggaaacaggaagaggggccagccucaggccccccaacaaacuggggcauucccaguuaaacuguuuguuccucaggguuuucaaggacaacaaccccuacagaaaauaccaccacuucagggagucagccaauuacaacaauccaacagcugucccgcgccacagcaggcagcaccgcaguagauuuauguuccacccaaauggucuuuuuacucccuggaaagcccccacaaaagauuccuagagggguauauggcccgcugccagaagggaggguaggccuuugagggagaucaagucuaaauuugaagggaguccaaauucauacugggguaauuuauucagauuauaaagggggaauucaguuagugaucagcuccacuguuccccggagugccaauccaggugauagaauugcucaauuacugcuuuugccuuauguuaaaauuggggaaaacaaaaaggaaagaacaggaggguuuggaaguaccaacccugcaggaaaagcugcuuauugggcuaaucaggucucagaggauagacccguguguacagucacuauucagggaaagaguuugaaggauuaguggauacccaggcugauguuucugucaucggcauagguacugccucagaaguguaucaaagugccaugauuuuacauuguccaggaucugauaaucaagaaaguacgguucagccugugaucacuucauuccaaucaauuuauggggccgagacuuguuacaacaauggcaugcagagauuacuaucccagccucccuauacagccccaggaauaaaaaaaucaugacuaaaaugggauagcucccuaaaaagggacuaggaaagaagucccaauugaggcugaaaaaaaucaaaaaagaaaaggaauagggcauccuuuuuaggagcggucacuguagagccuccaaaacccauuccauuaacuugggggaaaaaaaaacaacuguaugguaaaucagcagcgcuuccaaaacaaaaacuggaggcuuuacauuuauuagcaaagaaacaauuagaaaaaggacauugagccuucauuuucgccuuggaauucuguuuguaauucagaaaaaauccggcagauggcguauaaugccguaauucaacccaugggggcucucccaccccgguugcccucuccagccaugguccccuuuaauuauaauugaucugaaggauugcuuuuuuaccauuccucuggcaaaacaggauuuugaaaaauuugcuuuuaccacaccagccuaaauaauaaagaaccagccaccagguuucaguggaaaguauugccucagggaaugcuuaauaguucaacuauuugucagcucaagcucugcaaccaguuagagacaaguuuucagacuguuacaucguucacuauguugauauuuugugugcugcagaaacgagagacaaauuaauugaccguuacacauuucugcagacagagguugccaacgcgggacugacaauaacaucugauaagauucaaaccucuacuccuuuccguuacuugggaaugcagguagaggaaaggaaaauuaaaccacaaaaaauagaaauaagaaaagacacauuaaaagcauuaaaugaguuucaaaaguugcuaggagauacuaauuggauuuggagauauuaauuggauuuggccaacucuaggcauuccuacuuaugccaugucaaauuuguucucuuucuuaagaggggacucggaauuaaauagugaaagaacguuaacuccagaggcaacuaaagaaauuaaauuaauugaagaaaaaauucggucagcacaaguaaauagaauagaucacuuggccccacuccaaauuuugauuuuugcuacugcacauucccuaacaggcaucauuguucaaaauacagaucuuguggagugguccuuccuuccucacaguacaauuaagacuuuuacauuguacuuggaucaaauggcuacauuaauuggucagggaagauuaugaauaauaacauuguguggaaaugacccagauaaaaucacuguuccuuucaacaagcaacagguuagacaagccuuuaucaauucuggugcauggcagauuggucuugccgauuuugugggaauuauugacaaucguuaccccaaaacaaaaaucuuccaguuuuuaaaauugacuacuuggauuuuaccuaaaguuaccaaacauaagccuuuaaaaaaugcucuggcaguguuuacugaugguuccagcaauggaaaaguggcuuacaccgggccaaaagaaugagucaucaaaacucaguaucacuugacucaaagagcagaguugguugccgucauuacaguguuaacaagauuuuaaucagucuauuaacauuguaucagauucugcauauguaguacaggcuacaaaggauauugagagagcccuaaucaaauacauuauggaugaucaguuaaacccgcuguuuaauuuguuacaacaaaauguaagaaaaagaaauuucccauuuuauauuacucauauucgagcacacacuaauuuaccagggccuuuaacuaaagcaaaugaacaagcugacuugcuaguaucaucugcauucauggaagcacaagaacuucaugccuugacucauguaaaugcaauaggauuaaaaaauaaauuugauaucacauggaaacagacaaaaaauauuguacaacauugcacccagugucagauucuacaccuggccacucaggaggcaagaguuaaucccagaggucuauguccuaauguguuauggcaaauggaugucaugcacguaccuucauuuggaaaauugucauuuguccaugugacaguugauacuuauucacauuucauaugggcaaccugccagacaggagaaaguacuucccauguuaaaagacauuuauuaucuuguuuuccugucaugggaguuccagaaaaaguuaaaacagacaaugggccagguuacuguaguaaagcaguucaaaaauucuuaaaucaguggaaaauuacacauacaauaggaauucucuauaauucccaaggacaggccauaauugaaagaacuaauagaacacucaaagcucaauugguuaaacaaaaaaaaggaaaagacaggaguauaacacuccccagaugcaacuuaaucuagcacucuauacuuuaaauguuuuaaacauuuauagaaaucagaccacuaccucugcagaacaacaucuuacugguaaaaggaacagcccacaugaaggaaaacugauuugguggaaagauaauaaaaauaaaacaugggaaauggggaaggugauaacgugggggagagguuuugcuuguguuucaccaggagaaaaucagcuuccuguuuggauacccacuagacauuuaaaguucuacaaugaacucacuggagaugcaaagaaaaguguggagauggagacaccccaaucgacucgccagguaaacaaaauggugauaucagaagaacagaaaaaguugccuuccaucaaggaagcagaguugccaauauaggcacaauuaaagaagcugacacaguuagcuaaaaaaaaaagccuagagaauacaaaggugacaccaacuccagagaauaugcugcuugcagcucugaugauuguaucaacggugguaagucuucccaagucugcaggagcagcugcagcuaauuauacuuacugggccuaugugccuuucccacccuuaauucgggcaguuacauagauggauaauccuauugaaguagauguuaauaauagugcaugggugccuggccccacagaugacuguugcccugcccaaccugaagaaggaaugaugaugaauauuuccauuggguauccuuauccuccuguuugccuagggaaggcaccaggaugcuuaaugccuacaacccaaaauugguugguagaaguaccuacagucagugcuaccaguagauuuacuuaucacaugguaaguggaaugucacagauaaauaauuuacaggacccuucuuaucaaagaucauuacaauguaggccuaaggggaaggcuugccccaaggaaauucccaaagaaucaaaaagcccagaagucuuagucugcggagaauguguggcugauacugcaguguaguacaaaacaaugaauuuugaacuaugauagacugggucccuugaggccaauuauaucauaacuguacaggccagacucauucauguucacaggccccauccaucuggcccauuaauccagccuaugacggugauguaacugaaaggcuggaccagguuuauagaagguuagaaucacucuguccaaggaaauggggugaaaagggaauuucaucaccuugaccaaaguuaguccuguuacugguccugaacauccagaauuaggaagcuuacuguggccucacaccacauuagaauuuguucuggaaaucaagcuauaggaacaagagaucguaagucauauuauacuaucaaccuaaauuccagucugacaauuccuuugcaaaauuguguaaaacucccuuauauugcuaguuguaggaaaaacauaguuauuaaaccugauucccaaaccauaaucugugaaaauuguggaauguuuacuugcauugauuugacuuuuaauuggcagcaccguauucuacuaggaagagcaagagaggguguguggauccuuguguccauggaccgaccaugggaggcuucgcuauccauccauauuuuaacggaaguauuaaaaggaauucuaacuagauccaaaagauucauuuuuacuuugauggcagugauuaugggccucauugcagucacagcuacugcugcggcugcuggaauugcuuuacacuccucuguucaaacugcagaauacguaaaugauuggcaaaagaauuccucaaaauuguggaauucucagauccaaauagaucaaaaauuggcaaaccaaauuaaugaucuuagacaaacugucauuuggaugggagaggcucaugagcuuggaauaucuuuuucaguuacgaugugacuggaauacaucagauuuuuguguuacaccacaagccuauaaugagucugagcaucacugggacaugguuagaugccaucugcaaggaggagaagauaaucuuacuuuagacauuucaaaauuaaaagaauuuuuuuuuucuuugagacagagucucgcucugucgcccaggcuggagugcaguggcgugaucucagcucacugcaaguuccgccuccuggguuuacaccauucuccugccucagccucccaaguaguugggacuacaggagcccaccaccaugccuggcuaauuuuuuuuggguuuuuaauagagauggaguuucaccguguuagccaggauggucucgaucuccugaccuugugaucugcccaccuuggccucccaaagugcugggauuacagucgugagccaccgugcccagccaagaaaaaauuuuugaggcaucaaaagcccauuuaaauuuggugccaggaacggagacaaucgugaaagcugcugauagccucacaaaucuuaagccagucacuuggguuaaaagcaucagaaguuucacuauuguaaauuucauauuaauccuuguaugccuguucugucuguuguuagucuacagguguauccagcagcuccaaagagacagcaaccagcaagaaugggccauagugacgauggugguuuugucaaaaagaaaagggggggauauguaaggaaaagagagaucagacuuucacugugucuauguagaaaaggaagacauaagaaacuccauuuugaucuguacuaagaaaaauuguuuugccuugagaugcuguuaaucuguaacuuuagccccaacccugugcucacggaaacaugugcuguaagguuuaagggaucuagggcugugcaggauguaccuuguuaacaauauguuugcaggcaguauguuugguaaaagucaucgccauucuccauucucgauuaaccaggggcucaaugcacuguggaaagccacaggaaccucugcccaagaaagccuggcuguugugggaagucagggaccccgaauggagggaccagcuggugcugcaucaggaaacauaaauugugaagauuucuuggacauuuaucaguuuccaaaauuaauacuuuuauaauuucuuacaccugucuuacuuuaaucucuuaauccuguuaucuuuguaagcugaggauauacgucaccucaggaccacuauuguacaaauugauuguaaaacauguucacauguguuugaacaauaugaaaucagugcaccuugaaaaugaacagaauaacagugauuuuagggaacaaaggaagacaaccauaaggucugacugccugaggggucgggcaaaaagccauauuuuucuucuugcagagagccuauaaauggacgugcaaguaggagagauauugcuaaauucuuuuccuagcaaggaauauaauacuaagacccuagggaaagaauugcauuccuggggggaggucuauaaacggccgcucugggagugucuguccuaugugguugagauaaggacugagauacgcccuggucuccugcaguacccucaggcuuacuaggauugggaaaccccaguccugguaaauuugaggucaggccgguucuuugcucugaacccuguuuucuguuaagauguuuaucaagacaauacaugcaccgcugaacauagacccuuaucaggaguuucugauuuugcucugguccuguuucuucagaagcaugucaucuuugcucugccuucugcccuuugaagcaugugaucuuugugaccuacucccuguucauacaccccuccccuuuuaaaaucccuaauaaaaacuugcugguuuuguggcucaggggggcaucauggaccuaccaauacgugaugucacccccgguggcccagcugu

SEQ ID NO: 24 - RNA, 527 nt, HERV-K
gagauaggagaaaacugccuuagggcuggaggugggacaugcuggcggcaauacugcucuuuaaggcauugagauguuuauguauaugcacaucaaaagcacagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugugucucucaucc

SEQ ID NO: 25 - RNA, 527 nt, HERV-K
gagauaggagaaaacugccuuagggcuggaggugggacaugcuggcggcaauacugcucuuuaaggcauugagauguuuauguauaugcacaucaaaagcacagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugugggugaaggua

SEQ ID NO: 26 - RNA, 517 nt, HERV-K
gagauaggagaaaacugccuuagggcuggaggugggacaugcuggcggcaauacugcucuuuaaggcauugagauguuuauguauaugcacaucaaaagcacagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugug

SEQ ID NO: 27 - RNA, 10 nt, HERV-K
ucucucaucc

SEQ ID NO: 28 - RNA, 10 nt, HERV-K
ggugaaggua

SEQ ID NO: 29 - RNA, 1216 nt, HERV-K
uguaaggaaaagagagaucagacuuucacugugucuauguagaaaaggaagacauaagaaacuccauuuugaucuguacuaagaaaaauuguuuugccuugagaugcuguuaaucuguaacuuuagccccaacccugugcucacggaaacaugugcuguaagguuuaagggaucuagggcugugcaggauguaccuuguuaacaauauguuugcaggcaguauguuugguaaaagucaucgccauucuccauucucgauuaaccaggggcucaaugcacuguggaaagccacaggaaccucugcccaagaaagccuggcuguugugggaagucagggaccccgaauggagggaccagcuggugcugcaucaggaaacauaaauugugaagauuucuuggacauuuaucaguuuccaaaauuaauacuuuuauaauuucuuacaccugucuuacuuuaaucucuuaauccuguuaucuuuguaagcugaggauauacgucaccucaggaccacuauuguacaaauugauuguaaaacauguucacauguguuugaacaauaugaaaucagugcaccuugaaaaugaacagaauaacagugauuuuagggaacaaaggaagacaaccauaaggucugacugccugaggggucgggcaaaaagccauauuuuucuucuugcagagagccuauaaauggacgugcaaguaggagagauauugcuaaauucuuuuccuagcaaggaauauaauacuaagacccuagggaaagaauugcauuccuggggggaggucuauaaacggccgcucugggagugucuguccuaugugguugagauaaggacugagauacgcccuggucuccugcaguacccucaggcuuacuaggauugggaaaccccaguccugguaaauuugaggucaggccgguucuuugcucugaacccuguuuucuguuaagauguuuaucaagacaauacaugcaccgcugaacauagacccuuaucaggaguuucugauuuugcucugguccuguuucuucagaagcaugucaucuuugcucugccuucugcccuuugaagcaugugaucuuugugaccuacucccuguucauacaccccuccccuuuuaaaaucccuaauaaaaacuugcugguuuuguggcucaggggggcaucauggaccuaccaauacgugaugucacccccgguggcccagcugu

SEQ ID NO: 30 - RNA, 319 nt, HERV-K
uguaaggaaaagagagaucagacuuucacugugucuauguagaaaaggaagacauaagaaacuccauuuugaucuguacuaagaaaaauuguuuugccuugagaugcuguuaaucuguaacuuuagccccaacccugugcucacggaaacaugugcuguaagguuuaagggaucuagggcugugcaggauguaccuuguuaacaauauguuugcaggcaguauguuugguaaaagucaucgccauucuccauucucgauuaaccaggggcucaaugcacuguggaaagccacaggaaccucugcccaagaaagccuggc

SEQ ID NO: 31 - RNA, 897 nt, HERV-K
uguugugggaagucagggaccccgaauggagggaccagcuggugcugcaucaggaaacauaaauugugaagauuucuuggacauuuaucaguuuccaaaauuaauacuuuuauaauuucuuacaccugucuuacuuuaaucucuuaauccuguuaucuuuguaagcugaggauauacgucaccucaggaccacuauuguacaaauugauuguaaaacauguucacauguguuugaacaauaugaaaucagugcaccuugaaaaugaacagaauaacagugauuuuagggaacaaaggaagacaaccauaaggucugacugccugaggggucgggcaaaaagccauauuuuucuucuugcagagagccuauaaauggacgugcaaguaggagagauauugcuaaauucuuuuccuagcaaggaauauaauacuaagacccuagggaaagaauugcauuccuggggggaggucuauaaacggccgcucugggagugucuguccuaugugguugagauaaggacugagauacgcccuggucuccugcaguacccucaggcuuacuaggauugggaaaccccaguccugguaaauuugaggucaggccgguucuuugcucugaacccuguuuucuguuaagauguuuaucaagacaauacaugcaccgcugaacauagacccuuaucaggaguuucugauuuugcucugguccuguuucuucagaagcaugucaucuuugcucugccuucugcccuuugaagcaugugaucuuugugaccuacucccuguucauacaccccuccccuuuuaaaaucccuaauaaaaacuugcugguuuuguggcucaggggggcaucauggaccuaccaauacgugaugucacccccgguggcccagcugu

SEQ ID NO: 32 - DNA, 307 bp, HERV-K
ttaaaagaattttttttttctttgagacagagtctcgctctgtcgcccaggctggagtgcagtggcgtgatctcagctcactgcaagttccgcctcctgggtttacaccattctcctgcctcagcctcccaagtagttgggactacaggagcccaccaccatgcctggctaattttttttgggtttttaatagagatggagtttcaccgtgttagccaggatggtctcgatctcctgaccttgtgatctgcccaccttggcctcccaaagtgctgggattacagtcgtgagccaccgtgcccagcca

SEQ ID NO: 33 - DNA, 10 bp, HERV-K
catttcaaaa

SEQ ID NO: 34 - DNA, 10 bp, HERV-K
agaaaaaatt

SEQ ID NO: 35 - DNA, 10 bp, HERV-K
ttaaaagaat

SEQ ID NO: 36 - DNA, 20 bp, HERV-K
catttcaaaattaaaagaat

SEQ ID NO: 37 - DNA, 100 bp, HERV-K
tgttacaccacaagcctataatgagtctgagcatcactgggacatggttagatgccatctgcaaggaggagaagataatcttactttagacatttcaaaa

SEQ ID NO: 38 - DNA, 407 bp, HERV-K
tgttacaccacaagcctataatgagtctgagcatcactgggacatggttagatgccatctgcaaggaggagaagataatcttactttagacatttcaaaattaaaagaattttttttttctttgagacagagtctcgctctgtcgcccaggctggagtgcagtggcgtgatctcagctcactgcaagttccgcctcctgggtttacaccattctcctgcctcagcctcccaagtagttgggactacaggagcccaccaccatgcctggctaattttttttgggtttttaatagagatggagtttcaccgtgttagccaggatggtctcgatctcctgaccttgtgatctgcccaccttggcctcccaaagtgctgggattacagtcgtgagccaccgtgcccagcca

SEQ ID NO: 39 - DNA, 8 bp, HERV-K
aaaattaa

SEQ ID NO: 40 - DNA, 100 bp, HERV-K
agaaaaaatttttgaggcatcaaaagcccatttaaatttggtgccaggaacggagacaatcgtgaaagctgctgatagcctcacaaatcttaagccagtc

SEQ ID NO: 41 - DNA, 10 bp, HERV-K
tgcccagcca

SEQ ID NO: 42 - DNA, 110 bp, HERV-K
tgcccagccaagaaaaaatttttgaggcatcaaaagcccatttaaatttggtgccaggaacggagacaatcgtgaaagctgctgatagcctcacaaatcttaagccagtc

SEQ ID NO: 43 - DNA, 407 bp, HERV-K
ttaaaagaattttttttttctttgagacagagtctcgctctgtcgcccaggctggagtgcagtggcgtgatctcagctcactgcaagttccgcctcctgggtttacaccattctcctgcctcagcctcccaagtagttgggactacaggagcccaccaccatgcctggctaattttttttgggtttttaatagagatggagtttcaccgtgttagccaggatggtctcgatctcctgaccttgtgatctgcccaccttggcctcccaaagtgctgggattacagtcgtgagccaccgtgcccagccaagaaaaaatttttgaggcatcaaaagcccatttaaatttggtgccaggaacggagacaatcgtgaaagctgctgatagcctcacaaatcttaagccagtc

SEQ ID NO: 44 - DNA, 8 bp, HERV-K
gccaagaa

SEQ ID NO: 45 - DNA, 20 bp, HERV-K
tgcccagccaagaaaaaatt

SEQ ID NO: 46 - DNA, 100 bp, HERV-K
tctctcatccctcctgacgagaaatacccacaggtgtggaggggctggcccccttcatctgatgcccaatgtgggtgcctttctctagggtgaaggtact

SEQ ID NO: 47 - DNA, 617 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgtctctcatccctcctgacgagaaatacccacaggtgtggaggggctggcccccttcatctgatgcccaatgtgggtgcctttctctagggtgaaggtact

SEQ ID NO: 48 - DNA, 100 bp, HERV-K
ggtgaaggtactctacagtgtggtcattgaggacaagttgacgagagagtcccaagtacgtccacggtcagccttgcggtaagcttgtgtgcttagagga

SEQ ID NO: 49 - DNA, 617 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgggtgaaggtactctacagtgtggtcattgaggacaagttgacgagagagtcccaagtacgtccacggtcagccttgcggtaagcttgtgtgcttagagga

SEQ ID NO: 50 - DNA, 20 bp, HERV-K
gagccttgtgtctctcatcc

SEQ ID NO: 51 - DNA, 20 bp, HERV-K
gagccttgtgggtgaaggta

SEQ ID NO: 52 - DNA, 20 bp, HERV-K
aaagcctggctgttgtggga

SEQ ID NO: 53 - DNA, 48 bp, HERV-K
acagcgatggcgtctaattcaccagcaacacaggacgcggcgctgtat

SEQ ID NO: 54 - protein, 711 aa, HERV-K
MGQTESKYASYLSFIKILLRRGGVRASTENLITLFQTIEQFCPWFPEQGTLDLKDWEKIGKELKQANREGKIIPLTVWNDWAIIKATLEPFQTGEDIVSVSDAPKSCVTDCEEEAGTESQQGTESSHCKYVAESVMAQSTQNVDYSQLQEIIYPESSKLGEGGPESLGPSEPKPRSPSTPPPVVQMPVTLQPQTQVRQAQTPRENQVERDRVSIPAMPTQIQYPQYQPVENKTQPLVVYQYRLPTELQYRPPSEVQYRPQAVCPVPNSTAPYQQPTAMNSPATQDAALYPQPPTVRLNPTASRSGQGGALHAVIDEARKQGDLEAWRFLVILQLVQAGEETQVGAPARAETRCEPFTMKMLKDIKEGVKQYGSNSPYIRTLLDSIAHGNRLTPYDWEILAKSSLSSSQYLQFKTWWIDGVQEQVRKNQATKPTVNIDADQLLGTGPNWSTINQQSVMQNEAIEQVRAICLRAWGKIQDPGTAFPINSIRQGSKEPYPDFVARLQDAAQKSITDDNARKVIVELMAYENANPECQSAIKPLKGKVPAGVDVITEYVKACDGIGGAMHKAMLMAQAMRGLTLGGQVRTFGKKCYNCGQIGHLKRSCPQKQNIINQAITAKNKKPSGLCPKCGKAKHWANQCHSKFDKDGQPLSGNRKRGQPQAPQQTGAFPVKLFVPQGFQGQQPLQKIPPLQGVSQLQQSNSCPAPQQAAPQ

SEQ ID NO: 55 - protein, 23 aa, HERV-K
TQVRQAQTPRENQVERDRVSIPA

SEQ ID NO: 56 - protein, 15 aa, HERV-K
PTAMNSPATQDAALY

SEQ ID NO: 57 - DNA, 2145 bp, HERV-K
atggggcaaactgaaagtaaatatgcctcttatctcagctttattaaaattcttttaagaagagggggagttagagcttctacagaaaatctaattacgctatttcaaacaatagaacaattctgcccatggtttccagaacagggaactttagatctaaaagattgggaaaaaattggcaaagaattaaaacaagcaaatagggaaggtaaaatcatcccacttacagtatggaatgattgggccattattaaagcaactttagaaccatttcaaacaggagaagatattgtttcagtttctgatgcccctaaaagctgtgtaacagattgtgaagaagaggcagggacagaatcccagcaaggaacggaaagttcacattgtaaatatgtagcagagtctgtaatggctcagtcaacgcaaaatgttgactacagtcaattacaggagataatataccctgaatcatcaaaattgggggaaggaggtccagaatcattggggccatcagagcctaaaccacgatcgccatcaactcctcctcccgtggttcagatgcctgtaacattacaacctcaaacgcaggttagacaagcacaaaccccaagagaaaatcaagtagaaagggacagagtctctatcccggcaatgccaactcagatacagtatccacaatatcagccggtagaaaataagacccaaccgctggtagtttatcaataccggctgccaaccgagcttcagtatcggcctccttcagaggttcaatacagacctcaagcggtgtgtcctgtgccaaatagcacggcaccataccagcaacccacagcgatggcgtctaattcaccagcaacacaggacgcggcgctgtatcctcagccgcccactgtgagacttaatcctacagcatcacgtagtggacagggtggtgcactgcatgcagtcattgatgaagccagaaaacagggcgatcttgaggcatggcggttcctggtaattttacaactggtacaggccggggaagagactcaagtaggagcgcctgcccgagctgagactagatgtgaacctttcaccatgaaaatgttaaaagatataaaggaaggagttaaacaatatggatccaactccccttatataagaacattattagattccattgctcatggaaatagacttactccttatgactgggaaattttggccaaatcttccctttcatcctctcagtatctacagtttaaaacctggtggattgatggagtacaagaacaggtacgaaaaaatcaggctactaagcccactgttaatatagacgcagaccaattgttaggaacaggtccaaattggagcaccattaaccaacaatcagtgatgcagaatgaggctattgaacaagtaagggctatttgcctcagggcctggggaaaaattcaggacccaggaacagctttccctattaattcaattagacaaggctctaaagagccatatcctgactttgtggcaagattacaagatgctgctcaaaagtctattacagatgacaatgcccgaaaagttattgtagaattaatggcctatgaaaatgcaaatccagaatgtcagtcggccataaagccattaaaaggaaaagttccagcaggagttgatgtaattacagaatatgtgaaggcttgtgatgggattggaggagctatgcataaggcaatgctaatggctcaagcaatgagggggctcactctaggaggacaagttagaacatttgggaaaaaatgttataattgtggtcaaatcggtcatctgaaaaggagttgcccaggcttaaataaacagaatataataaatcaagctattacagcaaaaaataaaaagccatctggcctgtgtccaaaatgtggaaaagcaaaacattgggccaatcaatgtcattctaaatttgataaagatgggcaaccattgtctggaaacaggaagaggggccagcctcaggccccccaacaaactggggcattcccagttaaactgtttgttcctcagggttttcaaggacaacaacccctacagaaaataccaccacttcagggagtcagccaattacaacaatccaacagctgtcccgcgccacagcaggcagcaccgcag

SEQ ID NO: 58 - DNA, 927 bp, HERV-K
tgggcaaccattgtctggaaacaggaagaggggccagcctcaggccccccaacaaactggggcattcccagttaaactgtttgttcctcagggttttcaaggacaacaacccctacagaaaataccaccacttcagggagtcagccaattacaacaatccaacagctgtcccgcgccacagcaggcagcaccgcagtagatttatgttccacccaaatggtctttttactccctggaaagcccccacaaaagattcctagaggggtatatggcccgctgccagaagggagggtaggcctttgagggagatcaagtctaaatttgaagggagtccaaattcatactggggtaatttattcagattataaagggggaattcagttagtgatcagctccactgttccccggagtgccaatccaggtgatagaattgctcaattactgcttttgccttatgttaaaattggggaaaacaaaaaggaaagaacaggagggtttggaagtaccaaccctgcaggaaaagctgcttattgggctaatcaggtctcagaggatagacccgtgtgtacagtcactattcagggaaagagtttgaaggattagtggatacccaggctgatgtttctgtcatcggcataggtactgcctcagaagtgtatcaaagtgccatgattttacattgtccaggatctgataatcaagaaagtacggttcagcctgtgatcacttcattccaatcaatttatggggccgagacttgttacaacaatggcatgcagagattactatcccagcctccctatacagccccaggaataaaaaaatcatgactaaaatgggatagctccctaaaaagggactaggaaagaagtcccaattgaggctgaaaaaaatcaaaaaagaaaaggaatagggcatcctttttag

SEQ ID NO: 59 - protein, 24 aa, HERV-K
WATIVWKQEEGPASGPPTNWGIPS

SEQ ID NO: 60 - protein, 75 aa, HERV-K
TVCSSGFSRTTTPTENTTTSGSQPITTIQQLSRATAGSTAVDLCSTQMVFLLPGKPPQKIPRGVYGPLPEGRVGL

SEQ ID NO: 61 - protein, 178 aa, HERV-K
GRSSLNLKGVQIHTGVIYSDYKGGIQLVISSTVPRSANPGDRIAQLLLLPYVKIGENKKERTGGFGSTNPAGKAAYWANQVSEDRPVCTVTIQGKSLKDVDTQADVSVIGIGTASEVYQSAMILHCPGSDNQESTVQPVITSFIPINLWGRDLLQQWHAEITIPASKPRNKKIMTKMG

SEQ ID NO: 62 - protein, 28 aa, HERV-K
LPKKGLGKKEVPIEAEKNQKRKGIGHPF

SEQ ID NO: 63 - DNA, 651 bp, HERV-K
acatccagaattaggaagcttactgtggcctcacaccacattagaatttgttctggaaatcaagctataggaacaagagatcgtaagtcatattatactatcaacctaaattccagtctgacaattcctttgcaaaattgtgtaaaactcccttatattgctagttgtaggaaaaacatagttattaaacctgattcccaaaccataatctgtgaaaattgtggaatgtttacttgcattgatttgacttttaattggcagcaccgtattctactaggaagagcaagagagggtgtgtggatccttgtgtccatggaccgaccatgggaggcttcgctatccatccatattttaacggaagtattaaaaggaattctaactagatccaaaagattcatttttactttgatggcagtgattatgggcctcattgcagtcacagctactgctgcggctgctggaattgctttacactcctctgttcaaactgcagaatacgtaaatgattggcaaaagaattcctcaaaattgtggaattctcagatccaaatagatcaaaaattggcaaaccaaattaatgatcttagacaaactgtcatttggatgggagaggctcatgagcttggaatatctttttcagttacgatgtga

SEQ ID NO: 64 - protein, 216 aa, HERV-K
TSRIRKLTVASHHIRICSGNQAIGTRDRKSYYTINLNSSLTIPLQNCVKLPYIASCRKNIVIKPDSQTIICENCGMFTCIDLTFNWQHRILLGRAREGVWILVSMDRPWEASLSIHILTEVLKGILTRSKRFIFTLMAVIMGLIAVTATAAAAGIALHSSVQTAEYVNDWQKNSSKLWNSQIQIDQKLANQINDLRQTVIWMGEAHELGISFSVTM

SEQ ID NO: 65 - protein, 22 aa, HERV-K
HPELGSLLWPHTTLEFVLEIKL

SEQ ID NO: 66 - protein, 12 aa, HERV-K
EQEIVSHIILST

SEQ ID NO: 67 - protein, 3 aa, HERV-K
IPV

SEQ ID NO: 68 - protein, 7 aa, HERV-K
QFLCKIV

SEQ ID NO: 69 - protein, 11 aa, HERV-K
NSLILLVVGKT

SEQ ID NO: 70 - protein, 8 aa, HERV-K
LLNLIPKP

SEQ ID NO: 71 - protein, 12 aa, HERV-K
SVKIVECLLALI

SEQ ID NO: 72 - protein, 9 aa, HERV-K
LLIGSTVFY

SEQ ID NO: 73 - protein, 25 aa, HERV-K
EEQERVCGSLCPWTDHGRLRYPSIF

SEQ ID NO: 74 - protein, 3 aa, HERV-K
RKY

SEQ ID NO: 75 - protein, 3 aa, HERV-K
KEF

SEQ ID NO: 76 - protein, 9 aa, HERV-K
LDPKDSFLL

SEQ ID NO: 77 - protein, 2 aa, HERV-K
WQ

SEQ ID NO: 78 - protein, 27 aa, HERV-K
LWASLQSQLLLRLLELLYTPLFKLQNT

SEQ ID NO: 79 - protein, 16 aa, HERV-K
MIGKRIPQNCGILRSK

SEQ ID NO: 80 - protein, 32 aa, HERV-K
IKNWQTKLMILDKLSFGWERLMSLEYLFQLRC

SEQ ID NO: 81 - DNA, 249 bp, HERV-K
gtacaaaacaatgaattttgaactatgatagactgggtcccttgaggccaattatatcataactgtacaggccagactcattcatgttcacaggccccatccatctggcccattaatccagcctatgacggtgatgtaactgaaaggctggaccaggtttatagaaggttagaatcactctgtccaaggaaatggggtgaaaagggaatttcatcaccttgaccaaagttagtcctgttactggtcctg

SEQ ID NO: 82 - protein, 6 aa, HERV-K
VQNNEF

SEQ ID NO: 83 - protein, 7 aa, HERV-K
TMIDWVP

SEQ ID NO: 84 - protein, 58 aa, HERV-K
GQLYHNCTGQTHSCSQAPSIWPINPAYDGDVTERLDQVYRRLESLCPRKWGEKGISSP

SEQ ID NO: 85 - protein, 9 aa, HERV-K
PKLVLLLVL

SEQ ID NO: 86 - DNA, 1839 bp, HERV-K
atgtcaaatttgttctctttcttaagaggggactcggaattaaatagtgaaagaacgttaactccagaggcaactaaagaaattaaattaattgaagaaaaaattcggtcagcacaagtaaatagaatagatcacttggccccactccaaattttgatttttgctactgcacattccctaacaggcatcattgttcaaaatacagatcttgtggagtggtccttccttcctcacagtacaattaagacttttacattgtacttggatcaaatggctacattaattggtcagggaagattatgaataataacattgtgtggaaatgacccagataaaatcactgttcctttcaacaagcaacaggttagacaagcctttatcaattctggtgcatggcagattggtcttgccgattttgtgggaattattgacaatcgttaccccaaaacaaaaatcttccagtttttaaaattgactacttggattttacctaaagttaccaaacataagcctttaaaaaatgctctggcagtgtttactgatggttccagcaatggaaaagtggcttacaccgggccaaaagaatgagtcatcaaaactcagtatcacttgactcaaagagcagagttggttgccgtcattacagtgttaacaagattttaatcagtctattaacattgtatcagattctgcatatgtagtacaggctacaaaggatattgagagagccctaatcaaatacattatggatgatcagttaaacccgctgtttaatttgttacaacaaaatgtaagaaaaagaaatttcccattttatattactcatattcgagcacacactaatttaccagggcctttaactaaagcaaatgaacaagctgacttgctagtatcatctgcattcatggaagcacaagaacttcatgccttgactcatgtaaatgcaataggattaaaaaataaatttgatatcacatggaaacagacaaaaaatattgtacaacattgcacccagtgtcagattctacacctggccactcaggaggcaagagttaatcccagaggtctatgtcctaatgtgttatggcaaatggatgtcatgcacgtaccttcatttggaaaattgtcatttgtccatgtgacagttgatacttattcacatttcatatgggcaacctgccagacaggagaaagtacttcccatgttaaaagacatttattatcttgttttcctgtcatgggagttccagaaaaagttaaaacagacaatgggccaggttactgtagtaaagcagttcaaaaattcttaaatcagtggaaaattacacatacaataggaattctctataattcccaaggacaggccataattgaaagaactaatagaacactcaaagctcaattggttaaacaaaaaaaaggaaaagacaggagtataacactccccagatgcaacttaatctagcactctatactttaaatgttttaaacatttatagaaatcagaccactacctctgcagaacaacatcttactggtaaaaggaacagcccacatgaaggaaaactgatttggtggaaagataataaaaataaaacatgggaaatggggaaggtgataacgtgggggagaggttttgcttgtgtttcaccaggagaaaatcagcttcctgtttggatacccactagacatttaaagttctacaatgaactcactggagatgcaaagaaaagtgtggagatggagacaccccaatcgactcgccaggtaaacaaaatggtgatatcagaagaacagaaaaagttgccttccatcaaggaagcagagttgccaatatag

SEQ ID NO: 87 - protein, 79 aa, HERV-K
MNSLEMQRKVWRWRHPNRLASLQVYPAAPKRQQPARMGHSDDGGFVKKKRGGYVRKREIRLSLCLCRKGRHKKLHFDLY

SEQ ID NO: 88 - DNA, 237 bp, HERV-K
atgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtac

SEQ ID NO: 89 - DNA, 723 bp, HERV-K
  misc_feature 56, 69, 114, 117, 165, 168, 204, 208, 249, 284, 299,326, 328, 338, 340, 422, 429, 443, 450, 474, 505, 528, 533: n = A,T,C or G
  misc_feature 559: n = A,T,C or G
  misc_feature 56, 69, 114, 117, 165, 168, 204, 208, 249, 284, 299,326, 328, 338, 340, 422, 429, 443, 450, 474, 505, 528, 533: n = A,T,C or G
  misc_feature 559: n = A,T,C or G
ggcccactattgtacaaattgattgtaaaacatgttcacatgtgttgaacaatatnaaatcagggcccnttgaaaatgaacagaataacagtgattttagggaacaaaggaagncanccataaggtctgcctgcctgaggggtcgggcaaaaacccatatttttnttnttgcagagagcctataaatggacgtgcaagtagganaganattgctaaattcttttcctagcaaggaatataatactaagnccctagggaaagaattgcattcctggggggaggtntataaacggccgctntgggagtgtctgtcctatgtggttgananaaggactgananacgccctggtcgcctgcagtaccctcaggcttactaggattgggaaaccccagtcctggtaaatttgaggtcaggccggttntttgctntgaaccctgttttntgttaanatgtttatcaagacaatacgtgcnccgctgaacatagacccttatcaggagtttntgattttgctctggtcctgtttnttcanaagcatgtcatctttgctctgccttntgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaaaaaaaaaaa

SEQ ID NO: 90 - DNA, 765 bp, HERV-K
  misc_feature 2, 3, 5, 6, 24, 57, 68, 79, 86, 100, 102, 105, 111, 119,137, 160, 162, 163, 170, 213, 214, 215, 244, 250, 255: n = A,T,C or G
  misc_feature 262, 279, 286, 310, 313, 335, 338, 351, 356, 368, 370,378, 382, 385, 388, 399, 402, 409, 438, 449, 455, 459, 468: n = A,T,C or G
  misc_feature 469, 473, 475, 492, 495, 504, 509, 511, 513, 520, 525,527, 529, 547, 570, 592, 601, 610, 614, 622, 645, 648, 653: n = A,T,C or G
  misc_feature 722, 733: n = A,T,C or G
  misc_feature 2, 3, 5, 6, 24, 57, 68, 79, 86, 100, 102, 105, 111, 119,137, 160, 162, 163, 170, 213, 214, 215, 244, 250, 255: n = A,T,C or G
  misc_feature 262, 279, 286, 310, 313, 335, 338, 351, 356, 368, 370,378, 382, 385, 388, 399, 402, 409, 438, 449, 455, 459, 468: n = A,T,C or G
cnngnnccaaaattttgattttgnaaaaaaaaatttttcccccattgtggttttggncccaaataatnaaaaacccggnggccccntttgaaaaaatggncncanaaattncccgggtntttttttagggaacaaangggaagcccccccaataagggtntnncctcccntgagggggtgggggaaaaaaacccaattttttttttttttgcnnnagagccttaaaaatggcggtgcaagtagnaaaaanattgntaaattnttttcccagcaaggaanataattntaagcccctagggaaaaaattgcnttnttggggggaggtttataaacgnccnctctgggagtgtntgtcntatgtggttgananaaggattnaaanacnccntggtcgcctgnagnaccctcnggcttattaggattgggaaaccccagtcntggtaaatttnaggtcnggcnggttttttnntttnanccctgttttttgttaanatntttatcaanacaanangngccccgntgaanananacccttatcaggagtttntgattttgctcgggtcctgtttnttcaaaagcatgtcatttttgntttgccttntgccctttnaagnatgtgatntttgtgacctactccctgttcanacnccccnccccttttaaaatccctaataaaaacttgctggttttgtggctcaggggggcatcatggacctaccaanacgtgatgtcncccccggtggcccagctgtaaaaaaaaaaaaa

SEQ ID NO: 91 - DNA, 780 bp, HERV-K
  misc_feature 2, 7, 16, 17, 47, 67, 78, 82, 88, 92, 98, 110, 113, 121,129, 156, 173, 220, 224, 307, 385, 397, 432, 480, 501: n = A,T,C or G
  misc_feature 508, 525, 532, 543, 545, 586, 591, 610, 638: n = A,T,C or G
  misc_feature 2, 7, 16, 17, 47, 67, 78, 82, 88, 92, 98, 110, 113, 121,129, 156, 173, 220, 224, 307, 385, 397, 432, 480, 501: n = A,T,C or G
  misc_feature 508, 525, 532, 543, 545, 586, 591, 610, 638: n = A,T,C or G
ancccantttttggtnncaaaatttgaattgtaaaaaacaatggttnccaccattgttgttttgganccaaatattgnaaantccagnggcncccttngaaaaatggaancangaaataanccaggtgnttttttagggggaaccaaaaggaaagnccaccccataaagggtnttgacttgccttgagggggtcgggggcaaaaaaagccaatatttttntttntttgcagagagcctataaatggacgtgcaaagtaggaaaaatattgctaaattcttttcctagcaaggaatataatattaagnccctaggaaagaattgcattcctggggggaggtctataaacggccgctctgggagtgtctgtcctatgtggttaaganaaggattgaganacgcccctggtcgcctgcagtaccctcaggcttantaggattgggaaaccccagtcctggtaaatttgaggtcaggccggttntttgctttgaaccctgttttntgttaanatgtttatcaagacaanacgtgcnccgctgaacananacccttatcaggagtttctgattttgctctggtcctgtttnttcanaagcatgtcatctttgctntgccttctgccctttgaagcatgtgatntttgtgacctactccctgttcatacacccctccccttttaaaatccctaataaaaacttgctggttttgtggctcaggggggcatcatggacctaccaatacgtgatgtcacccccggtggcccagctgtaaaaaaaaaaaa

SEQ ID NO: 92 - protein, 8 aa, HERV-K
ESSKLSIT

SEQ ID NO: 93 - protein, 12 aa, HERV-K
LKEQSWLPSLQC

SEQ ID NO: 94 - protein, 270 aa, HERV-K
QDFNQSINIVSDSAYVVQATKDIERALIKYIMDDQLNPLFNLLQQNVRKRNFPFYITHIRAHTNLPGPLTKANEQADLLVSSAFMEAQELHALTHVNAIGLKNKFDITWKQTKNIVQHCTQCQILHLATQEARVNPRGLCPNVLWQMDVMHVPSFGKLSFVHVTVDTYSHFIWATCQTGESTSHVKRHLLSCFPVMGVPEKVKTDNGPGYCSKAVQKFLNQWKITHTIGILYNSQGQAIIERTNRTLKAQLVKQKKGKDRSITLPRCNLI

SEQ ID NO: 95 - protein, 98 aa, HERV-K
MSNLFSFLRGDSELNSTLTPEATKEIKLIEEKIRSAQVNRIDHLAPLQILIFATAHSLTGIIVQNTDLVEWSFLPHSTIKTFTLYLDQMATLIGQGRL

SEQ ID NO: 96 - protein, 92 aa, HERV-K
IITLCGNDPDKITVPFNKQQVRQAFINSGAWQIGLADFVGIIDNRYPKTKIFQFLKLTTWILPKVTKHKPLKNAVFTDGSSNGKVAYTGPKE

SEQ ID NO: 97 - protein, 138 aa, HERV-K
TKKRKRQEYNTPQMQLNLALYTLNVLNIYRNQTTTSAEQHLTGKRNSFGKLIWWKDNKNKTWEMGKVITWGRGFACVSPGENQLPVWIPTRHLKFYNELTGDAKKSVEMPQSTRQVNKMVISEEQKKLPSIKEAELPI

SEQ ID NO: 98 - protein, 79 aa, HERV-K [sequence omitted]

SEQ ID NO: 99 - DNA, 2078 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgtctctcatccctcctgacgagaaatacccacaggtgtggaggggctggcccccttcatctgatgcccaatgtgggtgcctttctctagggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 100 - DNA, 2112 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgtctctcatccctcctgacgagaaatacccacaggtgtggaggggctggcccccttcatctgatgcccaatgtgggtgcctttctctagggtgaaggtactctacagtgtggtcattgaggacaagttgacgagagagtcccaagtacgtccacggtcagccttgcggagaaaatcagcttcctgtttggatacccactagacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 101 - DNA, 1999 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgtctctcatccctcctgacgagaaatacccacaggtgtggaggggctggcccccttcatctgatgcccaatgtgggtgcctttctctagggtgaaggtactctacagtgtggtcattgaggacaagttgacgagagagtcccaagtacgtccacggtcagccttgcgtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 102 - DNA, 1911 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgggtgaaggtactctacagtgtggtcattgaggacaagttgacgagagagtcccaagtacgtccacggtcagccttgcgtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 103 - DNA, 1990 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 104 - DNA, 2024 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgggtgaaggtactctacagtgtggtcattgaggacaagttgacgagagagtcccaagtacgtccacggtcagccttgcggagaaaatcagcttcctgtttggatacccactagacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 105 - DNA, 2176 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccaggtaaacaaaatggtgatatcagaagaacagaaaaagttgccttccatcaaggaagcagagttgccaatataggcacaattaaagaagctgacacagttagctaaaaaaaaaagcctagagaatacaaaggtgacaccaactccagagaatatgctgcttgcagctctgatgattgtatcaacggtgtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 106 - DNA, 2210 bp, HERV-K
gagataggagaaaactgccttagggctggaggtgggacatgctggcggcaatactgctctttaaggcattgagatgtttatgtatatgcacatcaaaagcacagcacttttttctttaccttgtttatgatgcagagacatttgttcacatgttttcctgctggccctctccccactattaccctattgtcctgccacatccccctctccgagatggtagagataatgatcaataaatactgagggaactcagagaccggtgcggcgcgggtcctccatatgctgagcgccggtcccctgggcccacttttctttctctatactttgtctctgttgtctttcttttctcaagtctctcgttccacctgaggagaaatgcccacagctgtggaggcgcaggccactccatctggtgcccaacgtggatgcttttctctagggtgaagggactctcgagtgtggtcattgaggacaagtcaacgagagattcccgagtacgtctacagtgagccttgtgggtgaaggtactctacagtgtggtcattgaggacaagttgacgagagagtcccaagtacgtccacggtcagccttgcggagaaaatcagcttcctgtttggatacccactagacatttaaagttctacaatgaactcactggagatgcaaagaaaagtgtggagatggagacaccccaatcgactcgccaggtaaacaaaatggtgatatcagaagaacagaaaaagttgccttccatcaaggaagcagagttgccaatataggcacaattaaagaagctgacacagttagctaaaaaaaaaagcctagagaatacaaaggtgacaccaactccagagaatatgctgcttgcagctctgatgattgtatcaacggtgtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 107 - DNA, 1907 bp, HERV-K
ttttgcttgtgtttcaccaggagaaaatcagcttcctgtttggatacccactagacatttaaagttctacaatgaactcactggagatgcaaagaaaagtgtggagatggagacaccccaatcgactcgccaggtaaacaaaatggtgatatcagaagaacagaaaaagttgccttccatcaaggaagcagagttgccaatataggcacaattaaagaagctgacacagttagctaaaaaaaaaagcctagagaatacaaaggtgacaccaactccagagaatatgctgcttgcagctctgatgattgtatcaacggtggtaagtcttcccaagtctgcaggagcagctgcagctaattatacttactgggcctatgtgcctttcccacccttaattcgggcagttacatagatggataatcctattgaagtagatgttaataatagtgcatgggtgcctggccccacagatgactgttgccctgcccaacctgaagaaggaatgatgatgaatatttccattgggtatccttatcctcctgtttgcctagggaaggcaccaggatgcttaatgcctacaacccaaaattgtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 108 - DNA, 1959 bp, HERV-K
ttttgcttgtgtttcaccaggagaaaatcagcttcctgtttggatacccactagacatttaaagttctacaatgaactcactggagatgcaaagaaaagtgtggagatggagacaccccaatcgactcgccaggtaaacaaaatggtgatatcagaagaacagaaaaagttgccttccatcaaggaagcagagttgccaatataggcacaattaaagaagctgacacagttagctaaaaaaaaaagcctagagaatacaaaggtgacaccaactccagagaatatgctgcttgcagctctgatgattgtatcaacggtggtaagtcttcccaagtctgcaggagcagctgcagctaattatacttactgggcctatgtgcctttcccacccttaattcgggcagttacatagatggataatcctattgaagtagatgttaataatagtgcatgggtgcctggccccacagatgactgttgccctgcccaacctgaagaaggaatgatgatgaatatttccattgggtatccttatcctcctgtttgcctagggaaggcaccaggatgcttaatgcctacaacccaaaattggttggtagaagtacctacagtcagtgctaccagtagatttacttatcacatgtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 109 - DNA, 1936 bp, HERV-K
gagaagaaaaccaccctgtggctggaggtgagatatgctagcggcaatgctgctctgttactctttgctacactgagatgtttgggtggagagaagcataaatctggcctatgtgcacatctgggcacagaacctccccttgaacttgtgacacagattcctttgttcacatgttttcctgctgaccttctccccactatcgccctgttctcccaccgcattccccttgctgagatagtgaaaatagtaatctgtagataccaagggaactcagagaccatggccggtgcacatcctccgtacgctgagcgctggtcccctgggcccattgttctttctctatactttgtctctgtgtcttatttctttcctcagtctctcatccctcctgacgagaaatacccacaggtgtggaggggctggcccccttcatctgatgcccaatgtgggtgcctttctctagggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaattcttttcctagcaaggaatataatactaagaccctagggaaagaattgcattcctggggggaggtctataaacggccgctctgggagtgtctgtcctatgtggttgagataaggactgagatacgccctggtctcctgcagtaccctcaggcttactaggattgggaaaccccagtcctggtaaatttgaggtcaggccggttctttgctctgaaccctgttttctgttaagatgtttatcaagacaatacatgcaccgctgaacatagacccttatcaggagtttctgattttgctctggtcctgtttcttcagaagcatgtcatctttgctctgccttctgccctttgaagcatgtgatctttgtgacctactccctgttcatacacccctccccttttaaaatccctaataaaaacttgctggttttgtggctcaggggggcatcatggacctaccaatacgtgatgtcacccccggtggcccagctgtaaaa

SEQ ID NO: 110 - protein, 14 aa, HERV-K
TAMNSPATQDAALY

SEQ ID NO: 111 - DNA, 69 bp, HERV-K
acgcaggttagacaagcacaaaccccaagagaaaatcaagtagaaagggacagagtctctatcccggca

SEQ ID NO: 112 - DNA, 51 bp, HERV-K
cccacagcgatggcgtctaattcaccagcaacacaggacgcggcgctgtat

SEQ ID NO: 113 - DNA, 780 bp, HERV-K
  misc_feature 1, 8, 17, 18, 21, 29, 31, 34, 653, 687, 727, 728, 739,742, 774, 775, 776, 777, 780: n = A,T,C or G
  misc_feature 1, 8, 17, 18, 21, 29, 31, 34, 653, 687, 727, 728, 739,742, 774, 775, 776, 777, 780: n = A,T,C or G
ncggcctncggctgcgnntantcgacagnanggngggtaggccttattttagggagatcaagtctaaatttgaagggagtccaaattcatactggggtaatttattcagattataaagggggaattcagttagtgtcagctccactgttccccggagtgccaatccaggtgatagaattgctcaattactgcttttgccttatgttaaaattggggaaaacaaaacggaaagaacaggagggtttggaagtaccaaccctgcaggaaaagctgcttattgggctaatcaggtctcagaagatagacccgtgtgtacagtcactattcagggaaagagtttgaaggattagtggatacccaggctgattctatcatcggcataggtaccgcctcagaagtgtatcaaagtgccatgattttacattgtctaggatctgataatcaagaaagtacggttcagcctgtgatcacttcattccaatcaatttatggggccgagacttgttacaacaatggcatgcagagattactatcccagcctccctatacagccccaggaatcaaaaaatcatgactaaaatgggatagctccctaaaaagggactaggaaagaaagaagtcccaattgaggctgaaaaaatcaaaaaagaaangaatagggcatcctttttaggagcgtcactgtanagcctccaaacccattcattaacttgggaaaaaaaactgnntggtaaatcancanccgcttccaaaaaaaaaaaaaaaaaaaacccnnnnccn

SEQ ID NO: 114 - DNA, 1058 bp, HERV-K
atctttaccctgtataaacatctttctcttcccagtatttctaagcatgtgacaatgaatatgcaaaggaagcgcagcagtccaccaggtgtgggatatgtgtggcacaattcaagacaatgattaaacctccacttgatgttgcaaaagagattttgaaaaatttgctttcaccacaccagcctaaataataaagaaccagccaccaggtttcagtggaaagtattgcctcagggaatgcttaatagttcaactatttgtcagctcaagctctgcaaccagttagagacaagttttcagactgttacatcgttcactatgttgatattttgtgtgctgcagaaacgagagacaaattaattgaccgttacacatttctgcagacagaggttgccaacgcgggactgacaataacatctgataagattcaagcctctactcctttccgttacttgggaatgcaggtagaggaaaggaaaattaaaccacaaaaaaatagaaataagaaaagacacattaaaagcattaaatgagtttcaaaagttgctaggagatactaattggatttggagatattaattggatttggccaactctaggcattcctacttatgccatgtcaaatttgttctctttcttaagaggggactcggaattaaatagtgaaagaacgttaactccagaggcaactaaagaaattaaattaattgaagaaaaaattcggtcagcacaagtaaatagaatagatcacttggccccactccaaattttgatttttactactgcacattccctaacaggcatcattgttcaaaacacagatcttgtggagtggtccttccttcctcacagtacaattaagacttttacattgtacttggatcaaatggctacattaattggtcagggaagattatgaataataacattgtgtggaaatgacccagataaaatcactgttcctttcaacaagcaacaggttagacaagcctttatcaattctggtgcatggcagattggtcttgccgattttgtgggaattattgacaatcgttacc

SEQ ID NO: 115 - DNA, 842 bp, HERV-K
ccaaaagaatgagtcatcaaaactcagtatcacttgactcaaagagcagagttggttgccgtcattacagtgttaacaagattttaatcagtctattaacattgtatcagattctgcatatgtagtacaggctacaaaggatattgagagagccctaatcaaatacattatggatgatcagttaaacccgctgtttaatttgttacaacaaaatgtaagaaaaagaaatttcccattttatattactcatattcgagcacacactaatttaccagggcctttaactaaagcaaatgaacaagctgactcgctagtatcatctgcattcatggaagcacaagaccttcatgccttgactcatgtaaatgcaataggattaaaaaataaatttaatatcacatggaaacagacaaaaaatattgtacaacattgcacccagtgtcagattctacacctggccactcaggaggcaagagttaatcccagaggtctatgtcctaatgtgttatggcaaatggatgtcatgcacgtaccttcatttggaaaattgtcatttgtccatgtgacagttgatacttattcacatttcatatgggcaacctgccagacaggagaaagtacttcccatgttaagagacatttattatcttgttttcctgtcatgggagttccagaaaaagttaaaacagacaatgggccaggttactgtagtaaagcagttcaaaaattcttaaatcagtggaaaattacacatacaataggaattctctataattcccaaggacaggccataattgaaagaactaatagaacactcaaagctcaattggttaaacaaaaaa

SEQ ID NO: 116 - DNA, 661 bp, HERV-K
ccttacggccgggaagggccggaaagggagtcaagcaggagcgtctgtccgaacggaggctaggtaagaatatttcaccatgaaaatgttaaaagacataaaggaaggagctaaacaatatggacccaactctccttatatgagaacgttattagattccattgctcatggaaatagacttattccttatgattgggaaattttacctaaatcttccctttcaccctctcagtatctacagtttaaaacctggtggattgatggagtacaagaacaggtacggaaaaatcaggctacttatcctgttgttaatatagatgcagaccaattgctaggaacacgtccaaattggagcactattaaccaacaatcagtaatgcaaaatgaggctattgaacaactaggggctatttgcctcagggcctgggaaaagattcaggacccaggaaccagttagagacagttttcagactgttatatcattcattatgttgatgatattttgtgtgctgcagaaacaagagacaaattaattgacttttacatgtttctgcagacagaggttgcaaacacaggcctgacaatagcatctgataagattcagacctccactccttttaattatttgggaatgcaggtagaggaaagaaaaattaaacca

SEQ ID NO: 117 - DNA, 711 bp, HERV-K
ctgaaaaaaatcaaaaaagaaaaggaatagggcatcctttttaggagcggtcactgtagagcctccaaaacccattccattaacttgggggaaaaaaaaacaactgtatggtaaatcagcagcgcttccaaaacaaaaactggaggctttacatttattagcaaagaaacaattagaaaaaggacattgagccttcattttcgccttggaattctgtttgtaattcagaaaaaatccggcagatggcgtataatgccgtaattcaacccatgggggctctcccaccccggttgccctctccagccatggtcccctttaattataattgatctgaaggattgcttttttaccattcctctggcaaaacaggattttgagaaatttgcttttaccacaccagcctaaataataaagaaccagccaccaggtttcagtggaaagtattgcctcagggaatgcttaatagttcaactatttgtcagctcaagctctgcaaccagttagagacaagttttcagactgttacatcgttcactatgttgatattttgtgtgctgcagaaacgagagacaaattaattgaccgttacacatttctgcagacagaggttgccaacgcgggactgacaataacatctgataagattcaaacctctactcctttccgttacttgggaatgcaggtagaggaaaggaaaattaaaccacaaaa

SEQ ID NO: 118 - DNA, 838 bp, HERV-K
acaacaatggcatgcagagattactatcccagcctccctatacagccccaggaatcaaaaaatcatgactaaaatgggatagctccctaaaaagggactaggaaagaaagaagtcccaattgaggctgaaaaaaattaaaaaagaaaaggaatagggcatcctttttaggagcggtcactgtagagcctccaaaacccattccattaacttgggaaaaaaaaaactgtatggtaaatcagcagccgcttccaaaacaaaagctggaggccttacacttattagcaaagaaaccattagaaaaaggacattgagccttcattttcgccttggaattctgtttgtgattcagaaaaaatccggcagatggcgtatgctaactgagccattaatgccgtaattcaacccatgggggctctcccaccccggttgccctctccagccatggtcccctttaattataattgatctgaaggattgcttttttaccattcctctggcaaaacaggattttgaaaaatttgcttttaccacaccagcctaaataataaagaaccagccaccaggtttcagtggaaagtattgcctcagggaatgcttaatagttcaactatttgtcagctcaagctctgcaaccagttagagacaagttttcagactgttacatcgttcactatgttgatattttgtgtgctgcagaaacgagagacaaattaattgaccgttacacatttctgcagacagaggttgccaacgcggggctgacaataacatctgataagattcaaacctctactcctttccgttacttgggaatgcaggtagaggaaaggaaaatta

SEQ ID NO: 119 - DNA, 762 bp, HERV-K
cattagaaaaaggacattgagccttcattttcgccttggaattctgtttgtaattcagaaaaaatccggcagatggcgtatgctaactgagccattaatgccgtaattcaacccatgggggctctcccaccccggttgccctctccagccatggtcccctttaattataattgatctgaaggattgcttttttaccattcctctggcaaaacaggattttgaaaaatttgcttttaccacaccagcctaaataataaagaaccagccaccaggtttcagtggaaagtattgcctcagggaatgcttaatagttcaactatttgtcagctcaagctctgcaaccagttagagacaagttttcagactgttacatcgttcactatgttgatattttgtgtgctgcagaaacgagagacaaattaattgaccgttacacatttctgcagacagaggttgccaacgcgggactgacaataacatctgataagattcaaacctctactcctttccgttacttgggaatgcaggtagaggaaaggaaaattaaaccacaaaaaatagaaataagaaaagacacattaaaagcattaaatgagtttcaaaagttgctaggagatactaattggatttggagatattaattggatttggccaactctaggcattcctacttatgccatgtcaaatttgtactctttcttaagaggggactcggaattaaatagtgaaagaacgttaactccagaggcaactaaagaaaaa

SEQ ID NO: 120 - DNA, 25 bp, HERV-K
actgagataggagaaaactgcctta

SEQ ID NO: 121 - DNA, 25 bp, HERV-K
gataggagaaaactgccttagggct

SEQ ID NO: 122 - DNA, 25 bp, HERV-K
gagaaaactgccttagggctggagg

SEQ ID NO: 123 - DNA, 25 bp, HERV-K
aactgccttagggctggaggtggga

SEQ ID NO: 124 - DNA, 25 bp, HERV-K
ccttagggctggaggtgggacatgc

SEQ ID NO: 125 - DNA, 25 bp, HERV-K
gggctggaggtgggacatgctggcg

SEQ ID NO: 126 - DNA, 25 bp, HERV-K
ggaggtgggacatgctggcggcaat

SEQ ID NO: 127 - DNA, 25 bp, HERV-K
tgggacatgctggcggcaatactgc

SEQ ID NO: 128 - DNA, 25 bp, HERV-K
catgctggcggcaatactgctcttt

SEQ ID NO: 129 - DNA, 25 bp, HERV-K
tggcggcaatactgctctttaaggc

SEQ ID NO: 130 - DNA, 25 bp, HERV-K
gcaatactgctctttaaggcattga

SEQ ID NO: 131 - DNA, 25 bp, HERV-K
actgctctttaaggcattgagatgt

SEQ ID NO: 132 - DNA, 25 bp, HERV-K
tctttaaggcattgagatgtttatg

SEQ ID NO: 133 - DNA, 25 bp, HERV-K
aaggcattgagatgtttatgtatat

SEQ ID NO: 134 - DNA, 25 bp, HERV-K
attgagatgtttatgtatatgcaca

SEQ ID NO: 135 - DNA, 25 bp, HERV-K
gatgtttatgtatatgcacatcaaa

SEQ ID NO: 136 - DNA, 25 bp, HERV-K
ttatgtatatgcacatcaaaagcac

SEQ ID NO: 137 - DNA, 25 bp, HERV-K
tatatgcacatcaaaagcacagcac

SEQ ID NO: 138 - DNA, 25 bp, HERV-K
gcacatcaaaagcacagcacttttt

SEQ ID NO: 139 - DNA, 25 bp, HERV-K
tcaaaagcacagcacttttttcttt

SEQ ID NO: 140 - DNA, 25 bp, HERV-K
agcacagcacttttttctttacctt

SEQ ID NO: 141 - DNA, 25 bp, HERV-K
agcacttttttctttaccttgttta

SEQ ID NO: 142 - DNA, 25 bp, HERV-K
ttttttctttaccttgtttatgatg

SEQ ID NO: 143 - DNA, 25 bp, HERV-K
tctttaccttgtttatgatgcagag

SEQ ID NO: 144 - DNA, 25 bp, HERV-K
accttgtttatgatgcagagacatt

SEQ ID NO: 145 - DNA, 25 bp, HERV-K
gtttatgatgcagagacatttgttc

SEQ ID NO: 146 - DNA, 25 bp, HERV-K
tgatgcagagacatttgttcacatg

SEQ ID NO: 147 - DNA, 25 bp, HERV-K
cagagacatttgttcacatgttttc

SEQ ID NO: 148 - DNA, 25 bp, HERV-K
acatttgttcacatgttttcctgct

SEQ ID NO: 149 - DNA, 25 bp, HERV-K
tgttcacatgttttcctgctggccc

SEQ ID NO: 150 - DNA, 25 bp, HERV-K
acatgttttcctgctggccctctcc

SEQ ID NO: 151 - DNA, 25 bp, HERV-K
ttttcctgctggccctctccccact

SEQ ID NO: 152 - DNA, 25 bp, HERV-K
ctgctggccctctccccactattac

SEQ ID NO: 153 - DNA, 25 bp, HERV-K
ggccctctccccactattaccctat

SEQ ID NO: 154 - DNA, 25 bp, HERV-K
tctccccactattaccctattgtcc

SEQ ID NO: 155 - DNA, 25 bp, HERV-K
ccactattaccctattgtcctgcca

SEQ ID NO: 156 - DNA, 25 bp, HERV-K
attaccctattgtcctgccacatcc

SEQ ID NO: 157 - DNA, 25 bp, HERV-K
cctattgtcctgccacatccccctc

SEQ ID NO: 158 - DNA, 25 bp, HERV-K
tgtcctgccacatccccctctccga

SEQ ID NO: 159 - DNA, 25 bp, HERV-K
tgccacatccccctctccgagatgg

SEQ ID NO: 160 - DNA, 25 bp, HERV-K
catccccctctccgagatggtagag

SEQ ID NO: 161 - DNA, 25 bp, HERV-K
ccctctccgagatggtagagataat

SEQ ID NO: 162 - DNA, 25 bp, HERV-K
tccgagatggtagagataatgatca

SEQ ID NO: 163 - DNA, 25 bp, HERV-K
gatggtagagataatgatcaataaa

SEQ ID NO: 164 - DNA, 25 bp, HERV-K
tagagataatgatcaataaatactg

SEQ ID NO: 165 - DNA, 25 bp, HERV-K
ataatgatcaataaatactgaggga

SEQ ID NO: 166 - DNA, 25 bp, HERV-K
gatcaataaatactgagggaactca

SEQ ID NO: 167 - DNA, 25 bp, HERV-K
ataaatactgagggaactcagagac

SEQ ID NO: 168 - DNA, 25 bp, HERV-K
tactgagggaactcagagaccggtg

SEQ ID NO: 169 - DNA, 25 bp, HERV-K
agggaactcagagaccggtgcggcg

SEQ ID NO: 170 - DNA, 25 bp, HERV-K
actcagagaccggtgcggcgcgggt

SEQ ID NO: 171 - DNA, 25 bp, HERV-K
gagaccggtgcggcgcgggtcctcc

SEQ ID NO: 172 - DNA, 25 bp, HERV-K
cggtgcggcgcgggtcctccatatg

SEQ ID NO: 173 - DNA, 25 bp, HERV-K
cggcgcgggtcctccatatgctgag

SEQ ID NO: 174 - DNA, 25 bp, HERV-K
cgggtcctccatatgctgagcgccg

SEQ ID NO: 175 - DNA, 25 bp, HERV-K
cctccatatgctgagcgccggtccc

SEQ ID NO: 176 - DNA, 25 bp, HERV-K
atatgctgagcgccggtcccctggg

SEQ ID NO: 177 - DNA, 25 bp, HERV-K
ctgagcgccggtcccctgggcccac

SEQ ID NO: 178 - DNA, 25 bp, HERV-K
cgccggtcccctgggcccacttttc

SEQ ID NO: 179 - DNA, 25 bp, HERV-K
gtcccctgggcccacttttctttct

SEQ ID NO: 180 - DNA, 25 bp, HERV-K
ctgggcccacttttctttctctata

SEQ ID NO: 181 - DNA, 25 bp, HERV-K
cccacttttctttctctatactttg

SEQ ID NO: 182 - DNA, 25 bp, HERV-K
ttttctttctctatactttgtctct

SEQ ID NO: 183 - DNA, 25 bp, HERV-K
tttctctatactttgtctctgttgt

SEQ ID NO: 184 - DNA, 25 bp, HERV-K
ctatactttgtctctgttgtctttc

SEQ ID NO: 185 - DNA, 25 bp, HERV-K
ctttgtctctgttgtctttcttttc

SEQ ID NO: 186 - DNA, 25 bp, HERV-K
tctctgttgtctttcttttctcaag

SEQ ID NO: 187 - DNA, 25 bp, HERV-K
gttgtctttcttttctcaagtctct

SEQ ID NO: 188 - DNA, 25 bp, HERV-K
ctttcttttctcaagtctctcgttc

SEQ ID NO: 189 - DNA, 25 bp, HERV-K
ttttctcaagtctctcgttccacct

SEQ ID NO: 190 - DNA, 25 bp, HERV-K
tcaagtctctcgttccacctgagga

SEQ ID NO: 191 - DNA, 25 bp, HERV-K
tctctcgttccacctgaggagaaat

SEQ ID NO: 192 - DNA, 25 bp, HERV-K
cgttccacctgaggagaaatgccca

SEQ ID NO: 193 - DNA, 25 bp, HERV-K
cacctgaggagaaatgcccacagct

SEQ ID NO: 194 - DNA, 25 bp, HERV-K
gaggagaaatgcccacagctgtgga

SEQ ID NO: 195 - DNA, 25 bp, HERV-K
gaaatgcccacagctgtggaggcgc

SEQ ID NO: 196 - DNA, 25 bp, HERV-K
gcccacagctgtggaggcgcaggcc

SEQ ID NO: 197 - DNA, 25 bp, HERV-K
cagctgtggaggcgcaggccactcc

SEQ ID NO: 198 - DNA, 25 bp, HERV-K
gtggaggcgcaggccactccatctg

SEQ ID NO: 199 - DNA, 25 bp, HERV-K
ggcgcaggccactccatctggtgcc

SEQ ID NO: 200 - DNA, 25 bp, HERV-K
aggccactccatctggtgcccaacg

SEQ ID NO: 201 - DNA, 25 bp, HERV-K
actccatctggtgcccaacgtggat

SEQ ID NO: 202 - DNA, 25 bp, HERV-K
atctggtgcccaacgtggatgcttt

SEQ ID NO: 203 - DNA, 25 bp, HERV-K
gtgcccaacgtggatgcttttctct

SEQ ID NO: 204 - DNA, 25 bp, HERV-K
caacgtggatgcttttctctagggt

SEQ ID NO: 205 - DNA, 25 bp, HERV-K
tggatgcttttctctagggtgaagg

SEQ ID NO: 206 - DNA, 25 bp, HERV-K
gcttttctctagggtgaagggactc

SEQ ID NO: 207 - DNA, 25 bp, HERV-K
tctctagggtgaagggactctcgag

SEQ ID NO: 208 - DNA, 25 bp, HERV-K
agggtgaagggactctcgagtgtgg

SEQ ID NO: 209 - DNA, 25 bp, HERV-K
gaagggactctcgagtgtggtcatt

SEQ ID NO: 210 - DNA, 25 bp, HERV-K
gactctcgagtgtggtcattgagga

SEQ ID NO: 211 - DNA, 25 bp, HERV-K
tcgagtgtggtcattgaggacaagt

SEQ ID NO: 212 - DNA, 25 bp, HERV-K
tgtggtcattgaggacaagtcaacg

SEQ ID NO: 213 - DNA, 25 bp, HERV-K
tcattgaggacaagtcaacgagaga

SEQ ID NO: 214 - DNA, 25 bp, HERV-K
gaggacaagtcaacgagagattccc

SEQ ID NO: 215 - DNA, 25 bp, HERV-K
caagtcaacgagagattcccgagta

SEQ ID NO: 216 - DNA, 25 bp, HERV-K
caacgagagattcccgagtacgtct

SEQ ID NO: 217 - DNA, 25 bp, HERV-K
agagattcccgagtacgtctacagt

SEQ ID NO: 218 - DNA, 25 bp, HERV-K
ttcccgagtacgtctacagtgagcc

SEQ ID NO: 219 - DNA, 25 bp, HERV-K
gagtacgtctacagtgagccttgtg

SEQ ID NO: 220 - DNA, 25 bp, HERV-K
gagaaaatcagcttcctgtttggat

SEQ ID NO: 221 - DNA, 25 bp, HERV-K
aatcagcttcctgtttggataccca

SEQ ID NO: 222 - DNA, 25 bp, HERV-K
gcttcctgtttggatacccactaga

SEQ ID NO: 223 - DNA, 25 bp, HERV-K
ctgtttggatacccactagacattt

SEQ ID NO: 224 - DNA, 25 bp, HERV-K
acccactagacatttaaagttctac

SEQ ID NO: 225 - DNA, 25 bp, HERV-K
ctagacatttaaagttctacaatga

SEQ ID NO: 226 - DNA, 25 bp, HERV-K
ggtgaaggtactctacagtgtggtc

SEQ ID NO: 227 - DNA, 25 bp, HERV-K
aggtactctacagtgtggtcattga

SEQ ID NO: 228 - DNA, 25 bp, HERV-K
ctctacagtgtggtcattgaggaca

SEQ ID NO: 229 - DNA, 25 bp, HERV-K
cagtgtggtcattgaggacaagttg

SEQ ID NO: 230 - DNA, 25 bp, HERV-K
tggtcattgaggacaagttgacgag

SEQ ID NO: 231 - DNA, 25 bp, HERV-K
attgaggacaagttgacgagagagt

SEQ ID NO: 232 - DNA, 25 bp, HERV-K
ggacaagttgacgagagagtcccaa

SEQ ID NO: 233 - DNA, 25 bp, HERV-K
agttgacgagagagtcccaagtacg

SEQ ID NO: 234 - DNA, 25 bp, HERV-K
acgagagagtcccaagtacgtccac

SEQ ID NO: 235 - DNA, 25 bp, HERV-K
agagtcccaagtacgtccacggtca

SEQ ID NO: 236 - DNA, 25 bp, HERV-K
cccaagtacgtccacggtcagcctt

SEQ ID NO: 237 - DNA, 25 bp, HERV-K
gtacgtccacggtcagccttgcgac

SEQ ID NO: 238 - DNA, 25 bp, HERV-K
tccacggtcagccttgcgacattta

SEQ ID NO: 239 - DNA, 25 bp, HERV-K
ggtcagccttgcgacatttaaagtt

SEQ ID NO: 240 - DNA, 25 bp, HERV-K
gccttgcgacatttaaagttctaca

SEQ ID NO: 241 - DNA, 25 bp, HERV-K
gcgacatttaaagttctacaatgaa

SEQ ID NO: 242 - DNA, 25 bp, HERV-K
atttaaagttctacaatgaactcac

SEQ ID NO: 243 - DNA, 25 bp, HERV-K
aagttctacaatgaactcactggag

SEQ ID NO: 244 - DNA, 25 bp, HERV-K
ctacaatgaactcactggagatgca

SEQ ID NO: 245 - DNA, 25 bp, HERV-K
atgaactcactggagatgcaaagaa

SEQ ID NO: 246 - DNA, 25 bp, HERV-K
ctcactggagatgcaaagaaaagtg

SEQ ID NO: 247 - DNA, 25 bp, HERV-K
tggagatgcaaagaaaagtgtggag

SEQ ID NO: 248 - DNA, 25 bp, HERV-K
atgcaaagaaaagtgtggagatgga

SEQ ID NO: 249 - DNA, 25 bp, HERV-K
aagaaaagtgtggagatggagacac

SEQ ID NO: 250 - DNA, 25 bp, HERV-K
aagtgtggagatggagacaccccaa

SEQ ID NO: 251 - DNA, 25 bp, HERV-K
tggagatggagacaccccaatcgac

SEQ ID NO: 252 - DNA, 25 bp, HERV-K
atggagacaccccaatcgactcgcc

SEQ ID NO: 253 - DNA, 25 bp, HERV-K
gacaccccaatcgactcgccagtct

SEQ ID NO: 254 - DNA, 25 bp, HERV-K
cccaatcgactcgccagtctacagg

SEQ ID NO: 255 - DNA, 25 bp, HERV-K
tcgactcgccagtctacaggtgtat

SEQ ID NO: 256 - DNA, 25 bp, HERV-K
tcgccagtctacaggtgtatccagc

SEQ ID NO: 257 - DNA, 25 bp, HERV-K
agtctacaggtgtatccagcagctc

SEQ ID NO: 258 - DNA, 25 bp, HERV-K
acaggtgtatccagcagctccaaag

SEQ ID NO: 259 - DNA, 25 bp, HERV-K
tgtatccagcagctccaaagagaca

SEQ ID NO: 260 - DNA, 25 bp, HERV-K
ccagcagctccaaagagacagcaac

SEQ ID NO: 261 - DNA, 25 bp, HERV-K
agctccaaagagacagcaaccagca

SEQ ID NO: 262 - DNA, 25 bp, HERV-K
caaagagacagcaaccagcaagaat

SEQ ID NO: 263 - DNA, 25 bp, HERV-K
agacagcaaccagcaagaatgggcc

SEQ ID NO: 264 - DNA, 25 bp, HERV-K
gcaaccagcaagaatgggccatagt

SEQ ID NO: 265 - DNA, 25 bp, HERV-K
cagcaagaatgggccatagtgacga

SEQ ID NO: 266 - DNA, 25 bp, HERV-K
agaatgggccatagtgacgatggtg

SEQ ID NO: 267 - DNA, 25 bp, HERV-K
gggccatagtgacgatggtggtttt

SEQ ID NO: 268 - DNA, 25 bp, HERV-K
atagtgacgatggtggttttgtcaa

SEQ ID NO: 269 - DNA, 25 bp, HERV-K
gacgatggtggttttgtcaaaaaga

SEQ ID NO: 270 - DNA, 25 bp, HERV-K
tggtggttttgtcaaaaagaaaagg

SEQ ID NO: 271 - DNA, 25 bp, HERV-K
gttttgtcaaaaagaaaaggggggg

SEQ ID NO: 272 - DNA, 25 bp, HERV-K
gtcaaaaagaaaagggggggatatg

SEQ ID NO: 273 - DNA, 25 bp, HERV-K
aaagaaaagggggggatatgtaagg

SEQ ID NO: 274 - DNA, 25 bp, HERV-K
aaagggggggatatgtaaggaaaag

SEQ ID NO: 275 - DNA, 25 bp, HERV-K
gggggatatgtaaggaaaagagaga

SEQ ID NO: 276 - DNA, 25 bp, HERV-K
atatgtaaggaaaagagagatcaga

SEQ ID NO: 277 - DNA, 25 bp, HERV-K
taaggaaaagagagatcagactttc

SEQ ID NO: 278 - DNA, 25 bp, HERV-K
aaaagagagatcagactttcactgt

SEQ ID NO: 279 - DNA, 25 bp, HERV-K
agagatcagactttcactgtgtcta

SEQ ID NO: 280 - DNA, 25 bp, HERV-K
tcagactttcactgtgtctatgtag

SEQ ID NO: 281 - DNA, 25 bp, HERV-K
ctttcactgtgtctatgtagaaaag

SEQ ID NO: 282 - DNA, 25 bp, HERV-K
actgtgtctatgtagaaaaggaaga

SEQ ID NO: 283 - DNA, 25 bp, HERV-K
gtctatgtagaaaaggaagacataa

SEQ ID NO: 284 - DNA, 25 bp, HERV-K
tgtagaaaaggaagacataagaaac

SEQ ID NO: 285 - DNA, 25 bp, HERV-K
aaaaggaagacataagaaactccat

SEQ ID NO: 286 - DNA, 25 bp, HERV-K
gaagacataagaaactccattttga

SEQ ID NO: 287 - DNA, 25 bp, HERV-K
cataagaaactccattttgatctgt

SEQ ID NO: 288 - DNA, 25 bp, HERV-K
gaaactccattttgatctgtactaa

SEQ ID NO: 289 - DNA, 25 bp, HERV-K
tccattttgatctgtactaagaaaa

SEQ ID NO: 290 - DNA, 25 bp, HERV-K
tttgatctgtactaagaaaaattgt

SEQ ID NO: 291 - DNA, 25 bp, HERV-K
tctgtactaagaaaaattgttttgc

SEQ ID NO: 292 - DNA, 25 bp, HERV-K
actaagaaaaattgttttgccttga

SEQ ID NO: 293 - DNA, 25 bp, HERV-K
gaaaaattgttttgccttgagatgc

SEQ ID NO: 294 - DNA, 25 bp, HERV-K
attgttttgccttgagatgctgtta

SEQ ID NO: 295 - DNA, 25 bp, HERV-K
tttgccttgagatgctgttaatctg

SEQ ID NO: 296 - DNA, 25 bp, HERV-K
cttgagatgctgttaatctgtaact

SEQ ID NO: 297 - DNA, 25 bp, HERV-K
gatgctgttaatctgtaactttagc

SEQ ID NO: 298 - DNA, 25 bp, HERV-K
tgttaatctgtaactttagccccaa

SEQ ID NO: 299 - DNA, 25 bp, HERV-K
atctgtaactttagccccaaccctg

SEQ ID NO: 300 - DNA, 25 bp, HERV-K
taactttagccccaaccctgtgctc

SEQ ID NO: 301 - DNA, 25 bp, HERV-K
ttagccccaaccctgtgctcacgga

SEQ ID NO: 302 - DNA, 25 bp, HERV-K
cccaaccctgtgctcacggaaacat

SEQ ID NO: 303 - DNA, 25 bp, HERV-K
ccctgtgctcacggaaacatgtgct

SEQ ID NO: 304 - DNA, 25 bp, HERV-K
tgctcacggaaacatgtgctgtaag

SEQ ID NO: 305 - DNA, 25 bp, HERV-K
acggaaacatgtgctgtaaggttta

SEQ ID NO: 306 - DNA, 25 bp, HERV-K
aacatgtgctgtaaggtttaaggga

SEQ ID NO: 307 - DNA, 25 bp, HERV-K
gtgctgtaaggtttaagggatctag

SEQ ID NO: 308 - DNA, 25 bp, HERV-K
gtaaggtttaagggatctagggctg

SEQ ID NO: 309 - DNA, 25 bp, HERV-K
gtttaagggatctagggctgtgcag

SEQ ID NO: 310 - DNA, 25 bp, HERV-K
agggatctagggctgtgcaggatgt

SEQ ID NO: 311 - DNA, 25 bp, HERV-K
tctagggctgtgcaggatgtacctt

SEQ ID NO: 312 - DNA, 25 bp, HERV-K
ggctgtgcaggatgtaccttgttaa

SEQ ID NO: 313 - DNA, 25 bp, HERV-K
tgcaggatgtaccttgttaacaata

SEQ ID NO: 314 - DNA, 25 bp, HERV-K
gatgtaccttgttaacaatatgttt

SEQ ID NO: 315 - DNA, 25 bp, HERV-K
accttgttaacaatatgtttgcagg

SEQ ID NO: 316 - DNA, 25 bp, HERV-K
gttaacaatatgtttgcaggcagta

SEQ ID NO: 317 - DNA, 25 bp, HERV-K
caatatgtttgcaggcagtatgttt

SEQ ID NO: 318 - DNA, 25 bp, HERV-K
tgtttgcaggcagtatgtttggtaa

SEQ ID NO: 319 - DNA, 25 bp, HERV-K
gcaggcagtatgtttggtaaaagtc

SEQ ID NO: 320 - DNA, 25 bp, HERV-K
cagtatgtttggtaaaagtcatcgc

SEQ ID NO: 321 - DNA, 25 bp, HERV-K
tgtttggtaaaagtcatcgccattc

SEQ ID NO: 322 - DNA, 25 bp, HERV-K
ggtaaaagtcatcgccattctccat

SEQ ID NO: 323 - DNA, 25 bp, HERV-K
aagtcatcgccattctccattctcg

SEQ ID NO: 324 - DNA, 25 bp, HERV-K
atcgccattctccattctcgattaa

SEQ ID NO: 325 - DNA, 25 bp, HERV-K
cattctccattctcgattaaccagg

SEQ ID NO: 326 - DNA, 25 bp, HERV-K
tccattctcgattaaccaggggctc

SEQ ID NO: 327 - DNA, 25 bp, HERV-K
tctcgattaaccaggggctcaatgc

SEQ ID NO: 328 - DNA, 25 bp, HERV-K
attaaccaggggctcaatgcactgt

SEQ ID NO: 329 - DNA, 25 bp, HERV-K
ccaggggctcaatgcactgtggaaa

SEQ ID NO: 330 - DNA, 25 bp, HERV-K
ggctcaatgcactgtggaaagccac

SEQ ID NO: 331 - DNA, 25 bp, HERV-K
aatgcactgtggaaagccacaggaa

SEQ ID NO: 332 - DNA, 25 bp, HERV-K
actgtggaaagccacaggaacctct

SEQ ID NO: 333 - DNA, 25 bp, HERV-K
ggaaagccacaggaacctctgccca

SEQ ID NO: 334 - DNA, 25 bp, HERV-K
gccacaggaacctctgcccaagaaa

SEQ ID NO: 335 - DNA, 25 bp, HERV-K
aggaacctctgcccaagaaagcctg

SEQ ID NO: 336 - DNA, 25 bp, HERV-K
cctctgcccaagaaagcctggctgt

SEQ ID NO: 337 - DNA, 25 bp, HERV-K
tgtggggaaaagaaagagagatcag

SEQ ID NO: 338 - DNA, 25 bp, HERV-K
ggaaaagaaagagagatcagactgt

SEQ ID NO: 339 - DNA, 25 bp, HERV-K
agaaagagagatcagactgttactg

SEQ ID NO: 340 - DNA, 25 bp, HERV-K
gagagatcagactgttactgtgtct

SEQ ID NO: 341 - DNA, 25 bp, HERV-K
atcagactgttactgtgtctatgta

SEQ ID NO: 342 - DNA, 25 bp, HERV-K
actgttactgtgtctatgtagaaag

SEQ ID NO: 343 - DNA, 25 bp, HERV-K
tactgtgtctatgtagaaagaaata

SEQ ID NO: 344 - DNA, 25 bp, HERV-K
tgtctatgtagaaagaaatagacat

SEQ ID NO: 345 - DNA, 25 bp, HERV-K
atgtagaaagaaatagacataagag

SEQ ID NO: 346 - DNA, 25 bp, HERV-K
gaaagaaatagacataagagactcc

SEQ ID NO: 347 - DNA, 25 bp, HERV-K
aaatagacataagagactccatttt

SEQ ID NO: 348 - DNA, 25 bp, HERV-K
gacataagagactccattttgttct

SEQ ID NO: 349 - DNA, 25 bp, HERV-K
aagagactccattttgttctgtact

SEQ ID NO: 350 - DNA, 25 bp, HERV-K
actccattttgttctgtactaagaa

SEQ ID NO: 351 - DNA, 25 bp, HERV-K
attttgttctgtactaagaaaaatt

SEQ ID NO: 352 - DNA, 25 bp, HERV-K
gttctgtactaagaaaaattcttct

SEQ ID NO: 353 - DNA, 25 bp, HERV-K
gtactaagaaaaattcttctgcttt

SEQ ID NO: 354 - DNA, 25 bp, HERV-K
aagaaaaattcttctgctttgagat

SEQ ID NO: 355 - DNA, 25 bp, HERV-K
aaattcttctgctttgagatgctgt

SEQ ID NO: 356 - DNA, 25 bp, HERV-K
cttctgctttgagatgctgttaatc

SEQ ID NO: 357 - DNA, 25 bp, HERV-K
gctttgagatgctgttaatctgtaa

SEQ ID NO: 358 - DNA, 25 bp, HERV-K
gagatgctgttaatctgtaacccta

SEQ ID NO: 359 - DNA, 25 bp, HERV-K
gctgttaatctgtaaccctagcccc

SEQ ID NO: 360 - DNA, 25 bp, HERV-K
taatctgtaaccctagccccaaccc

SEQ ID NO: 361 - DNA, 25 bp, HERV-K
tgtaaccctagccccaaccctgtgc

SEQ ID NO: 362 - DNA, 25 bp, HERV-K
ccctagccccaaccctgtgctcaca

SEQ ID NO: 363 - DNA, 25 bp, HERV-K
gccccaaccctgtgctcacagaaac

SEQ ID NO: 364 - DNA, 25 bp, HERV-K
aaccctgtgctcacagaaacaggtg

SEQ ID NO: 365 - DNA, 25 bp, HERV-K
gaaacaggtgctgtgttgactcaag

SEQ ID NO: 366 - DNA, 25 bp, HERV-K
aggtgctgtgttgactcaaggttta

SEQ ID NO: 367 - DNA, 25 bp, HERV-K
ctgtgttgactcaaggtttaatgga

SEQ ID NO: 368 - DNA, 25 bp, HERV-K
ttgactcaaggtttaatggattcag

SEQ ID NO: 369 - DNA, 25 bp, HERV-K
atggattcagggctgtgcaggatgt

SEQ ID NO: 370 - DNA, 25 bp, HERV-K
ttcagggctgtgcaggatgtgcttt

SEQ ID NO: 371 - DNA, 25 bp, HERV-K
ggctgtgcaggatgtgctttgttaa

SEQ ID NO: 372 - DNA, 25 bp, HERV-K
tgcaggatgtgctttgttaaacaaa

SEQ ID NO: 373 - DNA, 25 bp, HERV-K
gatgtgctttgttaaacaaatgctt

SEQ ID NO: 374 - DNA, 25 bp, HERV-K
gctttgttaaacaaatgcttgaagg

SEQ ID NO: 375 - DNA, 25 bp, HERV-K
gttaaacaaatgcttgaaggcagca

SEQ ID NO: 376 - DNA, 25 bp, HERV-K
gttaagagtcatcaccactccctaa

SEQ ID NO: 377 - DNA, 25 bp, HERV-K
gagtcatcaccactccctaatctca

SEQ ID NO: 378 - DNA, 25 bp, HERV-K
atcaccactccctaatctcaagtaa

SEQ ID NO: 379 - DNA, 25 bp, HERV-K
gcagggacacaaacactgcggaagg

SEQ ID NO: 380 - DNA, 25 bp, HERV-K
gacacaaacactgcggaaggccgca

SEQ ID NO: 381 - DNA, 25 bp, HERV-K
aaacactgcggaaggccgcagggac

SEQ ID NO: 382 - DNA, 25 bp, HERV-K
ctgcggaaggccgcagggacctctg

SEQ ID NO: 383 - DNA, 25 bp, HERV-K
gaaggccgcagggacctctgcctag

SEQ ID NO: 384 - DNA, 25 bp, HERV-K
ccgcagggacctctgcctaggaaag

SEQ ID NO: 385 - DNA, 25 bp, HERV-K
gggacctctgcctaggaaagccagg

SEQ ID NO: 386 - DNA, 25 bp, HERV-K
ctctgcctaggaaagccaggtgttg

SEQ ID NO: 387 - DNA, 25 bp, HERV-K
cctaggaaagccaggtgttgtccaa

SEQ ID NO: 388 - DNA, 25 bp, HERV-K
gaaagccaggtgttgtccaaggttt

SEQ ID NO: 389 - DNA, 25 bp, HERV-K
ccaggtgttgtccaaggtttctccc

SEQ ID NO: 390 - DNA, 25 bp, HERV-K
tgttgtccaaggtttctccccatgt

SEQ ID NO: 391 - DNA, 25 bp, HERV-K
tccaaggtttctccccatgtgacag

SEQ ID NO: 392 - DNA, 25 bp, HERV-K
ggtttctccccatgtgacagtctga

SEQ ID NO: 393 - DNA, 25 bp, HERV-K
ctccccatgtgacagtctgaaatat

SEQ ID NO: 394 - DNA, 25 bp, HERV-K
catgtgacagtctgaaatatggcct

SEQ ID NO: 395 - DNA, 25 bp, HERV-K
tctgaaatatggcctcttgggaagg

SEQ ID NO: 396 - DNA, 25 bp, HERV-K
aatatggcctcttgggaagggaaag

SEQ ID NO: 397 - DNA, 25 bp, HERV-K
ggcctcttgggaagggaaagacctg

SEQ ID NO: 398 - DNA, 25 bp, HERV-K
cttgggaagggaaagacctgactgt

SEQ ID NO: 399 - DNA, 25 bp, HERV-K
gaagggaaagacctgactgtcccct

SEQ ID NO: 400 - DNA, 25 bp, HERV-K
ggcccgacacccgtaaagggtctgt

SEQ ID NO: 401 - DNA, 25 bp, HERV-K
gacacccgtaaagggtctgtgctga

SEQ ID NO: 402 - DNA, 25 bp, HERV-K
ccgtaaagggtctgtgctgaggatt

SEQ ID NO: 403 - DNA, 25 bp, HERV-K
gctgaggattagtaaaagaggaagg

SEQ ID NO: 404 - DNA, 25 bp, HERV-K
ggattagtaaaagaggaaggaaggc

SEQ ID NO: 405 - DNA, 25 bp, HERV-K
agtaaaagaggaaggaaggcctctt

SEQ ID NO: 406 - DNA, 25 bp, HERV-K
aaggcctctttgcagttgagataag

SEQ ID NO: 407 - DNA, 25 bp, HERV-K
ctctttgcagttgagataagaggaa

SEQ ID NO: 408 - DNA, 25 bp, HERV-K
tgcagttgagataagaggaaggcat

SEQ ID NO: 409 - DNA, 25 bp, HERV-K
ttgagataagaggaaggcatctgtc

SEQ ID NO: 410 - DNA, 25 bp, HERV-K
ataagaggaaggcatctgtctcctg

SEQ ID NO: 411 - DNA, 25 bp, HERV-K
aggaaggcatctgtctcctgctcat

SEQ ID NO: 412 - DNA, 25 bp, HERV-K
ggcatctgtctcctgctcatccctg

SEQ ID NO: 413 - DNA, 25 bp, HERV-K
ctgtctcctgctcatccctgggcaa

SEQ ID NO: 414 - DNA, 25 bp, HERV-K
tcctgctcatccctgggcaatggaa

SEQ ID NO: 415 - DNA, 25 bp, HERV-K
ctcatccctgggcaatggaatgtct

SEQ ID NO: 416 - DNA, 25 bp, HERV-K
ttgtatatgccatctactgagatag

SEQ ID NO: 417 - DNA, 25 bp, HERV-K
cgtctacagtgagccttgtgggtga

SEQ ID NO: 418 - DNA, 25 bp, HERV-K
acagtgagccttgtgggtgaaggta

SEQ ID NO: 419 - DNA, 25 bp, HERV-K
gagccttgtgggtgaaggtactcta

SEQ ID NO: 420 - DNA, 25 bp, HERV-K
ttgtgggtgaaggtactctacagtg

SEQ ID NO: 421 - DNA, 25 bp, HERV-K
gcccaagaaagcctggctgttgtgg

SEQ ID NO: 422 - DNA, 25 bp, HERV-K
agaaagcctggctgttgtgggaagt

SEQ ID NO: 423 - DNA, 25 bp, HERV-K
gcctggctgttgtgggaagtcaggg

SEQ ID NO: 424 - DNA, 25 bp, HERV-K
gctgttgtgggaagtcagggacccc

SEQ ID NO: 425 - DNA, 25 bp, HERV-K
tgtgggaagtcagggaccccgaatg

SEQ ID NO: 426 - DNA, 25 bp, HERV-K
gaagtcagggaccccgaatggaggg

SEQ ID NO: 427 - DNA, 25 bp, HERV-K
cagggaccccgaatggagggaccag

SEQ ID NO: 428 - DNA, 25 bp, HERV-K
accccgaatggagggaccagctggt

SEQ ID NO: 429 - DNA, 25 bp, HERV-K
gaatggagggaccagctggtgctgc

SEQ ID NO: 430 - DNA, 25 bp, HERV-K
gagggaccagctggtgctgcatcag

SEQ ID NO: 431 - DNA, 25 bp, HERV-K
accagctggtgctgcatcaggaaac

SEQ ID NO: 432 - DNA, 25 bp, HERV-K
ctggtgctgcatcaggaaacataaa

SEQ ID NO: 433 - DNA, 25 bp, HERV-K
gctgcatcaggaaacataaattgtg

SEQ ID NO: 434 - DNA, 25 bp, HERV-K
atcaggaaacataaattgtgaagat

SEQ ID NO: 435 - DNA, 25 bp, HERV-K
gaaacataaattgtgaagatttctt

SEQ ID NO: 436 - DNA, 25 bp, HERV-K
ataaattgtgaagatttcttggaca

SEQ ID NO: 437 - DNA, 25 bp, HERV-K
ttgtgaagatttcttggacatttat

SEQ ID NO: 438 - DNA, 25 bp, HERV-K
aagatttcttggacatttatcagtt

SEQ ID NO: 439 - DNA, 25 bp, HERV-K
ttcttggacatttatcagtttccaa

SEQ ID NO: 440 - DNA, 25 bp, HERV-K
ggacatttatcagtttccaaaatta

SEQ ID NO: 441 - DNA, 25 bp, HERV-K
tttatcagtttccaaaattaatact

SEQ ID NO: 442 - DNA, 25 bp, HERV-K
cagtttccaaaattaatacttttat

SEQ ID NO: 443 - DNA, 25 bp, HERV-K
tccaaaattaatacttttataattt

SEQ ID NO: 444 - DNA, 25 bp, HERV-K
aattaatacttttataatttcttac

SEQ ID NO: 445 - DNA, 25 bp, HERV-K
atacttttataatttcttacacctg

SEQ ID NO: 446 - DNA, 25 bp, HERV-K
tttataatttcttacacctgtctta

SEQ ID NO: 447 - DNA, 25 bp, HERV-K
aatttcttacacctgtcttacttta

SEQ ID NO: 448 - DNA, 25 bp, HERV-K
cttacacctgtcttactttaatctc

SEQ ID NO: 449 - DNA, 25 bp, HERV-K
acctgtcttactttaatctcttaat

SEQ ID NO: 450 - DNA, 25 bp, HERV-K
tcttactttaatctcttaatcctgt

SEQ ID NO: 451 - DNA, 25 bp, HERV-K
ctttaatctcttaatcctgttatct

SEQ ID NO: 452 - DNA, 25 bp, HERV-K
atctcttaatcctgttatctttgta

SEQ ID NO: 453 - DNA, 25 bp, HERV-K
ttaatcctgttatctttgtaagctg

SEQ ID NO: 454 - DNA, 25 bp, HERV-K
cctgttatctttgtaagctgaggat

SEQ ID NO: 455 - DNA, 25 bp, HERV-K
tatctttgtaagctgaggatatacg

SEQ ID NO: 456 - DNA, 25 bp, HERV-K
ttgtaagctgaggatatacgtcacc

SEQ ID NO: 457 - DNA, 25 bp, HERV-K
agctgaggatatacgtcacctcagg

SEQ ID NO: 458 - DNA, 25 bp, HERV-K
aggatatacgtcacctcaggaccac

SEQ ID NO: 459 - DNA, 25 bp, HERV-K
atacgtcacctcaggaccactattg

SEQ ID NO: 460 - DNA, 25 bp, HERV-K
tcacctcaggaccactattgtacaa

SEQ ID NO: 461 - DNA, 25 bp, HERV-K
tcaggaccactattgtacaaattga

SEQ ID NO: 462 - DNA, 25 bp, HERV-K
accactattgtacaaattgattgta

SEQ ID NO: 463 - DNA, 25 bp, HERV-K
tattgtacaaattgattgtaaaaca

SEQ ID NO: 464 - DNA, 25 bp, HERV-K
tacaaattgattgtaaaacatgttc

SEQ ID NO: 465 - DNA, 25 bp, HERV-K
attgattgtaaaacatgttcacatg

SEQ ID NO: 466 - DNA, 25 bp, HERV-K
ttgtaaaacatgttcacatgtgttt

SEQ ID NO: 467 - DNA, 25 bp, HERV-K
aaacatgttcacatgtgtttgaaca

SEQ ID NO: 468 - DNA, 25 bp, HERV-K
tgttcacatgtgtttgaacaatatg

SEQ ID NO: 469 - DNA, 25 bp, HERV-K
acatgtgtttgaacaatatgaaatc

SEQ ID NO: 470 - DNA, 25 bp, HERV-K
tgtttgaacaatatgaaatcagtgc

SEQ ID NO: 471 - DNA, 25 bp, HERV-K
gaacaatatgaaatcagtgcacctt

SEQ ID NO: 472 - DNA, 25 bp, HERV-K
atatgaaatcagtgcaccttgaaaa

SEQ ID NO: 473 - DNA, 25 bp, HERV-K
aaatcagtgcaccttgaaaatgaac

SEQ ID NO: 474 - DNA, 25 bp, HERV-K
agtgcaccttgaaaatgaacagaat

SEQ ID NO: 475 - DNA, 25 bp, HERV-K
accttgaaaatgaacagaataacag

SEQ ID NO: 476 - DNA, 25 bp, HERV-K
gaaaatgaacagaataacagtgatt

SEQ ID NO: 477 - DNA, 25 bp, HERV-K
tgaacagaataacagtgattttagg

SEQ ID NO: 478 - DNA, 25 bp, HERV-K
agaataacagtgattttagggaaca

SEQ ID NO: 479 - DNA, 25 bp, HERV-K
aacagtgattttagggaacaaagga

SEQ ID NO: 480 - DNA, 25 bp, HERV-K
tgattttagggaacaaaggaagaca

SEQ ID NO: 481 - DNA, 25 bp, HERV-K
ttagggaacaaaggaagacaaccat

SEQ ID NO: 482 - DNA, 25 bp, HERV-K
gaacaaaggaagacaaccataaggt

SEQ ID NO: 483 - DNA, 25 bp, HERV-K
aaggaagacaaccataaggtctgac

SEQ ID NO: 484 - DNA, 25 bp, HERV-K
agacaaccataaggtctgactgcct

SEQ ID NO: 485 - DNA, 25 bp, HERV-K
accataaggtctgactgcctgaggg

SEQ ID NO: 486 - DNA, 25 bp, HERV-K
aaggtctgactgcctgaggggtcgg

SEQ ID NO: 487 - DNA, 25 bp, HERV-K
ctgactgcctgaggggtcgggcaaa

SEQ ID NO: 488 - DNA, 25 bp, HERV-K
tgcctgaggggtcgggcaaaaagcc

SEQ ID NO: 489 - DNA, 25 bp, HERV-K
gaggggtcgggcaaaaagccatatt

SEQ ID NO: 490 - DNA, 25 bp, HERV-K
gtcgggcaaaaagccatatttttct

SEQ ID NO: 491 - DNA, 25 bp, HERV-K
gcaaaaagccatatttttcttcttg

SEQ ID NO: 492 - DNA, 25 bp, HERV-K
aagccatatttttcttcttgcagag

SEQ ID NO: 493 - DNA, 25 bp, HERV-K
atatttttcttcttgcagagagcct

SEQ ID NO: 494 - DNA, 25 bp, HERV-K
tttcttcttgcagagagcctataaa

SEQ ID NO: 495 - DNA, 25 bp, HERV-K
tcttgcagagagcctataaatggac

SEQ ID NO: 496 - DNA, 25 bp, HERV-K
cagagagcctataaatggacgtgca

SEQ ID NO: 497 - DNA, 25 bp, HERV-K
agcctataaatggacgtgcaagtag

SEQ ID NO: 498 - DNA, 25 bp, HERV-K
ataaatggacgtgcaagtaggagag

SEQ ID NO: 499 - DNA, 25 bp, HERV-K
tggacgtgcaagtaggagagatatt

SEQ ID NO: 500 - DNA, 25 bp, HERV-K
gtgcaagtaggagagatattgctaa

SEQ ID NO: 501 - DNA, 25 bp, HERV-K
agtaggagagatattgctaaattct

SEQ ID NO: 502 - DNA, 25 bp, HERV-K
gagagatattgctaaattcttttcc

SEQ ID NO: 503 - DNA, 25 bp, HERV-K
atattgctaaattcttttcctagca

SEQ ID NO: 504 - DNA, 25 bp, HERV-K
gctaaattcttttcctagcaaggaa

SEQ ID NO: 505 - DNA, 25 bp, HERV-K
attcttttcctagcaaggaatataa

SEQ ID NO: 506 - DNA, 25 bp, HERV-K
tttcctagcaaggaatataatacta

SEQ ID NO: 507 - DNA, 25 bp, HERV-K
tagcaaggaatataatactaagacc

SEQ ID NO: 508 - DNA, 25 bp, HERV-K
aggaatataatactaagaccctagg

SEQ ID NO: 509 - DNA, 25 bp, HERV-K
tataatactaagaccctagggaaag

SEQ ID NO: 510 - DNA, 25 bp, HERV-K
tactaagaccctagggaaagaattg

SEQ ID NO: 511 - DNA, 25 bp, HERV-K
agaccctagggaaagaattgcattc

SEQ ID NO: 512 - DNA, 25 bp, HERV-K
ctagggaaagaattgcattcctggg

SEQ ID NO: 513 - DNA, 25 bp, HERV-K
gaaagaattgcattcctggggggag

SEQ ID NO: 514 - DNA, 25 bp, HERV-K
aattgcattcctggggggaggtcta

SEQ ID NO: 515 - DNA, 25 bp, HERV-K
cattcctggggggaggtctataaac

SEQ ID NO: 516 - DNA, 25 bp, HERV-K
ctggggggaggtctataaacggccg

SEQ ID NO: 517 - DNA, 25 bp, HERV-K
gggaggtctataaacggccgctctg

SEQ ID NO: 518 - DNA, 25 bp, HERV-K
gtctataaacggccgctctgggagt

SEQ ID NO: 519 - DNA, 25 bp, HERV-K
taaacggccgctctgggagtgtctg

SEQ ID NO: 520 - DNA, 25 bp, HERV-K
ggccgctctgggagtgtctgtccta

SEQ ID NO: 521 - DNA, 25 bp, HERV-K
ctctgggagtgtctgtcctatgtgg

SEQ ID NO: 522 - DNA, 25 bp, HERV-K
ggagtgtctgtcctatgtggttgag

SEQ ID NO: 523 - DNA, 25 bp, HERV-K
gtctgtcctatgtggttgagataag

SEQ ID NO: 524 - DNA, 25 bp, HERV-K
tcctatgtggttgagataaggactg

SEQ ID NO: 525 - DNA, 25 bp, HERV-K
tgtggttgagataaggactgagata

SEQ ID NO: 526 - DNA, 25 bp, HERV-K
ttgagataaggactgagatacgccc

SEQ ID NO: 527 - DNA, 25 bp, HERV-K
ataaggactgagatacgccctggtc

SEQ ID NO: 528 - DNA, 25 bp, HERV-K
gactgagatacgccctggtctcctg

SEQ ID NO: 529 - DNA, 25 bp, HERV-K
agatacgccctggtctcctgcagta

SEQ ID NO: 530 - DNA, 25 bp, HERV-K
cgccctggtctcctgcagtaccctc

SEQ ID NO: 531 - DNA, 25 bp, HERV-K
tggtctcctgcagtaccctcaggct

SEQ ID NO: 532 - DNA, 25 bp, HERV-K
tcctgcagtaccctcaggcttacta

SEQ ID NO: 533 - DNA, 25 bp, HERV-K
cagtaccctcaggcttactaggatt

SEQ ID NO: 534 - DNA, 25 bp, HERV-K
ccctcaggcttactaggattgggaa

SEQ ID NO: 535 - DNA, 25 bp, HERV-K
aggcttactaggattgggaaacccc

SEQ ID NO: 536 - DNA, 25 bp, HERV-K
tactaggattgggaaaccccagtcc

SEQ ID NO: 537 - DNA, 25 bp, HERV-K
ggattgggaaaccccagtcctggta

SEQ ID NO: 538 - DNA, 25 bp, HERV-K
gggaaaccccagtcctggtaaattt

SEQ ID NO: 539 - DNA, 25 bp, HERV-K
accccagtcctggtaaatttgaggt

SEQ ID NO: 540 - DNA, 25 bp, HERV-K
agtcctggtaaatttgaggtcaggc

SEQ ID NO: 541 - DNA, 25 bp, HERV-K
tggtaaatttgaggtcaggccggtt

SEQ ID NO: 542 - DNA, 25 bp, HERV-K
aatttgaggtcaggccggttctttg

SEQ ID NO: 543 - DNA, 25 bp, HERV-K
gaggtcaggccggttctttgctctg

SEQ ID NO: 544 - DNA, 25 bp, HERV-K
caggccggttctttgctctgaaccc

SEQ ID NO: 545 - DNA, 25 bp, HERV-K
cggttctttgctctgaaccctgttt

SEQ ID NO: 546 - DNA, 25 bp, HERV-K
ctttgctctgaaccctgttttctgt

SEQ ID NO: 547 - DNA, 25 bp, HERV-K
ctctgaaccctgttttctgttaaga

SEQ ID NO: 548 - DNA, 25 bp, HERV-K
aaccctgttttctgttaagatgttt

SEQ ID NO: 549 - DNA, 25 bp, HERV-K
tgttttctgttaagatgtttatcaa

SEQ ID NO: 550 - DNA, 25 bp, HERV-K
tctgttaagatgtttatcaagacaa

SEQ ID NO: 551 - DNA, 25 bp, HERV-K
taagatgtttatcaagacaatacat

SEQ ID NO: 552 - DNA, 25 bp, HERV-K
tgtttatcaagacaatacatgcacc

SEQ ID NO: 553 - DNA, 25 bp, HERV-K
atcaagacaatacatgcaccgctga

SEQ ID NO: 554 - DNA, 25 bp, HERV-K
gacaatacatgcaccgctgaacata

SEQ ID NO: 555 - DNA, 25 bp, HERV-K
tacatgcaccgctgaacatagaccc

SEQ ID NO: 556 - DNA, 25 bp, HERV-K
gcaccgctgaacatagacccttatc

SEQ ID NO: 557 - DNA, 25 bp, HERV-K
gctgaacatagacccttatcaggag

SEQ ID NO: 558 - DNA, 25 bp, HERV-K
acatagacccttatcaggagtttct

SEQ ID NO: 559 - DNA, 25 bp, HERV-K
gacccttatcaggagtttctgattt

SEQ ID NO: 560 - DNA, 25 bp, HERV-K
ttatcaggagtttctgattttgctc

SEQ ID NO: 561 - DNA, 25 bp, HERV-K
aggagtttctgattttgctctggtc

SEQ ID NO: 562 - DNA, 25 bp, HERV-K
tttctgattttgctctggtcctgtt

SEQ ID NO: 563 - DNA, 25 bp, HERV-K
gattttgctctggtcctgtttcttc

SEQ ID NO: 564 - DNA, 25 bp, HERV-K
tgctctggtcctgtttcttcagaag

SEQ ID NO: 565 - DNA, 25 bp, HERV-K
tggtcctgtttcttcagaagcatgt

SEQ ID NO: 566 - DNA, 25 bp, HERV-K
ctgtttcttcagaagcatgtcatct

SEQ ID NO: 567 - DNA, 25 bp, HERV-K
tcttcagaagcatgtcatctttgct

SEQ ID NO: 568 - DNA, 25 bp, HERV-K
agaagcatgtcatctttgctctgcc

SEQ ID NO: 569 - DNA, 25 bp, HERV-K
catgtcatctttgctctgccttctg

SEQ ID NO: 570 - DNA, 25 bp, HERV-K
catctttgctctgccttctgccctt

SEQ ID NO: 571 - DNA, 25 bp, HERV-K
ttgctctgccttctgccctttgaag

SEQ ID NO: 572 - DNA, 25 bp, HERV-K
ctgccttctgccctttgaagcatgt

SEQ ID NO: 573 - DNA, 25 bp, HERV-K
ttctgccctttgaagcatgtgatct

SEQ ID NO: 574 - DNA, 25 bp, HERV-K
ccctttgaagcatgtgatctttgtg

SEQ ID NO: 575 - DNA, 25 bp, HERV-K
tgaagcatgtgatctttgtgaccta

SEQ ID NO: 576 - DNA, 25 bp, HERV-K
catgtgatctttgtgacctactccc

SEQ ID NO: 577 - DNA, 25 bp, HERV-K
gatctttgtgacctactccctgttc

SEQ ID NO: 578 - DNA, 25 bp, HERV-K
ttgtgacctactccctgttcataca

SEQ ID NO: 579 - DNA, 25 bp, HERV-K
acctactccctgttcatacacccct

SEQ ID NO: 580 - DNA, 25 bp, HERV-K
ctccctgttcatacacccctcccct

SEQ ID NO: 581 - DNA, 25 bp, HERV-K
tgttcatacacccctccccttttaa

SEQ ID NO: 582 - DNA, 25 bp, HERV-K
atacacccctccccttttaaaatcc

SEQ ID NO: 583 - DNA, 25 bp, HERV-K
cccctccccttttaaaatccctaat

SEQ ID NO: 584 - DNA, 25 bp, HERV-K
ccccttttaaaatccctaataaaaa

SEQ ID NO: 585 - DNA, 25 bp, HERV-K
tttaaaatccctaataaaaacttgc

SEQ ID NO: 586 - DNA, 25 bp, HERV-K
aatccctaataaaaacttgctggtt

SEQ ID NO: 587 - DNA, 25 bp, HERV-K
ctaataaaaacttgctggttttgtg

SEQ ID NO: 588 - DNA, 25 bp, HERV-K
aaaaacttgctggttttgtggctca

SEQ ID NO: 589 - DNA, 25 bp, HERV-K
cttgctggttttgtggctcaggggg

SEQ ID NO: 590 - DNA, 25 bp, HERV-K
tggttttgtggctcaggggggcatc

SEQ ID NO: 591 - DNA, 25 bp, HERV-K
ttgtggctcaggggggcatcatgga

SEQ ID NO: 592 - DNA, 25 bp, HERV-K
gctcaggggggcatcatggacctac

SEQ ID NO: 593 - DNA, 25 bp, HERV-K
ggggggcatcatggacctaccaata

SEQ ID NO: 594 - DNA, 25 bp, HERV-K
gcatcatggacctaccaatacgtga

SEQ ID NO: 595 - DNA, 25 bp, HERV-K
atggacctaccaatacgtgatgtca

SEQ ID NO: 596 - DNA, 25 bp, HERV-K
cctaccaatacgtgatgtcaccccc

SEQ ID NO: 597 - DNA, 25 bp, HERV-K
caatacgtgatgtcacccccggtgg

SEQ ID NO: 598 - DNA, 25 bp, HERV-K
cgtgatgtcacccccggtggcccag

SEQ ID NO: 599 - DNA, 25 bp, HERV-K
tgtcacccccggtggcccagctgta

SEQ ID NO: 600 - DNA, 25 bp, HERV-K
tgtgctcacagaaacaggtgctgtg

SEQ ID NO: 601 - DNA, 25 bp, HERV-K
tcacagaaacaggtgctgtgttgac

SEQ ID NO: 602 - DNA, 25 bp, HERV-K
tcaaggtttaatggattcagggctg

SEQ ID NO: 603 - DNA, 25 bp, HERV-K
gtttaatggattcagggctgtgcag

SEQ ID NO: 604 - DNA, 25 bp, HERV-K
acaaatgcttgaaggcagcaagctt

SEQ ID NO: 605 - DNA, 25 bp, HERV-K
tgcttgaaggcagcaagcttgttaa

SEQ ID NO: 606 - DNA, 25 bp, HERV-K
gaaggcagcaagcttgttaagagtc

SEQ ID NO: 607 - DNA, 25 bp, HERV-K
cagcaagcttgttaagagtcatcac

SEQ ID NO: 608 - DNA, 25 bp, HERV-K
agcttgttaagagtcatcaccactc

SEQ ID NO: 609 - DNA, 25 bp, HERV-K
cactccctaatctcaagtaagcagg

SEQ ID NO: 610 - DNA, 25 bp, HERV-K
cctaatctcaagtaagcagggacac

SEQ ID NO: 611 - DNA, 25 bp, HERV-K
tctcaagtaagcagggacacaaaca

SEQ ID NO: 612 - DNA, 25 bp, HERV-K
agtaagcagggacacaaacactgcg

SEQ ID NO: 613 - DNA, 25 bp, HERV-K
gacagtctgaaatatggcctcttgg

SEQ ID NO: 614 - DNA, 25 bp, HERV-K
gaaagacctgactgtcccctggccc

SEQ ID NO: 615 - DNA, 25 bp, HERV-K
acctgactgtcccctggcccgacac

SEQ ID NO: 616 - DNA, 25 bp, HERV-K
actgtcccctggcccgacacccgta

SEQ ID NO: 617 - DNA, 25 bp, HERV-K
cccctggcccgacacccgtaaaggg

SEQ ID NO: 618 - DNA, 25 bp, HERV-K
aagggtctgtgctgaggattagtaa

SEQ ID NO: 619 - DNA, 25 bp, HERV-K
tctgtgctgaggattagtaaaagag

SEQ ID NO: 620 - DNA, 25 bp, HERV-K
aagaggaaggaaggcctctttgcag

SEQ ID NO: 621 - DNA, 25 bp, HERV-K
gaaggaaggcctctttgcagttgag

SEQ ID NO: 622 - DNA, 25 bp, HERV-K
ccctgggcaatggaatgtcttggtg

SEQ ID NO: 623 - DNA, 25 bp, HERV-K
ggcaatggaatgtcttggtgtaaag

SEQ ID NO: 624 - DNA, 25 bp, HERV-K
tggaatgtcttggtgtaaagcctga

SEQ ID NO: 625 - DNA, 25 bp, HERV-K
tgtcttggtgtaaagcctgattgta

SEQ ID NO: 626 - DNA, 25 bp, HERV-K
tggtgtaaagcctgattgtatatgc

SEQ ID NO: 627 - DNA, 25 bp, HERV-K
taaagcctgattgtatatgccatct

SEQ ID NO: 628 - DNA, 25 bp, HERV-K
cctgattgtatatgccatctactga

SEQ ID NO: 629 - DNA, 25 bp, HERV-K
tatgccatctactgagataggagaa

SEQ ID NO: 630 - DNA, 25 bp, HERV-K
catctactgagataggagaaaactg

SEQ ID NO: 631 - DNA, 25 bp, HERV-K
gggctggaggtgggacatgctggcg

SEQ ID NO: 632 - DNA, 25 bp, HERV-K
ggaggtgggacatgctggcggcaat

SEQ ID NO: 633 - DNA, 25 bp, HERV-K
tgggacatgctggcggcaatactgc

SEQ ID NO: 634 - DNA, 25 bp, HERV-K
catgctggcggcaatactgctcttt

SEQ ID NO: 635 - DNA, 25 bp, HERV-K
tggcggcaatactgctctttaaggc

SEQ ID NO: 636 - DNA, 25 bp, HERV-K
gcaatactgctctttaaggcattga

SEQ ID NO: 637 - DNA, 25 bp, HERV-K
ttatgtatatgcacatcaaaagcac

SEQ ID NO: 638 - DNA, 25 bp, HERV-K
tatatgcacatcaaaagcacagcac

SEQ ID NO: 639 - DNA, 25 bp, HERV-K
gcacatcaaaagcacagcacttttt

SEQ ID NO: 640 - DNA, 25 bp, HERV-K
tcaaaagcacagcacttttttcttt

SEQ ID NO: 641 - DNA, 25 bp, HERV-K
agcacagcacttttttctttacctt

SEQ ID NO: 642 - DNA, 25 bp, HERV-K
agcacttttttctttaccttgttta

SEQ ID NO: 643 - DNA, 25 bp, HERV-K
accttgtttatgatgcagagacatt

SEQ ID NO: 644 - DNA, 25 bp, HERV-K
gtttatgatgcagagacatttgttc

SEQ ID NO: 645 - DNA, 25 bp, HERV-K
tgatgcagagacatttgttcacatg

SEQ ID NO: 646 - DNA, 25 bp, HERV-K
actcagagaccggtgcggcgcgggt

SEQ ID NO: 647 - DNA, 25 bp, HERV-K
gagaccggtgcggcgcgggtcctcc

SEQ ID NO: 648 - DNA, 25 bp, HERV-K
cggtgcggcgcgggtcctccatatg

SEQ ID NO: 649 - DNA, 25 bp, HERV-K
ctttgtctctgttgtctttcttttc

SEQ ID NO: 650 - DNA, 25 bp, HERV-K
tctctgttgtctttcttttctcaag

SEQ ID NO: 651 - DNA, 25 bp, HERV-K
tcaagtctctcgttccacctgagga

SEQ ID NO: 652 - DNA, 25 bp, HERV-K
tctctcgttccacctgaggagaaat

SEQ ID NO: 653 - DNA, 25 bp, HERV-K
cgttccacctgaggagaaatgccca

SEQ ID NO: 654 - DNA, 25 bp, HERV-K
cacctgaggagaaatgcccacagct

SEQ ID NO: 655 - DNA, 25 bp, HERV-K
gaggagaaatgcccacagctgtgga

SEQ ID NO: 656 - DNA, 25 bp, HERV-K
gaaatgcccacagctgtggaggcgc

SEQ ID NO: 657 - DNA, 25 bp, HERV-K
gcccacagctgtggaggcgcaggcc

SEQ ID NO: 658 - DNA, 25 bp, HERV-K
cagctgtggaggcgcaggccactcc

SEQ ID NO: 659 - DNA, 25 bp, HERV-K
gtggaggcgcaggccactccatctg

SEQ ID NO: 660 - DNA, 25 bp, HERV-K
ggcgcaggccactccatctggtgcc

SEQ ID NO: 661 - DNA, 25 bp, HERV-K
aggccactccatctggtgcccaacg

SEQ ID NO: 662 - DNA, 25 bp, HERV-K
actccatctggtgcccaacgtggat

SEQ ID NO: 663 - DNA, 25 bp, HERV-K
gcttttctctagggtgaagggactc

SEQ ID NO: 664 - DNA, 25 bp, HERV-K
tctctagggtgaagggactctcgag

SEQ ID NO: 665 - DNA, 25 bp, HERV-K
agggtgaagggactctcgagtgtgg

SEQ ID NO: 666 - DNA, 25 bp, HERV-K
gaagggactctcgagtgtggtcatt

SEQ ID NO: 667 - DNA, 25 bp, HERV-K
gactctcgagtgtggtcattgagga

SEQ ID NO: 668 - DNA, 25 bp, HERV-K
tgtggtcattgaggacaagtcaacg

SEQ ID NO: 669 - DNA, 25 bp, HERV-K
gagtacgtctacagtgagccttgtg

SEQ ID NO: 670 - DNA, 25 bp, HERV-K
cgtctacagtgagccttgtggtaag

SEQ ID NO: 671 - DNA, 25 bp, HERV-K
acagtgagccttgtggtaagcttgg

SEQ ID NO: 672 - DNA, 25 bp, HERV-K
gagccttgtggtaagcttgggcgct

SEQ ID NO: 673 - DNA, 25 bp, HERV-K
ttgtggtaagcttgggcgctcggaa

SEQ ID NO: 674 - DNA, 25 bp, HERV-K
cttgggcgctcggaagaagccaggg

SEQ ID NO: 675 - DNA, 25 bp, HERV-K
gcgctcggaagaagccagggttaat

SEQ ID NO: 676 - DNA, 25 bp, HERV-K
cggaagaagccagggttaatggggc

SEQ ID NO: 677 - DNA, 25 bp, HERV-K
gaagccagggttaatggggcaaact

SEQ ID NO: 678 - DNA, 25 bp, HERV-K
cagggttaatggggcaaactaaaag

SEQ ID NO: 679 - DNA, 25 bp, HERV-K
ggggcaaactaaaagtaaagtctct

SEQ ID NO: 680 - DNA, 25 bp, HERV-K
aaaagtaaagtctctcattccacct

SEQ ID NO: 681 - DNA, 25 bp, HERV-K
taaagtctctcattccacctgatga

SEQ ID NO: 682 - DNA, 25 bp, HERV-K
ggggcaggccaccccttcagggtag

SEQ ID NO: 683 - DNA, 25 bp, HERV-K
aggccaccccttcagggtagggtcc

SEQ ID NO: 684 - DNA, 25 bp, HERV-K
accccttcagggtagggtcccctcc

SEQ ID NO: 685 - DNA, 25 bp, HERV-K
ttcagggtagggtcccctccatgca

SEQ ID NO: 686 - DNA, 25 bp, HERV-K
ggtagggtcccctccatgcagacca

SEQ ID NO: 687 - DNA, 25 bp, HERV-K
ggtcccctccatgcagaccatagag

SEQ ID NO: 688 - DNA, 25 bp, HERV-K
cctccatgcagaccatagagcacag

SEQ ID NO: 689 - DNA, 25 bp, HERV-K
atgcagaccatagagcacaggtgtg

SEQ ID NO: 690 - DNA, 25 bp, HERV-K
gaccatagagcacaggtgtgcccca

SEQ ID NO: 691 - DNA, 25 bp, HERV-K
tagagcacaggtgtgccccaaagag

SEQ ID NO: 692 - DNA, 25 bp, HERV-K
cacaggtgtgccccaaagaggagca

SEQ ID NO: 693 - DNA, 25 bp, HERV-K
gtgtgccccaaagaggagcagagag

SEQ ID NO: 694 - DNA, 25 bp, HERV-K
ccccaaagaggagcagagagaagga

SEQ ID NO: 695 - DNA, 25 bp, HERV-K
aagaggagcagagagaaggagggag

SEQ ID NO: 696 - DNA, 25 bp, HERV-K
gagcagagagaaggagggagagggc

SEQ ID NO: 697 - DNA, 25 bp, HERV-K
gagagaaggagggagagggcccacg

SEQ ID NO: 698 - DNA, 25 bp, HERV-K
aaggagggagagggcccacgagaga

SEQ ID NO: 699 - DNA, 25 bp, HERV-K
gggagagggcccacgagagacttgg

SEQ ID NO: 700 - DNA, 25 bp, HERV-K
agggcccacgagagacttggaaatg

SEQ ID NO: 701 - DNA, 25 bp, HERV-K
ccacgagagacttggaaatgaatgg

SEQ ID NO: 702 - DNA, 25 bp, HERV-K
agagacttggaaatgaatggcagga

SEQ ID NO: 703 - DNA, 25 bp, HERV-K
cttggaaatgaatggcaggatttta

SEQ ID NO: 704 - DNA, 25 bp, HERV-K
aaatgaatggcaggattttaggcgc

SEQ ID NO: 705 - DNA, 25 bp, HERV-K
aatggcaggattttaggcgctggac

SEQ ID NO: 706 - DNA, 25 bp, HERV-K
caggattttaggcgctggacttggg

SEQ ID NO: 707 - DNA, 25 bp, HERV-K
ttttaggcgctggacttgggttcgg

SEQ ID NO: 708 - DNA, 25 bp, HERV-K
ggcgctggacttgggttcggggcac

SEQ ID NO: 709 - DNA, 25 bp, HERV-K
tggacttgggttcggggcacctggc

SEQ ID NO: 710 - DNA, 25 bp, HERV-K
ttgggttcggggcacctggcctttc

SEQ ID NO: 711 - DNA, 25 bp, HERV-K
ttcggggcacctggcctttccttgt

SEQ ID NO: 712 - DNA, 25 bp, HERV-K
ggcacctggcctttccttgtgtatt

SEQ ID NO: 713 - DNA, 25 bp, HERV-K
ctggcctttccttgtgtatttctcc

SEQ ID NO: 714 - DNA, 25 bp, HERV-K
ctttccttgtgtatttctcctactg

SEQ ID NO: 715 - DNA, 25 bp, HERV-K
cttgtgtatttctcctactgtctgc

SEQ ID NO: 716 - DNA, 25 bp, HERV-K
gtatttctcctactgtctgcctaac

SEQ ID NO: 717 - DNA, 25 bp, HERV-K
tctcctactgtctgcctaactattt

SEQ ID NO: 718 - DNA, 25 bp, HERV-K
aatacaataaaagaaaaccagcccc

SEQ ID NO: 719 - DNA, 25 bp, HERV-K
aataaaagaaaaccagcccctggtt

SEQ ID NO: 720 - DNA, 25 bp, HERV-K
aagaaaaccagcccctggttcttgt

SEQ ID NO: 721 - DNA, 25 bp, HERV-K
aaccagcccctggttcttgtggtgt

SEQ ID NO: 722 - DNA, 25 bp, HERV-K
gcccctggttcttgtggtgtttcca

SEQ ID NO: 723 - DNA, 25 bp, HERV-K
tggttcttgtggtgtttccaccctc

SEQ ID NO: 724 - DNA, 25 bp, HERV-K
cttgtggtgtttccaccctcccggg

SEQ ID NO: 725 - DNA, 25 bp, HERV-K
ggtgtttccaccctcccgggtcccc

SEQ ID NO: 726 - DNA, 25 bp, HERV-K
ttccaccctcccgggtccccgctgg

SEQ ID NO: 727 - DNA, 25 bp, HERV-K
ccctcccgggtccccgctggctgcc

SEQ ID NO: 728 - DNA, 25 bp, HERV-K
ccgggtccccgctggctgcctggct

SEQ ID NO: 729 - DNA, 25 bp, HERV-K
tccccgctggctgcctggcttcctc

SEQ ID NO: 730 - DNA, 25 bp, HERV-K
gctggctgcctggcttcctcccgca

SEQ ID NO: 731 - DNA, 25 bp, HERV-K
ctgcctggcttcctcccgcagctcc

SEQ ID NO: 732 - DNA, 25 bp, HERV-K
tggcttcctcccgcagctcctgctg

SEQ ID NO: 733 - DNA, 25 bp, HERV-K
tcctcccgcagctcctgctgtgtgt

SEQ ID NO: 734 - DNA, 25 bp, HERV-K
ccgcagctcctgctgtgtgtgtatg

SEQ ID NO: 735 - DNA, 25 bp, HERV-K
gctcctgctgtgtgtgtatgtgtgt

SEQ ID NO: 736 - DNA, 25 bp, HERV-K
tgctgtgtgtgtatgtgtgtgtgtg

SEQ ID NO: 737 - DNA, 25 bp, HERV-K
tgtgtgtatgtgtgtgtgtgtgcac

SEQ ID NO: 738 - DNA, 25 bp, HERV-K
gtatgtgtgtgtgtgtgcacatctg

SEQ ID NO: 739 - DNA, 25 bp, HERV-K
tgtgtgtgtgtgcacatctgtgggg

SEQ ID NO: 740 - DNA, 25 bp, HERV-K
gtgtgtgcacatctgtggggcgtat

SEQ ID NO: 741 - DNA, 25 bp, HERV-K
tgcacatctgtggggcgtatgtgtg

SEQ ID NO: 742 - DNA, 25 bp, HERV-K
atctgtggggcgtatgtgtgttcgt

SEQ ID NO: 743 - DNA, 25 bp, HERV-K
tggggcgtatgtgtgttcgtctttg

SEQ ID NO: 744 - DNA, 25 bp, HERV-K
cgtatgtgtgttcgtctttgtaatt

SEQ ID NO: 745 - DNA, 25 bp, HERV-K
gtgtgttcgtctttgtaattgaggc

SEQ ID NO: 746 - DNA, 25 bp, HERV-K
ttcgtctttgtaattgaggctgcag

SEQ ID NO: 747 - DNA, 25 bp, HERV-K
ctttgtaattgaggctgcagagtgg

SEQ ID NO: 748 - DNA, 25 bp, HERV-K
taattgaggctgcagagtggagaga

SEQ ID NO: 749 - DNA, 25 bp, HERV-K
gaggctgcagagtggagagagcagg

SEQ ID NO: 750 - DNA, 25 bp, HERV-K
tgcagagtggagagagcaggggttt

SEQ ID NO: 751 - DNA, 25 bp, HERV-K
agtggagagagcaggggttttctct

SEQ ID NO: 752 - DNA, 25 bp, HERV-K
agagagcaggggttttctctgggga

SEQ ID NO: 753 - DNA, 25 bp, HERV-K
gcaggggttttctctggggacccag

SEQ ID NO: 754 - DNA, 25 bp, HERV-K
ggttttctctggggacccagagaga

SEQ ID NO: 755 - DNA, 25 bp, HERV-K
tctctggggacccagagagaaggag

SEQ ID NO: 756 - DNA, 25 bp, HERV-K
ggggacccagagagaaggaggcgtt

SEQ ID NO: 757 - DNA, 25 bp, HERV-K
cccagagagaaggaggcgttttcac

SEQ ID NO: 758 - DNA, 25 bp, HERV-K
agagaaggaggcgttttcaccacag

SEQ ID NO: 759 - DNA, 25 bp, HERV-K
aggaggcgttttcaccacagccgaa

SEQ ID NO: 760 - DNA, 25 bp, HERV-K
gcgttttcaccacagccgaacaggg

SEQ ID NO: 761 - DNA, 25 bp, HERV-K
ttcaccacagccgaacagggcagga

SEQ ID NO: 762 - DNA, 25 bp, HERV-K
cacagccgaacagggcaggacccca

SEQ ID NO: 763 - DNA, 25 bp, HERV-K
ccgaacagggcaggaccccagcacc

SEQ ID NO: 764 - DNA, 25 bp, HERV-K
cagggcaggaccccagcacccggga

SEQ ID NO: 765 - DNA, 25 bp, HERV-K
caggaccccagcacccgggacccag

SEQ ID NO: 766 - DNA, 25 bp, HERV-K
ccccagcacccgggacccagcggga

SEQ ID NO: 767 - DNA, 25 bp, HERV-K
gcacccgggacccagcgggactttg

SEQ ID NO: 768 - DNA, 25 bp, HERV-K
cgggacccagcgggactttgccaag

SEQ ID NO: 769 - DNA, 25 bp, HERV-K
cccagcgggactttgccaaggggat

SEQ ID NO: 770 - DNA, 25 bp, HERV-K
cgggactttgccaaggggatggacc

SEQ ID NO: 771 - DNA, 25 bp, HERV-K
ctttgccaaggggatggacctggct

SEQ ID NO: 772 - DNA, 25 bp, HERV-K
ccaaggggatggacctggctgggcc

SEQ ID NO: 773 - DNA, 25 bp, HERV-K
gggatggacctggctgggccacgcg

SEQ ID NO: 774 - DNA, 25 bp, HERV-K
ggacctggctgggccacgcggctgt

SEQ ID NO: 775 - DNA, 25 bp, HERV-K
tggctgggccacgcggctgtttgtg

SEQ ID NO: 776 - DNA, 25 bp, HERV-K
gggccacgcggctgtttgtgtaggg

SEQ ID NO: 777 - DNA, 25 bp, HERV-K
acgcggctgtttgtgtagggaaaag

SEQ ID NO: 778 - DNA, 25 bp, HERV-K
gtagaaaaggaagacataaactcca

SEQ ID NO: 779 - DNA, 25 bp, HERV-K
aaaggaagacataaactccattttg

SEQ ID NO: 780 - DNA, 25 bp, HERV-K
aagacataaactccattttgagctg

SEQ ID NO: 781 - DNA, 25 bp, HERV-K
ataaactccattttgagctgtacta

SEQ ID NO: 782 - DNA, 25 bp, HERV-K
agaaaaattattttgccttgacctg

SEQ ID NO: 783 - DNA, 25 bp, HERV-K
aattattttgccttgacctgctgtt

SEQ ID NO: 784 - DNA, 25 bp, HERV-K
ttttgccttgacctgctgttaacct

SEQ ID NO: 785 - DNA, 25 bp, HERV-K
ccttgacctgctgttaacctgtaac

SEQ ID NO: 786 - DNA, 25 bp, HERV-K
acctgctgttaacctgtaactgtag

SEQ ID NO: 787 - DNA, 25 bp, HERV-K
ctgttaacctgtaactgtagcccca

SEQ ID NO: 788 - DNA, 25 bp, HERV-K
aacctgtaactgtagccccaaccct

SEQ ID NO: 789 - DNA, 25 bp, HERV-K
tgtagccccaaccctgtgctcaaag

SEQ ID NO: 790 - DNA, 25 bp, HERV-K
tttaagggatcaagggctgtacagg

SEQ ID NO: 791 - DNA, 25 bp, HERV-K
gggatcaagggctgtacaggatgtg

SEQ ID NO: 792 - DNA, 25 bp, HERV-K
caagggctgtacaggatgtgccttg

SEQ ID NO: 793 - DNA, 25 bp, HERV-K
atgtgccttgttaacaatgtgttta

SEQ ID NO: 794 - DNA, 25 bp, HERV-K
ccattctccattaatcaggggcacg

SEQ ID NO: 795 - DNA, 25 bp, HERV-K
ctccattaatcaggggcacgatgca

SEQ ID NO: 796 - DNA, 25 bp, HERV-K
ttaatcaggggcacgatgcactgcg

SEQ ID NO: 797 - DNA, 25 bp, HERV-K
gcacgatgcactgcggaaagccaca

SEQ ID NO: 798 - DNA, 25 bp, HERV-K
ccacagggacctctgcccgagaaag

SEQ ID NO: 799 - DNA, 25 bp, HERV-K
gggacctctgcccgagaaagcctgg

SEQ ID NO: 800 - DNA, 25 bp, HERV-K
ctctgcccgagaaagcctgggtatt

SEQ ID NO: 801 - DNA, 25 bp, HERV-K
gaaagcctgggtattgtccaaggct

SEQ ID NO: 802 - DNA, 25 bp, HERV-K
cctgggtattgtccaaggcttcccc

SEQ ID NO: 803 - DNA, 25 bp, HERV-K
gtattgtccaaggcttccccccact

SEQ ID NO: 804 - DNA, 25 bp, HERV-K
gtccaaggcttccccccactgagac

SEQ ID NO: 805 - DNA, 25 bp, HERV-K
aggcttccccccactgagacagcct

SEQ ID NO: 806 - DNA, 25 bp, HERV-K
ccactgagacagcctgagatacggc

SEQ ID NO: 807 - DNA, 25 bp, HERV-K
aggaaggcctccgtctcctgcatgt

SEQ ID NO: 808 - DNA, 25 bp, HERV-K
ggcctccgtctcctgcatgtccttg

SEQ ID NO: 809 - DNA, 25 bp, HERV-K
ccgtctcctgcatgtccttgggaat

SEQ ID NO: 810 - DNA, 25 bp, HERV-K
tcctgcatgtccttgggaatggaat

SEQ ID NO: 811 - DNA, 25 bp, HERV-K
catgtccttgggaatggaatgtctt

SEQ ID NO: 812 - DNA, 25 bp, HERV-K
ccttgggaatggaatgtcttggtgt

SEQ ID NO: 813 - DNA, 25 bp, HERV-K
ggaatgtcttggtgtaaaacccgat

SEQ ID NO: 814 - DNA, 25 bp, HERV-K
gtcttggtgtaaaacccgatagtac

SEQ ID NO: 815 - DNA, 25 bp, HERV-K
ggtgtaaaacccgatagtacattcc

SEQ ID NO: 816 - DNA, 25 bp, HERV-K
aaaacccgatagtacattccttcta

SEQ ID NO: 817 - DNA, 25 bp, HERV-K
ccgatagtacattccttctattctg

SEQ ID NO: 818 - DNA, 25 bp, HERV-K
agtacattccttctattctgagaga

SEQ ID NO: 819 - DNA, 25 bp, HERV-K
ttctattctgagagaagaaaaccac

SEQ ID NO: 820 - DNA, 25 bp, HERV-K
ttctgagagaagaaaaccaccctgt

SEQ ID NO: 821 - DNA, 25 bp, HERV-K
agagaagaaaaccaccctgtggctg

SEQ ID NO: 822 - DNA, 25 bp, HERV-K
gaggtgagatatgctagcggcaatg

SEQ ID NO: 823 - DNA, 25 bp, HERV-K
gagatatgctagcggcaatgctgct

SEQ ID NO: 824 - DNA, 25 bp, HERV-K
atgctagcggcaatgctgctctgtt

SEQ ID NO: 825 - DNA, 25 bp, HERV-K
tggcctatgtgcacatctgggcaca

SEQ ID NO: 826 - DNA, 25 bp, HERV-K
tatgtgcacatctgggcacagaacc

SEQ ID NO: 827 - DNA, 25 bp, HERV-K
gcacatctgggcacagaacctcccc

SEQ ID NO: 828 - DNA, 25 bp, HERV-K
tctgggcacagaacctccccttgaa

SEQ ID NO: 829 - DNA, 25 bp, HERV-K
gcacagaacctccccttgaacttgt

SEQ ID NO: 830 - DNA, 25 bp, HERV-K
gaacctccccttgaacttgtgacac

SEQ ID NO: 831 - DNA, 25 bp, HERV-K
tccccttgaacttgtgacacagatt

SEQ ID NO: 832 - DNA, 25 bp, HERV-K
ttgaacttgtgacacagattccttt

SEQ ID NO: 833 - DNA, 25 bp, HERV-K
cttgtgacacagattcctttgttca

SEQ ID NO: 834 - DNA, 25 bp, HERV-K
agattcctttgttcacatgttttcc

SEQ ID NO: 835 - DNA, 25 bp, HERV-K
ctccccactatcgccctgttctccc

SEQ ID NO: 836 - DNA, 25 bp, HERV-K
cactatcgccctgttctcccaccgc

SEQ ID NO: 837 - DNA, 25 bp, HERV-K
tcgccctgttctcccaccgcattcc

SEQ ID NO: 838 - DNA, 25 bp, HERV-K
ctgttctcccaccgcattccccttg

SEQ ID NO: 839 - DNA, 25 bp, HERV-K
ctcccaccgcattccccttgctgag

SEQ ID NO: 840 - DNA, 25 bp, HERV-K
tagtaatctgtagataccaagggaa

SEQ ID NO: 841 - DNA, 25 bp, HERV-K
atctgtagataccaagggaactcag

SEQ ID NO: 842 - DNA, 25 bp, HERV-K
tagataccaagggaactcagagacc

SEQ ID NO: 843 - DNA, 25 bp, HERV-K
accaagggaactcagagaccatggc

SEQ ID NO: 844 - DNA, 25 bp, HERV-K
gggaactcagagaccatggccggtg

SEQ ID NO: 845 - DNA, 25 bp, HERV-K
ctcagagaccatggccggtgcacat

SEQ ID NO: 846 - DNA, 25 bp, HERV-K
agaccatggccggtgcacatcctcc

SEQ ID NO: 847 - DNA, 25 bp, HERV-K
atggccggtgcacatcctccgtacg

SEQ ID NO: 848 - DNA, 25 bp, HERV-K
cggtgcacatcctccgtacgctgag

SEQ ID NO: 849 - DNA, 25 bp, HERV-K
ctgagcgctggtcccctgggcccat

SEQ ID NO: 850 - DNA, 25 bp, HERV-K
cgctggtcccctgggcccattgttc

SEQ ID NO: 851 - DNA, 25 bp, HERV-K
cctcagtctctcatccctcctgacg

SEQ ID NO: 852 - DNA, 25 bp, HERV-K
gtctctcatccctcctgacgagaaa

SEQ ID NO: 853 - DNA, 25 bp, HERV-K
tcatccctcctgacgagaaataccc

SEQ ID NO: 854 - DNA, 25 bp, HERV-K
aggggctggcccccttcatctgatg

SEQ ID NO: 855 - DNA, 25 bp, HERV-K
ctggcccccttcatctgatgcccaa

SEQ ID NO: 856 - DNA, 25 bp, HERV-K
tcatctgatgcccaatgtgggtgcc

SEQ ID NO: 857 - DNA, 25 bp, HERV-K
tgatgcccaatgtgggtgcctttct

SEQ ID NO: 858 - DNA, 25 bp, HERV-K
tttctctagggtgaaggtactctac

SEQ ID NO: 859 - DNA, 25 bp, HERV-K
ctagggtgaaggtactctacagtgt

SEQ ID NO: 860 - DNA, 25 bp, HERV-K
gtgaaggtactctacagtgtggtca

SEQ ID NO: 861 - DNA, 25 bp, HERV-K
ggtactctacagtgtggtcattgag

SEQ ID NO: 862 - DNA, 25 bp, HERV-K
tctacagtgtggtcattgaggacaa

SEQ ID NO: 863 - DNA, 25 bp, HERV-K
gacaagttgacgagagagtcccaag

SEQ ID NO: 864 - DNA, 25 bp, HERV-K
gttgacgagagagtcccaagtacgt

SEQ ID NO: 865 - DNA, 25 bp, HERV-K
cgagagagtcccaagtacgtccacg

SEQ ID NO: 866 - DNA, 25 bp, HERV-K
cttagaggaacccagggtaacgatg

SEQ ID NO: 867 - DNA, 25 bp, HERV-K
caaacaggagaagatattgtttcag

SEQ ID NO: 868 - DNA, 25 bp, HERV-K
aggagaagatattgtttcagtttct

SEQ ID NO: 869 - DNA, 25 bp, HERV-K
gatgcccctaaaagctgtgtaacag

SEQ ID NO: 870 - DNA, 25 bp, HERV-K
ccctaaaagctgtgtaacagattgt

SEQ ID NO: 871 - DNA, 25 bp, HERV-K
gaagaagaggcagggacagaatccc

SEQ ID NO: 872 - DNA, 25 bp, HERV-K
agaggcagggacagaatcccagcaa

SEQ ID NO: 873 - DNA, 25 bp, HERV-K
cagggacagaatcccagcaaggaac

SEQ ID NO: 874 - DNA, 25 bp, HERV-K
acagaatcccagcaaggaacggaaa

SEQ ID NO: 875 - DNA, 25 bp, HERV-K
atcccagcaaggaacggaaagttca

SEQ ID NO: 876 - DNA, 25 bp, HERV-K
tgactacagtcaattacaggagata

SEQ ID NO: 877 - DNA, 25 bp, HERV-K
acaggagataatataccctgaatca

SEQ ID NO: 878 - DNA, 25 bp, HERV-K
accacgatcgccatcaactcctcct

SEQ ID NO: 879 - DNA, 25 bp, HERV-K
gatcgccatcaactcctcctcccgt

SEQ ID NO: 880 - DNA, 25 bp, HERV-K
ccatcaactcctcctcccgtggttc

SEQ ID NO: 881 - DNA, 25 bp, HERV-K
aactcctcctcccgtggttcagatg

SEQ ID NO: 882 - DNA, 25 bp, HERV-K
cctcaaacgcaggttagacaagcac

SEQ ID NO: 883 - DNA, 25 bp, HERV-K
aacgcaggttagacaagcacaaacc

SEQ ID NO: 884 - DNA, 25 bp, HERV-K
agacaagcacaaaccccaagagaaa

SEQ ID NO: 885 - DNA, 25 bp, HERV-K
agcacaaaccccaagagaaaatcaa

SEQ ID NO: 886 - DNA, 25 bp, HERV-K
ccaagagaaaatcaagtagaaaggg

SEQ ID NO: 887 - DNA, 25 bp, HERV-K
agaaaatcaagtagaaagggacaga

SEQ ID NO: 888 - DNA, 25 bp, HERV-K
atcaagtagaaagggacagagtctc

SEQ ID NO: 889 - DNA, 25 bp, HERV-K
gtagaaagggacagagtctctatcc

SEQ ID NO: 890 - DNA, 25 bp, HERV-K
aagggacagagtctctatcccggca

SEQ ID NO: 891 - DNA, 25 bp, HERV-K
tatcccggcaatgccaactcagata

SEQ ID NO: 892 - DNA, 25 bp, HERV-K
cggcaatgccaactcagatacagta

SEQ ID NO: 893 - DNA, 25 bp, HERV-K
aaaataagacccaaccgctggtagt

SEQ ID NO: 894 - DNA, 25 bp, HERV-K
aagacccaaccgctggtagtttatc

SEQ ID NO: 895 - DNA, 25 bp, HERV-K
cgctggtagtttatcaataccggct

SEQ ID NO: 896 - DNA, 25 bp, HERV-K
gtagtttatcaataccggctgccaa

SEQ ID NO: 897 - DNA, 25 bp, HERV-K
ttatcaataccggctgccaaccgag

SEQ ID NO: 898 - DNA, 25 bp, HERV-K
aataccggctgccaaccgagcttca

SEQ ID NO: 899 - DNA, 25 bp, HERV-K
cggctgccaaccgagcttcagtatc

SEQ ID NO: 900 - DNA, 25 bp, HERV-K
gccaaccgagcttcagtatcggcct

SEQ ID NO: 901 - DNA, 25 bp, HERV-K
ccgagcttcagtatcggcctccttc

SEQ ID NO: 902 - DNA, 25 bp, HERV-K
cttcagtatcggcctccttcagagg

SEQ ID NO: 903 - DNA, 25 bp, HERV-K
gtatcggcctccttcagaggttcaa

SEQ ID NO: 904 - DNA, 25 bp, HERV-K
ggcctccttcagaggttcaatacag

SEQ ID NO: 905 - DNA, 25 bp, HERV-K
ccttcagaggttcaatacagacctc

SEQ ID NO: 906 - DNA, 25 bp, HERV-K
caccataccagcaacccacagcgat

SEQ ID NO: 907 - DNA, 25 bp, HERV-K
gcaacccacagcgatggcgtctaat

SEQ ID NO: 908 - DNA, 25 bp, HERV-K
ccacagcgatggcgtctaattcacc

SEQ ID NO: 909 - DNA, 25 bp, HERV-K
gcgatggcgtctaattcaccagcaa

SEQ ID NO: 910 - DNA, 25 bp, HERV-K
ggcgtctaattcaccagcaacacag

SEQ ID NO: 911 - DNA, 25 bp, HERV-K
ctaattcaccagcaacacaggacgc

SEQ ID NO: 912 - DNA, 25 bp, HERV-K
tcaccagcaacacaggacgcggcgc

SEQ ID NO: 913 - DNA, 25 bp, HERV-K
agcaacacaggacgcggcgctgtat

SEQ ID NO: 914 - DNA, 25 bp, HERV-K
cacaggacgcggcgctgtatcctca

SEQ ID NO: 915 - DNA, 25 bp, HERV-K
gacgcggcgctgtatcctcagccgc

SEQ ID NO: 916 - DNA, 25 bp, HERV-K
ggcgctgtatcctcagccgcccact

SEQ ID NO: 917 - DNA, 25 bp, HERV-K
atcacgtagtggacagggtggtgca

SEQ ID NO: 918 - DNA, 25 bp, HERV-K
gtagtggacagggtggtgcactgca

SEQ ID NO: 919 - DNA, 25 bp, HERV-K
ggacagggtggtgcactgcatgcag

SEQ ID NO: 920 - DNA, 25 bp, HERV-K
gggtggtgcactgcatgcagtcatt

SEQ ID NO: 921 - DNA, 25 bp, HERV-K
gtgcactgcatgcagtcattgatga

SEQ ID NO: 922 - DNA, 25 bp, HERV-K
aaatggtctttttactccctggaaa

SEQ ID NO: 923 - DNA, 25 bp, HERV-K
gtctttttactccctggaaagcccc

SEQ ID NO: 924 - DNA, 25 bp, HERV-K
agggagggtaggcctttgagggaga

SEQ ID NO: 925 - DNA, 25 bp, HERV-K
gggtaggcctttgagggagatcaag

SEQ ID NO: 926 - DNA, 25 bp, HERV-K
ggcctttgagggagatcaagtctaa

SEQ ID NO: 927 - DNA, 25 bp, HERV-K
caggaaaagctgcttattgggctaa

SEQ ID NO: 928 - DNA, 25 bp, HERV-K
aaagctgcttattgggctaatcagg

SEQ ID NO: 929 - DNA, 25 bp, HERV-K
tgtttctgtcatcggcataggtact

SEQ ID NO: 930 - DNA, 25 bp, HERV-K
ctgtcatcggcataggtactgcctc

SEQ ID NO: 931 - DNA, 25 bp, HERV-K
atcggcataggtactgcctcagaag

SEQ ID NO: 932 - DNA, 25 bp, HERV-K
ggttcagcctgtgatcacttcattc

SEQ ID NO: 933 - DNA, 25 bp, HERV-K
agcctgtgatcacttcattccaatc

SEQ ID NO: 934 - DNA, 25 bp, HERV-K
gtgatcacttcattccaatcaattt

SEQ ID NO: 935 - DNA, 25 bp, HERV-K
cacttcattccaatcaatttatggg

SEQ ID NO: 936 - DNA, 25 bp, HERV-K
agggactaggaaagaagtcccaatt

SEQ ID NO: 937 - DNA, 25 bp, HERV-K
ctaggaaagaagtcccaattgaggc

SEQ ID NO: 938 - DNA, 25 bp, HERV-K
ccattccattaacttgggggaaaaa

SEQ ID NO: 939 - DNA, 25 bp, HERV-K
ccattaacttgggggaaaaaaaaac

SEQ ID NO: 940 - DNA, 25 bp, HERV-K
aacttgggggaaaaaaaaacaactg

SEQ ID NO: 941 - DNA, 25 bp, HERV-K
gggggaaaaaaaaacaactgtatgg

SEQ ID NO: 942 - DNA, 25 bp, HERV-K
aaaacaactgtatggtaaatcagca

SEQ ID NO: 943 - DNA, 25 bp, HERV-K
aactgtatggtaaatcagcagcgct

SEQ ID NO: 944 - DNA, 25 bp, HERV-K
tatggtaaatcagcagcgcttccaa

SEQ ID NO: 945 - DNA, 25 bp, HERV-K
taaatcagcagcgcttccaaaacaa

SEQ ID NO: 946 - DNA, 25 bp, HERV-K
cagcagcgcttccaaaacaaaaact

SEQ ID NO: 947 - DNA, 25 bp, HERV-K
attagaaaaaggacattgagccttc

SEQ ID NO: 948 - DNA, 25 bp, HERV-K
aaaaaggacattgagccttcatttt

SEQ ID NO: 949 - DNA, 25 bp, HERV-K
attttcgccttggaattctgtttgt

SEQ ID NO: 950 - DNA, 25 bp, HERV-K
cgccttggaattctgtttgtaattc

SEQ ID NO: 951 - DNA, 25 bp, HERV-K
aaatccggcagatggcgtataatgc

SEQ ID NO: 952 - DNA, 25 bp, HERV-K
cggcagatggcgtataatgccgtaa

SEQ ID NO: 953 - DNA, 25 bp, HERV-K
gatggcgtataatgccgtaattcaa

SEQ ID NO: 954 - DNA, 25 bp, HERV-K
tttgcttttaccacaccagcctaaa

SEQ ID NO: 955 - DNA, 25 bp, HERV-K
ttttaccacaccagcctaaataata

SEQ ID NO: 956 - DNA, 25 bp, HERV-K
aatagttcaactatttgtcagctca

SEQ ID NO: 957 - DNA, 25 bp, HERV-K
ttcaactatttgtcagctcaagctc

SEQ ID NO: 958 - DNA, 25 bp, HERV-K
ctatttgtcagctcaagctctgcaa

SEQ ID NO: 959 - DNA, 25 bp, HERV-K
ttttcagactgttacatcgttcact

SEQ ID NO: 960 - DNA, 25 bp, HERV-K
agactgttacatcgttcactatgtt

SEQ ID NO: 961 - DNA, 25 bp, HERV-K
ctctactcctttccgttacttggga

SEQ ID NO: 962 - DNA, 25 bp, HERV-K
tcggaattaaatagtgaaagaacgt

SEQ ID NO: 963 - DNA, 25 bp, HERV-K
atagtgaaagaacgttaactccaga

SEQ ID NO: 964 - DNA, 25 bp, HERV-K
gaaagaacgttaactccagaggcaa

SEQ ID NO: 965 - DNA, 25 bp, HERV-K
ttttgctactgcacattccctaaca

SEQ ID NO: 966 - DNA, 25 bp, HERV-K
ctactgcacattccctaacaggcat

SEQ ID NO: 967 - DNA, 25 bp, HERV-K
gcacattccctaacaggcatcattg

SEQ ID NO: 968 - DNA, 25 bp, HERV-K
ttccctaacaggcatcattgttcaa

SEQ ID NO: 969 - DNA, 25 bp, HERV-K
attattgacaatcgttaccccaaaa

SEQ ID NO: 970 - DNA, 25 bp, HERV-K
tgacaatcgttaccccaaaacaaaa

SEQ ID NO: 971 - DNA, 25 bp, HERV-K
atcgttaccccaaaacaaaaatctt

SEQ ID NO: 972 - DNA, 25 bp, HERV-K
acctaaagttaccaaacataagcct

SEQ ID NO: 973 - DNA, 25 bp, HERV-K
acataagcctttaaaaaatgctctg

SEQ ID NO: 974 - DNA, 25 bp, HERV-K
agcctttaaaaaatgctctggcagt

SEQ ID NO: 975 - DNA, 25 bp, HERV-K
gggccaaaagaatgagtcatcaaaa

SEQ ID NO: 976 - DNA, 25 bp, HERV-K
aaaagaatgagtcatcaaaactcag

SEQ ID NO: 977 - DNA, 25 bp, HERV-K
aatgagtcatcaaaactcagtatca

SEQ ID NO: 978 - DNA, 25 bp, HERV-K
gtcatcaaaactcagtatcacttga

SEQ ID NO: 979 - DNA, 25 bp, HERV-K
caaaactcagtatcacttgactcaa

SEQ ID NO: 980 - DNA, 25 bp, HERV-K
ctcagtatcacttgactcaaagagc

SEQ ID NO: 981 - DNA, 25 bp, HERV-K
tatcacttgactcaaagagcagagt

SEQ ID NO: 982 - DNA, 25 bp, HERV-K
cttgactcaaagagcagagttggtt

SEQ ID NO: 983 - DNA, 25 bp, HERV-K
tggttgccgtcattacagtgttaac

SEQ ID NO: 984 - DNA, 25 bp, HERV-K
gccgtcattacagtgttaacaagat

SEQ ID NO: 985 - DNA, 25 bp, HERV-K
caggctacaaaggatattgagagag

SEQ ID NO: 986 - DNA, 25 bp, HERV-K
tacaaaggatattgagagagcccta

SEQ ID NO: 987 - DNA, 25 bp, HERV-K
aggatattgagagagccctaatcaa

SEQ ID NO: 988 - DNA, 25 bp, HERV-K
attgagagagccctaatcaaataca

SEQ ID NO: 989 - DNA, 25 bp, HERV-K
ttatggatgatcagttaaacccgct

SEQ ID NO: 990 - DNA, 25 bp, HERV-K
tcagttaaacccgctgtttaatttg

SEQ ID NO: 991 - DNA, 25 bp, HERV-K
taaacccgctgtttaatttgttaca

SEQ ID NO: 992 - DNA, 25 bp, HERV-K
ttgactcatgtaaatgcaataggat

SEQ ID NO: 993 - DNA, 25 bp, HERV-K
cattgcacccagtgtcagattctac

SEQ ID NO: 994 - DNA, 25 bp, HERV-K
agtgtcagattctacacctggccac

SEQ ID NO: 995 - DNA, 25 bp, HERV-K
cagattctacacctggccactcagg

SEQ ID NO: 996 - DNA, 25 bp, HERV-K
tctacacctggccactcaggaggca

SEQ ID NO: 997 - DNA, 25 bp, HERV-K
acctggccactcaggaggcaagagt

SEQ ID NO: 998 - DNA, 25 bp, HERV-K
gccactcaggaggcaagagttaatc

SEQ ID NO: 999 - DNA, 25 bp, HERV-K
atggcaaatggatgtcatgcacgta

SEQ ID NO: 1000 - DNA, 25 bp, HERV-K
aaatggatgtcatgcacgtaccttc

SEQ ID NO: 1001 - DNA, 25 bp, HERV-K
gatgtcatgcacgtaccttcatttg

SEQ ID NO: 1002 - DNA, 25 bp, HERV-K
catgcacgtaccttcatttggaaaa

SEQ ID NO: 1003 - DNA, 25 bp, HERV-K
gttccagaaaaagttaaaacagaca

SEQ ID NO: 1004 - DNA, 25 bp, HERV-K
agaaaaagttaaaacagacaatggg

SEQ ID NO: 1005 - DNA, 25 bp, HERV-K
aagttaaaacagacaatgggccagg

SEQ ID NO: 1006 - DNA, 25 bp, HERV-K
aaaacagacaatgggccaggttact

SEQ ID NO: 1007 - DNA, 25 bp, HERV-K
agacaatgggccaggttactgtagt

SEQ ID NO: 1008 - DNA, 25 bp, HERV-K
ccaggttactgtagtaaagcagttc

SEQ ID NO: 1009 - DNA, 25 bp, HERV-K
aaacaaaaaaaaggaaaagacagga

SEQ ID NO: 1010 - DNA, 25 bp, HERV-K
aaggaaaagacaggagtataacact

SEQ ID NO: 1011 - DNA, 25 bp, HERV-K
aaagacaggagtataacactcccca

SEQ ID NO: 1012 - DNA, 25 bp, HERV-K
cactacctctgcagaacaacatctt

SEQ ID NO: 1013 - DNA, 25 bp, HERV-K
aaaataaaacatgggaaatggggaa

SEQ ID NO: 1014 - DNA, 25 bp, HERV-K
aaaacatgggaaatggggaaggtga

SEQ ID NO: 1015 - DNA, 25 bp, HERV-K
taaagttctacaatgaactcactgg

SEQ ID NO: 1016 - DNA, 25 bp, HERV-K
ttctacaatgaactcactggagatg

SEQ ID NO: 1017 - DNA, 25 bp, HERV-K
caatgaactcactggagatgcaaag

SEQ ID NO: 1018 - DNA, 25 bp, HERV-K
aactcactggagatgcaaagaaaag

SEQ ID NO: 1019 - DNA, 25 bp, HERV-K
actggagatgcaaagaaaagtgtgg

SEQ ID NO: 1020 - DNA, 25 bp, HERV-K
agatgcaaagaaaagtgtggagatg

SEQ ID NO: 1021 - DNA, 25 bp, HERV-K
caaagaaaagtgtggagatggagac

SEQ ID NO: 1022 - DNA, 25 bp, HERV-K
aaaagtgtggagatggagacacccc

SEQ ID NO: 1023 - DNA, 25 bp, HERV-K
tgtggagatggagacaccccaatcg

SEQ ID NO: 1024 - DNA, 25 bp, HERV-K
agatggagacaccccaatcgactcg

SEQ ID NO: 1025 - DNA, 25 bp, HERV-K
gagacaccccaatcgactcgccagg

SEQ ID NO: 1026 - DNA, 25 bp, HERV-K
accccaatcgactcgccaggtaaac

SEQ ID NO: 1027 - DNA, 25 bp, HERV-K
aatcgactcgccaggtaaacaaaat

SEQ ID NO: 1028 - DNA, 25 bp, HERV-K
ggtgatatcagaagaacagaaaaag

SEQ ID NO: 1029 - DNA, 25 bp, HERV-K
tatcagaagaacagaaaaagttgcc

SEQ ID NO: 1030 - DNA, 25 bp, HERV-K
gaagaacagaaaaagttgccttcca

SEQ ID NO: 1031 - DNA, 25 bp, HERV-K
acagaaaaagttgccttccatcaag

SEQ ID NO: 1032 - DNA, 25 bp, HERV-K
aaaagttgccttccatcaaggaagc

SEQ ID NO: 1033 - DNA, 25 bp, HERV-K
ttgccttccatcaaggaagcagagt

SEQ ID NO: 1034 - DNA, 25 bp, HERV-K
ttccatcaaggaagcagagttgcca

SEQ ID NO: 1035 - DNA, 25 bp, HERV-K
tcaaggaagcagagttgccaatata

SEQ ID NO: 1036 - DNA, 25 bp, HERV-K
gaagcagagttgccaatataggcac

SEQ ID NO: 1037 - DNA, 25 bp, HERV-K
agagttgccaatataggcacaatta

SEQ ID NO: 1038 - DNA, 25 bp, HERV-K
tgccaatataggcacaattaaagaa

SEQ ID NO: 1039 - DNA, 25 bp, HERV-K
atataggcacaattaaagaagctga

SEQ ID NO: 1040 - DNA, 25 bp, HERV-K
cacagttagctaaaaaaaaaagcct

SEQ ID NO: 1041 - DNA, 25 bp, HERV-K
aaaaaagcctagagaatacaaaggt

SEQ ID NO: 1042 - DNA, 25 bp, HERV-K
agcctagagaatacaaaggtgacac

SEQ ID NO: 1043 - DNA, 25 bp, HERV-K
agagaatacaaaggtgacaccaact

SEQ ID NO: 1044 - DNA, 25 bp, HERV-K
atacaaaggtgacaccaactccaga

SEQ ID NO: 1045 - DNA, 25 bp, HERV-K
aaggtgacaccaactccagagaata

SEQ ID NO: 1046 - DNA, 25 bp, HERV-K
gacaccaactccagagaatatgctg

SEQ ID NO: 1047 - DNA, 25 bp, HERV-K
gaatatgctgcttgcagctctgatg

SEQ ID NO: 1048 - DNA, 25 bp, HERV-K
atcaacggtggtaagtcttcccaag

SEQ ID NO: 1049 - DNA, 25 bp, HERV-K
cggtggtaagtcttcccaagtctgc

SEQ ID NO: 1050 - DNA, 25 bp, HERV-K
gtaagtcttcccaagtctgcaggag

SEQ ID NO: 1051 - DNA, 25 bp, HERV-K
tcttcccaagtctgcaggagcagct

SEQ ID NO: 1052 - DNA, 25 bp, HERV-K
ccttaattcgggcagttacatagat

SEQ ID NO: 1053 - DNA, 25 bp, HERV-K
attcgggcagttacatagatggata

SEQ ID NO: 1054 - DNA, 25 bp, HERV-K
ggcagttacatagatggataatcct

SEQ ID NO: 1055 - DNA, 25 bp, HERV-K
tagtgcatgggtgcctggccccaca

SEQ ID NO: 1056 - DNA, 25 bp, HERV-K
catgggtgcctggccccacagatga

SEQ ID NO: 1057 - DNA, 25 bp, HERV-K
gtgcctggccccacagatgactgtt

SEQ ID NO: 1058 - DNA, 25 bp, HERV-K
tggccccacagatgactgttgccct

SEQ ID NO: 1059 - DNA, 25 bp, HERV-K
gcccaacctgaagaaggaatgatga

SEQ ID NO: 1060 - DNA, 25 bp, HERV-K
cattgggtatccttatcctcctgtt

SEQ ID NO: 1061 - DNA, 25 bp, HERV-K
ggtatccttatcctcctgtttgcct

SEQ ID NO: 1062 - DNA, 25 bp, HERV-K
ccttatcctcctgtttgcctaggga

SEQ ID NO: 1063 - DNA, 25 bp, HERV-K
cctacagtcagtgctaccagtagat

SEQ ID NO: 1064 - DNA, 25 bp, HERV-K
agtcagtgctaccagtagatttact

SEQ ID NO: 1065 - DNA, 25 bp, HERV-K
catggtaagtggaatgtcacagata

SEQ ID NO: 1066 - DNA, 25 bp, HERV-K
tcattacaatgtaggcctaagggga

SEQ ID NO: 1067 - DNA, 25 bp, HERV-K
acaatgtaggcctaaggggaaggct

SEQ ID NO: 1068 - DNA, 25 bp, HERV-K
gtaggcctaaggggaaggcttgccc

SEQ ID NO: 1069 - DNA, 25 bp, HERV-K
caaaaagcccagaagtcttagtctg

SEQ ID NO: 1070 - DNA, 25 bp, HERV-K
agcccagaagtcttagtctgcggag

SEQ ID NO: 1071 - DNA, 25 bp, HERV-K
agaagtcttagtctgcggagaatgt

SEQ ID NO: 1072 - DNA, 25 bp, HERV-K
tcttagtctgcggagaatgtgtggc

SEQ ID NO: 1073 - DNA, 25 bp, HERV-K
gtctgcggagaatgtgtggctgata

SEQ ID NO: 1074 - DNA, 25 bp, HERV-K
gtggctgatactgcagtgtagtaca

SEQ ID NO: 1075 - DNA, 25 bp, HERV-K
tgatactgcagtgtagtacaaaaca

SEQ ID NO: 1076 - DNA, 25 bp, HERV-K
ctgcagtgtagtacaaaacaatgaa

SEQ ID NO: 1077 - DNA, 25 bp, HERV-K
ttttgaactatgatagactgggtcc

SEQ ID NO: 1078 - DNA, 25 bp, HERV-K
aactatgatagactgggtcccttga

SEQ ID NO: 1079 - DNA, 25 bp, HERV-K
tgatagactgggtcccttgaggcca

SEQ ID NO: 1080 - DNA, 25 bp, HERV-K
ggtcccttgaggccaattatatcat

SEQ ID NO: 1081 - DNA, 25 bp, HERV-K
cttgaggccaattatatcataactg

SEQ ID NO: 1082 - DNA, 25 bp, HERV-K
attatatcataactgtacaggccag

SEQ ID NO: 1083 - DNA, 25 bp, HERV-K
atcataactgtacaggccagactca

SEQ ID NO: 1084 - DNA, 25 bp, HERV-K
aactgtacaggccagactcattcat

SEQ ID NO: 1085 - DNA, 25 bp, HERV-K
tacaggccagactcattcatgttca

SEQ ID NO: 1086 - DNA, 25 bp, HERV-K
tggcccattaatccagcctatgacg

SEQ ID NO: 1087 - DNA, 25 bp, HERV-K
cattaatccagcctatgacggtgat

SEQ ID NO: 1088 - DNA, 25 bp, HERV-K
atccagcctatgacggtgatgtaac

SEQ ID NO: 1089 - DNA, 25 bp, HERV-K
gcctatgacggtgatgtaactgaaa

SEQ ID NO: 1090 - DNA, 25 bp, HERV-K
tgacggtgatgtaactgaaaggctg

SEQ ID NO: 1091 - DNA, 25 bp, HERV-K
atagaaggttagaatcactctgtcc

SEQ ID NO: 1092 - DNA, 25 bp, HERV-K
aggttagaatcactctgtccaagga

SEQ ID NO: 1093 - DNA, 25 bp, HERV-K
agaatcactctgtccaaggaaatgg

SEQ ID NO: 1094 - DNA, 25 bp, HERV-K
cactctgtccaaggaaatggggtga

SEQ ID NO: 1095 - DNA, 25 bp, HERV-K
tgtccaaggaaatggggtgaaaagg

SEQ ID NO: 1096 - DNA, 25 bp, HERV-K
tcatcaccttgaccaaagttagtcc

SEQ ID NO: 1097 - DNA, 25 bp, HERV-K
accttgaccaaagttagtcctgtta

SEQ ID NO: 1098 - DNA, 25 bp, HERV-K
gaccaaagttagtcctgttactggt

SEQ ID NO: 1099 - DNA, 25 bp, HERV-K
cctgaacatccagaattaggaagct

SEQ ID NO: 1100 - DNA, 25 bp, HERV-K
acatccagaattaggaagcttactg

SEQ ID NO: 1101 - DNA, 25 bp, HERV-K
cagaattaggaagcttactgtggcc

SEQ ID NO: 1102 - DNA, 25 bp, HERV-K
ttaggaagcttactgtggcctcaca

SEQ ID NO: 1103 - DNA, 25 bp, HERV-K
ttctggaaatcaagctataggaaca

SEQ ID NO: 1104 - DNA, 25 bp, HERV-K
tcctttgcaaaattgtgtaaaactc

SEQ ID NO: 1105 - DNA, 25 bp, HERV-K
tgcaaaattgtgtaaaactccctta

SEQ ID NO: 1106 - DNA, 25 bp, HERV-K
aactcccttatattgctagttgtag

SEQ ID NO: 1107 - DNA, 25 bp, HERV-K
gttattaaacctgattcccaaacca

SEQ ID NO: 1108 - DNA, 25 bp, HERV-K
taaacctgattcccaaaccataatc

SEQ ID NO: 1109 - DNA, 25 bp, HERV-K
ctgattcccaaaccataatctgtga

SEQ ID NO: 1110 - DNA, 25 bp, HERV-K
tcccaaaccataatctgtgaaaatt

SEQ ID NO: 1111 - DNA, 25 bp, HERV-K
aaccataatctgtgaaaattgtgga

SEQ ID NO: 1112 - DNA, 25 bp, HERV-K
taatctgtgaaaattgtggaatgtt

SEQ ID NO: 1113 - DNA, 25 bp, HERV-K
tgtgaaaattgtggaatgtttactt

SEQ ID NO: 1114 - DNA, 25 bp, HERV-K
aaattgtggaatgtttacttgcatt

SEQ ID NO: 1115 - DNA, 25 bp, HERV-K
gtggaatgtttacttgcattgattt

SEQ ID NO: 1116 - DNA, 25 bp, HERV-K
gcaccgtattctactaggaagagca

SEQ ID NO: 1117 - DNA, 25 bp, HERV-K
gtattctactaggaagagcaagaga

SEQ ID NO: 1118 - DNA, 25 bp, HERV-K
ctactaggaagagcaagagagggtg

SEQ ID NO: 1119 - DNA, 25 bp, HERV-K
aggaagagcaagagagggtgtgtgg

SEQ ID NO: 1120 - DNA, 25 bp, HERV-K
atccttgtgtccatggaccgaccat

SEQ ID NO: 1121 - DNA, 25 bp, HERV-K
gaccgaccatgggaggcttcgctat

SEQ ID NO: 1122 - DNA, 25 bp, HERV-K
accatgggaggcttcgctatccatc

SEQ ID NO: 1123 - DNA, 25 bp, HERV-K
gggaggcttcgctatccatccatat

SEQ ID NO: 1124 - DNA, 25 bp, HERV-K
gcttcgctatccatccatattttaa

SEQ ID NO: 1125 - DNA, 25 bp, HERV-K
gctatccatccatattttaacggaa

SEQ ID NO: 1126 - DNA, 25 bp, HERV-K
ttgatggcagtgattatgggcctca

SEQ ID NO: 1127 - DNA, 25 bp, HERV-K
ggcagtgattatgggcctcattgca

SEQ ID NO: 1128 - DNA, 25 bp, HERV-K
cagaatacgtaaatgattggcaaaa

SEQ ID NO: 1129 - DNA, 25 bp, HERV-K
tacgtaaatgattggcaaaagaatt

SEQ ID NO: 1130 - DNA, 25 bp, HERV-K
tcatttggatgggagaggctcatga

SEQ ID NO: 1131 - DNA, 25 bp, HERV-K
tggatgggagaggctcatgagcttg

SEQ ID NO: 1132 - DNA, 25 bp, HERV-K
gggagaggctcatgagcttggaata

SEQ ID NO: 1133 - DNA, 25 bp, HERV-K
tctttttcagttacgatgtgactgg

SEQ ID NO: 1134 - DNA, 25 bp, HERV-K
ttcagttacgatgtgactggaatac

SEQ ID NO: 1135 - DNA, 25 bp, HERV-K
ttacgatgtgactggaatacatcag

SEQ ID NO: 1136 - DNA, 25 bp, HERV-K
atcagatttttgtgttacaccacaa

SEQ ID NO: 1137 - DNA, 25 bp, HERV-K
atttttgtgttacaccacaagccta

SEQ ID NO: 1138 - DNA, 25 bp, HERV-K
tgtgttacaccacaagcctataatg

SEQ ID NO: 1139 - DNA, 25 bp, HERV-K
atggttagatgccatctgcaaggag

SEQ ID NO: 1140 - DNA, 25 bp, HERV-K
tagatgccatctgcaaggaggagaa

SEQ ID NO: 1141 - DNA, 25 bp, HERV-K
gccatctgcaaggaggagaagataa

SEQ ID NO: 1142 - DNA, 25 bp, HERV-K
ctgcaaggaggagaagataatctta

SEQ ID NO: 1143 - DNA, 25 bp, HERV-K
taaatttggtgccaggaacggagac

SEQ ID NO: 1144 - DNA, 25 bp, HERV-K
ttggtgccaggaacggagacaatcg

SEQ ID NO: 1145 - DNA, 25 bp, HERV-K
gccaggaacggagacaatcgtgaaa

SEQ ID NO: 1146 - DNA, 25 bp, HERV-K
gaacggagacaatcgtgaaagctgc

SEQ ID NO: 1147 - DNA, 25 bp, HERV-K
gagacaatcgtgaaagctgctgata

SEQ ID NO: 1148 - DNA, 25 bp, HERV-K
gcctcacaaatcttaagccagtcac

SEQ ID NO: 1149 - DNA, 25 bp, HERV-K
acaaatcttaagccagtcacttggg

SEQ ID NO: 1150 - DNA, 25 bp, HERV-K
tcttaagccagtcacttgggttaaa

SEQ ID NO: 1151 - DNA, 25 bp, HERV-K
agccagtcacttgggttaaaagcat

SEQ ID NO: 1152 - DNA, 25 bp, HERV-K
ttgggttaaaagcatcagaagtttc

SEQ ID NO: 1153 - DNA, 25 bp, HERV-K
agcatcagaagtttcactattgtaa

SEQ ID NO: 1154 - DNA, 25 bp, HERV-K
agctccaaagagacagcaaccagca

SEQ ID NO: 1155 - DNA, 25 bp, HERV-K
caaagagacagcaaccagcaagaat

SEQ ID NO: 1156 - DNA, 25 bp, HERV-K
agacagcaaccagcaagaatgggcc

SEQ ID NO: 1157 - DNA, 25 bp, HERV-K
gcaaccagcaagaatgggccatagt

SEQ ID NO: 1158 - DNA, 25 bp, HERV-K
cagcaagaatgggccatagtgacga

SEQ ID NO: 1159 - DNA, 25 bp, HERV-K
agaatgggccatagtgacgatggtg

SEQ ID NO: 1160 - DNA, 25 bp, HERV-K
gggccatagtgacgatggtggtttt

SEQ ID NO: 1161 - DNA, 25 bp, HERV-K
atagtgacgatggtggttttgtcaa

SEQ ID NO: 1162 - DNA, 25 bp, HERV-K
gacgatggtggttttgtcaaaaaga

SEQ ID NO: 1163 - DNA, 25 bp, HERV-K
gtcaaaaagaaaagggggggatatg

SEQ ID NO: 1164 - DNA, 25 bp, HERV-K
aaagaaaagggggggatatgtaagg

SEQ ID NO: 1165 - DNA, 25 bp, HERV-K
aaagggggggatatgtaaggaaaag

SEQ ID NO: 1166 - DNA, 25 bp, HERV-K
taaggaaaagagagatcagactttc

SEQ ID NO: 1167 - DNA, 25 bp, HERV-K
aaaagagagatcagactttcactgt

SEQ ID NO: 1168 - DNA, 25 bp, HERV-K
ctttcactgtgtctatgtagaaaag

SEQ ID NO: 1169 - DNA, 25 bp, HERV-K
actaagaaaaattgttttgccttga

SEQ ID NO: 1170 - DNA, 25 bp, HERV-K
tgctcacggaaacatgtgctgtaag

SEQ ID NO: 1171 - DNA, 25 bp, HERV-K
acggaaacatgtgctgtaaggttta

SEQ ID NO: 1172 - DNA, 25 bp, HERV-K
aacatgtgctgtaaggtttaaggga

SEQ ID NO: 1173 - DNA, 25 bp, HERV-K
gtgctgtaaggtttaagggatctag

SEQ ID NO: 1174 - DNA, 25 bp, HERV-K
tgcaggatgtaccttgttaacaata

SEQ ID NO: 1175 - DNA, 25 bp, HERV-K
accttgttaacaatatgtttgcagg

SEQ ID NO: 1176 - DNA, 25 bp, HERV-K
caatatgtttgcaggcagtatgttt

SEQ ID NO: 1177 - DNA, 25 bp, HERV-K
tgtttgcaggcagtatgtttggtaa

SEQ ID NO: 1178 - DNA, 25 bp, HERV-K
gcaggcagtatgtttggtaaaagtc

SEQ ID NO: 1179 - DNA, 25 bp, HERV-K
attaaccaggggctcaatgcactgt

SEQ ID NO: 1180 - DNA, 25 bp, HERV-K
ggctcaatgcactgtggaaagccac

SEQ ID NO: 1181 - DNA, 25 bp, HERV-K
aatgcactgtggaaagccacaggaa

SEQ ID NO: 1182 - DNA, 25 bp, HERV-K
ggaaagccacaggaacctctgccca

SEQ ID NO: 1183 - DNA, 25 bp, HERV-K
gccacaggaacctctgcccaagaaa

SEQ ID NO: 1184 - DNA, 25 bp, HERV-K
aggaacctctgcccaagaaagcctg

SEQ ID NO: 1185 - DNA, 2016 bp, HERV-K
agcttacaaaacaaaatggggcaaactgaaagtaaatatgcctcttatctcagctttattaaaattcttttaaaaagagggggagttagagtatctacaaaaaatctaatcaagctatttcaaataatagaacaattttgcccatggtttccagaacaaggaactttagatctaaaagattggaaaagaattggcgaggaactaaaacaagcaggtagaaagggtaatatcattccacttacagtatggaatgattgggccattattaaagcagctttagaaccatttcaaacaaaagaagatagcgtttcagtttctgatgcccctggaagctgtgtaatagattgtaatgaaaagacagggagaaaatcccagaaagaaacagaaagtttacattgcgaatatgtaacagagccagtaatggctcagtcaacgcaaaatgttgactataatcaattacagggggtgatatatcctgaaacgttaaaattagaaggaaaaggtccagaattagtggggccatcagagtctaaaccacgagggccaagtcctcttccagcaggtcaggtgcccgtaacattacaacctcaaacgcaggttaaagaaaataagacccaaccgccagtagcttatcaatactggccgccggctgaacttcagtatctgccacccccagaaagtcagtatggatatccaggaatgcccccagcactacagggcagggcgccatatcctcagccgcccactgtgagacttaatcctacagcatcacgtagtggacaaggtggtacactgcacgcagtcattgatgaagccagaaaacagggagatcttgaggcatggcggttcctggtaattttacaactggtacaggccggggaagagactcaagtaggagcgcctgcccgagctgagactagatgtgaacctttcaccatgaaaatgttaaaagatataaaggaaggagttaaacaatatggatccaactccccttatataagaacattattagattccattgctcatggaaatagacttactccttatgactgggaaagtttggccaaatcttccctttcatcctctcagtatctacagtttaaaacctggtggattgatggagtacaagaacaggtacgaaaaaatcaggctactaagcccactgttaatatagacgcagaccaattgttaggaacaggtccaaattggagcaccattaaccaacaatcagtgatgcagaatgaggctattgaacaagtaagggctatttgcctcagggcctggggaaaaattcaggacccaggaacagctttccctattaattcaattagacaaggctctaaagagccatatcctgactttgtggcaagattacaagatgctgctcaaaagtctattacagatgacaatgcccgaaaagttattgtagaattaatggcctatgaaaatgcaaatccagaatgtcagtcggccataaagccattaaaaggaaaagttccagcaggagttgatgtaattacagaatatgtgaaggcttgtgatgggattggaggagctatgcataaggcaatgctaatggctcaagcaatgagggggctcactctaggaggacaagttagaacatttgggaaaaaatgttataattgtggtcaaatcggtcatctgaaaaggagttgcccagtcttaaataaacagaatataataaatcaagctattacagcaaaaaataaaaagccatctggcctgtgtccaaaatgtggaaaaggaaaacattgggccaatcaatgtcattctaaatttgataaggatgggcaaccattgtcgggaaacaggaagaggggccagcctcaggccccccaacaaactggggcattcccagttcaactgtttgttcctcagggttttcaaggacaacaacccctacagaaaataccaccacttcagggagtcagccaattacaacaatccaacagctgtcccgcgccacagcaggcagcaccgcagtagtaagtcgac

SEQ ID NO: 1186 - protein, 663 aa, HERV-K
MGQTESKYASYLSFIKILLKRGGVRVSTKNLIKLFQIIEQFCPWFPEQGTLDLKDWKRIGEELKQAGRKGNIIPLTVWNDWAIIKAALEPFQTKEDSVSVSDAPGSCVIDCNEKTGRKSQKETESLHCEYVTEPVMAQSTQNVDYNQLQGVIYPETLKLEGKGPELVGPSESKPRGPSPLPAGQVPVTLQPQTQVKENKTQPPVAYQYWPPAELQYLPPPESQYGYPGMPPALQGRAPYPQPPTVRLNPTASRSGQGGTLHAVIDEARKQGDLEAWRFLVILQLVQAGEETQVGAPARAETRCEPFTMKMLKDIKEGVKQYGSNSPYIRTLLDSIAHGNRLTPYDWESLAKSSLSSSQYLQFKTWWIDGVQEQVRKNQATKPTVNIDADQLLGTGPNWSTINQQSVMQNEAIEQVRAICLRAWGKIQDPGTAFPINSIRQGSKEPYPDFVARLQDAAQKSITDDNARKVIVELMAYENANPECQSAIKPLKGKVPAGVDVITEYVKACDGIGGAMHKAMLMAQAMRGLTLGGQVRTFGKKCYNCGQIGHLKRSCPVLNKQNIINQAITAKNKKPSGLCPKCGKGKHWANQCHSKFDKDGQPLSGNRKRGQPQAPQQTGAFPVQLFVPQGFQGQQPLQKIPPLQGVSQLQQSNSCPAPQQAAPQ

SEQ ID NO: 1187 - DNA, 2172 bp, HERV-K
agcttacaaaacaaaatggggcaaactgaaagtaaatatgcctcttatctcagctttattaaaattcttttaagaagagggggagttagagcttctacagaaaatctaattacgctatttcaaacaatagaacaattctgcccatggtttccagaacagggaactttagatctaaaagattgggaaaaaattggcaaagaattaaaacaagcaaatagggaaggtaaaatcatcccacttacagtatggaatgattgggccattattaaagcaactttagaaccatttcaaacaggagaagatattgtttcagtttctgatgcccctaaaagctgtgtaacagattgtgaagaagaggcagggacagaatcccagcaaggaacggaaagttcacattgtaaatatgtagcagagtctgtaatggctcagtcaacgcaaaatgttgactacagtcaattacaggagataatataccctgaatcatcaaaattgggggaaggaggtccagaatcattggggccatcagagcctaaaccacgatcgccatcaactcctcctcccgtggttcagatgcctgtaacattacaacctcaaacgcaggttagacaagcacaaaccccaagagaaaatcaagtagaaagggacagagtctctatcccggcaatgccaactcagatacagtatccacaatatcagccggtagaaaataagacccaaccgctggtagtttatcaataccggctgccaaccgagcttcagtatcggcctccttcagaggttcaatacagacctcaagcggtgtgtcctgtgccaaatagcacggcaccataccagcaacccacagcgatggcgtctaattcaccagcaacacaggacgcggcgctgtatcctcagccgcccactgtgagacttaatcctacagcatcacgtagtggacagggtggtgcactgcatgcagtcattgatgaagccagaaaacagggcgatcttgaggcatggcggttcctggtaattttacaactggtacaggccggggaagagactcaagtaggagcgcctgcccgagctgagactagatgtgaacctttcaccatgaaaatgttaaaagatataaaggaaggagttaaacaatatggatccaactccccttatataagaacattattagattccattgctcatggaaatagacttactccttatgactgggaaattttggccaaatcttccctttcatcctctcagtatctacagtttaaaacctggtggattgatggagtacaagaacaggtacgaaaaaatcaggctactaagcccactgttaatatagacgcagaccaattgttaggaacaggtccaaattggagcaccattaaccaacaatcagtgatgcagaatgaggctattgaacaagtaagggctatttgcctcagggcctggggaaaaattcaggacccaggaacagctttccctattaattcaattagacaaggctctaaagagccatatcctgactttgtggcaagattacaagatgctgctcaaaagtctattacagatgacaatgcccgaaaagttattgtagaattaatggcctatgaaaatgcaaatccagaatgtcagtcggccataaagccattaaaaggaaaagttccagcaggagttgatgtaattacagaatatgtgaaggcttgtgatgggattggaggagctatgcataaggcaatgctaatggctcaagcaatgagggggctcactctaggaggacaagttagaacatttgggaaaaaatgttataattgtggtcaaatcggtcatctgaaaaggagttgcccagtcttaaataaacagaatataataaatcaagctattacagcaaaaaataaaaagccatctggcctgtgtccaaaatgtggaaaaggaaaacattgggccaatcaatgtcattctaaatttgataaggatgggcaaccattgtcgggaaacaggaagaggggccagcctcaggccccccaacaaactggggcattcccagttcaactgtttgttcctcagggttttcaaggacaacaacccctacagaaaataccaccacttcagggagtcagccaattacaacaatccaacagctgtcccgcgccacagcaggcagcaccgcagtagtaagtcgac

SEQ ID NO: 1188 - protein, 713 aa, HERV-K
MGQTESKYASYLSFIKILLRRGGVRASTENLITLFQTIEQFCPWFPEQGTLDLKDWEKIGKELKQANREGKIIPLTVWNDWAIIKATLEPFQTGEDIVSVSDAPKSCVTDCEEEAGTESQQGTESSHCKYVAESVMAQSTQNVDYSQLQEIIYPESSKLGEGGPESLGPSEPKPRSPSTPPPVVQMPVTLQPQTQVRQAQTPRENQVERDRVSIPAMPTQIQYPQYQPVENKTQPLVVYQYRLPTELQYRPPSEVQYRPQAVCPVPNSTAPYQQPTAMNSPATQDAALYPQPPTVRLNPTASRSGQGGALHAVIDEARKQGDLEAWRFLVILQLVQAGEETQVGAPARAETRCEPFTMKMLKDIKEGVKQYGSNSPYIRTLLDSIAHGNRLTPYDWEILAKSSLSSSQYLQFKTWWIDGVQEQVRKNQATKPTVNIDADQLLGTGPNWSTINQQSVMQNEAIEQVRAICLRAWGKIQDPGTAFPINSIRQGSKEPYPDFVARLQDAAQKSITDDNARKVIVELMAYENANPECQSAIKPLKGKVPAGVDVITEYVKACDGIGGAMHKAMLMAQAMRGLTLGGQVRTFGKKCYNCGQIGHLKRSCPVLNKQNIINQAITAKNKKPSGLCPKCGKGKHWANQCHSKFDKDGQPLSGNRKRGQPQAPQQTGAFPVQLFVPQGFQGQQPLQKIPPLQGVSQLQQSNSCPAPQQAAPQ

SEQ ID NO: 1189 - protein, 15 aa, Artificial Sequence
  V5 tag
GGKPIPNPLLGLDST

SEQ ID NO: 1190 - DNA, 962 bp, HERV-K
tgtggggaaaagcaagagagatcagattgtcactgtatctgtgtagaaagaagtagacatgggagactccattttgttatgtactaagaaaaattcttctgccttgagattctgtgaccttacccccaaccccgtgctctctgaaacatgtgctgtgtcaaactcagggttaaatggattaagggcggtgcaggatgtgctttgttaaacagatgcttgaaggcagcatgctccttaagagtcatcaccactccctaatctcaagtacccagggacacaaacactgcggaaggccgcagggacctctgcctaggaaagccaggtattgtccaaggtttctccccatgtgatagtctgaaatatggcctcgtgggaagggaaagacctgaccgtcccccagcccgacacccgtaaagggtctgtgctgaggaggattagtaaaagaggaaggcatgcctcttgcagttgagacaagaggaaggcatctgtctcctgcccgtccctgggcaatggaatgtctcggtataaaaccggattgtacgttccatctactgagatagggaaaaaccgccttagggctggaggtgggacctgcgggcagcaatactgctttttaaagcattgagatgtttatgtgtatgcatatctaaaagcacagcacttaatcctttaccttgtctatgatgcaaagatctttgttcacgtgtttgtctgctgaccctctccccactattgtcttgtgaccctgacacatccccctctcggagaaacacccacgaatgaccaataaatactaaagggaactcagaggctggcgggatcctccatatgctgaacgctggttccccgggcccccttatttctttctctacactttgtctctgtgtctttttctttcctaagtctctcgttccaccttacgagaaacacccacaggtgtggaggggcaacccacccctaca

SEQ ID NO: 1191 - DNA, 364 bp, HERV-K
gggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgttctgtactaagaaaaattgttttgccttgagatgctgt

SEQ ID NO: 1192 - DNA, 25 bp, HERV-K
ctcgttccacctgaggagaaatgcc

SEQ ID NO: 1193 - DNA, 20 bp, HERV-K
gaggcgcaggccactccatc

SEQ ID NO: 1194 - DNA, 27 bp, HERV-K
cttgtcctcaatgaccacactgtagag

SEQ ID NO: 1195 - DNA, 24 bp, HERV-K
agagtaccttcacccacaaggctc

SEQ ID NO: 1196 - DNA, 1010 bp, HERV-K
tgtagggaaaagaaagagagatcacactgttactgtgtctatgtagaaaaaggaagacataagaaactccattttgatctgtactaagaaaaattcttctgctttgaaatgctattaatctgtaaccctagccccaaccctgtgctcacagaaacatgcgctgtattgactcaaggttaatggatttagggctgtgcaggatgtgctttgttaacaatgtgtttgaaggcagtatgcttggtaaaggtcatcgccattctccagtcttgagtacccagggacacaatgcactgtggaaagccatggggacctctgcccaagaaagcctgggtgttgtccaggcttccccacactgagacagcctgagatgtggcctcgttggaagggaaagaccttacattatagtcccccagccggacacccataaaaggtctgtgctgaggaggattactgaaagaggaaggcctctttgcagttaagaggaaagcatctgtctcatgatcccctgggaatggaatgtcttggtgtaaaacctgatcgtacattctatttactgagataggagaaaaccgccctatggctggaggtgagacatgctggtggcaataccgatctttactgcacggcaatactgatctttactgcactgagatgtttatgtaaagttaaacataaatctagcctacgtgcacattcaggcatagcacctttccttaaacttatttatgacacagagtcttttgttcacgtgttttcctgttgaccctctctccaccattaccctatagtcctgccacatccccctcactgagatagtagagataatgatcaataaatactgagggaattcagaaaccagtgccggtgcaggtcctcacttgctgagtgccggtcccctgggcccacttttcttcctctatgctttacctctgtgtcttatttcttttctcagtctctcgtctccaccttgcgagaaatacccacaggtgtggaggggctggcccccttca

SEQ ID NO: 1197 - RNA, 11100 nt, HERV-K
uuauguauaugcacaucaaaagcacagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugugguaagcuugggcgcucggaagaagccaggguuaauggggcaaacuaaaaguaaagucucucauuccaccugaugagaaacacccagagguguggaggggcaggccaccccuucaggguaggguccccuccaugcagaccauagagcacaggugugccccaaagaggagcagagagaaggagggagagggcccacgagagacuuggaaaugaauggcaggauuuuaggcgcuggacuuggguucggggcaccuggccuuuccuuguguauuucuccuacugucugccuaacuauuuaauacaauaaaagaaaaccagccccugguucuugugguguuuccacccucccggguccccgcuggcugccuggcuuccucccgcagcuccugcuguguguguaugugugugugugugcacaucuguggggcguauguguguucgucuuuguaauugaggcugcagaguggagagagcagggguuuucucuggggacccagagagaaggaggcguuuucaccacagccgaacagggcaggaccccagcacccgggacccagcgggacuuugccaaggggauggaccuggcugggccacgcggcuguuuguguagggaaaagaaagagagaucacacuguuacugugucuauguagaaaaggaagacauaaacuccauuuugagcuguacuaagaaaaauuauuuugccuugaccugcuguuaaccuguaacuguagccccaacccugugcucaaagaaacaugugcuguauggaaucaagguuuaagggaucaagggcuguacaggaugugccuuguuaacaauguguuuacaggcaguaugcuugguaaaagucaucgccauucuccauucuccauuaaucaggggcacgaugcacugcggaaagccacagggaccucugcccgagaaagccuggguauuguccaaggcuuccccccacugagacagccugagauacggccucgugggaagggaaagaccugaccgucccccagcccgacacccguaaagggucugugcugaggaggauuaguaaaaggggaaggccucuugcaguugagauaagaggaaggccuccgucuccugcauguccuugggaauggaaugucuugguguaaaacccgauaguacauuccuucuauucugagagaagaaaaccacccuguggcuggaggugagauaugcuagcggcaaugcugcucuguuacucuuugcuacacugagauguuuggguggagagaagcauaaaucuggccuaugugcacaucugggcacagaaccuccccuugaacuugugacacagauuccuuuguucacauguuuuccugcugaccuucuccccacuaucgcccuguucucccaccgcauuccccuugcugagauagugaaaauaguaaucuguagauaccaagggaacucagagaccauggccggugcacauccuccguacgcugagcgcugguccccugggcccauuguucuuucucuauacuuugucucugugucuuauuucuuuccucagucucucaucccuccugacgagaaauacccacagguguggaggggcuggcccccuucaucugaugcccaaugugggugccuuucucuagggugaagguacucuacaguguggucauugaggacaaguugacgagagagucccaaguacguccacggucagccuugcgguaagcuugugugcuuagaggaacccaggguaacgauggggcaaacugaaaguaaauaugccucuuaucucagcuuuauuaaaauucuuuuaagaagagggggaguuagagcuucuacagaaaaucuaauuacgcuauuucaaacaauagaacaauucugcccaugguuuccagaacagggaacuuuagaucuaaaagauugggaaaaaauuggcaaagaauuaaaacaagcaaauagggaagguaaaaucaucccacuuacaguauggaaugauugggccauuauuaaagcaacuuuagaaccauuucaaacaggagaagauauuguuucaguuucugaugccccuaaaagcuguguaacagauugugaagaagaggcagggacagaaucccagcaaggaacggaaaguucacauuguaaauauguagcagagucuguaauggcucagucaacgcaaaauguugacuacagucaauuacaggagauaauauacccugaaucaucaaaauugggggaaggagguccagaaucauuggggccaucagagccuaaaccacgaucgccaucaacuccuccucccgugguucagaugccuguaacauuacaaccucaaacgcagguuagacaagcacaaaccccaagagaaaaucaaguagaaagggacagagucucuaucccggcaaugccaacucagauacaguauccacaauaucagccgguagaaaauaagacccaaccgcugguaguuuaucaauaccggcugccaaccgagcuucaguaucggccuccuucagagguucaauacagaccucaagcgguguguccugugccaaauagcacggcaccauaccagcaacccacagcgauggcgucuaauucaccagcaacacaggacgcggcgcuguauccucagccgcccacugugagacuuaauccuacagcaucacguaguggacaggguggugcacugcaugcagucauugaugaagccagaaaacagggcgaucuugaggcauggcgguuccugguaauuuuacaacugguacaggccggggaagagacucaaguaggagcgccugcccgagcugagacuagaugugaaccuuucaccaugaaaauguuaaaagauauaaaggaaggaguuaaacaauauggauccaacuccccuuauauaagaacauuauuagauuccauugcucauggaaauagacuuacuccuuaugacugggaaauuuuggccaaaucuucccuuucauccucucaguaucuacaguuuaaaaccugguggauugauggaguacaagaacagguacgaaaaaaucaggcuacuaagcccacuguuaauauagacgcagaccaauuguuaggaacagguccaaauuggagcaccauuaaccaacaaucagugaugcagaaugaggcuauugaacaaguaagggcuauuugccucagggccuggggaaaaauucaggacccaggaacagcuuucccuauuaauucaauuagacaaggcucuaaagagccauauccugacuuuguggcaagauuacaagaugcugcucaaaagucuauuacagaugacaaugcccgaaaaguuauuguagaauuaauggccuaugaaaaugcaaauccagaaugucagucggccauaaagccauuaaaaggaaaaguuccagcaggaguugauguaauuacagaauaugugaaggcuugugaugggauuggaggagcuaugcauaaggcaaugcuaauggcucaagcaaugagggggcucacucuaggaggacaaguuagaacauuugggaaaaaauguuauaauuguggucaaaucggucaucugaaaaggaguugcccaggcuuaaauaaacagaauauaauaaaucaagcuauuacagcaaaaaauaaaaagccaucuggccuguguccaaaauguggaaaagcaaaacauugggccaaucaaugucauucuaaauuugauaaagaugggcaaccauugucuggaaacaggaagaggggccagccucaggccccccaacaaacuggggcauucccaguuaaacuguuuguuccucaggguuuucaaggacaacaaccccuacagaaaauaccaccacuucagggagucagccaauuacaacaauccaacagcugucccgcgccacagcaggcagcaccgcaguagauuuauguuccacccaaauggucuuuuuacucccuggaaagcccccacaaaagauuccuagagggguauauggcccgcugccagaagggaggguaggccuuugagggagaucaagucuaaauuugaagggaguccaaauucauacugggguaauuuauucagauuauaaagggggaauucaguuagugaucagcuccacuguuccccggagugccaauccaggugauagaauugcucaauuacugcuuuugccuuauguuaaaauuggggaaaacaaaaaggaaagaacaggaggguuuggaaguaccaacccugcaggaaaagcugcuuauugggcuaaucaggucucagaggauagacccguguguacagucacuauucagggaaagaguuugaaggauuaguggauacccaggcugauguuucugucaucggcauagguacugccucagaaguguaucaaagugccaugauuuuacauuguccaggaucugauaaucaagaaaguacgguucagccugugaucacuucauuccaaucaauuuauggggccgagacuuguuacaacaauggcaugcagagauuacuaucccagccucccuauacagccccaggaauaaaaaaaucaugacuaaaaugggauagcucccuaaaaagggacuaggaaagaagucccaauugaggcugaaaaaaaucaaaaaagaaaaggaauagggcauccuuuuuaggagcggucacuguagagccuccaaaacccauuccauuaacuugggggaaaaaaaaacaacuguaugguaaaucagcagcgcuuccaaaacaaaaacuggaggcuuuacauuuauuagcaaagaaacaauuagaaaaaggacauugagccuucauuuucgccuuggaauucuguuuguaauucagaaaaaauccggcagauggcguauaaugccguaauucaacccaugggggcucucccaccccgguugcccucuccagccaugguccccuuuaauuauaauugaucugaaggauugcuuuuuuaccauuccucuggcaaaacaggauuuugaaaaauuugcuuuuaccacaccagccuaaauaauaaagaaccagccaccagguuucaguggaaaguauugccucagggaaugcuuaauaguucaacuauuugucagcucaagcucugcaaccaguuagagacaaguuuucagacuguuacaucguucacuauguugauauuuugugugcugcagaaacgagagacaaauuaauugaccguuacacauuucugcagacagagguugccaacgcgggacugacaauaacaucugauaagauucaaaccucuacuccuuuccguuacuugggaaugcagguagaggaaaggaaaauuaaaccacaaaaaauagaaauaagaaaagacacauuaaaagcauuaaaugaguuucaaaaguugcuaggagauacuaauuggauuuggagauauuaauuggauuuggccaacucuaggcauuccuacuuaugccaugucaaauuuguucucuuucuuaagaggggacucggaauuaaauagugaaagaacguuaacuccagaggcaacuaaagaaauuaaauuaauugaagaaaaaauucggucagcacaaguaaauagaauagaucacuuggccccacuccaaauuuugauuuuugcuacugcacauucccuaacaggcaucauuguucaaaauacagaucuuguggagugguccuuccuuccucacaguacaauuaagacuuuuacauuguacuuggaucaaauggcuacauuaauuggucagggaagauuaugaauaauaacauuguguggaaaugacccagauaaaaucacuguuccuuucaacaagcaacagguuagacaagccuuuaucaauucuggugcauggcagauuggucuugccgauuuugugggaauuauugacaaucguuaccccaaaacaaaaaucuuccaguuuuuaaaauugacuacuuggauuuuaccuaaaguuaccaaacauaagccuuuaaaaaaugcucuggcaguguuuacugaugguuccagcaauggaaaaguggcuuacaccgggccaaaagaaugagucaucaaaacucaguaucacuugacucaaagagcagaguugguugccgucauuacaguguuaacaagauuuuaaucagucuauuaacauuguaucagauucugcauauguaguacaggcuacaaaggauauugagagagcccuaaucaaauacauuauggaugaucaguuaaacccgcuguuuaauuuguuacaacaaaauguaagaaaaagaaauuucccauuuuauauuacucauauucgagcacacacuaauuuaccagggccuuuaacuaaagcaaaugaacaagcugacuugcuaguaucaucugcauucauggaagcacaagaacuucaugccuugacucauguaaaugcaauaggauuaaaaaauaaauuugauaucacauggaaacagacaaaaaauauuguacaacauugcacccagugucagauucuacaccuggccacucaggaggcaagaguuaaucccagaggucuauguccuaauguguuauggcaaauggaugucaugcacguaccuucauuuggaaaauugucauuuguccaugugacaguugauacuuauucacauuucauaugggcaaccugccagacaggagaaaguacuucccauguuaaaagacauuuauuaucuuguuuuccugucaugggaguuccagaaaaaguuaaaacagacaaugggccagguuacuguaguaaagcaguucaaaaauucuuaaaucaguggaaaauuacacauacaauaggaauucucuauaauucccaaggacaggccauaauugaaagaacuaauagaacacucaaagcucaauugguuaaacaaaaaaaaggaaaagacaggaguauaacacuccccagaugcaacuuaaucuagcacucuauacuuuaaauguuuuaaacauuuauagaaaucagaccacuaccucugcagaacaacaucuuacugguaaaaggaacagcccacaugaaggaaaacugauuugguggaaagauaauaaaaauaaaacaugggaaauggggaaggugauaacgugggggagagguuuugcuuguguuucaccaggagaaaaucagcuuccuguuuggauacccacuagacauuuaaaguucuacaaugaacucacuggagaugcaaagaaaaguguggagauggagacaccccaaucgacucgccagguaaacaaaauggugauaucagaagaacagaaaaaguugccuuccaucaaggaagcagaguugccaauauaggcacaauuaaagaagcugacacaguuagcuaaaaaaaaaagccuagagaauacaaaggugacaccaacuccagagaauaugcugcuugcagcucugaugauuguaucaacggugguaagucuucccaagucugcaggagcagcugcagcuaauuauacuuacugggccuaugugccuuucccacccuuaauucgggcaguuacauagauggauaauccuauugaaguagauguuaauaauagugcaugggugccuggccccacagaugacuguugcccugcccaaccugaagaaggaaugaugaugaauauuuccauuggguauccuuauccuccuguuugccuagggaaggcaccaggaugcuuaaugccuacaacccaaaauugguugguagaaguaccuacagucagugcuaccaguagauuuacuuaucacaugguaaguggaaugucacagauaaauaauuuacaggacccuucuuaucaaagaucauuacaauguaggccuaaggggaaggcuugccccaaggaaauucccaaagaaucaaaaagcccagaagucuuagucugcggagaauguguggcugauacugcaguguaguacaaaacaaugaauuuugaacuaugauagacugggucccuugaggccaauuauaucauaacuguacaggccagacucauucauguucacaggccccauccaucuggcccauuaauccagccuaugacggugauguaacugaaaggcuggaccagguuuauagaagguuagaaucacucuguccaaggaaauggggugaaaagggaauuucaucaccuugaccaaaguuaguccuguuacugguccugaacauccagaauuaggaagcuuacuguggccucacaccacauuagaauuuguucuggaaaucaagcuauaggaacaagagaucguaagucauauuauacuaucaaccuaaauuccagucugacaauuccuuugcaaaauuguguaaaacucccuuauauugcuaguuguaggaaaaacauaguuauuaaaccugauucccaaaccauaaucugugaaaauuguggaauguuuacuugcauugauuugacuuuuaauuggcagcaccguauucuacuaggaagagcaagagaggguguguggauccuuguguccauggaccgaccaugggaggcuucgcuauccauccauauuuuaacggaaguauuaaaaggaauucuaacuagauccaaaagauucauuuuuacuuugauggcagugauuaugggccucauugcagucacagcuacugcugcggcugcuggaauugcuuuacacuccucuguucaaacugcagaauacguaaaugauuggcaaaagaauuccucaaaauuguggaauucucagauccaaauagaucaaaaauuggcaaaccaaauuaaugaucuuagacaaacugucauuuggaugggagaggcucaugagcuuggaauaucuuuuucaguuacgaugugacuggaauacaucagauuuuuguguuacaccacaagccuauaaugagucugagcaucacugggacaugguuagaugccaucugcaaggaggagaagauaaucuuacuuuagacauuucaaaauuaaaagaauuuuuuuuuucuuugagacagagucucgcucugucgcccaggcuggagugcaguggcgugaucucagcucacugcaaguuccgccuccuggguuuacaccauucuccugccucagccucccaaguaguugggacuacaggagcccaccaccaugccuggcuaauuuuuuuuggguuuuuaauagagauggaguuucaccguguuagccaggauggucucgaucuccugaccuugugaucugcccaccuuggccucccaaagugcugggauuacagucgugagccaccgugcccagccaagaaaaaauuuuugaggcaucaaaagcccauuuaaauuuggugccaggaacggagacaaucgugaaagcugcugauagccucacaaaucuuaagccagucacuuggguuaaaagcaucagaaguuucacuauuguaaauuucauauuaauccuuguaugccuguucugucuguuguuagucuacagguguauccagcagcuccaaagagacagcaaccagcaagaaugggccauagugacgauggugguuuugucaaaaagaaaagggggggauauguaaggaaaagagagaucagacuuucacugugucuauguagaaaaggaagacauaagaaacuccauuuugaucuguacuaagaaaaauuguuuugccuugagaugcuguuaaucuguaacuuuagccccaacccugugcucacggaaacaugugcuguaagguuuaagggaucuagggcugugcaggauguaccuuguuaacaauauguuugcaggcaguauguuugguaaaagucaucgccauucuccauucucgauuaaccaggggcucaaugcacuguggaaagccacaggaaccucugcccaagaaagccuggcuguugugggaagucagggaccccgaauggagggaccagcuggugcugcaucaggaaacauaaauugugaagauuucuuggacauuuaucaguuuccaaaauuaauacuuuuauaauuucuuacaccugucuuacuuuaaucucuuaauccuguuaucuuuguaagcugaggauauacgucaccucaggaccacuauuguacaaauugauuguaaaacauguucacauguguuugaacaauaugaaaucagugcaccuugaaaaugaacagaauaacagugauuuuagggaacaaaggaagacaaccauaaggucugacugccugaggggucgggcaaaaagccauauuuuucuucuugcagagagccuauaaauggacgugcaaguaggagagauauugcuaaauucuuuuccuagcaaggaauauaauacuaagacccuagggaaagaauugcauuccuggggggaggucuauaaacggccgcucugggagugucuguccuaugugguugagauaaggacugagauacgcccuggucuccugcaguacccucaggcuuacuaggauugggaaaccccaguccugguaaauuugaggucaggccgguucuuugcucugaacccuguuuucuguuaagauguuuaucaagacaauacaugcaccgcugaacauagacccuuaucaggaguuucugauuuugcucugguccuguuucuucagaagcaugucaucuuugcucugccuucugcccuuugaagcaugugaucuuugugaccuacucccuguucauacaccccuccccuuuuaaaaucccuaauaaaaacuugcugguuuuguggcucaggggggcaucauggaccuaccaauacgugaugucacccccgguggcccagcugu

SEQ ID NO: 1198 - RNA, 11077 nt, HERV-K
acagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugugguaagcuugggcgcucggaagaagccaggguuaauggggcaaacuaaaaguaaagucucucauuccaccugaugagaaacacccagagguguggaggggcaggccaccccuucaggguaggguccccuccaugcagaccauagagcacaggugugccccaaagaggagcagagagaaggagggagagggcccacgagagacuuggaaaugaauggcaggauuuuaggcgcuggacuuggguucggggcaccuggccuuuccuuguguauuucuccuacugucugccuaacuauuuaauacaauaaaagaaaaccagccccugguucuugugguguuuccacccucccggguccccgcuggcugccuggcuuccucccgcagcuccugcuguguguguaugugugugugugugcacaucuguggggcguauguguguucgucuuuguaauugaggcugcagaguggagagagcagggguuuucucuggggacccagagagaaggaggcguuuucaccacagccgaacagggcaggaccccagcacccgggacccagcgggacuuugccaaggggauggaccuggcugggccacgcggcuguuuguguagggaaaagaaagagagaucacacuguuacugugucuauguagaaaaggaagacauaaacuccauuuugagcuguacuaagaaaaauuauuuugccuugaccugcuguuaaccuguaacuguagccccaacccugugcucaaagaaacaugugcuguauggaaucaagguuuaagggaucaagggcuguacaggaugugccuuguuaacaauguguuuacaggcaguaugcuugguaaaagucaucgccauucuccauucuccauuaaucaggggcacgaugcacugcggaaagccacagggaccucugcccgagaaagccuggguauuguccaaggcuuccccccacugagacagccugagauacggccucgugggaagggaaagaccugaccgucccccagcccgacacccguaaagggucugugcugaggaggauuaguaaaaggggaaggccucuugcaguugagauaagaggaaggccuccgucuccugcauguccuugggaauggaaugucuugguguaaaacccgauaguacauuccuucuauucugagagaagaaaaccacccuguggcuggaggugagauaugcuagcggcaaugcugcucuguuacucuuugcuacacugagauguuuggguggagagaagcauaaaucuggccuaugugcacaucugggcacagaaccuccccuugaacuugugacacagauuccuuuguucacauguuuuccugcugaccuucuccccacuaucgcccuguucucccaccgcauuccccuugcugagauagugaaaauaguaaucuguagauaccaagggaacucagagaccauggccggugcacauccuccguacgcugagcgcugguccccugggcccauuguucuuucucuauacuuugucucugugucuuauuucuuuccucagucucucaucccuccugacgagaaauacccacagguguggaggggcuggcccccuucaucugaugcccaaugugggugccuuucucuagggugaagguacucuacaguguggucauugaggacaaguugacgagagagucccaaguacguccacggucagccuugcgguaagcuugugugcuuagaggaacccaggguaacgauggggcaaacugaaaguaaauaugccucuuaucucagcuuuauuaaaauucuuuuaagaagagggggaguuagagcuucuacagaaaaucuaauuacgcuauuucaaacaauagaacaauucugcccaugguuuccagaacagggaacuuuagaucuaaaagauugggaaaaaauuggcaaagaauuaaaacaagcaaauagggaagguaaaaucaucccacuuacaguauggaaugauugggccauuauuaaagcaacuuuagaaccauuucaaacaggagaagauauuguuucaguuucugaugccccuaaaagcuguguaacagauugugaagaagaggcagggacagaaucccagcaaggaacggaaaguucacauuguaaauauguagcagagucuguaauggcucagucaacgcaaaauguugacuacagucaauuacaggagauaauauacccugaaucaucaaaauugggggaaggagguccagaaucauuggggccaucagagccuaaaccacgaucgccaucaacuccuccucccgugguucagaugccuguaacauuacaaccucaaacgcagguuagacaagcacaaaccccaagagaaaaucaaguagaaagggacagagucucuaucccggcaaugccaacucagauacaguauccacaauaucagccgguagaaaauaagacccaaccgcugguaguuuaucaauaccggcugccaaccgagcuucaguaucggccuccuucagagguucaauacagaccucaagcgguguguccugugccaaauagcacggcaccauaccagcaacccacagcgauggcgucuaauucaccagcaacacaggacgcggcgcuguauccucagccgcccacugugagacuuaauccuacagcaucacguaguggacaggguggugcacugcaugcagucauugaugaagccagaaaacagggcgaucuugaggcauggcgguuccugguaauuuuacaacugguacaggccggggaagagacucaaguaggagcgccugcccgagcugagacuagaugugaaccuuucaccaugaaaauguuaaaagauauaaaggaaggaguuaaacaauauggauccaacuccccuuauauaagaacauuauuagauuccauugcucauggaaauagacuuacuccuuaugacugggaaauuuuggccaaaucuucccuuucauccucucaguaucuacaguuuaaaaccugguggauugauggaguacaagaacagguacgaaaaaaucaggcuacuaagcccacuguuaauauagacgcagaccaauuguuaggaacagguccaaauuggagcaccauuaaccaacaaucagugaugcagaaugaggcuauugaacaaguaagggcuauuugccucagggccuggggaaaaauucaggacccaggaacagcuuucccuauuaauucaauuagacaaggcucuaaagagccauauccugacuuuguggcaagauuacaagaugcugcucaaaagucuauuacagaugacaaugcccgaaaaguuauuguagaauuaauggccuaugaaaaugcaaauccagaaugucagucggccauaaagccauuaaaaggaaaaguuccagcaggaguugauguaauuacagaauaugugaaggcuugugaugggauuggaggagcuaugcauaaggcaaugcuaauggcucaagcaaugagggggcucacucuaggaggacaaguuagaacauuugggaaaaaauguuauaauuguggucaaaucggucaucugaaaaggaguugcccaggcuuaaauaaacagaauauaauaaaucaagcuauuacagcaaaaaauaaaaagccaucuggccuguguccaaaauguggaaaagcaaaacauugggccaaucaaugucauucuaaauuugauaaagaugggcaaccauugucuggaaacaggaagaggggccagccucaggccccccaacaaacuggggcauucccaguuaaacuguuuguuccucaggguuuucaaggacaacaaccccuacagaaaauaccaccacuucagggagucagccaauuacaacaauccaacagcugucccgcgccacagcaggcagcaccgcaguagauuuauguuccacccaaauggucuuuuuacucccuggaaagcccccacaaaagauuccuagagggguauauggcccgcugccagaagggaggguaggccuuugagggagaucaagucuaaauuugaagggaguccaaauucauacugggguaauuuauucagauuauaaagggggaauucaguuagugaucagcuccacuguuccccggagugccaauccaggugauagaauugcucaauuacugcuuuugccuuauguuaaaauuggggaaaacaaaaaggaaagaacaggaggguuuggaaguaccaacccugcaggaaaagcugcuuauugggcuaaucaggucucagaggauagacccguguguacagucacuauucagggaaagaguuugaaggauuaguggauacccaggcugauguuucugucaucggcauagguacugccucagaaguguaucaaagugccaugauuuuacauuguccaggaucugauaaucaagaaaguacgguucagccugugaucacuucauuccaaucaauuuauggggccgagacuuguuacaacaauggcaugcagagauuacuaucccagccucccuauacagccccaggaauaaaaaaaucaugacuaaaaugggauagcucccuaaaaagggacuaggaaagaagucccaauugaggcugaaaaaaaucaaaaaagaaaaggaauagggcauccuuuuuaggagcggucacuguagagccuccaaaacccauuccauuaacuugggggaaaaaaaaacaacuguaugguaaaucagcagcgcuuccaaaacaaaaacuggaggcuuuacauuuauuagcaaagaaacaauuagaaaaaggacauugagccuucauuuucgccuuggaauucuguuuguaauucagaaaaaauccggcagauggcguauaaugccguaauucaacccaugggggcucucccaccccgguugcccucuccagccaugguccccuuuaauuauaauugaucugaaggauugcuuuuuuaccauuccucuggcaaaacaggauuuugaaaaauuugcuuuuaccacaccagccuaaauaauaaagaaccagccaccagguuucaguggaaaguauugccucagggaaugcuuaauaguucaacuauuugucagcucaagcucugcaaccaguuagagacaaguuuucagacuguuacaucguucacuauguugauauuuugugugcugcagaaacgagagacaaauuaauugaccguuacacauuucugcagacagagguugccaacgcgggacugacaauaacaucugauaagauucaaaccucuacuccuuuccguuacuugggaaugcagguagaggaaaggaaaauuaaaccacaaaaaauagaaauaagaaaagacacauuaaaagcauuaaaugaguuucaaaaguugcuaggagauacuaauuggauuuggagauauuaauuggauuuggccaacucuaggcauuccuacuuaugccaugucaaauuuguucucuuucuuaagaggggacucggaauuaaauagugaaagaacguuaacuccagaggcaacuaaagaaauuaaauuaauugaagaaaaaauucggucagcacaaguaaauagaauagaucacuuggccccacuccaaauuuugauuuuugcuacugcacauucccuaacaggcaucauuguucaaaauacagaucuuguggagugguccuuccuuccucacaguacaauuaagacuuuuacauuguacuuggaucaaauggcuacauuaauuggucagggaagauuaugaauaauaacauuguguggaaaugacccagauaaaaucacuguuccuuucaacaagcaacagguuagacaagccuuuaucaauucuggugcauggcagauuggucuugccgauuuugugggaauuauugacaaucguuaccccaaaacaaaaaucuuccaguuuuuaaaauugacuacuuggauuuuaccuaaaguuaccaaacauaagccuuuaaaaaaugcucuggcaguguuuacugaugguuccagcaauggaaaaguggcuuacaccgggccaaaagaaugagucaucaaaacucaguaucacuugacucaaagagcagaguugguugccgucauuacaguguuaacaagauuuuaaucagucuauuaacauuguaucagauucugcauauguaguacaggcuacaaaggauauugagagagcccuaaucaaauacauuauggaugaucaguuaaacccgcuguuuaauuuguuacaacaaaauguaagaaaaagaaauuucccauuuuauauuacucauauucgagcacacacuaauuuaccagggccuuuaacuaaagcaaaugaacaagcugacuugcuaguaucaucugcauucauggaagcacaagaacuucaugccuugacucauguaaaugcaauaggauuaaaaaauaaauuugauaucacauggaaacagacaaaaaauauuguacaacauugcacccagugucagauucuacaccuggccacucaggaggcaagaguuaaucccagaggucuauguccuaauguguuauggcaaauggaugucaugcacguaccuucauuuggaaaauugucauuuguccaugugacaguugauacuuauucacauuucauaugggcaaccugccagacaggagaaaguacuucccauguuaaaagacauuuauuaucuuguuuuccugucaugggaguuccagaaaaaguuaaaacagacaaugggccagguuacuguaguaaagcaguucaaaaauucuuaaaucaguggaaaauuacacauacaauaggaauucucuauaauucccaaggacaggccauaauugaaagaacuaauagaacacucaaagcucaauugguuaaacaaaaaaaaggaaaagacaggaguauaacacuccccagaugcaacuuaaucuagcacucuauacuuuaaauguuuuaaacauuuauagaaaucagaccacuaccucugcagaacaacaucuuacugguaaaaggaacagcccacaugaaggaaaacugauuugguggaaagauaauaaaaauaaaacaugggaaauggggaaggugauaacgugggggagagguuuugcuuguguuucaccaggagaaaaucagcuuccuguuuggauacccacuagacauuuaaaguucuacaaugaacucacuggagaugcaaagaaaaguguggagauggagacaccccaaucgacucgccagguaaacaaaauggugauaucagaagaacagaaaaaguugccuuccaucaaggaagcagaguugccaauauaggcacaauuaaagaagcugacacaguuagcuaaaaaaaaaagccuagagaauacaaaggugacaccaacuccagagaauaugcugcuugcagcucugaugauuguaucaacggugguaagucuucccaagucugcaggagcagcugcagcuaauuauacuuacugggccuaugugccuuucccacccuuaauucgggcaguuacauagauggauaauccuauugaaguagauguuaauaauagugcaugggugccuggccccacagaugacuguugcccugcccaaccugaagaaggaaugaugaugaauauuuccauuggguauccuuauccuccuguuugccuagggaaggcaccaggaugcuuaaugccuacaacccaaaauugguugguagaaguaccuacagucagugcuaccaguagauuuacuuaucacaugguaaguggaaugucacagauaaauaauuuacaggacccuucuuaucaaagaucauuacaauguaggccuaaggggaaggcuugccccaaggaaauucccaaagaaucaaaaagcccagaagucuuagucugcggagaauguguggcugauacugcaguguaguacaaaacaaugaauuuugaacuaugauagacugggucccuugaggccaauuauaucauaacuguacaggccagacucauucauguucacaggccccauccaucuggcccauuaauccagccuaugacggugauguaacugaaaggcuggaccagguuuauagaagguuagaaucacucuguccaaggaaauggggugaaaagggaauuucaucaccuugaccaaaguuaguccuguuacugguccugaacauccagaauuaggaagcuuacuguggccucacaccacauuagaauuuguucuggaaaucaagcuauaggaacaagagaucguaagucauauuauacuaucaaccuaaauuccagucugacaauuccuuugcaaaauuguguaaaacucccuuauauugcuaguuguaggaaaaacauaguuauuaaaccugauucccaaaccauaaucugugaaaauuguggaauguuuacuugcauugauuugacuuuuaauuggcagcaccguauucuacuaggaagagcaagagaggguguguggauccuuguguccauggaccgaccaugggaggcuucgcuauccauccauauuuuaacggaaguauuaaaaggaauucuaacuagauccaaaagauucauuuuuacuuugauggcagugauuaugggccucauugcagucacagcuacugcugcggcugcuggaauugcuuuacacuccucuguucaaacugcagaauacguaaaugauuggcaaaagaauuccucaaaauuguggaauucucagauccaaauagaucaaaaauuggcaaaccaaauuaaugaucuuagacaaacugucauuuggaugggagaggcucaugagcuuggaauaucuuuuucaguuacgaugugacuggaauacaucagauuuuuguguuacaccacaagccuauaaugagucugagcaucacugggacaugguuagaugccaucugcaaggaggagaagauaaucuuacuuuagacauuucaaaauuaaaagaauuuuuuuuuucuuugagacagagucucgcucugucgcccaggcuggagugcaguggcgugaucucagcucacugcaaguuccgccuccuggguuuacaccauucuccugccucagccucccaaguaguugggacuacaggagcccaccaccaugccuggcuaauuuuuuuuggguuuuuaauagagauggaguuucaccguguuagccaggauggucucgaucuccugaccuugugaucugcccaccuuggccucccaaagugcugggauuacagucgugagccaccgugcccagccaagaaaaaauuuuugaggcaucaaaagcccauuuaaauuuggugccaggaacggagacaaucgugaaagcugcugauagccucacaaaucuuaagccagucacuuggguuaaaagcaucagaaguuucacuauuguaaauuucauauuaauccuuguaugccuguucugucuguuguuagucuacagguguauccagcagcuccaaagagacagcaaccagcaagaaugggccauagugacgauggugguuuugucaaaaagaaaagggggggauauguaaggaaaagagagaucagacuuucacugugucuauguagaaaaggaagacauaagaaacuccauuuugaucuguacuaagaaaaauuguuuugccuugagaugcuguuaaucuguaacuuuagccccaacccugugcucacggaaacaugugcuguaagguuuaagggaucuagggcugugcaggauguaccuuguuaacaauauguuugcaggcaguauguuugguaaaagucaucgccauucuccauucucgauuaaccaggggcucaaugcacuguggaaagccacaggaaccucugcccaagaaagccuggcuguugugggaagucagggaccccgaauggagggaccagcuggugcugcaucaggaaacauaaauugugaagauuucuuggacauuuaucaguuuccaaaauuaauacuuuuauaauuucuuacaccugucuuacuuuaaucucuuaauccuguuaucuuuguaagcugaggauauacgucaccucaggaccacuauuguacaaauugauuguaaaacauguucacauguguuugaacaauaugaaaucagugcaccuugaaaaugaacagaauaacagugauuuuagggaacaaaggaagacaaccauaaggucugacugccugaggggucgggcaaaaagccauauuuuucuucuugcagagagccuauaaauggacgugcaaguaggagagauauugcuaaauucuuuuccuagcaaggaauauaauacuaagacccuagggaaagaauugcauuccuggggggaggucuauaaacggccgcucugggagugucuguccuaugugguugagauaaggacugagauacgcccuggucuccugcaguacccucaggcuuacuaggauugggaaaccccaguccugguaaauuugaggucaggccgguucuuugcucugaacccuguuuucuguuaagauguuuaucaagacaauacaugcaccgcugaacauagacccuuaucaggaguuucugauuuugcucugguccuguuucuucagaagcaugucaucuuugcucugccuucugcccuuugaagcaugugaucuuugugaccuacucccuguucauacaccccuccccuuuuaaaaucccuaauaaaaacuugcugguuuuguggcucaggggggcaucauggaccuaccaauacgugaugucacccccgguggcccagcugu

SEQ ID NO: 1199 - RNA, 450 nt, HERV-K
uuauguauaugcacaucaaaagcacagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugugucucucaucc

SEQ ID NO: 1200 - RNA, 450 nt, HERV-K
uuauguauaugcacaucaaaagcacagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugugggugaaggua

SEQ ID NO: 1201 - RNA, 440 nt, HERV-K
uuauguauaugcacaucaaaagcacagcacuuuuuucuuuaccuuguuuaugaugcagagacauuuguucacauguuuuccugcuggcccucuccccacuauuacccuauuguccugccacaucccccucuccgagaugguagagauaaugaucaauaaauacugagggaacucagagaccggugcggcgcggguccuccauaugcugagcgccgguccccugggcccacuuuucuuucucuauacuuugucucuguugucuuucuuuucucaagucucucguuccaccugaggagaaaugcccacagcuguggaggcgcaggccacuccaucuggugcccaacguggaugcuuuucucuagggugaagggacucucgaguguggucauugaggacaagucaacgagagauucccgaguacgucuacagugagccuugug

SEQ ID NO: 1202 - DNA, 26 bp, Artificial Sequence
  RT-PCR oligonucleotide
agagaaaagcctccacgttgggcacc

SEQ ID NO: 1203 - DNA, 18 bp, Artificial Sequence
  RT-PCR oligonucleotide
gtaggggtgggttgcccc

SEQ ID NO: 1204 - DNA, 27 bp, Artificial Sequence
  RT-PCR oligonucleotide
aaaccgccttagggctggaggtgggac

SEQ ID NO: 1205 - DNA, 18 bp, Artificial Sequence
  RT-PCR oligonucleotide
tgcgggcagcaatactgc

SEQ ID NO: 1206 - DNA, 28 bp, Artificial Sequence
  RT-PCR oligonucleotide
taaagcactgagatgtttatgtgtatgc

SEQ ID NO: 1207 - DNA, 24 bp, Artificial Sequence
  RT-PCR oligonucleotide
gcacagcacttaatcctttacatt

SEQ ID NO: 1208 - DNA, 22 bp, Artificial Sequence
  RT-PCR oligonucleotide
gtttgtctgctgaccctctccc